(19) United States
(12) Patent Application Publication
(10) Pub. No.: US 2026/0183344 A1
(43) Pub. Date: Jul. 2, 2026

(54) IMPROVED SALMONELLA VECTORED THERAPIES FOR TREATMENT OF CANCER

(71) Applicant: UNIVERSITY OF FLORIDA RESEARCH FOUNDATION, INCORPORATED, Gainesville, FL (US)

(72) Inventors: Roy CURTISS, III, Gainesville, FL (US); Shifeng WANG, Gainesville, FL (US)

(21) Appl. No.: 18/870,052

(22) PCT Filed: Jun. 5, 2023
(86) PCT No.: PCT/US2023/024484

§ 371 Date: Nov. 27, 2024

Related U.S. Application Data
(60) Provisional application No. 63/348,492, filed on Jun. 3, 2022.

(51) Int. Cl. A61K 35/74; A61P 35/00; C07K 14/255; C07K 14/52; C07K 14/705; C12N 1/20; C12N 15/74; C12R 1/42

(52) U.S. Cl. CPC A61K 35/74; A61P 35/00; C07K 14/255; C07K 14/52; C07K 14/70521; C12N 1/20; C12N 15/74; C07K 2319/07; C12N 2800/10; C12R 2001/42

(57) ABSTRACT

A genetically modified Salmonella cell (GMSC) engineered to exhibit specific targeting to cells and regulated delayed lysis in vivo, the GMSC comprising a first heterologous nucleic acid that encodes a first gene product that causes the GMSC to be selectively localized to and/or internalized by a target cell in vivo and a second heterologous nucleic acid that encodes a second gene product that facilitates killing of the target cells following internalization.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 1, 2025, is named “10457-532US1.xml” and is 223,304 bytes in size. The sequence listing contained in this .XML file is part of the specification and is hereby incorporated by reference herein in its entirety.

### BACKGROUND

Cancer represents a diversity of disease states characterized by unregulated proliferation of cells that are either freely multiplying in blood and/or lymph or organized into tumor masses. After cardiovascular disease, cancer ranks as the second most common cause of death in the US (1).

Bacteria have been used to target cancers since Coley's observation over 100 years ago that tumors regressed in cancer patients infected with Streptococcus pyogenes (2, 3). Later, he used killed S. pyogenes, known as Coley's toxin, to treat cancer patients. Unfortunately, the trials of using bacteria as cancer therapy agents stopped for almost 70 years. After Malmgren demonstrated that Clostridium tetani could survive and replicate in necrotic tumors in 1955, studies using bacteria as cancer therapy recommenced and now are widespread in preclinical and clinical studies (4, 5). Many bacteria have been investigated for their anti-cancer ability, including Bifidobacterium infantis (6), Escherichia coli (7), C. tetani, Listeria monocytogenes (8) and Salmonella Typhimurium. While obligate anaerobes, such as Bifidobacterium and Clostridium, are highly effective at accumulating and replicating in necrotic tumors, they do not grow in viable tumor tissues, which limits their efficacy as anti-cancer agents. S. Typhimurium is a facultative anaerobe, which can survive and grow in anoxic regions as well as in viable oxygenic regions of tumors. Salmonella also has ability to identify and penetrate tumors by detecting small molecules such as serine and aspartate in tumors, and accumulates in tumors that contain free amino acids, purines and pyrimidines that facilitate Salmonella growth. As Salmonella are easily genetically manipulated, and attenuated Salmonella still retain their tumor-targeting and natural tumor-regressing capabilities, they became safe enough to evaluate in tumor-bearing mice and humans. Therefore, S. Typhimurium is widely investigated as an anti-cancer agent (see (5)). Currently, many researchers use S. Typhimurium VNP20009 or its derivatives as the anti-cancer agent or as a vector to investigate efficacy of anti-cancer activities (see (5, 9)). While VNP20009 carrying a purine auxotrophic mutation (purl) and lipid A mutation (msbB) and its derivatives demonstrated good anti-tumor efficacy in mice, anti-cancer efficacy in human trials was not achieved in phase I clinical trials in patients with metastatic melanoma and renal carcinoma (10, 11). In VNP20009-immunized dogs with a variety of malignant tumors, bacterial colonization of tumors was observed but only 4 of 35 dogs tested were completely cured (12). Even intratumoral injection in humans with cancer only led to colonization in 2 out of 3 patients (13). The reasons for failure in human clinical trials may be that the parent of VNP20009 is not highly virulent and its genetic construction is not precise. VNP20009 is derived from ATCC 14028, which does not show high virulence and invasiveness compared to other S. Typhimurium strains (14) and we demonstrated that an attenuated aroA mutant of 14028 was not as immunogenic and did not induce as high protective immune levels as did an isogenic derivative of the S. Typhimurium UK-1 strain (15) and furthermore was not as effective as a UK-1-derived strain in ablating colorectal tumors in mice (16). In addition, construction of VPN20009 is based on UV- and Tn10 transposon-induced mutations, which may result in other mutations and over-attenuation (17). It has been shown that the design method causes strain VPN20009 lost chemotactic ability (18). Also, the msbB mutation in VNP20009 is a bad choice because it leads to production of penta-acylated lipid A, which is a good pro-inflammatory stimulator in mice, but is an antagonist to inhibit stimulating human innate immunity (19-22). The second S. Typhimurium strain widely used for cancer therapy is A1-R, which is also derived from ATCC 14028 and is a leu-arg auxotroph (23, 24). Notable, the parent of A1-R (25), A1 is screened through nitrosoguanidine mutagenesis (24). A1-R exhibited good tumor-seeking features and has antitumor efficacy against major types of cancer in mice (24-26), but no clinical trials in humans or dogs have been performed. The 3^{rd} strain is VXM01, which is based on the S. Typhi strain Ty21a vaccine carrying an eukaryotic expression plasmid for VEGFR2, could induce anti-angiogenic activity when delivered by the oral route in pancreatic cancer. But only 1 of 13 patients showed an improved clinical outcome (27). The 4^{th} strain tested was χ4550 delivering IL-2 to induce responses in dogs and humans, respectively (28-30). All the these strains lack specific tumor targeting ability although VPN20009 and A1-R preferentially colonize tumors. Nevertheless, the results showed their targeting ability is not enough for high efficacy.

Salmonella has ability to regress tumors because of its natural toxicity and can also be used as vectors to deliver other anti-cancer molecules including cytotoxic agents such as Cytolysin A (ClyA), FAS ligand (FasL) and TNF-related apoptosis-inducing ligand (TRAIL), cytokines such as IL-2, and tumor antigens such as 3urviving and other factors such as tyrosinase which enhance its anti-cancer effectiveness (see (5, 9). FasL and TRAIL are belonging to the TNFα family. FasL specifically induces apoptosis in cells that possess the FAS receptor and TRAIL is cytotoxic to many cancer cells via death receptor pathways, which activate caspases 8 and 3 (31, 32). ClyA is a bacterial toxin inducing apoptosis and when delivered by S. Typhimurium reduced tumor growth in mice (33, 34). Cytokine IL-2 is widely investigated for its anti-cancer ability because IL-2 can activate the cytolytic function of NK cells and promotes lymphocyte proliferation (35-37). Cytotoxic agents and cytokines can induce apoptosis or stimulate immune cells to directly kill cancer cells, while tumor antigens such as 3urviving function to sensitize the immune system to fight against cancer cells. Survivin is a member of the inhibitor-of-apoptosis protein family involved in regulation of apoptosis and T-cell responses in anti-tumor immunity. It is over-expressed in many tumor cells. Blocking 3urviving function is thus a promising anti-tumor therapeutic method via induction of immune responses against (38-41).

In 1981, a patent application was filed on use of attenuated derivatives of pathogenic bacteria to deliver recombinant protective antigens from heterologous pathogens to induce protective immunity to the pathogens whose antigens were delivered by the vaccine construct. Salmonella was the chosen pathogen and it has been continuously improved and perfected as a means for using Salmonella as an antigen and DNA vaccine delivery vector (42, 43). Traditionally, rendering live vaccines safe to be unable to cause adverse effects or disease symptoms has been accompanied with decreased immunogenicity because of the lessened abilities of the attenuated live vaccine to be invasive to colonize lymphoid tissues and/or with reduced abilities to multiply and/or persist to induce an adequate immune response unless administered in multiple doses (44). We have recently invented multiple means to eliminate all these problems that limit live vaccine efficacy by genetically programing the recombinant attenuated Salmonella vaccines (RASVs) to display the same as or even better infection proficiencies than wild-type Salmonella at the time of RASV administration. We thus invented means to increase invasiveness of RASVs (45) and enhance their ability to better survive against host-defense barriers encountered during mucosal delivery (46-49). These modifications coupled with engineering strains with regulated delayed attenuation (50, 51) and regulated delayed antigen synthesis (52-55) enable the vaccine constructed strains to colonize internal tissues almost to the same extent as wild-type virulent Salmonella but without causing any disease symptoms (50, 51, 53, 56, 57). The RASVs are also designed to persist in these effector lymphoid tissues to serve as factories for the continuous synthesis and delivery of recombinant protective protein antigens (53). These protein antigens are encoded by pathogen genes to induce protective immunity against the pathogen. Alternatively, the recombinant protein might exert a physiological activity altering a host physiological or immunological activity. In either case, the protein is encoded by codon-optimized sequences to enhance mRNA stability and efficiency of transcription and translation in Salmonella (58-60). Since immune responses against recombinant proteins are improved by secretion of antigens rather than their retention in the RASV cytosol (61, 62), we perfected use of type 3 and type 2 secretion systems (T3SS & T2SS) (52, 55, 63) to export proteins out of the RASV or into the periplasmic space to enhance production of outer membrane vesicles that are highly immunogenic (49, 64). In addition, we developed vaccine constructs with regulated delayed lysis in vivo to release in specified cell compartments a bolus of recombinant proteins (45, 52, 65) or a DNA vaccine designed for maximal import to the nucleus for efficient high-level transcription and translation of encoded sequences (45). We observed in multiple recent studies that higher levels of induced protective immunity can be induced by vaccine strains displaying the regulated delayed lysis phenotype than by strains not undergoing lysis (45, 52, 65-68). We have engineered strains to eliminate or decrease synthesis of serotype-specific LPS O-antigen (69, 70) and other immune-dominant surface antigens to reduce inducing immune responses to Salmonella. Nevertheless, prior immunity including maternal immunity (56, 57) enhances success in immunizing neonates and individuals previously immunized with a different strain. We now term these much-improved vaccine vector strains as Protective Immunity Enhanced Salmonella Vaccine (PIESV) vector strains. Based on accumulated results demonstrating complete biological containment and safety of our self-destructing PIESV vectors encoding for delivery of protective antigens from various bacterial, viral and parasite pathogens in newborn, pregnant, malnourished and immune deficient SCID mice, in multiple studies with mice, chickens, pigs and in a human phase 1 trial with no adverse events, bacteremias or shedding in vaccinated human volunteers of viable recombinant vaccine cells in stools over a 12-day period at oral doses of 10¹⁰ CFU (56, 57, 71-73), the NIH Office of Science Policy and Recombinant Advisor Committee granted permission to us to evaluate our genetically modified vaccines at Biosafety level 1 containment and under settings simulating commercial rearing for farm animals and in out-patients for human trials. This reclassification was also approved by the University Florida Institutional Biosafety Committee.

We also discovered that these PIESV strains are superior adjuvants in recruiting innate immunity. We subsequently have been designing these adjuvant S. Typhimurium UK-1 derived strains as Self-Destructing Attenuated Adjuvant Salmonella (SDAAS) strains to serve as adjuvants to recruit innate immune responses and enhance induction of immunity induced by subunit, killed, live attenuated and live vectored vaccines.

### BRIEF DESCRIPTION OF DRAWINGS

FIG. 1. Plasmid maps of the cloning vector pYA3342, the suicide vector pRE112 and the regulated delayed lysis DNA vaccine vector pYA4545.

FIG. 2. Plasmid maps of the regulated delayed lysis cloning vectors pG8R110 (with T3SS) and pG8R114 (with T2SS) and the plasmid vectors pYA4090 encoding synthesis of GFP and pYA4685 encoding synthesis of EGFP.

FIG. 3. Plasmid map of pG8R314 encoding OmpA with PLZ4 insert (ompAΩplz4).

FIG. 4. Plasmid map of pG8R315 as suicide vector for insertion of sequence encoding ompA with PLZ4 insertion (ompAΩplz4) into the S. Typhimurium chromosome.

FIG. 5. Plasmid maps of pG8R319 derived from pG8R314 by insertion of eukaryotic expression cassette and pG8R320 derived from pYA4545 by insertions of a prokaryotic expression cassette to express sequence encoding ompA with PLZ4.

FIG. 6. Salmonella with PLZ4 peptide exposed on surface is attracted to and invades into bladder cancer cells.

FIG. 7. Plasmid maps of pG8R321, pG8R322, pG8R323 and pG8R324 all derived from pG8R319 by insertion of the nucleotide sequences encoding human CXCL11, Mouse CXCL11, KillerRed fused to the neuromodulin N-terminal sequence and KillerRed fused to mitochondrial targeting signals, respectively.

FIG. 8. Plasmid maps of pG8R325, pG8R326, pG8R327 and pG8R328 all derived from pG8R320 by insertion of the nucleotide sequences encoding human CXCL11, Mouse CXCL11, Killer Red fused to the neuromodulin N-terminal sequence and Killer Red fused to mitochondrial targeting signals, respectively.

FIG. 9. Plasmid maps of pG8R341 derived from pG8R314 by insertion of sequence encoding GFP3 as an operon fusion and pG8R342 derived from pG8R320 by insertion of sequence encoding EGFP.

FIG. 10. Plasmid maps of multicistronic pG8R343 and pG8R344 derived from pG8R320 by insertion of sequences encoding KillerRed fused to the neuromodulin N-terminal sequence and human CXCL11 or mouse CXCL11. P2A peptide is used to separate KillerRed and CXCL11 FIG. 11. Plasmid maps of pG8R345 derived from pG8R320 by insertion of sequences encoding HLAB leading and tail peptides and pG8R346 derived from pG8R345 by insertion of sequence encoding EGFP.

FIG. 12. Plasmid maps of pG8R347, pG8R348, pG8R349 and pG8R350. pG8R347 is derived from pG8R320 by insertion of sequences for 5′ HLA, HLA leading and Tail peptides and 3′ HLA. pG8R348, pG8R349 and pG8R350 are derived from pG8R347 by insertion of sequence encoding EGFP, neo-antigen BBM963 and MB49, respectively.

FIG. 13. Plasmid pG8R361 derived from pG8R320 by insertion of sequence from P_{EF1α} promoter.

FIG. 14. Plasmid maps of pG8R362, pG8R363, pG8R364 and pG8R365. pG8R362 and pG8R363 are derived from pG8R320 by insertion of sequence encoding HAC-PD1 fused with human CXCL11 and HAC-PD1 fused with mouse CXCL11, respectively. pG8R364 and pG8R365 are derived from pG8R361 by insertion of sequence encoding HAC-PD1 fused with human CXCL11, HAC-PD1 fused with mouse CXCL11, respectively.

FIG. 15. Plasmid maps of pG8R366 derived from pG8R320 by insertion of sequence encoding IL2 secretion signal (IL2 SS) and pG8R367 and pG8R368 derived from pG8R366 by insertion of sequence encoding HAC-PD1 and human CXCL11 and HAC-PD1 and mouse CXCL11, respectively.

FIG. 16. Plasmid maps of pG8R372, pG8R373, pG8R374 and pG8R375 derived from pG8R320 by insertion of sequence encoding IL2 SS fused with HAC-PD1. IL2 SS fused with HAC-PD1 and EGFP, human CXCL11 fused with EGFP, mouse CXCL11 fused with EGFP, respectively.

FIG. 17. Plasmid maps of pG8R380 derived from pG8R320 by insertion of sequence encoding operon fusion of OmpA with LHRH insertion and GFP and pG8R381 derived from pG8R380 by insertion of OmpA with LHRH insertion, respectively.

FIG. 18. Plasmid maps of pG8R382, pG8R383 and pG8R384 derived from pG8R320 by insertion of sequence encoding haPD1-IgG, haPD1-IgG and KillerRed fused with neuromodulin N terminal sequence, and KillerRed fused with neuromodulin N terminal sequence and haPD1-IgG, respectively.

FIG. 19. Plasmid maps of pG8R385 derived from pG8R320 by insertion of sequence encoding operon fusion of OmpA with Her2 scFv insertion and GFP and pG8R386 derived from pG8R385 by insertion of OmpA with Her2 scFv insertion, respectively.

FIG. 20. Plasmid maps of pG8R388 and pG8R389 derived from pG8R320 by insertion of sequence for P_{trc} promoter and P_{trc} promoter and optimized Bla secretion signal.

FIG. 21. Plasmid maps of pG8R390, pG8R391, and pG8R418. pG8R390 is derived from pG8R381 by insertion of sequence encoding KillerRed fused with neuromodulin N terminal sequence and pG8R391 is derived from pG8R386 by insertion of sequence encoding KillerRed fused with neuromodulin N terminal sequence. pG8R418 is derived from pG8R385 by insertion of sequence encoding KillerRed fused with neuromodulin N terminal sequence.

FIG. 22. The attachment and invasion of bladder cancer cells with strains derived from χ12614 with O-antigen mutations. (A) Genotypic characterization of strains using primers specific for waaL, waaG, waaC, ompA and ompAΩplz4. Lane 1, χ3761; lane 2, χ12614; lane 3, χ12812; lane 4, χ12813; lane 5, χ12814. (B) LPS gel profile of strains. All strains are grown in LB media. (C) The percentage of attachment and invasion of strains in human bladder cancer cell line 5637 and mouse bladder cancer cell line MB49. All strains carry plasmid pG8R314 with multiple copies of ompAΩplz4. ****, P<0.0001, compared with other strains.

FIG. 23. The attachment and invasion of bladder cancer cells with strains derived from χ12619 with O-antigen mutations. All strains have only one copy of ompAΩplz4 in their chromosome. (A) Genotypic characterization of strains using primers specific for asdA, pagP, pagL, lpxR, waaL, waaG, waaC, ompA and ompAΩplz4. Lane 1, χ3761; lane 2, χ12518; lane 3, χ12619; lane 4, χ12808; lane 5, χ12809; lane 4, χ12810; lane 5, χ12811. (B) The growth of strains on LB, LB+ arabinose (0.1%), LB+DAP (50 ug/ml), LB+D-alanine (50 ug/ml) plates. (C) LPS gel profile of the strains. All strains are grown in LB media with 0.1% arabinose. (D) The percentage of attachment and invasion of strains in human bladder cancer cell line 5637 and mouse bladder cancer cell line MB49. *, P<0.05, **, P<0.01, ***P<0.001, ****, P<0.0001, compared with other LPS mutation strains FIG. 24. Display of OmpAΩHer2 ScFV on the bacterial surface. Salmonella outer membrane proteins (OMPs) were isolated and subjected to SDS-PAGE gel and western blot with anti-His antibody. 1, χ12417; 2, χ12417(pG8R385); 3, χ12417(pG8R418); 4, χ12417(pG8R391).

FIG. 25. Strains carrying ompAΩher2 ScFV display higher attachment and invasion to Her2 over expression cell SKRB-3. ****, P<0.0001.

FIG. 26. Production of KillerRed in HEK293T cells transfected with plasmid pG8R327. The frames show the KillerRed red fluorescent signals from four different samples (EVOS FL, RFP channel)

FIG. 27. KillerRed kills HEK293T cell. Time zero is set immediately after irradiation with green light. Time 10 is set immediately after excitation for 10 min (EVOS FL, RFP channel)

FIG. 28. KillerRed kills HEK293T cell excitation for 10 and 20 minutes. Time zero is set immediately after irradiation with green light. Time 10 and 20 are set immediately after excitation for 10 min or 20 mins (EVOS FL, RFP channel)

### DEFINITIONS

As used herein the specification, “a” or “an” may mean one or more, unless clearly indicated otherwise. As used herein in the claims, when used in conjunction with the word “comprising,” the words “a” or “an” may mean one or more than one.

The term “administering” or “administration” of an agent as used herein means providing the agent to a subject using any of the various methods or delivery systems for administering agents or pharmaceutical compositions known to those skilled in the art. Agents described herein may be administered by oral, intradermal, intravenous, intramuscular, intraocular, intranasal, intrapulmonary, epidermal, subcutaneous, mucosal, or transcutaneous administration.

The terms “animal host” or “subject” as used interchangeably hereinto refer to a human or nonhuman mammal or a vertebrate animal into which a genetically modified Salmonella cell has been administered. In a specific embodiment, the subject is a human.

The terms “attenuated” or “attenuation” as used herein refer to the process of rendering certain pathogen virulence attributes needed to cause diseases less able to cause such disease symptoms. In one example, attenuation involves imparting an attenuation mutation in the pathogen.

The term “attenuating mutation” refers to a mutation imparted into a pathogen that reduces infectivity, virulence, toxicity, induction of disease symptoms, and/or impairment of a subject upon administration of the pathogen (e.g. PIESV strain). Examples of attenuating mutations include those mutations that facilitate lysis in vivo (e.g. impairing synthesis of essential constituents of peptidoglycan layer), reduce or impair synthesis of LPS or other cell-surface components, and one or more mutations that provide auxotrophy (e.g. dependence on an amino acid, purine, pyrimidine, or vitamin for growth).

The term “balanced-lethal vector-host” refers to a host Salmonella cell into which a plasmid vector has been introduced such that survival of the host cell is dependent on the maintenance of the plasmid vector and loss of the plasmid vector results in death of the host Salmonella cell. (See Nakayama, K., Kelly, S. & Curtiss, R. Construction of an ASD⁺ Expression-Cloning Vector: Stable Maintenance and High Level Expression of Cloned Genes in a Salmonella Vaccine Strain. Nat Biotechnol 6, 693-697 (1988) or Galán J E, Nakayama K, Curtiss R 3^{rd}. Cloning and characterization of the asd gene of Salmonella typhimurium: use in stable maintenance of recombinant plasmids in Salmonella vaccine strains. Gene. 1990 Sep. 28; 94(1):29-35, whose teachings are incorporated by reference).

The term “biologically active fragment” or “biologically active variant” refers to a fragment or variant of a sequence that maintains its biological activity. In the context of H. pylori antigen sequences, a biologically active fragment or biologically active variant is a fragment or variant of an antigen amino acid sequence that elicits an immune response in a host.

The term “Cancer Cell Targeting Salmonella strain” or “CCTS strain” refers to a strain of Salmonella that has one or more attenuating mutations and expresses a gene product that causes selective localization and/or internalization of cells of the CCTS strain by a cancer cell.

As used herein, “codon” means, interchangeably, (i) a triplet of ribonucleotides in an mRNA which is translated into an amino acid in a polypeptide or a code for initiation or termination of translation, or (ii) a triplet of deoxyribonucleotides in a gene whose complementary triplet is transcribed into a triplet of ribonucleotides in an mRNA which, in turn, is translated into an amino acid in a polypeptide or a code for initiation or termination of translation. Thus, for example, 5′-TCC-3′ and 5′-UCC-3′ are both “codons” for serine, as the term “codon” is used herein.

The term “codon optimized” or “codon optimization” as used herein refers to enhancing the ability of the antigen encoding sequence to be expressed in the Salmonella strain by selecting codons that are used for highly expressed genes in Salmonella. Such codon optimization also includes changing the GC content of the antigen encoding sequence to be similar to that used for Salmonella (i.e., ˜52% GC). In addition, the codon optimization can also be used to enhance the stability of the mRNA encoded by the antigen encoding sequence so as to be less likely to be degraded by Rnases.

The term “delayed attenuation” as used herein refers to a means of gene regulation such that the attenuation attribute is not expressed during growth of the vaccine strain or during its administration to an animal host but is not expressed after the CCTS strain enters the animal host and is manifest as a consequence of vaccine cell division in vivo with gradual dilution of the virulence gene product by at least half at each cell division in vivo.

The term “gene product” refers to a transcript (RNA) or expressed polypeptide encoded by a heterologous gene or nucleic acid that has been introduced into a genetically modified Salmonella cell. In typical embodiments, the gene product causes selected localization to a target cell. The gene product may also cause cytotoxicity to the target cell upon internalization of the genetically modified Salmonella cell and/or cause a targeted immune response to target cells.

A “genetically modified Salmonella cell” or “GMSC” refers to a Salmonella cell that comprises an attenuating mutation and/or into which a heterologous gene or nucleic acid, e.g., an exogenous nucleic acid that is foreign to the Salmonella cell, has been introduced.

The term “operably linked” as used herein means that one nucleic acid sequence is linked to another nucleic acid sequence, and therefore the function or expression thereof is influenced by the linked nucleic acid sequence.

As used herein, the term “percentage of sequence identity” or “percent sequence identity” may refer to the value determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa. The term “about” with respect to a numerical value of a sequence length means the stated value with a +/−variance of up to 1-5 percent. For example, about 30 contiguous nucleotides means a range of 27-33 contiguous nucleotides, or any range in between. The term “about” with respect to a numerical value of percentage of sequence identity means the stated percentage value with a +/−variance of up to 1-3 percent rounded to the nearest integer. For example, about 90% sequence identity means a range of 87-93%. However, the percentage of sequence identity cannot exceed 100 percent. Thus, about 98% sequence identity means a range of 95-100%.

The term “regulated delayed lysis” refers to a construction in which the expression of one or more genes specifying synthesis of peptidoglycan precursors such as but not limited to diaminopimelic acid and muramic acid are regulated by a sugar-dependent process such that the genes are expressed in the presence of a sugar such as but not limited to arabinose or rhamnose supplied during cultivation of the strain and cease to be expressed in vivo since the sugar is absent to result in lysis as a consequence of cell division of the CCTS strain in vivo. The genes conferring the regulated delayed lysis phenotype may be either chromosomal and/or plasmid encoded.

The term “regulated delayed lysis plasmid” refers to a construction in which the expression of one or more genes specifying synthesis of peptidoglycan precursors such as but not limited to diaminopimelic acid and muramic acid that are regulated by a sugar-dependent process are located on a plasmid vector encoding synthesis of one or more foreign antigens or gene products.

The term “Salmonella cell” refers to a cell of a Salmonella species or serotype. Examples of a Salmonella serotype include Salmonella Typhimurium and Salmonella Enteritidis. In a more specific embodiment, the Salmonella serotype is S. Typhimurium UK-1.

The term “sequence identity” or “identity,” as used herein in the context of two polynucleotides or polypeptides, refers to the residues in the sequences of the two molecules that are the same when aligned for maximum correspondence over a specified comparison window.

As used herein, the term “targeted immune response” refers to a response by a subject's immune system against target cells. Immune responses include both cell-mediated immune responses (responses mediated by antigen-specific T cells and non-specific cells of the immune system) and humoral immune responses (responses mediated by antibodies present in the plasma lymph, and tissue fluids and secreted onto mucosal surfaces).

The term “target cell” refers to a cell of a subject that is of a type to which a genetically modified Salmonella cell is designed for selective localization and/or internalization. Selective localization refers to increased migration of the genetically modified Salmonella cell to a target cell over other cells in a subject. Selective internalization refers to increased internalization of the genetically modified Salmonella cell in the target cell over other cells in the subject. Increased localization to and/or increased internalization means an increase of at least 5%, 10%, 20%, 30%, 40%, 50%, 60%, 70% or more respective to target cells as opposed to other cells in a subject. Typically, a target cell internalizes the genetically modified Salmonella cell by active invasion or endocytosis or phagocytosis. In exemplified embodiments, the target cell is a cancer cell and the genetically modified Salmonella cell is of a CCTS strain that is selectively internalized by the cancer cell over other cells in the subject. In alternative embodiments, the genetically modified Salmonella cell is engineered to localize at a tumor microenvironment where cancer cells are present without necessarily being internalized into a cancer cell.

The term “variant” as used herein refers to a nucleic acid sequence or amino acid sequence that possesses at least about 85, 90, 95, 96, 97, 98 or 99 percent sequence identity to another nucleic acid sequence or amino acid sequence, respectively.

Other relevant definitions are provided infra.

### DESCRIPTION

Disclosed herein are embodiments directed to designing, constructing and evaluating Cancer Cell Targeting Salmonella (CCTS) strains. CCTS strain embodiments have (i) ability to directly destroy tumor cells, (ii) deliver cargoes that cause tumor cells to self-destruct, (iii) deliver cargoes that enhance abilities to treat tumor cells, and/or (iv) directly and/or indirectly stimulate host immune responses to repress tumor cell growth, metastases and cell death. A potentially desirable feature involves rapid self-destruction of CCTS cells that enables their use for repeat treatments of subjects. A unique attribute of these newly designed and constructed CCTS strains is their ability to simultaneously synthesize and deliver protein cargoes to cancer cells but to also deliver DNA vaccines encoding other effective proteins to be synthesized by the tumor cells to their detriment.

The foregoing attributes are achieved by introducing numerous deletion and deletion-insertion mutations to enable and endow the desired phenotypic properties to the strains constructed. These mutations and their associated phenotypes are listed in Table 1 and the suicide vectors needed for their insertion into plasmids and the S. Typhimurium chromosome are listed in Table 2. The distribution of genetic deletion and deletion-insertion mutations and the redundancy in critical modifications ensure both stability and safety of these CCTS strains.

Examples of genotypes of CCTS strains are listed in Table 3.

TABLE 1
Mutations and associated phenotypes in S. Typhimurium CCTS strains^{a} It is noted that the
genes can be inactivated or deleted in multiple ways to confer the same phenotypic traits.
Also, though certain allele numbers are indicated elsewhere herein for certain mutations, reference
to a certain allele is not limiting and the mutations can be executed in other alleles.

| Genotype | Phenotype |
|---|---|
| ΔaroA | encodes the first enzyme in the pathway to synthesize aromatic |
| | amino acids and derived vitamins (74) |
| ΔasdA | deletes gene for aspartate semialdehyde dehydrogenase |
| | essential for synthesis of diaminopimelic acid (DAP) necessary |
| | for peptidoglycan synthesis (75) |
| ΔP_{asdA}::TT araC P_{araBAD} asdA | makes synthesis of AsdA dependent on presence of |
| | arabinose |
| ΔP_{asdA}::TT rhaRS P_{rhaBAD} asdA | makes synthesis of AsdA dependent on presence of |
| | rhamnose |
| ΔasdA::TT araC P_{araBAD} c2 | inactivates asdA and makes synthesis of C2 repressor |
| | dependent on arabinose (76, 77) |
| Δalr and ΔdadB | deletes the genes for two alanine racemases essential for |
| | synthesis of D-alanine necessary for peptidoglycan synthesis |
| | (78) |
| ΔP_{dadB}::TT araC P_{araBAD} dadB | makes synthesis of DadB dependent on presence of |
| | arabinose |
| ΔP_{dadB}::TT rhaRS P_{rhaBAD} dadB | makes synthesis of DadB dependent on presence of |
| | rhamnose |
| ΔP_{murA}::TT araC P_{araBAD} murA | makes synthesis of MurA, the first enzyme in the |
| | synthesis of muramic acid, dependent on arabinose in growth |
| | medium and ceases synthesis in vivo due to absence of |
| | arabinose (50, 65) |
| ΔP_{fur}::TT araC P_{araBAD} fur | makes synthesis of the Fur repressor protein |
| | dependent on arabinose in growth medium that ceases in vivo to |
| | result in high-level synthesis of all iron regulated proteins to |
| | result in attenuation (50, 79) |
| ΔmntR | eliminates gene for repressor MntR that regulates MntR- and |
| | some Fur-regulated genes for manganese and iron acquisition, |
| | respectively |
| ΔP_{mntR}::TT araC P_{araBAD} mntR | makes synthesis of the MntR repressor protein |
| | dependent on arabinose in growth medium that ceases in vivo to |
| | result in high-level synthesis of all manganese regulated proteins |
| | to contribute to attenuation |
| Δcya | encodes enzyme for adenylate cyclase |
| Δcrp | encodes adenylate cyclase catabolite represor protein |
| ΔaraBAD::TT | deletion of genes to eliminate arabinose catabolismwith TT |
| | inserted to prevent transcription of downstream genes (80-84) |
| ΔaraCBAD100::TT | Deletion of all genes in the ara operon |
| ΔrhaBADSR | deletion of genes to eliminate rhamnose catabolismo (85, 86) |
| ΔpagP::Pₗₚₚ lpxE | mutation causes regulated delayed in vivo synthesis of the codon- |
| | optimized lpxE gene from Francisella tularensis to cause |
| | synthesis of the non-toxic adjuvant form of LPS lipid A lipid A |
| | (MPLA) (66) |
| ΔpxR::Pₗₚₚ lpxF mutation | causes regulated delayed in vivo synthesis of the codon- |
| | optimized lpxF gene from Francisella tularensis to cause |
| | synthesis of LPS with only the 1′-phosphoryl group (21) |
| ΔpagL and ΔlpxR | eliminates two means by which Salmonella alters LPS |
| | components in vivo to decrease recruitment of innate immunity |
| | by interaction with TLR4 (20) |
| ΔeptA | prevents addition of ethanolamine to lipid A (87, 88) |
| ΔarnT | prevents addition of 4-amino-4-deoxy-L-arabinose (L-Ara4N) |
| | groups to lipid A (89) |
| ΔfliC | deletes gene specifying synthesis of the phase I flagellin FliC |
| | (79, 90-92) |
| ΔfljB | deletes gene specifying synthesis of the phase II flagellin FljB |
| | (79, 90-92) |
| ΔfliC180 | specifies a truncated FliC protein containing TLR5 recognition |
| | domain and CD4 epitope (93) |
| Δ(hin-fljBA) | locks in expression of gene for phase I FliC flagellin and |
| | precludes synthesis of phase II FljB flagellin (94-100) |
| Δ(agfG-agfC) | deletes two operons specifying thin aggregative fimbriae (curli) |
| | and an activator for synthesis and export of cellulose and other |
| | exopolysaccharides (101) |
| ΔP_{saf5}::P_{murA} safA | causes constitutive synthesis of Saf fimbriae that facilitate spleen |
| | colonization (102) |
| ΔP_{stc}::P_{murA} stcA | causes constitutive synthesis of Stc fimbriae that facilitate spleen |
| | colonization (102) |
| ΔfimH | encodes the adhesin tip on Type 1 fimbriae (103, 104) |
| ΔompA | specifies synthesis of a very prevalent outer membrane protein |
| | (105) |
| ΔsopB | a protein secreted by the Salmonella SPI-I that can cause |
| | intestinal inflammation (106-108) |
| ΔpabA & ΔpabB | Encode two enzyme subunits of the enzyme synthesizing p- |
| | amino benzoic acid (109, 110) |
| Δpmi | eliminates phosphomannose isomerase that precludes synthesis |
| | of GDP-mannose that is needed for LPS O-antigen synthesis |
| | (70, 79) |
| ΔwaaL & ΔpagL::TT araC P_{araBAD} waaL | make synthesis of the WaaL enzyme that couples O-antigen |
| (or ΔpagL::TT rhaRS P_{rhaBAD} waaL) | to the LPS core synthesis (22) dependent on presence of arabinose |
| | (or rhamnose) |
| ΔwbaP | encodes enzyme that couples LPS core to LPS O-antigen (22, |
| | 111-114) |
| ΔwaaC | encodes enzyme necessary for assembly of the LPS inner core |
| | (111-114) |
| ΔwaaG | encodes enzymes essential fir assembly of the outer LPS core |
| | (22, 111-114) |
| Δ(wza-wcaM) | eliminates 20 genes encoding enzymes needed for synthesis of |
| | colanic acid, LPS capsular antigen and other polysaccharides to |
| | facilitate lysis, enhance immunogenicity and inhibit biofilm |
| | formation (115, 116) |
| ΔrelA | uncouples growth regulation from a dependence on protein |
| | synthesis (117, 118) |
| ΔrelA::araC P_{BAD} lacI TT and | makes synthesis of lacI that represses gene expression |
| Δ(traM-traX)::araC P_{araBAD} lacI | controlled by Pₜᵣₑ dependent on presence of arabinose (53, 119) |
| | with either inactivation of relA gene (75, 76) or deletion |
| | of genes encoding conjugational plasmid transfer in Salmonella |
| | virulence plasmid (120) |
| ΔspoT | eliminates gene for synthesis of ppGpp (117, 121, 122) |
| ΔspvRABCD | deletes Salmonella plasmid virulence genes encoding regulatory |
| | activator-repressor (R) and four genes conferring invasiveness |
| | and virulence; when the spvABCD genes are over expressed |
| | increase invasiveness and virulence |
| ΔcysG175::Pₛₚᵥ spvABCD | inserts spv operon without the R gene specifying the |
| | repressor-activator into a deletion of the cysG gene under control |
| | of a promoter not regulated by SpvR |
| ΔP_{hilA}::P_{traΔlacO} hilA | constitutive P_{trc} regulated synthesis of HilA that increases |
| | expression of SPI-1 genes for invasion of epithelial cells (45) |
| ΔrecF | reduces inter- and intra-plasmidic recombination (78, 123-125) |
| ΔendA | deletes gene encoding endonuclease I to prevent degradation of |
| | released DNA vaccine (82) |
| ΔsifA | enables Salmonella to escape from the SCV to enter the cytosol |
| | (126, 127) |
| ΔsseL | eliminates a gene that enables Salmonella to induce pyroptosis |
| | (82) |
| ΔtlpA | eliminates a gene that enables Salmonella to induce pyroptosis |
| | (82) |

^{a}Δ = deletion; TT = transcription terminator; P = promoter

TABLE 2
Suicide vectors for constructing the mutations in Table 1

| | | |
|---|---|---|
| Genotype | Suicide Vector | Marker |

A. Deletion and deletion-insertion mutations
to facilitate regulated delayed lysis in vivo

| | | |
|---|---|---|
| ΔP_{murA25}::TT araC P_{araBAD} murA | pYA4686 | Cm |
| ΔasdA33 | pYA3736 | Cm |
| ΔP_{asdA55}::TT araC P_{araBAD} asdA | pG8R71 | Cm |
| ΔP_{asdA88}::TT rhaRS P_{rhaBAD1} asdA | pG8R354 | Cm |
| Δalr-3 | pYA3667 | Cm |
| ΔdadB4 | pYA3668 | Cm |
| ΔP_{dadB66}::TT araC P_{araBAD} dadB | pG8R73 | Cm |
| ΔP_{dadB22}::TT rhaRS P_{rhaBAD1} dadB | pG8R352 | Cm |
| Δ(wza-wcaM)-8 | pYA4368 | Cm |
| ΔrelA1123 | pYA3679 | Cm |

B. Mutations enabling regulation of genes that might
be present on plasmid vectorsin conjunction with
strains undergoing regulated delayed lysis in vivo

| | | |
|---|---|---|
| ΔrelA197::araC P_{araBAD} lacI TT | pYA4064 | Cm |
| ΔasdA27::TT araC P_{araBAD} c2 | pYA4138 | Cm |
| Δ(traM-traX)-36::araC P_{araBAD} lacI TT | pG8R329 | Cm |
| Δ(traM-traX)-41::araC P_{araBAD} lacI TT | pG8R397 | Cm |

C. Mutations conferring attenuation of virulence

| | | |
|---|---|---|
| ΔaroA21419 | pYA3600 | Cm |
| Δcya-27 | pMEG080 | Te |
| Δcrp-27 | pMEG084 | Tet |
| ΔpabA1516 | pMEG147 | Tet |
| ΔpabB232 | pYA3438 | Cm |

D. Mutations conferring regulated delayed attenuation
and over production of iron and manganese-regulated
proteins to confer cross-protective immunity

| | | |
|---|---|---|
| ΔP_{fur33}::TT araC P_{araBAD} fur | pYA3722 | Cm |
| ΔP_{mntR44}::TT araC P_{araBAD} mntR | pG8R227 | Cm |

E. Mutations altering synthesis of LPS components

| | | |
|---|---|---|
| Δpmi-2426 | pYA3546 | Tet |
| ΔpagP8 | pYA4288 | Cm |
| ΔpagP81::Pₗₚₚ lpxE | pYA4295 | Cm |
| ΔpagL7 | pYA4284 | Cm |
| ΔlpxR9 | pYA4287 | Cm |
| ΔlpxR93::Pₗₚₚ lpxF | pYA4289 | Cm |
| ΔarnT6 | pYA4286 | Cm |
| ΔeptA4 | pYA4283 | Cm |
| ΔwaaC41 | pYA5473 | Cm |
| ΔwaaG42 | pYA4896 | Cm |
| ΔwaaL46 | pYA4900 | Cm |
| ΔwbaP45 | pYA4899 | Cm |
| ΔpagL19::TT araC P_{araBAD1} waaL | pYA5468 | Cm |
| ΔpagL64::TT rhaRS P_{rhaBAD1} waaL1 | pYA5377 | Cm |
| ΔpagL38::TT rhaRS P_{rhaBAD1} waaL2 | pG8R296 | Cm |
| ΔpagL18::TT araC P_{araBAD1} waaC | pYA5458 | Cm |
| ΔpagL21::TT araC P_{araBAD1} waaG | pYA5462 | Cm |

F. Mutations blocking catabolismof sugars

| | | |
|---|---|---|
| ΔaraBAD65::TT | pYA4811 | Cm |
| ΔrhaBADSR515 | pG8R272 | Cm |
| ΔaraCBAD100::TT | pG8R392 | Cm |

G. Mutations altering synthesis of flagellar components

| | | |
|---|---|---|
| ΔfliC180 | pYA3729 | Cm |
| ΔfliC2426 | pYA3702 | Cm |
| ΔfljB217 | pYA3548 | Tet |
| Δ(hin-fljBA)-209 | pG8R306 | Cm |

H. Mutations altering synthesis of fimbrial components

| | | |
|---|---|---|
| Δ(agfG-agfC)-999 | pYA4941 | Cm |
| ΔP_{stc53}::P_{murA} StcA53 | pYA5053 | Cm |
| ΔstcABCD | pYA5007 | Tet |
| ΔP_{saf55}::P_{murA} safA55 | pYA5055 | Cm |
| ΔsafABCD | pYA4586 | Tet |
| ΔfimH1019 | pYA3545 | Tet |

I. Mutations eliminating or altering outer membrane proteins

| | | |
|---|---|---|
| ΔompA11 | pYA4757 | Tet |
| ompA3Ωplz4 | pG8R315 | Cm |

J. Mutations decreasing inflammation and enhancing mucosal immunity

| | | |
|---|---|---|
| ΔsopB1925 | pYA3733 | Cm |

K. Mutations eliminating or diminishing effective immunogenicity

| | | |
|---|---|---|
| ΔsifA26 | pYA3716 | Cm |

L. Mutations decreasing/delaying onset of pyroptosis

| | | |
|---|---|---|
| ΔsseL116 | pYA4621 | Cm |
| ΔtlpA181 | pYA4620 | Cm |

M. Mutations leading to degradation of DNA within Salmonella cells

| | | |
|---|---|---|
| ΔrecA62 | pYA4680 | Cm |
| ΔrecF126 | pYA3886 | Cm |
| ΔendA2311 | pYA3652 | Cm |

N. Mutations altering invasion

| | | |
|---|---|---|
| ΔP_{hilA}::P_{traΔlacO} hilA | pYA4681 | Cm |

^{a}Δ = deletion; TT = transcription terminator; P = promoter

TABLE 3
Genotypes of CCTS strains generated that have been
used in past research on anti-cancer therapies.

| Strain | genotype | Refs |
|---|---|---|
| χ4550 | Δcya-1 Δcrp-1 ΔasdA1 (Δzhf-4::Tn10) (from χ4064 STm SR-11) | (30, 35, |
| | | 128-130) |
| χ8133 | Δcya-27 Δcrp-27 ΔasdA16 | (63) |
| χ11091 | ΔpabA1516 ΔpabB232 ΔasdA16 ΔmsbB48 ΔpagL7 ΔpagP81::Pₗₚₚ | (20) |
| | lpxE ΔlpxR93::Pₗₚₚ lpxF | |
| χ12342 | ΔwaaG42 ΔpagL21::TT araC P_{araBAD} waaG ΔlpxR9 ΔpagP8 (also | (16) |
| | with (16) ΔaroA21419) | |
| BCT2 | ΔpabA1516 ΔpabB232 ΔasdA16 ΔmsbB48 ΔpagL7 ΔpagP81::Pₗₚₚ | (131) |
| | lpxE ΔlpxR93::Pₗₚₚ lpxF ΔfimH ΔfliC ΔfljB ΔrfaL (=waaL) ΔpgtEp | |
| | (from χ11091) | |

### Treatment Methods

The genetically modified Salmonella cells described herein and therapeutic compositions comprising the same may be used in methods to treat cancer, to attenuate the growth of a tumor or to regress a tumor. The methods described herein may be used to treat or attenuate the growth of any cancer or tumor type. Cancers and tumor types that may be treated or attenuated using the methods described herein include but are not limited to bone cancer, bladder cancer, brain cancer, breast cancer, cancer of the urinary tract, carcinoma, cervical cancer, colon cancer, esophageal cancer, gastric cancer, head and neck cancer, hepatocellular cancer, liver cancer, lung cancer, lymphoma and leukemia, melanoma, ovarian cancer, pancreatic cancer, pituitary cancer, prostate cancer, rectal cancer, renal cancer, sarcoma, testicular cancer, thyroid cancer, and uterine cancer. In addition, the methods may be used to treat tumors that are malignant (e.g., primary or metastatic cancers) or benign (e.g., hyperplasia, cyst, pseudocyst, hematoma, and benign neoplasm).

In some embodiments, a method for treating cancer may include administering a therapeutically effective amount of genetically modified Salmonella cells described herein or therapeutic compositions comprising the same to a subject who has cancer.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

A “therapeutically effective amount,” “effective amount” or “effective dose” is an amount of a composition (e.g., a therapeutic composition or cells) that produces a desired therapeutic effect in a subject, such as preventing or treating a target condition or alleviating symptoms associated with the condition. The precise therapeutically effective amount is an amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy 21^{st} Edition, Univ. of Sciences in Philadelphia (USIP), Lippincott Williams & Wilkins, Philadelphia, Pa., 2005.

The therapeutic compositions described herein may be administered by any suitable route of administration. A “route of administration” may refer to any administration pathway known in the art, including but not limited to aerosol, enteral, nasal, ophthalmic, oral, parenteral, rectal, transdermal (e.g., topical cream or ointment, patch), or vaginal. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. In one embodiment, the tumor antigen vaccines described herein (e.g., an SVN or CO-SVN Salmonella-based vaccine and associated expression plasmids) are administered orally and the compositions that disrupt tumor-derived immune suppression described herein (e.g., YS1646-shSTAT3 Y51646-shIDO1, YS1646-shArg1 or YS1646-shiNOS) are administered intravenously.

### EXAMPLES

### Example 1. Materials and Methods

a. Bacterial strains, media and bacterial growth. All CCTS strains are derived from the highly virulent S. Typhimurium UK-1 strain (132) since attenuated S. Typhimurium UK-1 strains will induce protective immunity to challenge with all S. Typhimurium strains whereas other S. Typhimurium strains attenuated with the same mutations often cannot induce protective immunity to some S. Typhimurium strains and definitely not to virulent UK-1 (133, 134). LB broth and agar (135) and Purple broth (PB) (Difco), which is devoid of arabinose (Ara), mannose (Man) and rhamnose (Rha), are used as complex media for propagation, phenotypic analyses and plating. MacConkey agar with 0.5% lactose (Lac) and 0.1% Ara, 0.1% rhamnose and 0.1% mannose (if needed) are used to enumerate bacteria recovered from mice or other animals. Bacterial growth is monitored spectrophotometrically and by plating for colony counts.

b. Molecular and genetic procedures. Methods for DNA isolation, restriction enzyme digestion, DNA cloning and use of PCR for construction and verification of bacterial strains and vectors are standard (136). DNA sequence analyses are performed commercially. All oligonucleotide and/or gene syntheses are done commercially with codon optimization to enhance translational efficiency in humans or Salmonella and stabilize mRNA to “destroy” RNase E cleavage sites (59, 60) to prolong mRNA half-life. Plasmids are evaluated by DNA sequencing and ability to specify synthesis of proteins using gel electrophoresis and western blot analyses. Expression of sequences encoded in DNA vaccine vectors is monitored after electroporation into Vero cells and using antibodies specific to DNA vaccine encoded proteins. Methods for generating mutant strains are described in previous publications (137-145) and in Examples below using the suicide vector delivery strain χ7213 (thi-1 thr-1 leuB6 glnV44 fhuA21 lacY1 recA1 RP4-2-Tc::Mu λpir ΔasdA4 Δzhf-2::Tn10). Recombinant plasmid constructs are transformed into E. coli χ6212 (F-λ-φ80 Δ(lacZYA-argF) endA1 recA1 hsdR17 deoR thi-1 glnV44 gyrA96 relA1 ΔasdA4) with selection for AsdA⁺ for initial characterization prior to electroporation into CCTS strains.

c. Selection of targeting and effector proteins. Selection of proteins that facilitate targeting to cancer cells or constitute cargo proteins with desired biological effects to be encoded on regulated lysis plasmid vectors for synthesis and delivery by CCTS strains or to be encoded on DNA vaccine vectors for expression in the inoculated animal host are based on prior discoveries and evidence of well-established activities in the published literature.

d. CCTS strain characterization. CCTS constructs are evaluated in comparison with vector-control strains for stability of plasmid maintenance, integrity and protein synthesis ability when CCTSs are grown in the presence of arabinose and DAP and with and without IPTG for 50 generations. The IPTG dependence of protein synthesis to overcome the Lac repression of the P_{trc} promoter is also verified. IPTG-induced cultures are incubated with chloramphenicol to arrest protein synthesis to determine whether plasmid-specified proteins are stable during the next 4 h. If not, the nucleotide sequence is altered to eliminate protease cleavage sites (with subsequent comparison of both constructs for induction of immune responses). Measurement of LPS core and O-antigen is performed after electrophoresis using silver-stained gels (146). Final CCTS constructs are evaluated for bile sensitivity, acid tolerance and ability to survive in sera with and without complement (143-145) and for sensitivity to antibiotics used to treat Salmonella infections.

e. Cell culture methods. Some tumors are caused by cancer cells with specific targetable receptors or that possess phenotypic properties that can be used to attract specially designed CCTS strains with specific targeting attributes. For example, bladder tumor cells uniquely display a receptor that can bind to a targeting peptide termed PLZ4 (amino acid sequence: CQDGRMGFC) that is absent on normal uroepithelial cells and other cell types throughout the body. Nanoparticles coated with PLZ4 specifically target bladder tumor cells but not to other cancer cell types (147-150). This targeting is observed for bladder tumor cells from mice, dogs and humans (151). CCTS strains displaying PLZ4 can be evaluated by their differential ability to attach to and invade the bladder tumor cell lines 5637, TCCSUP, and T24 (151). Methods for evaluating the abilities of Salmonella cells to attach to, invade into and survive in cells in culture are well established (152). These methods can be modified as needed for CCTS strains targeting other tumor cell types.

f. Cell imaging. Some plasmids have genes encoding fluorescent proteins enabling synthesis of GFP in Salmonella or EGFP or mCherry in animal cells. The fluorescent protein in bacteria or cells will be visualized using the EVOS Automated Cell Imaging System (ThermoFisher Scientific). The Cell Plasma Membrane Staining Kit—Orange Fluorescence—Cytopainter (ab219941, Abcam) was used to label cell membranes. The acquired image was processed using ImageJ software (153).

### Example 2. Construction of Mutant S. Typhimurium Strains with Deletions of the ompA Gene to Enable Display of Altered OmpA Proteins with Inserted Peptides Enabling Targeting to Specific Tumor Cells

Pan and associates have defined a nine amino acid peptide CQDGRMGFC (SEQ ID NO: 154) termed PLZ4 (U.S. Pat. No. 10,335,365) that targets a specific receptor present on bladder tumor cells (151). A number of S. Typhimurium strains with anti-tumor attributes have been constructed to display PLZ4 to preferentially and specifically target bladder tumor cells. The objective was to insert the sequence for PLZ4 into one of the exposed outer loops of the OmpA protein. The OmpA protein was selected since it is the most abundant OMP in the Salmonella outer membrane (105) and could be specified on a plasmid replicon to increase its relative quantity in relation to other OMPs.

To construct a strain to test the validity and feasibility of our approach, we generated a derivative of χ12341 to insert the ΔompA11 deletion mutation using the suicide vector pYA4757 (Table 2) to yield the strain χ12417 (Table 4). The ΔompA11 mutation deletes the entire ompA open reading frame including the start to stop codon sequence. χ12341 (73, 154) was selected since its viability and virulence are dependent on the supply of three sugars that can be supplied during culture but that are totally absent in animal tissues and since it cannot synthesize LPS O-antigen in vivo thus exposing the outer membrane proteins to enable better and more efficient interactions with eukaryotic cell surfaces in the in vivo environment.

After demonstrating that χ12417 harboring a multi-copy plasmid encoding the ompAΩplz4 fusion could adhere to bladder tumor cells displaying the receptor for PLZ4 (see below), studies were commenced to evaluate S. Typhimurium strains with a diversity of properties for use with a diversity on new plasmid vectors encoding for synthesis of attributes that contribute to tumor therapy, tumor cell destruction and/or to recruit host immunity to target tumor antigens, etc., in addition to tumor cell adherence. All these S. Typhimurium strains listed in Table 4 were constructed using the suicide vectors listed in Table 2 to introduce the mutations described in Table 1. Many were derived from Protective Immunity Enhanced Salmonella Vaccine (PIESV) strains of Self-Destructing Attenuated Adjuvant Salmonella (SDAAS) strains that have been described (PCT/US21/61814 and WO 2021/222696 A1, respectively), which are incorporated herein in their entirety.

TABLE 4
S. Typhimurium strains constructed and evaluated as CCTS strains.

χ12414 ΔwaaG42 ΔpagL21::TT araC P_{araBAD} waaG ΔlpxR9 ΔpagP8 ΔeptA4 ΔarnT6
Δpmi-2426 ΔrelA197::araC P_{araBAD} lacI TT ΔompA11
χ12417 ΔP_{murA25}::TT araC P_{BAD} murA ΔwaaL46 Δpmi-2426
ΔasdA27::TT araC P_{BAD} c2 ΔpagL64::TT rhaRS P_{rhaBAD} waaL Δ(wza-wcaM)-8
ΔrelA197::araC P_{BAD} lacI TT ΔrecF126 ΔsifA26 ΔompA11 (from χ12341)
χ12447 ΔP_{murA25}::TT araC P_{araBAD} murA ΔasdA27::TT araC P_{araBAD} c2 Δpmi-2426 Δ(wza-
wcaM)-8 ΔrecF126 ΔsifA26 ΔwaaL46 ΔpagL64::TT rhaRS P_{rhaBAD} waaL ΔendA2113
ΔrelA1123 ΔsseL116 ΔtlpA181 ΔompA11 (from χ12388)
χ12452 ΔP_{murA25}::TT araC P_{BAD} murA ΔwaaL46 ΔpagL64::TT rhaRS P_{rhaBAD} waaL Δpmi-
2426 ΔasdA27::TT araC P_{BAD} c2 ΔpagL64::TT rhaRS P_{rhaBAD} waaL Δ(wza-wcaM)-8
ΔrelA197::araC P_{BAD} lacI TT ΔrecF126 ΔsifA26 ΔompA11 ΔsopB1925 (from χ12417)
χ12485 ΔwaaL46 ΔpagL64::TT rhaRS P_{rhaBAD} waaL Δpmi-2426 ΔP_{fur33}::TT araC P_{araBAD}
fur ΔasdA33 ΔrelA197::araC P_{BAD} lacI TT Δ(wza-wcaM)-8 ΔP_{tolR67::}::TT araC P_{araBAD} tolR
ΔompA11 (from χ12473)
χ12494 ΔP_{murA25}::TT araC P_{araBAD} murA ΔasdA27::TT araC P_{araBAD} c2 Δpmi-2426 Δ(wza-
wcaM)-8 ΔrecF126 ΔsifA26 ΔwaaL46 ΔpagL64::TT rhaRS P_{rhaBAD} waaL ΔendA2113
ΔrelA1123 ΔsseL116 ΔtlpA181 ΔompA11 ΔsopB1925 (from χ12447)
χ12508 ΔP_{murA25}::TT araC P_{araBAD} murA ΔasdA27::TT araC P_{araBAD} c2 Δpmi-2426 Δ(wza-
wcaM)-8 ΔrelA197::araC P_{araBAD} lacI TT ΔrecF126 ΔsifA26 ΔwbaP45 ΔpagL14::TT araC
P_{araBAD} wbaP ΔlpxR9 ΔpagP8 ΔompA11 (from χ12449)
χ12529 ΔP_{murA25}::TT araC P_{araBAD} murA ΔasdA27::TT araC P_{araBAD} c2 Δpmi-2426
ΔwaaL46 ΔpagL64::TT rhaRS P_{rhaBAD1} waaL1 Δ(wza-wcaM)-8
ΔrelA197::araC P_{araBAD} lacI TT ΔrecF126 ΔsifA26 ΔompA11 ΔaraBAD65::TT
Δ_{rhaBAD}SR515 (from χ12425)
χ12614 ΔasdA33 ΔompA11 (from χ8958)
χ12627 ΔwaaL46 ΔpagL64::TT rhaRS P_{rhaBAD} waaL Δpmi-2426 ΔP_{fur33}::TT araC P_{araBAD}
fur ΔasdA33 ΔrelA197::araC P_{BAD} lacI TT Δ(wza-wcaM)-8 ΔP_{tolR67::}::TT araC P_{araBAD} tolR
ΔompA11 ΔpagP81::Pₗₚₚ lpxE (from χ12485)
χ12628 ΔwaaL46 ΔpagL64::TT rhaRS P_{rhaBAD} waaL Δpmi-2426 ΔP_{fur33}::TT araC P_{araBAD}
fur ΔasdA33 ΔrelA197::araC P_{BAD} lacI TT Δ(wza-wcaM)-8 ΔP_{tolR67::}::TT araC P_{araBAD} tolR
ΔompA11 ΔpagP81::Pₗₚₚ lpxE ΔlpxR9 (from χ12627)
χ12632 ΔwaaL46 ΔpagL64::TT rhaRS P_{rhaBAD} waaL Δpmi-2426 ΔP_{fur33}::TT araC P_{araBAD}
fur ΔasdA33 ΔrelA197::araC P_{BAD} lacI TT Δ(wza-wcaM)-8 ΔP_{tolR67::}::TT araC P_{araBAD} tolR
ΔompA11 ΔpagP81::Pₗₚₚ lpxE ΔlpxR9 ΔeptA4 ΔarnT6 (from χ12631)
χ12654 ΔP_{murA25}::TT araC P_{BAD} murA ΔwaaL46 Δpmi-2426
ΔasdA27::TT araC P_{BAD} c2 ΔpagL64::TT rhaRS P_{rhaBAD} waaL Δ(wza-wcaM)-8
ΔrelA197::araC P_{BAD} lacI TT ΔrecF126 ΔsifA26 ΔompA11 ΔendA2311 (from χ12417)
χ12655 ΔP_{murA25}: TT araC P_{araBAD} murA ΔasdA27: TT araC P_{araBAD} c2 Δ(wza-wcaM)-8
ΔrecF126 ΔsifA26 ΔwaaL46 ΔpagL64::TT rhaRS P_{rhaBAD} waaL ΔendA2113 ΔrelA1123
ΔsseL116 ΔtlpA181 ΔompA11 (from χ12563)
χ12656 ΔP_{murA25}::TT araC P_{araBAD} murA ΔasdA27::TT araC P_{araBAD} c2 Δ(wza-wcaM)-8
ΔrelA197::araC P_{araBAD} lacI TT ΔrecF126 ΔsifA26 ΔwaaL46 ΔpagL38::TT rhaRS P_{rhaBAD2}
waaL2 ΔaraBAD65::TT ΔrhaBADSR515 ΔpagP8 ΔlpxR9 ΔompA11 (from χ12569)
χ12657 ΔP_{murA25}::TT araC P_{araBAD} murA ΔasdA27::TT araC P_{araBAD} c2 Δ(wza-wcaM)-8
ΔrecF126 ΔsifA26 ΔwaaL46 ΔpagL38::TT rhaRS P_{rhaBAD2} waaL2 ΔendA2113 ΔrelA1123
ΔsseL116 ΔtlpA181 ΔompA11 (from χ12601)
χ12658 ΔP_{murA25}::TT araC P_{araBAD} murA ΔasdA27::TT araC P_{araBAD} c2 Δ(wza-wcaM)-8
ΔrecF126 ΔsifA26 ΔwaaL46 ΔpagL38::TT rhaRS P_{rhaBAD2} waaL2 ΔaraBAD65::TT
ΔrhaBADSR515 ΔpagP8 ΔlpxR9 ΔrelA1123 ΔompA11 (pSTUK201 Δ(traM-traX)-
36::araC P_{araBAD} lacI TT) (from χ12615)
χ12667 ΔP_{murA25}::TT araC P_{araBAD} murA ΔasdA27::TT araC P_{araBAD} c2 Δ(wza-wcaM)-8
ΔrecF126 ΔsifA26 ΔwaaL46 ΔpagL38::TT rhaRS P_{rhaBAD2} waaL2 ΔaraBAD65::TT
ΔrhaBADSR515 ΔpagP8 ΔlpxR9 ΔrelA1123 ΔompA11(pSTUK206 Δ(traM-traX)-
41::araC P_{araBAD} lacI TT) (from χ12663)
χ12733 ΔP_{murA25}::TT araC P_{BAD} murA ΔwaaL46 Δpmi-2426
ΔasdA27: TT araC P_{BAD} c2 ΔpagL64:TT rhaRS P_{rhaBAD} waaL Δ(wza-wcaM)-8 ΔrecF126
ΔsifA26 ΔompA11 ΔendA2311 ΔrelA1123 (from χ12654)
χ12734 ΔP_{murA25}::TT araC P_{BAD} murA ΔwaaL46 ΔasdA27::TT araC P_{BAD} c2 ΔpagL64::TT
rhaRS P_{rhaBAD} waaL Δ(wza-wcaM)-8 ΔrelA197::araC P_{BAD} lacI TT ΔrecF126 ΔsifA26
ΔompA11 ΔendA2311 pmi+ (from χ12654)
χ12735 ΔP_{murA25}::TT araC P_{araBAD} murA ΔasdA33 ΔwaaL46 ΔpagL38::TT rhaRS P_{rhaBAD2}
waaL2 Δ(wza-wcaM)-8 ΔrelA1123 ΔrecF126 ΔsifA26 ΔendA2113 ΔsseL116 ΔtlpA181
ΔrhaBADSR515 ΔaraBAD65::TT ΔompA11 (from χ12729)
χ12736 ΔP_{murA25}::TT araC P_{araBAD} murA ΔasdA33 ΔwaaL46 ΔpagL64::TT rhaRS P_{rhaBAD1}
waaL1 Δ(wza-wcaM)-8 ΔrelA1123 ΔrecF126 ΔsifA26 ΔendA2113 ΔsseL116 ΔtlpA181
ΔrhaBADSR515 ΔaraBAD65::TT ΔompA11 (from χ12730)
χ12748 ΔP_{murA25}::TT araC P_{araBAD} murA ΔasdA27::TT araC P_{BAD} c2 Δ(wza-wcaM)-8
ΔrelA197::araC P_{araBAD} lacI TT ΔrecF126 ΔsifA26 ΔwaaL46 ΔpagL64::TT rhaRS P_{rhaBAD1} waaL
ΔompA11 ΔsopB1925 (from χ12452)
χ12750 ΔP_{murA25}::TT araC P_{araBAD} murA ΔasdA33 ΔwaaL46 Δ(wza-wcaM)-8 ΔrecF126 ΔsifA26
ΔaraBAD65::TT ΔrhaBADSR515 ΔpagL38::TT rhaRS P_{rhaBAD2} waaL2 ΔpagP8 ΔlpxR9
ΔrelA1123 (pSTUK206 Δ(traM-traX)-41::araC P_{araBAD} lacI TT) ΔompA11 (from χ12688)
χ12751 ΔP_{murA25}::TT araC P_{araBAD} murA ΔasdA33 ΔwaaL46 Δ(wza-wcaM)-8 ΔrecF126 ΔsifA26
ΔaraBAD65::TT ΔrhaBADSR515 ΔpagL38::TT rhaRS P_{rhaBAD2} waaL2 ΔpagP8 ΔlpxR9
ΔrelA1123 (pSTUK206 Δ(traM-traX)-41::araC P_{araBAD} lacI TT) ΔompA11 ΔsopB1925 (from
χ12750)
χ12753 ΔP_{murA25}::TT araC P_{araBAD} murA ΔasdA33 ΔwaaL46 Δ(wza-wcaM)-8 ΔrecF126 ΔsifA26
ΔaraBAD65::TT ΔrhaBADSR515 ΔpagL38::TT rhaRS P_{rhaBAD2} waaL2 ΔpagP81::Pₗₚₚ lpxE
ΔlpxR9 ΔrelA1123 (pSTUK206 Δ(traM-traX)-41::araC P_{araBAD} lacI TT) ΔompA11 (from χ12702)
χ12754 ΔP_{murA25}::TT araC P_{araBAD} murA ΔasdA33 ΔwaaL46 Δ(wza-wcaM)-8 ΔrecF126 ΔsifA26
ΔaraBAD65::TT ΔrhaBADSR515 ΔpagL38::TT rhaRS P_{rhaBAD2} waaL2 ΔpagP81::Pₗₚₚ lpxE
ΔlpxR9 ΔrelA1123 (pSTUK206 Δ(traM-traX)-41::araC P_{araBAD} lacI TT) ΔompA11 ΔsopB1925
(from χ12753)
χ12755 ΔP_{murA25}::TT araC P_{BAD} murA ΔwaaL46 ΔasdA27::TT araC P_{BAD} c2 ΔpagL64::TT
rhaRS P_{rhaBAD} waaL Δ(wza-wcaM)-8 ΔrecF126 ΔsifA26 ΔompA11 ΔendA2311
ΔrelA1123 (from χ12734)
χ12756 ΔwaaG42 ΔpagL21::TT araC P_{araBAD} waaG ΔlpxR9 ΔpagP8 ΔeptA4 ΔarnT6
Δpmi-2426 ΔrelA197::araC P_{araBAD} lacI TT ΔompA11 ΔrelA1123 (from χ12414)
χ12775 ΔPmurA25::TT araC P_{BAD} murA ΔwaaL46 ΔasdA27::TT araC P_{BAD} c2 ΔpagL64::TT
rhaRS P_{rhaBAD} waaL Δ(wza-wcaM)-8 ΔrecF126 ΔsifA26 ΔompA11 ΔendA2311
ΔrelA1123 (pSTUK206 Δ(traM-traX)-41::araC P_{araBAD} lacI TT) (from χ12755)
χ12776 ΔwaaG42 ΔpagL21::TT araC P_{araBAD} waaG ΔlpxR9 ΔpagP8 ΔeptA4 ΔarnT6
Δpmi-2426 ΔrelA197::araC P_{araBAD} lacI TT ΔompA11 ΔrelA1123 (pSTUK206 Δ(traM-
traX)-41::araC P_{araBAD} lacI TT) (from χ12756)
χ12838 ΔP_{murA25}::TT araC P_{BAD} murA ΔwaaL46 ΔpagL64::TT rhaRS P_{rhaBAD} waaL1
Δ(wza-wcaM)-8 ΔrecF126 ΔsifA26 ΔompA11 ΔendA2311 ΔrelA1123 ΔasdA33 (from
χ12775)
χ12846 ΔwaaG42 ΔpagL21::TT araC P_{araBAD} waaG ΔlpxR9 ΔpagP8 ΔeptA4 ΔarnT6
Δpmi-2426 ΔrelA197::araC P_{araBAD} lacI TT ΔompA11 ΔrelA1123 (pSTUK206 Δ(traM-
traX)-41::araC P_{araBAD} lacI TT) ΔsifA26 (from χ12776)

We also constructed strains in which the ompAΩplz4 fusion replaced the wild-type chromosomal ompA gene to use as comparative controls with but one copy of the fusion. These strains constructed using the suicide vector pG8R315 (Table 2) are listed in Table 5. One of the examples is χ12619. The mutations ΔwaaL46, ΔwaaG42 and ΔwaaC41 were introduced into strain χ12619 to generate a family of strains differing in the presence of the LPS O-antigen, LPS O-antigen and outer LPS core and O-antigen and outer and inner LPS core, respectively.

We also constructed strain χ12614 with the ΔasdA33 ΔompA11 deletion mutations. This strain can be transformed with a plasmid encoding ompAΩplz4 to compare the effects of surface modification in Salmonella that affect the targeting ability of Salmonella. The plasmids could be pG8R341, or any plasmid carrying ompAΩplz4 fusion, or other ompA fused with varied targeting peptide sequences. The mutations ΔwaaL46, ΔwaaG42 and ΔwaaC41 were introduced into χ12614 to generate a series of strains analogous to those generated in χ12619 resulting in defects in O-antigen, outer core and inner core, respectively. These strains are also listed in Table 5.

TABLE 5
S. Typhimurium strains constructed with the ompAΩplz4 fusion

χ12518 Δalr-3 ΔP_{dadB66}::TT araC P_{araBAD} dadB ΔP_{asdA5}::TT araC P_{araBAD} asdA ΔfliC180
ΔpagP81::Pₗₚₚ lpxE ΔpagL7 ΔlpxR9 (from χ12516)
χ12542 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔwaaC41
χ12617 ompAΩplz4 (from χ3761)
χ12618 ΔrelA4 ΔspoT1 ΔasdA27::TT araC P_{araBAD} c2 ompAΩplz4 (from χ11001)
χ12619 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 (from χ12518)
χ12808 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaL46 (from χ12619)
χ12809 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaG42 (from χ12619)
χ12810 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaC41 (from χ12619)
χ12811 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔwaaC41 ompAΩplz4 (from χ12542)
χ12614 ΔasdA33 ΔompA11 (from χ8958)
χ12812 ΔasdA33 ΔompA11 ΔwaaL46 (from χ12614)
χ12813 ΔasdA33 ΔompA11 ΔwaaG42 (from χ12614)
χ12814 ΔasdA33 ΔompA11 ΔwaaC41 (from χ12614)

### Example 3. Construction of Plasmid Vectors for Use and Evaluation in Candidate CCTS Strains

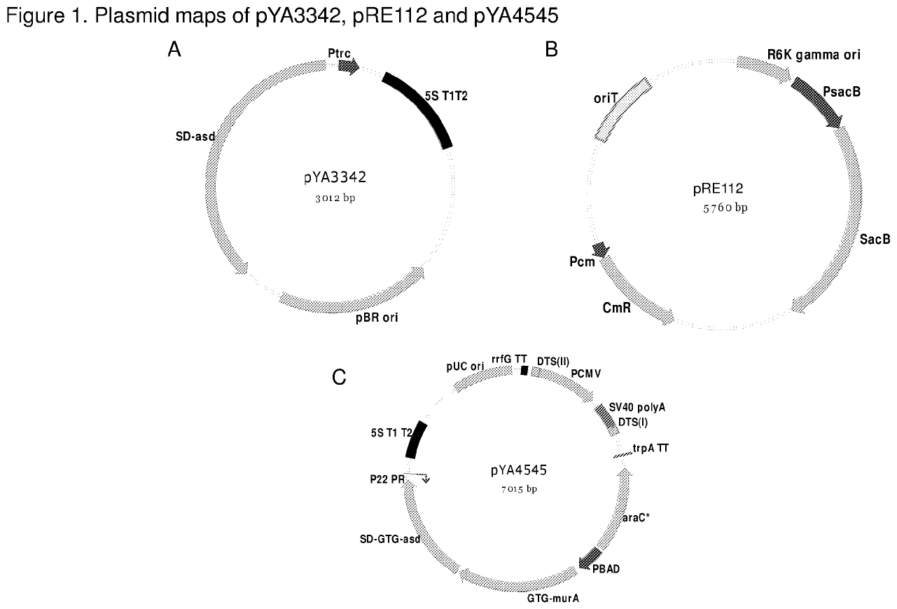

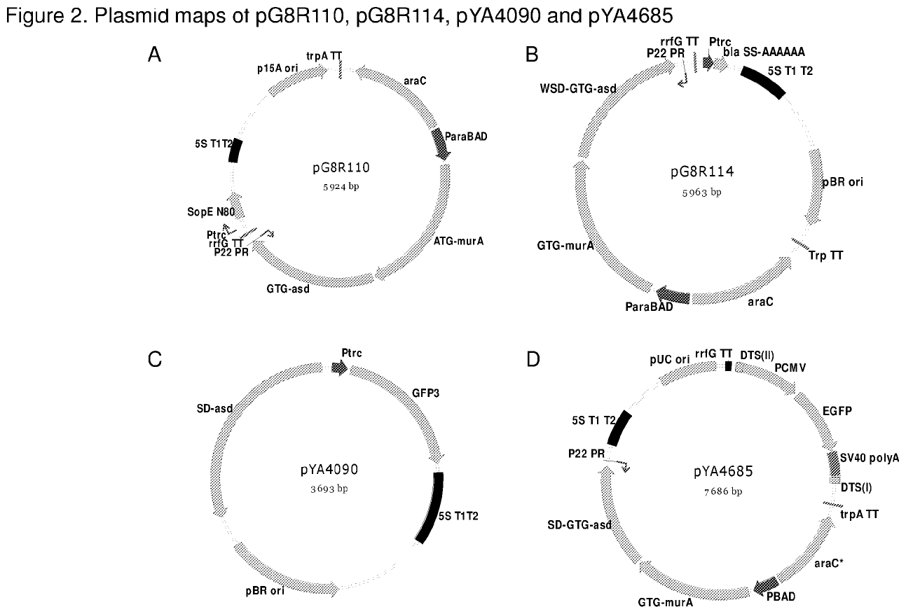

FIGS. 1 and 2 depict the plasmids used as parents or for component segments of derived and constructed plasmids. The derived and constructed plasmids are listed and described in Table 6 with their use and evaluation described in subsequent Examples. Table 7 lists all the nucleotide primers used to construct the plasmids listed in Table 6. A unique and original feature of many of the plasmids designed and constructed is the ability to encode proteins that are synthesized by the CCTS strain to be displayed during targeting and attaching to, invading into and acting within tumor cells in vivo prior to display of regulated lysis within the tumor cell to release the plasmid now serving as a DNA vaccine with unique features to be directed to the nucleus for transcription of encoded sequences that yield products after mRNA translation that exhibit anti-tumor activities. These products with their features as described in later Examples might kill the tumor cell, cause the tumor cell to kill itself (i.e., commit suicide) and/or attract host immune responses that inhibit tumor cell growth and metastases.

The ability to design and effectively use these newly designed dual function hybrid plasmids is dependent on using CCTS delivery strains that are engineered to express a regulated delayed lysis in vivo phenotype that is a composite function of regulated expression of both chromosomal and plasmid encoded genes.

TABLE 6
Lists of plasmids generated for use in anti-tumor research^{a}.

| | | | | Gene | | | |
|---|---|---|---|---|---|---|---|
| | | | | Prokaryotic | Eukaryotic | | Parent |
| Plasmid | Replicon | Marker | Promoter | expression | expression | Linker | plasmid |
| pG8R314 | pBR | asd | P_{trc} | ompAΩPLZ4 | | AC-PLZ4- | pYA3342 |
| | | | | | | CG | |
| pG8R315 | R6K | Cm | | ompAΩPLZ4 | | AC-PLZ4- | pRE112 |
| | | | | | | CG | |
| pG8R319 | pBR | asd | P_{trc} | ompAΩPLZ4 | | AC-PLZ4- | pG8R314 |
| | | | P_{CMV} | | | CG | |
| pG8R320 | pUC | araC | P_{trc} | ompAΩPLZ4 | | AC-PLZ4- | pYA4545 |
| | | P_{BAD} | P_{CMV} | | | CG | |
| | | murA | | | | | |
| | | asd | | | | | |
| pG8R321 | pBR | asd | P_{trc} | ompAΩPLZ4 | human | AC-PLZ4- | pG8R319 |
| | | | P_{CMV} | | CXCL11 | CG | |
| pG8R322 | pBR | asd | P_{trc} | ompAΩPLZ4 | mouse | AC-PLZ4- | pG8R319 |
| | | | P_{CMV} | | CXCL11 | CG | |
| pG8R323 | pBR | asd | P_{trc} | ompAΩPLZ4 | KillerRed | AC-PLZ4- | pG8R319 |
| | | | P_{CMV} | | Mem | CG | |
| pG8R324 | pBR | asd | P_{trc} | ompAΩPLZ4 | KillerRed | AC-PLZ4- | pG8R319 |
| | | | P_{CMV} | | Mito | CG | |
| pG8R325 | pUC | araC | P_{trc} | ompAΩPLZ4 | human | AC-PLZ4- | pG8R320 |
| | | P_{BAD} | P_{CMV} | | CXCL11 | CG | |
| | | murA | | | | | |
| | | asd | | | | | |
| pG8R326 | pUC | araC | P_{trc} | ompAΩPLZ4 | mouse | AC-PLZ4- | pG8R320 |
| | | P_{BAD} | P_{CMV} | | CXCL11 | CG | |
| | | murA | | | | | |
| | | asd | | | | | |
| pG8R327 | pUC | araC | P_{trc} | ompAΩPLZ4 | KillerRed | AC-PLZ4- | pG8R320 |
| | | P_{BAD} | P_{CMV} | | Mem | CG | |
| | | murA | | | | | |
| | | asd | | | | | |
| pG8R328 | pUC | araC | P_{trc} | ompAΩPLZ4 | KillerRed | AC-PLZ4- | pG8R320 |
| | | P_{BAD} | P_{CMV} | | Mito | CG | |
| | | murA | | | | | |
| | | asd | | | | | |
| pYA4090 | pBR | asd | P_{trc} | GFP3 | | | pYA3342 |
| pYA4685 | pUC | araC | P_{CMV} | | EGFP | | pYA4545 |
| | | P_{BAD} | | | | | |
| | | murA | | | | | |
| | | asd | | | | | |
| pG8R341 | pBR | Asd | P_{trc} | ompAΩPLZ4 | | | pG8R314 |
| | | | | SD GFP | | | |
| pG8R342 | pUC | araC | P_{trc} | ompAΩPLZ4 | EGFP | | pG8R320 |
| | | P_{BAD} | P_{CMV} | | | | |
| | | murA | | | | | |
| | | asd | | | | | |
| pG8R343 | pUC | araC | P_{trc} | ompAΩPLZ4 | KillerRED- | GSG P2A | pG8R320 |
| | | P_{BAD} | P_{CMV} | | memo- | | |
| | | murA | | | Human | | |
| | | asd | | | CXCL11 | | |
| pG8R344 | pUC | araC | P_{trc} | ompAΩPLZ4 | KillerRED- | GSG P2A | pG8R320 |
| | | P_{BAD} | P_{CMV} | | memo- | | |
| | | murA | | | Mouse | | |
| | | asd | | | CXCL11 | | |
| pG8R345 | pUC | araC | P_{trc} | ompAΩPLZ4 | HLAB | | pG8R320 |
| | | P_{BAD} | P_{CMV} | | leading and | | |
| | | murA | | | tail peptide | | |
| | | asd | | | | | |
| pG8R346 | pUC | araC | P_{trc} | ompAΩPLZ4 | HLAB- | | pG8R345 |
| | | P_{BAD} | P_{CMV} | | leading and | | |
| | | murA | | | tail peptide | | |
| | | asd | | | EGFP | | |
| pG8R347 | pUC | araC | P_{trc} | ompAΩPLZ4 | HLAB | | pG8R320 |
| | | P_{BAD} | P_{CMV} | | leading and | | |
| | | murA | | | tail peptide | | |
| | | asd | | | with 5′ and | | |
| | | | | | 3′ | | |
| pG8R348 | pUC | araC | P_{trc} | ompAΩPLZ4 | HLAB | | pG8R347 |
| | | P_{BAD} | P_{CMV} | | leading and | | |
| | | murA | | | tail peptide | | |
| | | asd | | | with 5′ and | | |
| | | | | | 3′ + EGFP | | |
| pG8R349 | pUC | araC | P_{trc} | ompAΩPLZ4 | HLAB | | pG8R347 |
| | | P_{BAD} | P_{CMV} | | leading and | | |
| | | murA | | | tail peptide | | |
| | | asd | | | with 5′ and | | |
| | | | | | 3′ BBN963 | | |
| pG8R350 | pUC | araC | P_{trc} | ompAΩPLZ4 | HLAB | | pG8R347 |
| | | P_{BAD} | P_{CMV} | | leading and | | |
| | | murA | | | tail peptide | | |
| | | asd | | | with 5′ and | | |
| | | | | | 3′ + MB49 | | |
| pG8R361 | pUC | araC | P_{trc} | ompAΩPLZ4 | | | pG8R320 |
| | | P_{BAD} | P_{EF1α} | | | | |
| | | murA | | | | | |
| | | asd | | | | | |
| pG8R362 | pUC | araC | P_{trc} | ompAΩPLZ4 | HAC-PD1- | 36 AA | pG8R320 |
| | | P_{BAD} | P_{CMV} | | hCXCL11 | Linker | |
| | | murA | | | | | |
| | | asd | | | | | |
| pG8R363 | pUC | araC | P_{trc} | ompAΩPLZ4 | HAC-PD1- | 36 AA | pG8R320 |
| | | P_{BAD} | P_{CMV} | | mCXCL11 | Linker | |
| | | murA | | | | | |
| | | asd | | | | | |
| pG8R364 | pUC | araC | P_{trc} | ompAΩPLZ4 | HAC-PD1- | 36 AA | pG8R361 |
| | | P_{BAD} | P_{EF1α} | | hCXCL11 | Linker | |
| | | murA | | | | | |
| | | asd | | | | | |
| pG8R365 | pUC | araC | P_{trc} | ompAΩPLZ4 | HAC-PD1- | 36 AA | pG8R361 |
| | | P_{BAD} | P_{EF1α} | | mCXCL11 | Linker | |
| | | murA | | | | | |
| | | asd | | | | | |
| pG8R366 | pUC | araC | P_{trc} | ompAΩPLZ4 | IL2 SS | | pG8R320 |
| | | P_{BAD} | P_{CMV} | | | | |
| | | murA | | | | | |
| | | asd | | | | | |
| pG8R367 | pUC | araC | P_{trc} | ompAΩPLZ4 | IL2 SS- | 36 AA | pG8R366 |
| | | P_{BAD} | P_{CMV} | | HAC-PD1- | Linker | |
| | | murA | | | hCXCL11 | | |
| | | asd | | | | | |
| pG8R368 | pUC | araC | P_{trc} | ompAΩPLZ4 | IL2 SS- | 36 AA | pG8R366 |
| | | P_{BAD} | P_{CMV} | | HAC-PD1- | Linker | |
| | | murA | | | mCXCL11 | | |
| | | asd | | | | | |
| pG8R372 | pUC | araC | P_{trc} | ompAΩPLZ4 | IL2 SS- | | pG8R320 |
| | | P_{BAD} | P_{CMV} | | HAC-PD1 | | |
| | | murA | | | | | |
| | | asd | | | | | |
| pG8R373 | pUC | araC | P_{trc} | ompAΩPLZ4 | IL2 SS- | PPVAT(SEQ | pG8R320 |
| | | P_{BAD} | P_{CMV} | | HAC-PD1- | ID NO: 155) | |
| | | murA | | | EGFP | Linker | |
| | | asd | | | | | |
| pG8R374 | pUC | araC | P_{trc} | ompAΩPLZ4 | hCXCL11- | PPVAT(SEQ | pG8R320 |
| | | P_{BAD} | P_{CMV} | | EGFP | ID NO: 155) | |
| | | murA | | | | linker | |
| | | asd | | | | | |
| pG8R375 | pUC | araC | P_{trc} | ompAΩPLZ4 | mCXCL11- | PPVAT(SEQ | pG8R320 |
| | | P_{BAD} | P_{CMV} | | EGFP | ID NO: 155) | |
| | | murA | | | | linker | |
| | | asd | | | | | |
| pG8R380 | pUC | araC | P_{trc} | ompAΩLHRH- | | | pG8R320 |
| | | P_{BAD} | P_{CMV} | GFP | | | |
| | | murA | | | | | |
| | | asd | | | | | |
| pG8R381 | pUC | araC | P_{trc} | ompAΩLHRH | | | pG8R380 |
| | | P_{BAD} | P_{CMV} | | | | |
| | | murA | | | | | |
| | | asd | | | | | |
| pG8R382 | pUC | araC | P_{trc} | ompAΩPLZ4 | haPD1-IgG | | pG8R320 |
| | | P_{BAD} | P_{CMV} | | | | |
| | | murA | | | | | |
| | | asd | | | | | |
| pG8R383 | pUC | araC | P_{trc} | ompAΩPLZ4 | haPD1- | GSG P2A | pG8R320 |
| | | P_{BAD} | P_{CMV} | | IgG- | peptide | |
| | | murA | | | KillerRed- | | |
| | | asd | | | memo | | |
| pG8R384 | pUC | araC | P_{trc} | ompAΩPLZ4 | KillerRed- | GSG P2A | pG8R320 |
| | | P_{BAD} | P_{CMV} | | memo | peptide | |
| | | murA | | | haPD1- | | |
| | | asd | | | IgG- | | |
| pG8R385 | pUC | araC | P_{trc} | ompAΩHer2 | | | pG8R320 |
| | | P_{BAD} | P_{CMV} | scFv-GFP | | | |
| | | murA | | | | | |
| | | asd | | | | | |
| pG8R386 | pUC | araC | P_{trc} | ompAΩHer2 | | | pG8R385 |
| | | P_{BAD} | P_{CMV} | scFv | | | |
| | | murA | | | | | |
| | | asd | | | | | |
| pG8R388 | pUC | araC | P_{trc} | | | | pG8R320 |
| | | P_{BAD} | P_{CMV} | | | | |
| | | murA | | | | | |
| | | asd | | | | | |
| pG8R389 | pUC | araC | P_{trc} | BIa AAAAAA | | | pG8R320 |
| | | P_{BAD} | P_{CMV} | | | | |
| | | murA | | | | | |
| | | asd | | | | | |
| pG8R390 | pUC | araC | P_{trc} | ompAΩLHRH | KillerRed- | | pG8R381 |
| | | P_{BAD} | P_{CMV} | | mem | | |
| | | murA | | | | | |
| | | asd | | | | | |
| pG8R391 | pUC | araC | P_{trc} | ompAΩHer2 | KillerRed- | | pG8R386 |
| | | P_{BAD} | P_{CMV} | scFv | mem | | |
| | | murA | | | | | |
| | | asd | | | | | |
| pG8R418 | pUC | araC | P_{trc} | ompAΩHer2 | KillerRed- | | pG8R385 |
| | | P_{BAD} | P_{CMV} | scFv-GFP | mem | | |
| | | murA | | | | | |
| | | asd | | | | | |

^{a}The sizes of all plasmids in number of nucleotide bases is indicated for each plasmid in the accompanying Figures diagramming the plasmids listed in this Table 6.

TABLE 7
List of all the nucleotide primers used to construct the plasmids listed in
Table 6.

| SEQ ID | | |
|---|---|---|
| NO: | Name | Sequence |
| 54 | OmpA-s | 5′ |
| | | GATAACAATTTCACACAGGAAACAGACCATGAAAAAGACAG |
| | | CTATCGC 3′ |
| 55 | OmpA-PLZ4-a | 5′ |
| | | GAAACCCATACGACCGTCCTGGCACGCGCCAGGGACGTTA |
| | | GACTTG 3′ |
| 56 | OmpA-PLZ4-s | 5′ |
| | | CCAGGACGGTCGTATGGGTTTCTGCGGTGGCCCGTCTACTA |
| | | AAGACCAC 3′ |
| 57 | OmpA-SacIHindIII- | 5′ |
| | a | GCCAAAACAGCCAAGCTTGAGCTCATTAAGCCTGCGGCTGA |
| | | GTTAC 3′ |
| 58 | OmpA-XbaI-s | 5′ CCCAGCAGTCTAGAATGAAAAAGACAGCTATCGC 3′ |
| 59 | rrfGTT-s | 5′ |
| | | CTGCAAAGAGATGTGCGGATCTCTAGATTATGCGAAAGGC |
| | | 3′ |
| 60 | trpTT-a | 5′ |
| | | CAACAGCTCATTTCAGAATGGAAGAAAAAAAAGCCCGCTCA |
| | | TTAG 3′ |
| 61 | trpTT-s | 5′ |
| | | CTAATGAGCGGGCTTTTTTTTCTTCCATTCTGAAATGAGCTG |
| | | TTG 3′ |
| 62 | rrfGTT-a | 5′ GCCTTTCGCATAATCTAGAGATCCGCACATCTCTTTGCAG |
| | | 3′ |
| 63 | pYA4545-TT- | 5′ |
| | BstBI-s | GTAACTCAGCCGCAGGCTTAATGAGCTTCGAAACAGATTAA |
| | | ATCAGAACGCAGAAGCG 3′ |
| 64 | pYA4545-TT-a1 | 5′ |
| | | AAAAAAAACCCCGCCCTGTCAGGGGGGGGGTTTTTTTTTCC |
| | | TACGCTCACCCATCAATTG 3′ |
| 65 | pYA4545-TT-BclI- | 5′ |
| | a | GATTAATTGTCAACAGCTCATTTCAGAATGATCAAAAAAAAC |
| | | CCCGCCCTGTCAGGGGC 3′ |
| 66 | Ptrc-BclI-s | 5 |
| | | CGCCCCTGACAGGGGGGGGTTTTTTTTGATCATTCTGAAAT |
| | | GAGCTGTTGAC 3′ |
| 67 | ompA-BstEI-a | 5′ |
| | | CTTCTGCGTTCTGATTTAATCTGTTTCGAAGCTCATTAAGCC |
| | | TGCGGCTGAG 3′ |
| 68 | Human-CXCL11- | 5′ |
| | KpnI-s | CGTTTAAACTTAAGCTTGGTACCGCCATGAGTGTGAAGGGC |
| | | ATGGC 3′ |
| 69 | Human-CXCL11- | 5′ |
| | Not-a | GTCTGCTCGAAGCATTCTCGAGCGGCCGCTTAAAAATTCTTT |
| | | CTTTCAAC 3′ |
| 70 | Mouse-CXCL11- | 5′ |
| | KpnI-s | GCGTTTAAACTTAAGCTTGGTACCGCCATGAACAGGAAGGT |
| | | CACAGC 3′ |
| 71 | Mouse-CXCL11- | 5′ |
| | NotI-a | CTCGAAGCATTCTCGAGCGGCCGCTTACATGTTTTGACGCC |
| | | TTAAAAAATTC 3′ |
| 72 | KillerRed-Mem- | 5′ |
| | KpnI-s | GCGTTTAAACTTAAGCTTGGTACCGCCACCATGCTGTGCTG |
| | | TATGAGAAGAACCAAAC 3′ |
| 73 | KillerRed- | 5′ |
| | NotIXhoI-a | GCTCGAAGCATTCTCGAGCGGCCGCTTTAATCCTCGTCGCT |
| | | ACCG 3′ |
| 74 | KillerRed-Mito- | 5′ |
| | KpnI-s | GCGTTTAAACTTAAGCTTGGTACCGCCACCATGTCCGTCCT |
| | | GACGCCGCTGC 3′ |
| 75 | SD-GFP-SacI-gs | 5′ |
| | | CGCAGGCTTAATGAGCTCAAGGAACAGTCAATGAGTAAAGG |
| | | AGAAGAAC 3′ |
| 76 | GFP-HindIII-ga | 5′ |
| | | CATCCGCCAAAACAGCCAAGCTTATTATTTGTATAGTTCATC |
| | | CATGC 3′ |
| 77 | EGFP-KpnI-gs | 5′ |
| | | GTTTAAACTTAAGCTTGGTACCACCAAAATGGTGAGCAAGG |
| | | GCGAG 3′ |
| 78 | EGFP-XhoI-ga | 5′ |
| | | CTGCTCGAAGCATTCTCGAGTTACTTGTACAGCTCGTCCAT |
| | | G 3′ |
| 79 | KillerRed-C-P2A- | 5′ |
| | a1 | GCCTGCTTCAGCAGGCTGAAGTTAGTAGCTCCGCTTCCATC |
| | | CTCGTCGCTACCGATGG 3′ |
| 80 | KillerRed-C-P2A- | 5′ |
| | a2 | AGGTCCAGGGTTCTCCTCCACGTCGCCAGCCTGCTTCAGCA |
| | | GGCTGAAG 3′ |
| 81 | P2A-Mouse | 5′ |
| | CXCL11-s | CGACGTGGAGGAGAACCCTGGACCTATGAACAGGAAGGTC |
| | | ACAGC 3′ |
| 82 | P2A-Human | 5′ |
| | CXCL11-s | CGACGTGGAGGAGAACCCTGGACCTATGAGTGTGAAGGGC |
| | | ATGGC 3′ |
| 83 | HLAB-Leading-gs | 5′ |
| | | CTTAAGCTTGGTACGCCGCCACCATGCTGGTCATGGCGCCC |
| | | CG 3′ |
| 84 | HLAB-Leading- | 5′ |
| | MCS-ga | CCTAGGCCCGGGCCCGGTACCGGAGCCGGCCCAGGTCTC |
| | | GG 3′ |
| 85 | HLAB-tail-MCS-gs | 5′ |
| | | GGTACCGGGCCCGGGCCTAGGGGCCTGGCTGTCCTGGCA |
| | | G 3′ |
| 86 | HLAB-tail-XhoI-ga | 5′ |
| | | CTGCTCGAAGCATTCTCGAGTCAAGCTGTGAGAGACACATC |
| | | 3′ |
| 87 | EGFP(HLAB)- | 5′ |
| | Kpnl-gs | CTGGGCCGGCTCCGGTACCATGGTGAGCAAGGGCGAGGA |
| | | G 3′ |
| 88 | EGFP(HLAB)- | 5′ |
| | Avril-ga | CTAGGACAGCCAGGCCCCTAGGCTTGTACAGCTCGTCCATG |
| | | CCG 3′ |
| 89 | HLAB-5′ Leading- | 5′ |
| | gs | GGCTAGCGTTTAAACTTAAGCTTGGTACAATTTGTAATACGA |
| | | CTCACTATAGGGCGGCCG 3′ |
| 90 | HLAB-3′ tail-XhoI- | 5′ |
| | ga | CTGCTCGAAGCATTCTCGAGGTACGACTATGGAACCGCGGC |
| | | CG 3′ |
| 91 | EGFP(HLAB)- | 5′ CTGGGCCGGCTCCGGTACCATGGTGAGCAAGGGCGAGG |
| | KpnI-gs | AG 3′ |
| 92 | EGFP(HLAB)- | 5′ |
| | AvrII-ga | CTAGGACAGCCAGGCCCCTAGGCTTGTACAGCTCGTCCATG |
| | | CCG 3′ |
| 93 | 4545- | 5′ |
| | (ForPEF1a)KpnI | GAGGTACCTGCAGGCCCGGGGCGGCCGCTCGAGAATGCTT |
| | XmaINotIXhoI-s | CG 3′ |
| 94 | 4545(ForPEF1a)- | 5′ CGGGCACCGGAGCGGAAAGTCCCCGGAAAGTCCCCGCC |
| | a2 | 3′ |
| 95 | PEF1a-s | 5′ CTTTCCGGGGACTTTCCGCTCCGGTGCCCGTCAGTGG 3′ |
| 96 | PEF1a-KpnI-a | 5′ |
| | | CCCGGGCCTGCAGGTACCTCACGACACCTGAAATGGAAG 3′ |
| 97 | HAC-PD1-KpnI-s | 5′ |
| | | GCGTTTAAACTTAAGCTTGGTACCGCCATGGATTCCCCAGA |
| | | TAGACCATG 3′ |
| 98 | HAC-PD1-Linker- | 5′ |
| | a1 | CCTCGCTTCCTCCGCCTTCACTTCCACCGCCCTCACTGCCG |
| | | CCGCCGGAGCCGCCTCTTTCAGTGACTCTCAATTC 3′ |
| 99 | HAC-PD1-Linker- | 5′ |
| | a2 | GCTTCCGCCGCCGCTGCCTCCACCCTCAGACCCGCCTCCT |
| | | TCGGAGCCTCCTCCCTCGCTTCCTCCGCCTTCAC 3′ |
| 100 | Linker-mCXCL11- | 5′ CAGCGGCGGCGGAAGCATGAACAGGAAGGTCACAGC 3′ |
| | s | |
| 101 | Linker-hCXCL11-s | 5′ CAGCGGCGGCGGAAGCATGAGTGTGAAGGGCATGGC 3′ |
| 102 | IL-2-s2 | 5′ |
| | | GTTTAAACTTAAGCTTGGTACGCCACCATGTACAGGATGCAA |
| | | CTCCTGTCTTGCATTGCACTAAGTCTTGCAC 3′ |
| 103 | IL-2-a2 | 5′GACTAGTGGATCCGAGCTCGGTACCTGCACTGTTTGTGAC |
| | | AAGTGCAAGACTTAGTGCAATGCAAGACAGGAG 3′ |
| 104 | HAC-PD1-a | 5′ |
| | | CTCGAAGCATTCTCGAGCGGCCGCTTATCTTTCAGTGACTC |
| | | TCAATTC 3′ |
| 105 | HAC-PD1-linker- | 5′ GGTGGCGACCGGTGGTCTTTCAGTGACTCTCAATTC 3′ |
| | EGFP-a | |
| 106 | HAC-PD1-linker- | 5′ CCACCGGTCGCCACCATGGTGAGCAAGGGCGAGGAG 3′ |
| | EGFP-s | |
| 107 | C terminal EGFP- | 5′ |
| | XhoINotI-a | CTCGAAGCATTCTCGAGCGGCCGCTTACTTGTACAGCTCGT |
| | | CCATGCC 3′ |
| 108 | hCXCL11-EGFP-a | 5′ GGTGGCGACCGGTGGAAAATTCTTTCTTTCAACTTTTTTG |
| | | 3′ |
| 109 | mCXCL11-EGFP-a | 5′ |
| | | GGTGGCGACCGGTGGCATGTTTTGACGCCTTAAAAAATTC |
| | | 3′ |
| 110 | LHRH-a | 5′ |
| | | CGCAGGCCATAGCTCCAATGTTCGCACGCGCCAGGGACGT |
| | | TAG 3′ |
| 111 | LHRH-s | 5′ |
| | | GAGCTATGGCCTGCGTCCGGGCTGCGGTGGCCCGTCTACT |
| | | AAAG 3′ |
| 112 | OmpAGFP-BstBI-a | 5′ |
| | | CTCATGGTGACGAGCCTTCGAATCATTAAGCCTGCGGCTG |
| | | 3′ |
| 113 | GFP-s | 5′ GGCTCGTCACCATGAGTAAAGGAGAAGAACTTTTC 3′ |
| 114 | GFP-BstBI-a | 5′ |
| | | CGTTCTGATTTAATCTGTTTCGAATTATTTGTATAGTTCATC |
| | | 3′. |
| 115 | haPD1IgG-KpnI-s | 5′ |
| | | CGTTTAAACTTAAGCTTGGTACCGCCATGGGCTGGTCCTGT |
| | | ATCATC 3′ |
| 116 | haPD1IgG-NotI-a | 5′ |
| | | GTCTGCTCGAAGCATTCTCGAGCGGCCGCTTATTTACCTGG |
| | | AGTCCGG 3′. |
| 117 | P2A-KillerRed- | 5′ |
| | Mem-s | CGACGTGGAGGAGAACCCTGGACCTATGCTGTGCTGTATGA |
| | | GAAG 3′ |
| 118 | P2A-gs | 5′ |
| | | CTAACTTCAGCCTGCTGAAGCAGGCTGGCGACGTGGAGGA |
| | | GAACCCTG 3′ |
| 119 | P2A-haPD1IgG-s | 5′ |
| | | GGAGAACCCTGGACCTATGGGCTGGTCCTGTATCATCGTG |
| | | 3′ |
| 120 | OmpA-Her2-a | 5′ |
| | | CCTCTGCCCCAGACTGGCCAGGGACGTTAGACTTGGTGTC |
| | | 3′ |
| 121 | OmpA-Her2-s | 5′ CATCACCATCACCATGGCCCGTCTACTAAAGACCAC 3′ |
| 122 | Her2-s | 5′ CAGTCTGGGGCAGAGGTGAAAAAG 3′ |
| 123 | Her2His-a | 5′ ATGGTGATGGTGATGATGAGATCC 3′ |
| 124 | haPD1IgG-C-P2A- | 5′ |
| | a | GCAGGCTGAAGTTAGTAGCTCCGCTTCCTTTACCTGGAGTC |
| | | CGGGAGAAG 3′. |
| 125 | 4545Ptrc-a | 5′ |
| | | CTGATTTAATCTGTTTCGAACCTGCAGGGGCCCGGGCCGCG |
| | | GAGTACTCCTAGGGTCTGTTTCCTGTGTGAAATTG 3′ |
| 126 | 4545PtrcBlaAAA-a | 5′ |
| | | CTGATTTAATCTGTTTCGAACCTGCAGGGGCCCGGGCCGCG |
| | | GAGTACTCCTAGGTTCAGCATCTTTTACTTTCAC 3′ |

### Example 4. Insertion of the Nucleotide Sequence Encoding the Nine Amino Acids of PLZ4 into the Third Exposed Loop of the S. Typhimurium ompA Gene and Construction of Plasmids to Encode Synthesis of this Fusion or Insert it into the S. Typhimurium Chromosome

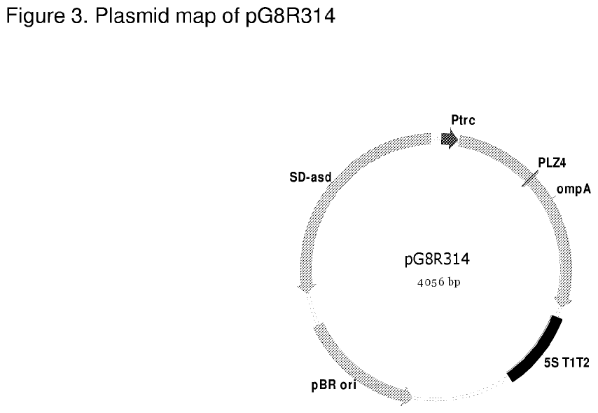

The pG8R314 plasmid (FIG. 3) with the ompAΩplz4 fusion was constructed by amplifying a 454 bp fragment of the S. Typhimurium UK-1 (χ3761) chromosome using primers OmpA-s and OmpA-PLZ4-a and a 707 bp fragment with primers OmpA-PLZ4-s and OmpA-SacIHindIII-a. These two fragments were cloned into plasmid pYA3342 (FIG. 1A) cut with NcoI/HindIII to generate plasmid pG8R314. Note that the sequence encoding the PLZ4 peptide was introduced by both primers OmpA-PLZ4-a and OmpA-PLZ4-s. This plasmid has a gene encoding the PLZ4 peptide inserted into the third exposed loop of the OmpA protein enabling expression of the ompAΩplz4 insertion mutated gene in Salmonella. The PLZ4 peptide was flanked with 2 cysteines forming a disulfide linkage to facilitate its exposure on the loop 3.

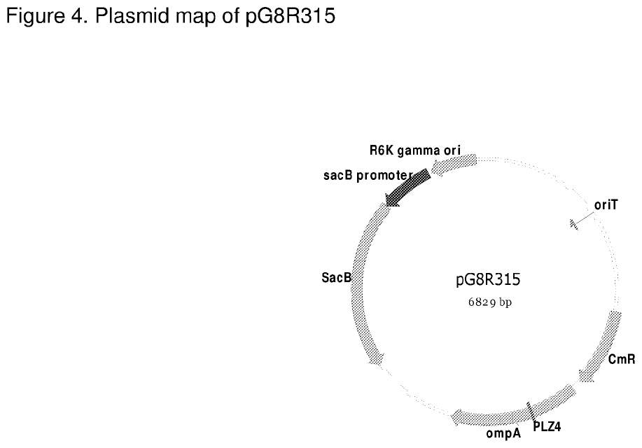

To construct the suicide vector pG8R315 (FIG. 4), the plasmid pG8R314 was used as the template to generate a 1.1 kb fragment encoding synthesis of OmpAΩPLZ4. This fragment was amplified with primers OmpA-XbaI-s and OmpA-SacIHindIII-a and cut with XbaI/SacI. The fragment was then inserted into suicide plasmid pRE112 (FIG. 1B) cut with XbaI/SacI to generate plasmid pG8R315. This suicide vector is then used to introduce the ompAΩplz4 mutation into the chromosome of the S. Typhimurium strains listed in Table 5.

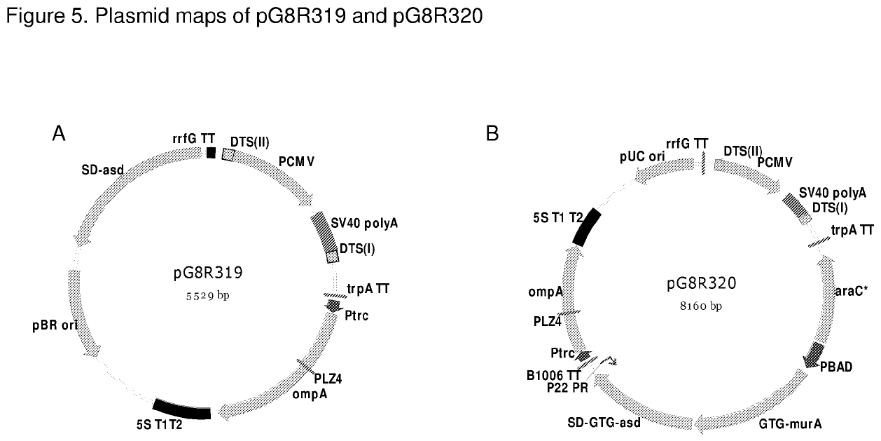

For the construction of pG8R319 (FIG. 5A), we fused two DNA fragments. With plasmid pYA4545 (FIG. 1C) as a template, a 1,549 bp fragment containing rrfG TT-PCMV-SV40 polyA Trp TT was amplified with primers rrfGTT-s and trpTT-a. With plasmid pG8R314 (FIG. 3) as a template, a 4,085 bp fragment that includes the whole pG8R314 plasmid was amplified with primers trpTT-s and rrfGTT-a. The two fragments were assembled to generate plasmid pG8R319. The balanced-lethal plasmid has a pBR on and could express ompAΩplz4 in Salmonella and has a P_{cmv} promoter to be used for gene expression in eukaryotic cells. The P_{cmv} and P_{trc} ompAΩplz4 are separated by the trpA TT.

For the construction of pG8R320 (FIG. 5B), we used plasmid pYA4545 (FIG. 1C) as a template to generate a 8 kb fragment containing the entire pYA4545 plasmid that was amplified with primers pYA4545-TT-BstBI-s and pYA4545-TT-a1 and then extended by PCR with primers pYA4545-TT-BstBI-s and pYA4545-TT-BcII-a. Then with plasmid pG8R314 (FIG. 3) as a template, a 1,242 bp fragment containing P_{trc}-ompA was amplified with primers P_{trc}-BcII-s and ompA-BstEI-a. The two fragments were then assembled to generated plasmid pG8R320 (FIG. 5B). This regulated delayed lysis plasmid has a pUC on and can express ompAΩplz4 in Salmonella to display the synthesized OmpAΩPLZ4 on the cell surface and after lysis of the cell employs the P_{cmv} promoter to express an inserted gene sequence in eukaryotic cells. The P_{cmv} and P_{trc} ompAΩplz4 are separated in the dual plasmid vector pG8R320 by the regulated delayed lysis cassette araC P_{araBAD} GTG murA GTG asdA.

### Example 5. Display of OmpAΩPLZ4 on the Bacterial Cell Surface Enables S. Typhimurium Cells to Preferentially Attach to Bladder Tumor Cells

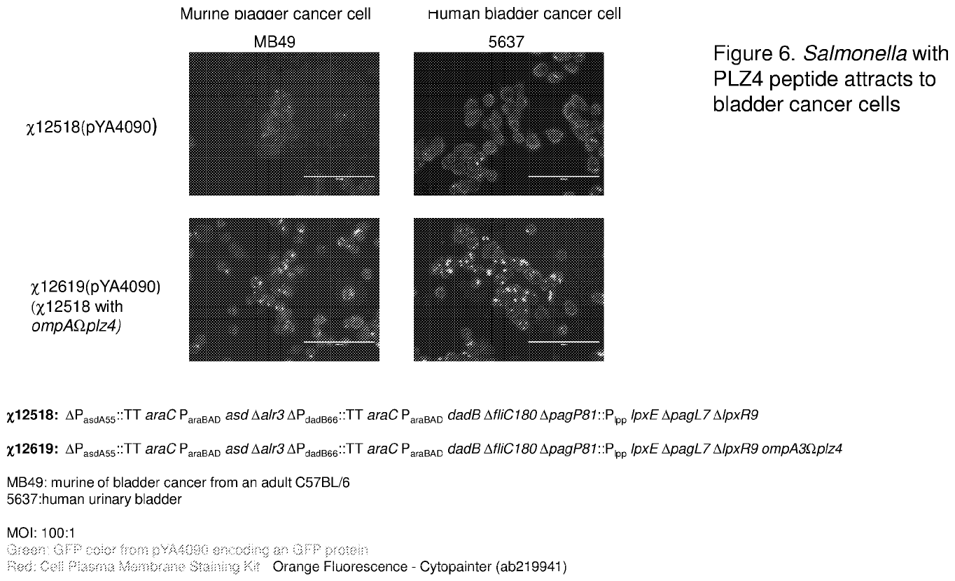

The ompAΩplz4 mutation was introduced into strain χ12518 to generate strain χ12619 using suicide vector χ7213(pG8R315). Both strains were transformed with plasmid pYA4090 to enable tagging the bacteria with the GFP protein. Overnight cultures of χ12518(pYA4090) and χ12619(pYA4090) were diluted into LB broth with 0.1% arabinose and grown until OD600 reached 0.9. The bacteria were washed once with PBS and then used to infect MB49 murine bladder cancer cells and 5637 human bladder cancer cells at MOI 1:100 for 1 hour. The MB49 membrane was stained with Cell Plasma Membrane Staining Kit—Orange Fluorescence—Cytopainter (ab219941, Abcam). As shown in FIG. 6, the strain χ12619 with OmpAΩPLZ4 is more attracted to and invades the bladder cancer cells much better than the strain χ12518 without the OmpAΩPLZ4 fusion.

### Example 6. Construction of Dual Plasmids to Cause CCTS Strains to Target Bladder Tumor Cells and then Express Proteins that Synthesize the CXCL11 Chemokine that Attract Cells of the Immune System

Although the quantity of immune cells in the bladder is not well studied, the bladder has γδ, CD4 and CD8 T cells, macrophages, dendritic cells and NK cells (155). Notably, there is no report of CD8 T cells in the mouse bladder (155). Thus, it is important to recruit T cells to the bladder to potentiate immunotherapy of bladder cancer. CXCL11 functions by binding to the receptors CXCR3 predominantly, as well as CXCR7 (156-160). CXCR3 is expressed on immune cells, such as activated T cells, NK and NKT cells, DCs, but not on naive T cells (161), and a variety of non-immune cells, such as astrocytes, fibroblasts, endothelial cells, epithelial muscle cells, and cancer cells (162, 163). CXCR7 is expressed on multiple immune cells, such as T cells, monocytes, DC cells, B cell and NK cells (164). CXCL11 has diverse functions including inhibiting angiogenesis, increasing immune cell migration, affecting proliferation of different cell types, stimulation of IFN-γ production by immune cells, suppressing M2 macrophage polarization, playing a role in fibroblast directed carcinoma invasion, increasing adhesion and invasion properties, facilitating the migration of certain immune cells, and serving as an adjuvant to anti-cancer therapies (160, 165). Although CXCL11 mainly works for immune cell migration, differentiation and activation, it could promote cancer cell proliferation and metastasis. Intratumor delivery of CXCL11 has been shown to enhance the efficacy of T-cell infiltration, adoptive T-cell therapy and vaccine efficacy (166-169). Locally produced CXCL11 in tumor cells will mediate the recruitment of T cells and NK cells to the tumor site to combat tumor development and growth. This will reduce the global toxicity related to overproduction of CXCL11 in non-tumor sites. For these reasons, we determined that the synthesis of CXCL11 by CCST cells would be optimal if the chemokine was synthesized by tumor cells rather than into the environment if synthesized and delivered by the CCST cells being used for combatting bladder cancer.

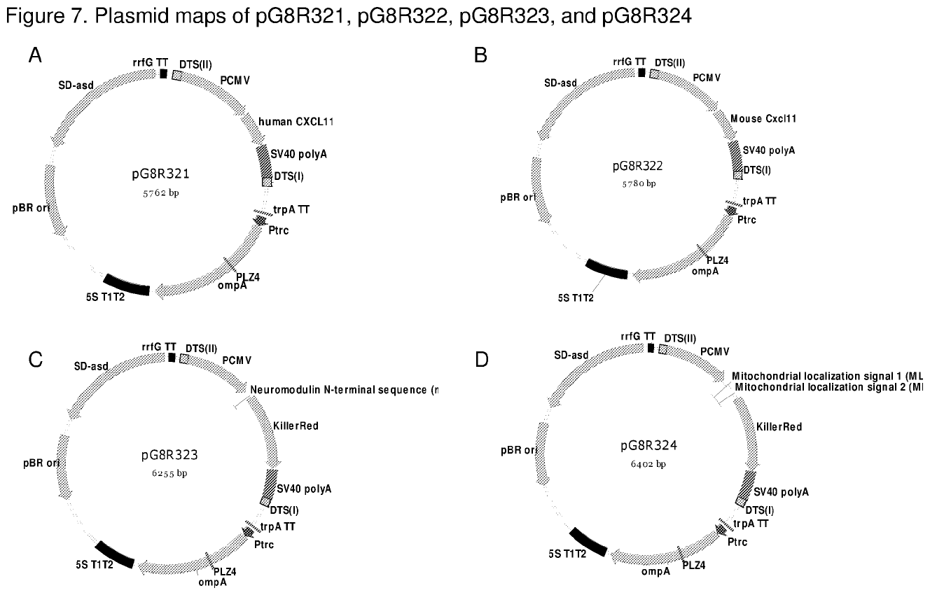

FIG. 7 displays diagrams of pG8R321 (A) and pG8R322 (B) that express ompAΩplz4 in Salmonella to display the synthesized OmpAΩPLZ4 on the cell surface and after plasmid release of the plasmid in tumor cells employs the P_{cmv} promoter to express the human and mouse CXCL11 chemokines, respectively. To construct pG8R321, we used the CXCL11 (NM_005409) Human Tagged ORF Clone (human CXCL11 (Myc-DDK-tagged), ORIGENE Cat #RC210320) as a template to amplify the gene encoding human CXCL11 with primers Human-CXCL11-KpnI-s and Human-CXCL11-Not-a, which was inserted into plasmid pG8R319 cut with KpnI/NotI. The balanced-lethal vector-host combination specifically targets human bladder cancer cells due to the display of the OmpAΩPLZ4 surface protein fusion to induce synthesis of the human CXC11 after invasion into tumor cells to release pG8R321.

pG8R322 was similarly constructed using CXCL11 (NM_019494) Mouse Tagged ORF Clone (mouse CXCL11 (Myc-DDK-tagged), ORIGENE Cat #MR222244) as the template to amplify the gene encoding mouse CXCL11 with primers Mouse-CXCL11-KpnI-s and Mouse-CXCL11-NotI-a. This sequence was then inserted into plasmid pG8R319 cut with KpnI/NotI to generate plasmid pG8R322. The balanced-lethal vector-host targets mouse bladder cancer cells due to the display of the OmpAΩPLZ4 surface protein fusion to induce synthesis of the murine CXC11 after invasion into tumor cells to release pG8R322.

### Example 7. Construction of Dual Plasmids to Cause CCTS Strains to Target Bladder Tumor Cells and then Express a Gene Sequence Encoding for Synthesis of KillerRed to Potentiate Tumor Cell Killing

Photodynamic therapy is an important therapeutic treatment for cancer and other diseases. KillerRed is the first engineered photosensitizer with light-induced cytotoxicity that could be used for precise light-induced cell killing and target protein inactivation (170-175). Upon light activation, KillerRed will produce toxic reactive oxygen species to use for photodynamic therapy against cancer. Plasmid pG8R323 (FIG. 7C) carries the gene encoding a membrane-targeting KillerRed by fusing with Neuromodulin N-terminal sequence (KillerRed mem thereafter) while plasmid pG8R324 (FIG. 7D) carries the gene encoding a mitochondria targeting KillerRed by fusion with mitochondrial location signals (KillerRed mito thereafter). Both plasmids are balanced-lethal plasmids. These vectors specify expression of the ompAΩplz4 gene in Salmonella to display the synthesized OmpAΩPLZ4 on the cell surface and after release from the CCST cell in the tumor cell employs the P_{cmv} promoter to express the KillerRed encoding genes to kill tumor cell when induced with light.

To construct pG8R323 (FIG. 7C), we used plasmid pCS2-NXE+mem-KillerRed (Addgene Cat #45761) as a template to amplify the gene encoding KillerRed mem using primers KillerRed-Mem-KpnI-s and KillerRed-NotIXhoI-a. The sequence was then inserted into plasmid pG8R319 (FIG. 5A) cut with KpnI/NotI to generate plasmid pG8R323. The balanced-lethal vector-host construct with pG8R323 specifies synthesis of a membrane-targeted KillerRed.

To construct pG8R324 (FIG. 7D) specifying KillerRed-dMito, we used pKillerRed-dMito (EVROGEN cat #FP964) as a template by amplifying the gene encoding KillerRed mito with primers KillerRed-Mito-KpnI-s and KillerRed-NotIXhoI-a and inserting into plasmid pG8R319 (FIG. 5A) cut with KpnI/NotI to generate plasmid pG8R324. The balanced-lethal vector-host construct carries a mitochondria-targeted KillerRed. KillerRed localized on cellular membranes can be used for effective light-induced cell killing by light-induced production of reactive oxygen species and can also be used to detect tumor cells infected with the CCST cells.

### Example 8. Construction of CCST Strains with Regulated Delayed Lysis with Regulated Delayed Lysis High Copy Number Plasmid Vectors Encoding Display of the OmpAΩPLZ4 Surface Protein Fusion and In Situ Synthesis of CXCL11 and KillerRed

The plasmids pG8R321, pG8R322, pG8R323 and pG8R324 (FIG. 7) all have the moderate copy number pBR on and specify the balanced-lethal phenotype using plasmid encoded expression of the asdA gene. We previously determined during the development of means for DNA vaccine delivery by Salmonella (45) that use of high copy number plasmids with pUC on acting as DNA vaccines were more effectively delivered by Salmonella cells undergoing lysis after invasion into host cells. To further validate this belief in developing optimal means for using CCST strains, we constructed versions of the pG8R321, pG8R322, pG8R323 and pG8R324 plasmids with the high copy number pUC on and with regulated delayed lysis attributes to use in Salmonella vector strains also displaying the regulated delayed lysis phenotype (see Table 4).

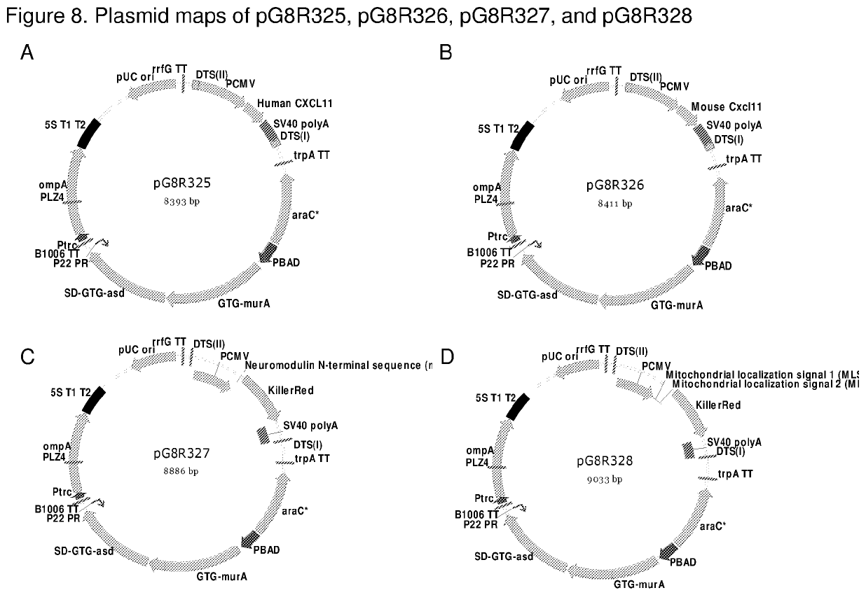

To construct pG8R325 (FIG. 8A) encoding human CXCL11, we amplified a sequence from CXCL11 (NM_005409) Human Tagged ORF Clone (human CXCL11 (Myc-DDK-tagged), ORIGENE Cat #RC210320) as a template with primers Human-CXCL11-KpnI-s and Human-CXCL11-NotI-a and cloned into plasmid pG8R320 (FIG. 5B) cut with KpnI/NotI to generate plasmid pG8R325. The lysis vector carries a human CXC11 gene.

To construct pG8R326 (FIG. 8B) encoding mouse CXCL11, we amplified a sequence from CXCL11 (NM_019494) Mouse Tagged ORF Clone (mouse CXCL11 (Myc-DDK-tagged), ORIGENE Cat #MR222244) as a template with primers Mouse-CXCL11-KpnI-s and Mouse-CXCL11-NotI-a to insert into plasmid pG8R320 (FIG. 5B) cut with KpnI/NotI. The pG8R326 lysis vector carries a mouse CXC11 gene. CXCL11 is chemotactic for interleukin-activated T-cells but not unstimulated T-cells, neutrophils or monocytes. It is the dominant ligand for CXCR3.

To construct pG8R327 (FIG. 8C), we used pCS2-NXE+mem-KillerRed (Addgene Cat #45761) as a template to amplify the gene encoding KillerRed mem with primers KillerRed-Mem-KpnI-s and KillerRed-NotIXhoI-a to insert into plasmid pG8R320 (FIG. 5B) cut with KpnI/NotI. The pG8R327 lysis vector carries a membrane-targeted KillerRed.

To construct pG8R328 (FIG. 8D), we used pKillerRed-dMito (EVROGEN cat #FP964) as a template to amplify a sequence encoding KillerRed mito using primers KillerRed-Mito-KpnI-s and KillerRed-NotIXhoI-a to insert into plasmid pG8R320 (FIG. 5B) cut with KpnI/NotI. The lysis vector pG8R328 specifies synthesis of the mitochondria-targeted KillerRed. KillerRed localized on cellular membranes can be used for effective light-induced cell killing by light-induced production of reactive oxygen species.

### Example 9. Construction of Plasmid Vectors Encoding Synthesis of GFP or EGFP to Track Salmonella Extracellularly and Intracellularly to Evaluate the Targeting Ability of CCTS Strains to Bladder Tumors

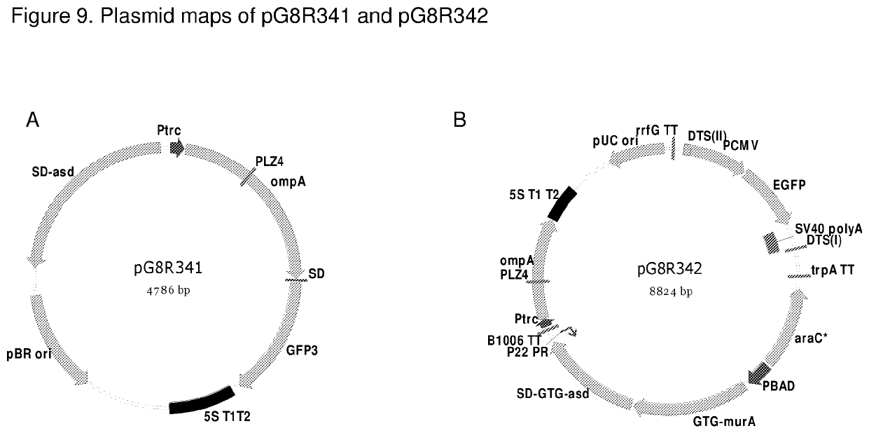

FIG. 9 displays the diagrams of plasmids pG8R341 (FIG. 9A) and pG8R342 (FIG. 9B) used to tag Salmonella with fluorescent proteins. The balanced-lethal plasmid pG8R341 carries a P_{trc} promoter which can express the operon fusion of ompAΩplz4 and gfp that enables GFP production in the Salmonella cytosol and displays the synthesized OmpAΩPLZ4 on the cell surface of Salmonella. P_{trc} is a prokaryotic promoter that can express at high level protein synthesis under both anaerobic and aerobic conditions and is repressed by Lac (176). Salmonella strains carrying this plasmid in vivo in the absence of arabinose to preclude synthesis of Lac will produce GFP in Salmonella cells present extracellularly and intracellularly. This regulated delayed lysis plasmid pG8R342 (FIG. 9B) has a pUC on and can express the ompAΩplz4 in Salmonella to display the synthesized OmpAΩPLZ4 on the cell surface and after lysis of the cell within host cells employs the P_{cmv} promoter to express an inserted egfp in eukaryotic cells. The EGFP is only produced when Salmonella is inside the mammalian cells.

To construct pG8R341 (FIG. 9A), we used plasmid pYA4090 as a template to amplify the gene encoding GFP3 using primers SD-GFP-SacI-gs and GFP-HindIII-ga. The sequence was inserted into plasmid pG8R314 (FIG. 3) cut with Sac/HindIII to generate plasmid pG8R341. The balanced-lethal plasmid uses GFP to track Salmonella with OmpAΩPLZ4.

To construct pG8R342 (FIG. 9B), we used plasmid pYA4685 as a template to amplify the gene encoding EGFP using primers EGFP-KpnI-gs and EGFP-XhoI-ga. The sequence was inserted into plasmid pG8R320 (FIG. 5B) cut with KpnI/XhoI to generate plasmid pG8R342. The lysis plasmid uses EGFP to track Salmonella within mammalian cells.

### Example 10. Construction of Dual Plasmids to Cause CCTS Strains to Target Bladder Tumor Cells and then Express Gene Sequences Encoding KillerRed to Potentiate Tumor Cell Killing and CXCL11 to Attract Immune Cells

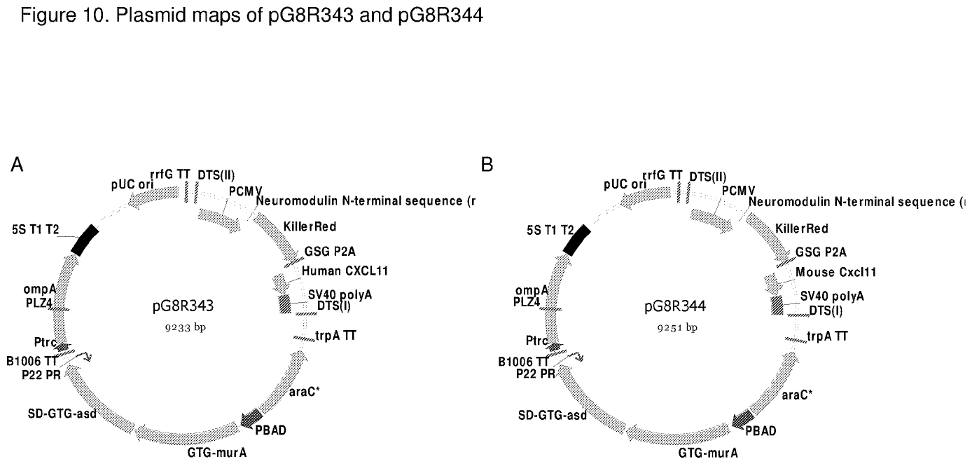

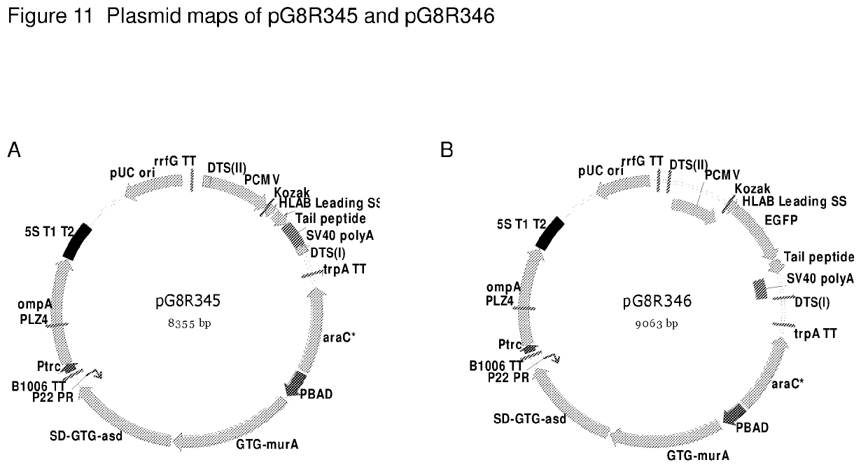

A construction that can kill cancer cells and recruit immune cells to tumor cells will have synergic effect to benefit bladder cancer therapy. FIG. 10 displays diagrams of such constructions, balanced-lethal plasmids with regulated delayed lysis attributes pG8R343 (FIG. 9A) and pG8R344 (FIG. 9B) with pUC on that express ompAΩplz4 in Salmonella to display the synthesized OmpAΩPLZ4 on the cell surface and after lysis of the bacterial cell employs the P_{cmv} promoter to express genes encoding KillerRed mem that kill tumor cells and human or mouse CXCL11 that recruit immune cells to combat bladder tumors. To enable co-expression of genes encoding KillerRed mem and human or mouse CXCL11 in mammalian cells, a P2A peptide (177-179) was introduced between the genes encoding KillerRed mem and human or mouse CXCL11 to enable ribosome skip to enable synthesis of a peptide bond at the C-terminus of a 2A element leading to cleavage between the end of the 2A sequence and the CXCL11 peptide downstream (177-179).

To construct pG8R343 (FIG. 10A), we fused two fragments encoding KillerRed mem P2A and human CXCL11. We used plasmid pCS2-NXE+mem-KillerRed (Addgene plasmid #45761) as a template to amplify the gene encoding KillerRed mem using primers KillerRed-Mem-KpnI-s and KillerRed-C-P2A-a1. The fragment was used as a template and amplified with primers KillerRed-Mem-KpnI-s and KillerRed-C-P2A-a2 to include the sequence encoding P2A. We then used CXCL11 (NM_005409) Human Tagged ORF Clone (human CXCL11 (Myc-DDK-tagged), ORIGENE Cat #RC210320) as a template to amplify the gene encoding human CXCL11 using primers P2A-Human CXCL11-s and Human-CXCL11-NotI-a. The KillerRed Mem-P2A and human CXCL11 fusion sequence was inserted into plasmid pG8R320 (FIG. 5B) cut with KpnI/NotI to generate plasmid pG8R343. The lysis plasmid carries genes encoding KillerRed mem and human CXCL11. The two genes were separated by a P2A peptide (177-179) which can induce cleavage at the C-terminal of the P2A peptide to enable production of both KillerRed mem and human CXCL11 after Salmonella invasion into tumor cells and lysis to release pG8R343 and enable synthesis of KillerRed mem and human CXCL11.

To construct pG8R344 (FIG. 10B), we fused two fragments for KillerRed mem P2A and mouse CXCL11. The KillerRed mem P2A was generated as above for plasmid pG8R343. We used CXCL11 (NM_019494) Mouse Tagged ORF Clone (mouse CXCL11(Myc-DDK-tagged), ORIGENE Cat #MR222244) as a template to amplify the gene encoding mouse CXCL11 using primers P2A-Mouse CXCL11-s and Mouse-CXCL11-NotI-a. The KillerRed Mem-P2A and mouse CXCL11 fusion was inserted into plasmid pG8R320 (FIG. 5B) cut with KpnI/NotI to generate plasmid pG8R344. The lysis plasmid carries genes encoding KillerRed mem and mouse CXCL11. The two genes were separated by a P2A peptide (177-179) which can induce cleavage at the C-terminal of the P2A peptide to enable production of both KillerRed mem and mouse CXCL11 after Salmonella invasion into tumor cells and lysis to release pG8R344 and enable synthesis of KillerRed mem and mouse CXCL11.

### Example 11. Construction of Dual Plasmids to Cause CCTS Strains to Target Bladder Tumor Cells and then after Invasion Express a Gene Sequence Fused with an HLA Peptide Encoding Sequence to Potentiate Immune Responses to Tumor Cells

Many factors can affect vaccine-induced immune responses. Antigens can be linked to lysosomal or endosomal targeting signals to route the antigen into a MHC class II processing compartment to improve CD4⁺ T cell responses. A chimeric protein fused with the N-terminal leader peptide with an MHC class I trafficking signal (tail peptide) attached to the C-terminal end of an antigen can strongly improve the presentation of MHC class I and class II epitopes in human and murine dendritic cells, leading to efficient expansion of antigen specific CD4⁺ and CD8⁺ T cells and their effector functions (180). We thus generated plasmid pG8R345 (FIG. 11 A) specifying HLAB leading and tail peptides to enable an inserted antigen to be presented to MHC class I and class II pathways.

To construct pG8R345 (FIG. 11A), we used HLAB (HLA-B) (NM_005514) Human Untagged Clone (ORIGENE Cat #SC124484) as a template to amplify the gene encoding the HLA leading peptide using primers HLAB-Leading-gs and HLAB-Leading-MCS-ga and HLA tail peptide using primers HLAB-tail-MCS-gs and HLAB-tail-XhoI-ga. The sequence generated encoding the above two fragments were inserted into plasmid pG8R320 (FIG. 5B) cut with KpnI/XhoI to generate plasmid pG8R345. This plasmid has sequences for HLAB leading and tail peptides which could be used to coupling a selected antigen to MHC Class I Trafficking Signals to increase antigen presentation efficiency (180). This regulated delayed lysis plasmid pG8R345 has a pUC on and can express ompAΩplz4 in Salmonella to display the synthesized OmpAΩPLZ4 on the cell surface and after tumor cell invasion and lysis of the CCST cell employs the P_{cmv} promoter to express the HLA-antigen encoding gene in eukaryotic tumor cells.

We then inserted egfp into plasmid pG8R345 to generate plasmid pG8R346 (FIG. 11 B). The egfp is under the control of PCMV which can be expressed in eukaryotic cells. To construct pG8R346, we used plasmid pYA4685 (FIG. 2D) as a template to amplify the gene encoding EGFP using primers EGFP(HLAB)-KpnI-gs and EGFP(HLAB)-AvrII-ga. The sequence was inserted into plasmid pG8R345 (FIG. 11A) cut with KpnI/AvrII to generate plasmid pG8R346. The lysis plasmid carries a gene encoding EGFP and could be used to track the CCTS strain in mammalian cells.

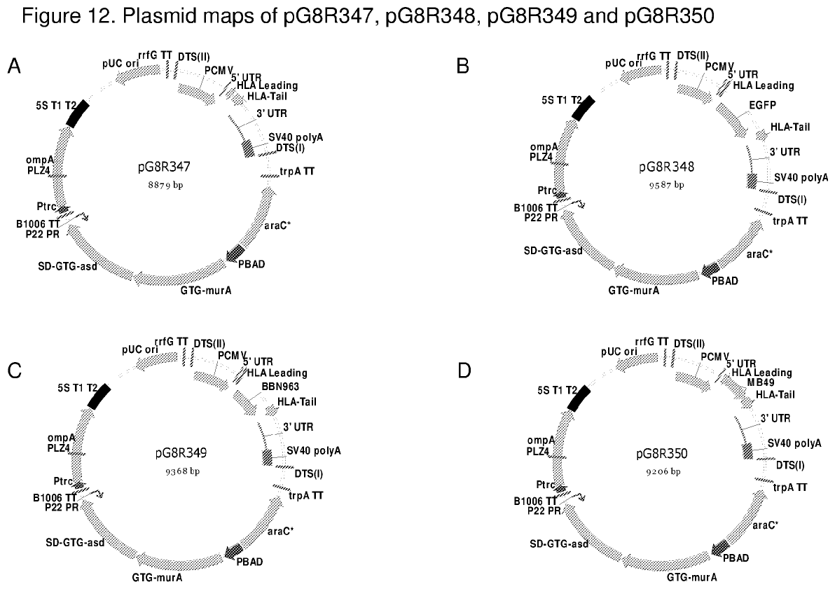

The 5′ and 3′ terminal nucleotide sequences of eukaryotic genes affect the translation of the gene (181-183). Thus, we generated plasmid pG8R347 (FIG. 12A) that includes the 5′ and 3′ terminal nucleotide sequence of HLAB gene.

To construct pG8R347, we used HLAB (HLA-B) (NM_005514) Human Untagged Clone (ORIGENE Cat #SC124484) as a template to amply the sequence of the 5′ terminal of HLAB using primers HLAB-5′ Leading-gs and HLAB-Leading-MCS-ga and the 3′ terminal of HLAB using primers HLAB-tail-MCS-gs and HLAB-3′ tail-XhoI-ga. The above two fragments were cloned into pG8R320(FIG. 5B) cut with KpnI/XhoI to generate pG8R347. The lysis plasmid has sequences for HLAB 5′ terminus, leading and tail peptides and 3′ terminus which could be used to coupling antigen to MHC Class I Trafficking Signals to increase antigen presentation efficiency (180). The inclusion of 5′ and 3′ termini of HLAB increases the transcript stability and translational efficiency (184).

Similar, we inserted the egfp gene into plasmid pG8R347 to generate plasmid pG8R348 (FIG. 12B). To construct plasmid pG8R348, we used plasmid pYA4685 as a template to amplify the gene encoding EGFP using primers encoding EGFP was amplified with primers EGFP(HLAB)-KpnI-gs and EGFP(HLAB)-AvrII-ga and cloned into plasmid pG8R347 (FIG. 12A) cut with KpnI/AvrII to generate plasmid pG8R348. The lysis plasmid carries a gene encoding EGFP and could be used to track the Salmonella in mammalian cells, although this will require replacing the ompAΩplz4 construction that enables targeting to bladder tumor cells with sequences specifying a protein to target other cell types.

Tumor neoantigens can be presented by major histocompatibility complex proteins and recognized by T cells to induce anti-tumor immune responses. This approach has been used as therapeutic vaccines in preclinical models to promote tumor specific T-cell responses (185-188). Tumor neoantigens are derived from mutated proteins that lead to the generation of novel immune epitopes that are foreign to the body (189, 190). Vaccines targeting tumor neoantigens are a promising strategy for personalized cancer immunotherapy (188, 190-197). Due to the complex immune tolerance mechanisms in tumors, neoantigen based tumor vaccines are normally combined with immune checkpoint inhibitors. Clinical trials with this combination therapy demonstrated that the induction of neoantigen-specific CD4⁺ and CD8⁺ T cell responses and cytotoxic vaccine-induced T cells, had some efficacy in treating bladder cancer (188, 191, 198). Mouse derived BBN963 (199) and MB49 (200, 201) cell lines are commonly used as an in vitro and in vivo model of bladder cancer. Neoantigens have been identified in these two cell lines (202). These neoantigens were cloned into vector pG8R347 to generate plasmid pG8R349 (FIG. 12C) and pG8R350 (FIG. 12D) to be used in vaccine trials.

To construct pG8R349, the gene encoding neo-antigen BBN963 (202) was cut from with plasmid pUC57-BBN963 with KpnI/AvrII and cloned into plasmid pG8R347 (FIG. 12A) cut with same enzymes to generate plasmid pG8R349. The lysis plasmid carries a gene encoding neo-antigen BBN963.

To construct pG8R350, the gene encoding neo-antigen MB49 (202) was cut from with plasmid pJET1.2-MB49 with KpnI/AvrII and cloned into plasmid pG8R347 (FIG. 12A) cut with same enzymes to generate plasmid pG8R350. The lysis plasmid carries a gene encoding neo-antigen MB49.

### Example 12. Construction of Dual Plasmids to Cause CCTS Strains to Target Bladder Tumor Cells and then Express a Gene Sequence Under the Control of P_{EF1α} Promoter

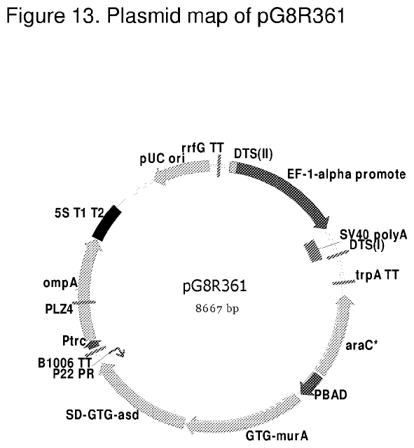

The viral derived PCMV promoter is a strong promoter that has been widely used to express genes in eukaryotic cells as in DNA or viral vectors, such as adenovirus. However, it could be silenced in certain cell types due to methylation, leading to considerable variability gene expression in different cell types (203, 204). Human elongation factor-1α is a constitutive human promoter that can drive ectopic gene expression homogeneously and persistently in vivo and in vitro (205-207). It can replace PCMV when PCMV has diminished activity due to being silenced. We therefore generated the regulated delayed lysis plasmid pG8R361 (FIG. 13) that has a pUC on and can express ompAΩplz4 in Salmonella to display the synthesized OmpAΩPLZ4 on the cell surface and after lysis of the CCTS cell employs the P_{EF1α} promoter to express inserted genes in eukaryotic cells.

For the construction of pG8R361 (FIG. 13), we fused two fragments. We used plasmid pG8R320 (FIG. 5B) as a template to generate a 7.4 kb fragment with primers 4545-(ForPEF1a) KpnIXmaINotIXhoI-s and 4545(ForPEF1a)-a2. Then, with plasmid pLVX EF1α IRES Puro N (BEI NR52973) as a template, a fragment containing the P_{EF1α} promoter was amplified with primers PEF1a-s and PEF1a-KpnI-a. The two fragments were then assembled into plasmid pG8R361. The regulated delayed lysis plasmid has a pUC on and can express ompAΩplz4 in Salmonella to display the synthesized OmpAΩPLZ4 on the cell surface to target bladder cancer cells and after lysis of the CCTS cell employs the P_{EF1α} promoter to express an inserted gene sequence in eukaryotic cells (208-212), in this case into bladder tumor cells. The P_{EF1α} and P_{trc} ompAΩplz4 in the dual plasmid vector pG8R361 are separated by the regulated delayed lysis cassette araC P_{araBAD} GTG murA GTG asdA.

### Example 13. Construction of Dual Plasmids to Cause CCTS Strains to Target Bladder Tumor Cells and then Express a Gene Sequence Encoding HAC-PD1 to Block PD1L1 and Activate T Cells and CXCL11 to Recruit Immune Cells

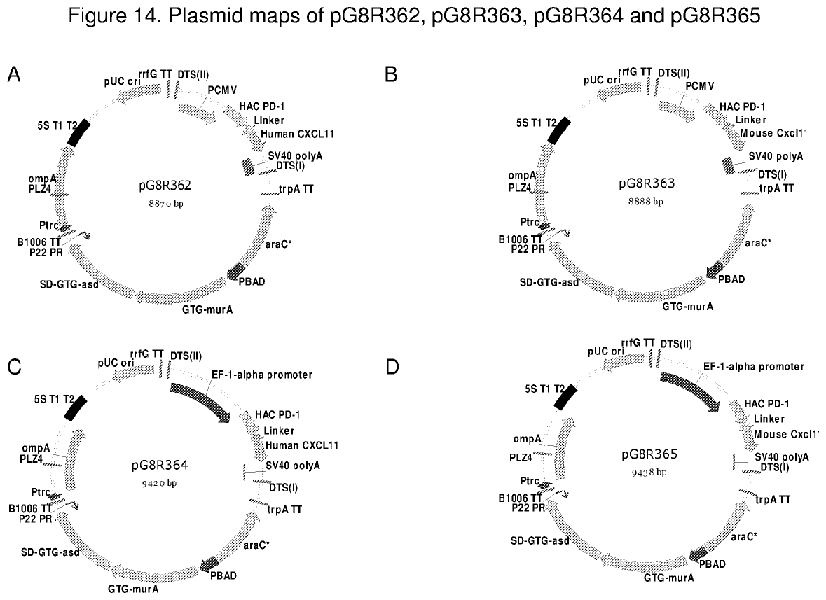

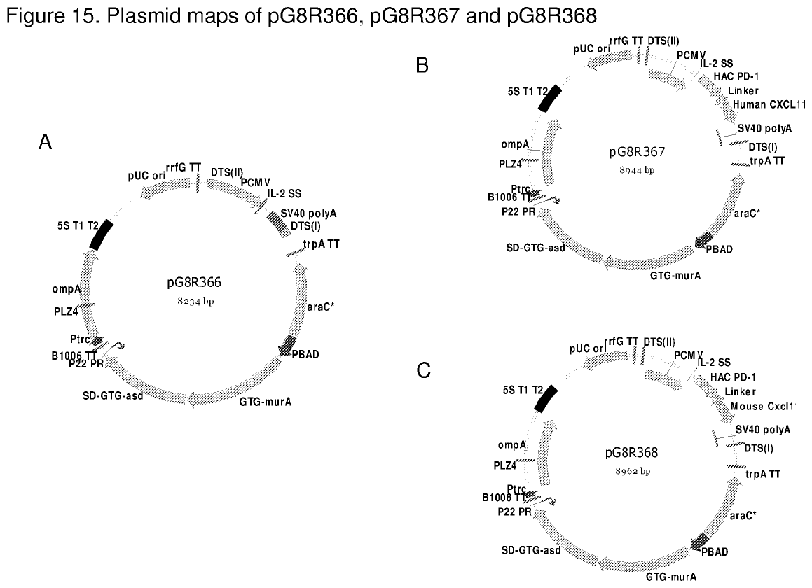

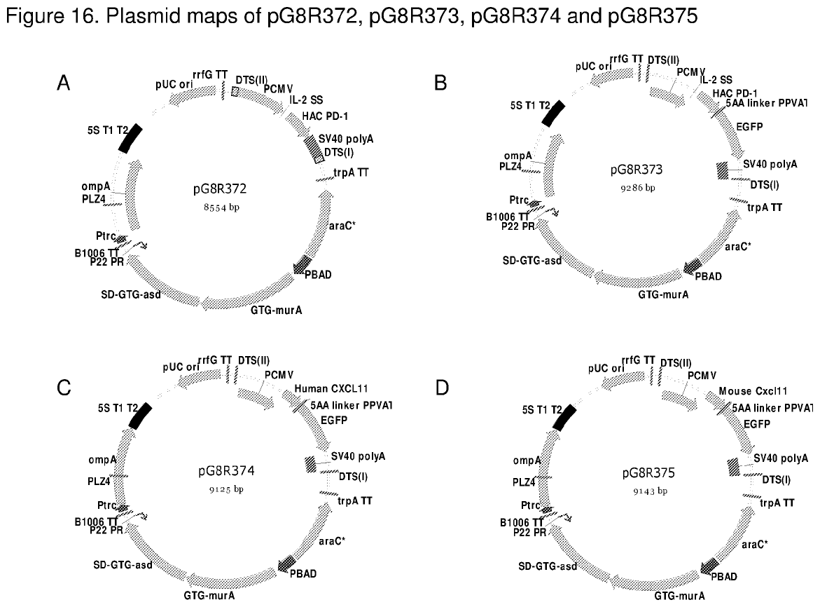

The interaction between the Programmed cell death protein-1 (PD-1) and programmed cell death ligand-1 (PD-L1) functions as a T cell checkpoint to regulate T cell responses. Cancer cells upregulate the levels of PD-L1 to evade immune detection and elimination (213). Monoclonal antibodies blocking PD1 and PDL1 have been approved as effective immunotherapies against different tumors (214-219). However, use of antibodies have inherent limitations that include poor and slow distribution within hypoxic regions of large tumors (220, 221) and immune-related adverse events, such as Fc-mediated cytotoxic immune responses (222) and severe cytokine associated inflammatory and immunological process (223, 224). For monoclonal antibodies against PD1/PLD1, they can also reduce circulation of T cell numbers in patients (225-227). To overcome these shortcomings, a soluble fragment of the PD1 ectodomain, the high-affinity consensus (HAC)-PD1, was identified as an alternative agent that exhibits improved antitumor responses and avoids antibody limitations (228, 229). The HAC-PD1 (228, 229) has an over 40,000-fold higher affinity for PD-L1 than native PD1 (229) and 32- and 12-times higher affinity than the FDA-approved anti-PD-L1 antibodies atezolizumab and durvalumab, respectively (230). At the same dose and schedule through intratumoral injection, it is also more effective than an anti-PD1 antibody in inducing anti-cancer immunity (229). Multiple vectors carrying the gene encoding HAC-PD1 are depicted in FIGS. 14, 15 and 16. FIG. 14 depicts plasmids that contain sequences encoding HAC-PD1 combined with CXCL11 for combinational immunotherapy. FIG. 15 depicts plasmids that contain sequences encoding IL2 SS-HAC-PD1 and CXCL11 for combinational immunotherapy. FIG. 16 depicts plasmids that carry sequences encoding HAC-PD1, HAC-PD1 and EGFP, CXCL11 and EGFP. All these plasmids display the synthesized OmpAΩPLZ4 on the cell surface to target bladder cancer cells and after lysis of the CCTS cell employ the P_{CMV} or P_{EF1α} promoter to express the inserted eukaryotic genes in bladder tumor cells.

To construct plasmid pG8R362 (FIG. 14A), we used plasmid pMaI-HAC-PD1 (228, 229) as a template to amplify the gene encoding HAC-PD1 using primers HAC-PD1-KpnI-s and HAC-PD1-Linker-a1 and then extended by PCR with primers HAC-PD1-KpnI-s and HAC-PD1-Linker-a2. Then with CXCL11 (NM_005409) Human Tagged ORF Clone (human CXCL11(Myc-DDK-tagged), ORIGENE Cat #RC210320) as a template, the gene encoding human CXCL11 was amplified with primers Linker-hCXCL11-s and Human-CXCL11-NotI-a. The two fragments were inserted into plasmid pG8R320 (FIG. 5B) cut with KpnI/NotI to generate plasmid pG8R362. The lysis plasmid carries the gene encoding HAC-PD1 fused with human CXCL11 with a 36 amino acid linker under the control of a P_{CMV} promoter. The 36 aa linker GGS(GGGSE)₅(GGGS)₂ (SEQ ID NO: 133) was inserted to enable fusion of HAC-PD1 and human CXCL11 (231). The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the fused gene sequence.

To construct plasmid pG8R363 (FIG. 14B), we used plasmid pMaI-HAC-PD1 (228, 229) as a template to amplify the gene encoding HAC-PD1 using primers HAC-PD1-KpnI-s and HAC-PD1-Linker-a1 and then extended by PCR with primers HAC-PD1-KpnI-s and HAC-PD1-Linker-a2′). Then with Cxcl11 (NM_019494) Mouse Tagged ORF Clone (mouse CXCL11 (Myc-DDK-tagged), ORIGENE Cat #MR222244) as a template, the gene encoding mouse CXCL11 was amplified with primers Linker-mCXCL11-s and Mouse-CXCL11-NotI-a. The two fragments were inserted into plasmid pG8R320 (FIG. 5B) cut with KpnI/NotI to generate plasmid pG8R363. The lysis plasmid carries a gene encoding HAC-PD1 and fused with mouse CXCL11 with a 36 amino acid linker under the control of a P_{CMV} promoter. The 36 aa linker GGS(GGGSE)₅(GGGS)₂ (SEQ ID NO: 133) was inserted to enable fusion of HAC-PD1 and mouse CXCL11 (231). The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the fused gene sequence.

To generate plasmid pG8R364 (FIG. 14C), a fragment encoding HAC-PD1-hCXCL11 was cut from plasmid pG8R362 (FIG. 14A) with KpnI/NotI and cloned into plasmid pG8R361 (FIG. 13) cut with the same enzymes to generate plasmid pG8R364. The lysis plasmid carries a gene encoding HAC-PD1 that was fused with human CXCL11 using a 36 amino acid linker under the control of the P_{EF1α} promoter. The regulated delayed lysis plasmid has a pUC on and can express ompAΩplz4 in Salmonella to display the synthesized OmpAΩPLZ4 on the cell surface and after lysis of the CCTS cell employs the P_{EF1α} promoter to express the fused gene sequence.

To generate plasmid pG8R365 (FIG. 14D), a fragment encoding HAC-PD1-mCXCL11 was cut from plasmid pG8R363 (FIG. 14B) with KpnI/NotI and cloned into plasmid pG8R361 (FIG. 13) cut with the same enzymes to generate plasmid pG8R365. The lysis plasmid carries a gene encoding HAC-PD1 that was fused with mouse CXCL11 using a 36 amino acid linker under the control of the P_{EF1α} promoter. The regulated delayed lysis plasmid has a pUC on and can express ompAΩplz4 in Salmonella to display the synthesized OmpAΩPLZ4 on the cell surface and after lysis of the CCTS cell employs the P_{EF1α} promoter to express the fused gene sequence.

Secretion of proteins increases the levels of therapeutic molecules that can significantly enhance the efficacy of therapy at the site of the disease. The IL2 signal peptide is one of the most commonly used secretion facilitating sequences used for protein production in gene therapy research (232-234). To increase the secretion of HAC-PD1, we first generated plasmid pG8R366 (FIG. 15A) that carries the sequence encoding the IL2 secretion signal. We amplified the IL2 SS using primers IL2-s2 and IL2-a2. The sequence was inserted into plasmid pG8R320 (FIG. 5B) cut with KpnI to generate plasmid pG8R366. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express any gene sequence fused to the IL2 secretion signal sequence. We then inserted HAC-PD1 with human and mouse CXCL11 into pG8R366 to generate plasmids pG8R367 (FIG. 15B) and pG8R368 (FIG. 15C), respectively.

To generate plasmid pG8R367, the fragment encoding HAC-PD1-hCXCL11 was cut from plasmid pG8R362 (FIG. 14A) with KpnI/NotI and inserted into plasmid pG8R366 (FIG. 15A) cut with the same enzymes to generate plasmid pG8R367. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the gene sequence encoding HAC-PD1-human CXCL11 fused to the IL2 secretion signal.

To generate plasmid pG8R368, the fragment encoding HAC-PD1-mCXCL11 was cut from plasmid pG8R363 (FIG. 14B) with KpnI/NotI and inserted into plasmid pG8R366 (FIG. 15A) cut with the same enzymes to generate plasmid pG8R368. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the gene sequence encoding HAC-PD1-mouse CXCL11 fused to the IL2 secretion signal.

We also generated plasmid pG8R372 (FIG. 16A) which only has HAC-PD1 fused to the IL2 secretion signal. To generated plasmid pG8R372, we used plasmid pG8R367 (FIG. 15B) as a template to amplify the fragment encoding IL2 SS and HAC-PD1 with primers IL2-s2 and HAC-PD1-a. The fragment was inserted into plasmid pG8R320 (FIG. 5B) cut with KpnI/NotI to generate plasmid pG8R372. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the gene sequence encoding HAC-PD1-fused to the IL2 secretion signal.

To facilitate tracking of HAC-PD1 in mammalian cells, we tagged HAC-PD1 with EGFP to generate plasmid pG8R373 (FIG. 16B). For the construction of plasmid pG8R373, we used plasmid pG8R367 (FIG. 15B) as a template to amplify the fragment encoding IL2 SS-HAC-PD1 fusion using primers IL2-s2 and HAC-PD1-linker-EGFP-a. And then with plasmid pYA4685 (FIG. 2D) as a template, the gene encoding EGFP was amplified with primers HAC-PD1-linker-EGFP-s and C terminal EGFP-XhoINotI-a. The two fragments were inserted into plasmid pG8R320 (FIG. 5B) cut with KpnI/NotI to generate plasmid pG8R373. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the gene encoding fusion of IL2 secretion signal-HAC-PD1-fused and EGFP.

Similarly, we generate pG8R374 (FIG. 16C) and pG8R375 (FIG. 16D) in which CXCL11 fused with EGFP to track the behavior of CXCL11. To generate plasmid pG8R374, we used CXCL11 (NM_005409) Human Tagged ORF Clone (human CXCL11 (Myc-DDK-tagged), ORIGENE Cat #RC210320) as a template to amplify the gene encoding human CXCL11 using primers Human-CXCL11-KpnI-s and hCXCL11-EGFP-a. And then with plasmid pYA4685 (FIG. 2D) as a template, the gene encoding EGFP was amplified with primers HAC-PD1-linker-EGFP-s and C-terminal EGFP-XhoINotI-a. The two fragments were inserted into plasmid pG8R320 (FIG. 5B) cut with KpnI/NotI to generate plasmid pG8R374. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the gene encoding the fusion of human CXCL11 and EGFP.

To generate plasmid pG8R375 (FIG. 16D), we used CXCL11 (NM_019494) Mouse Tagged ORF Clone (mouse CXCL11 (Myc-DDK-tagged), ORIGENE Cat #MR222244) as a template to amplify the gene encoding mouse CXCL11 using primers Mouse-CXCL11-KpnI-s and mCXCL11-EGFP-a. And then with plasmid pYA4685 (FIG. 2D) as a template, the gene encoding EGFP was amplified with primers HAC-PD1-linker-EGFP-s and C-terminal EGFP-XhoINotI-a. The 2 fragments were inserted into plasmid pG8R320 (FIG. 5B) cut with KpnI/NotI to generate plasmid pG8R375. The regulated delayed lysis plasmid has a pUC on and can express ompAΩplz4 in Salmonella to display the synthesized OmpAΩPLZ4 on the cell surface and after lysis of the CCTS cell in situ employs the P_{cmv} promoter to express the gene encoding fusion of mouse CXCL11 and EGFP.

### Example 14. Insertion of the Nucleotide Sequence Encoding the Ten Amino Acids of the Luteinizing Hormone-Releasing Hormone (LHRH) Peptide Binding to the LHRH Receptor into the Third Exposed Loop of the S. Typhimurium ompA Gene to Cause CCTS Strains to Target Endometrial, Bladder, Ovarian, Prostate and Breast Tumors with Overexpression of LHRH Receptors

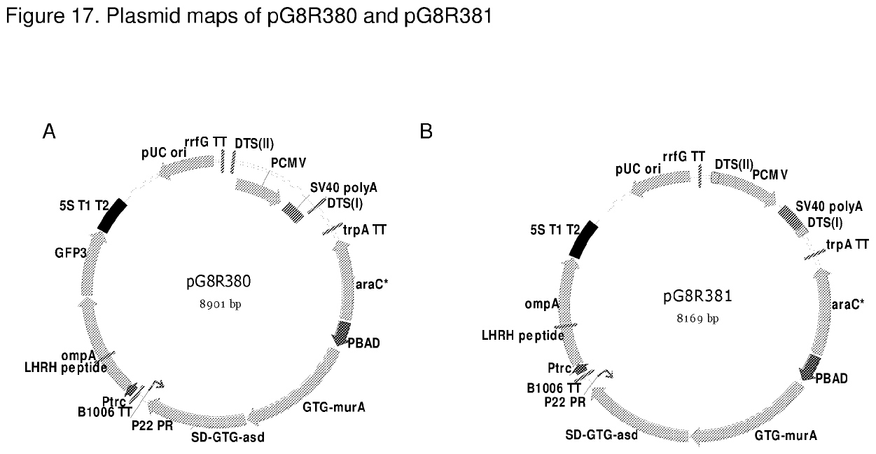

Luteinizing hormone-releasing hormone (LHRH) receptors are overexpressed in many cancers, including endometrial, bladder, ovarian, prostate and breast cancers (235-242), while limited in normal healthy tissues. LHRH has been employed to efficiently guide anticancer and imaging agents to cancer cells, thereby increasing the amount of these substances in tumors, but limiting delivery to normal tissues to reduce unnecessary exposure and toxicity (235, 241, 243, 244). We thus generated regulated delayed lysis plasmids pG8R380 (FIG. 17A) and pG8R381 (FIG. 17B) that have a pUC on and can express ompAΩlhrh in Salmonella to display the synthesized OmpAΩLHRH on the cell surface to target endometrial, bladder, ovarian, prostate and breast cancers and after lysis of the CCTS cell in the invaded tumor cell employ the P_{cmv} promoter to express a selected gene of importance to tumor therapy or identification.

To construct plasmid pG8R380 (FIG. 17A), we used plasmid pG8R320 (FIG. 5B) as a template to amplified a 540 bp fragment using primers P_{trc}-BcII-s and LHRH-a and a 707 bp fragment using primes LHRH-s and OmpAGFP-BstBI-a. And then with plasmid pYA4090 (FIG. 2C) as a template, the gene encoding GFP was amplified with primer GFP-s and GFP-BstBI-a. The above 3 fragments were then inserted into plasmid pG8R320 (FIG. 5B) cut with BcII/BstBI to generate plasmid pG8R380. The regulated delayed lysis plasmid has a pUC on and can express the ompAΩlhrh and gfp gene sequences in Salmonella to display the synthesized OmpAΩLHRH on the cell surface and GFP in the cytosol and after lysis of the CCTS cell employs the P_{cmv} promoter to express a selected gene to specify synthesis of a desired gene product.

To construct plasmid pG8R381 (FIG. 17B), the plasmid pG8R380 was cut with BstBI to remove the gene encoding GFP. The 8.1 kb fragment was self-ligated to generate plasmid pG8R381. The regulated delayed lysis plasmid has a pUC on and can express ompAΩlhrh and gfp in Salmonella to display the synthesized OmpAΩLHRH on the cell surface and after lysis of the CCTS cell employs the P_{cmv} promoter to express a selected gene to specify synthesis of a desired gene product.

### Example 15. Construction of Dual Plasmids to Cause CCTS Strains to Target Bladder Tumor Cells and then Express a Gene Sequence Encoding haPD1-IgG to Block PD1L1 Inactivation of T Cell Functions and a Gene Sequence Encoding KillerRed Mem to Kill Tumor Cells

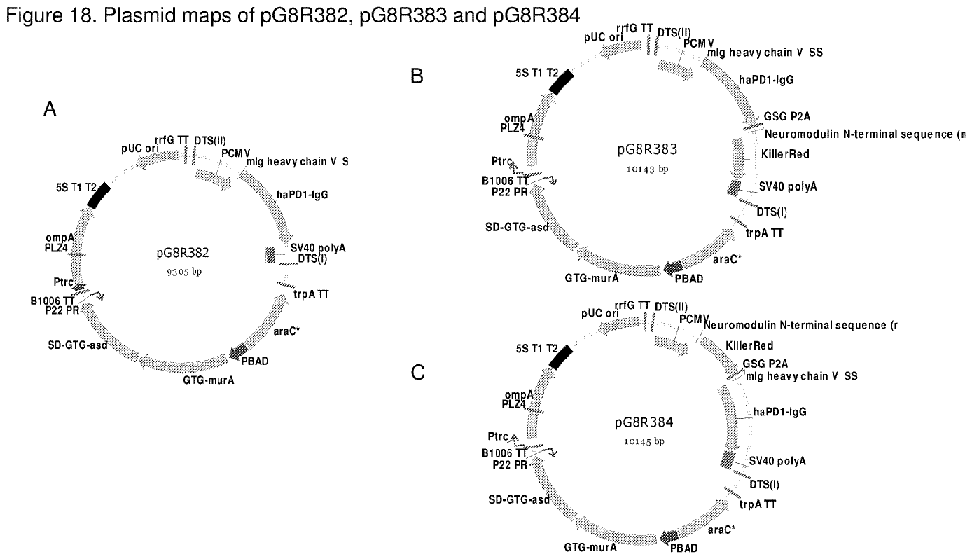

The HAC-PD1 (228, 229) has a 40,000-fold higher affinity for inactivating PD-L1 than native PD1 (229) and a 32- and 12-times higher inactivating ability than the FDA-approved anti-PD-L1 antibodies atezolizumab and durvalumab, respectively (230). HAC-PD1 is also more potent than an anti-PD1 antibody in inducing anti-cancer immunity using the same dose and schedule for intratumor injection (229). However, HAC-PD1, due to its small size, can leak from cells and thus elicit undesired immune responses against normal tissues. Furthermore, HAC-PD1 has a relatively short half-life and thus requires daily intratumoral injections (229). Thus, a HAC-PD1-IgG chimeric protein (haPD1-IgG, thereafter) can retain HAC-PD1 activity in tumors with a prolonged half-life and enhanced efficacy. Since IgG has a long half-life, use of the chimeric fusion protein can reduce the need for frequent administration. We therefore generated plasmids pG8R382 (FIG. 18A), pG8R383 (FIG. 18B) and pG8R384 (FIG. 18C) carrying a sequence encoding haPD1-IgG.

To construct plasmid pG8R382 (FIG. 18A), we used plasmid pCMV3-haPD1-IgG (Sinobiological, Project number BWH2-P) as a template to amplify the gene encoding haPD1-IgG using primers haPD1IgG-KpnI-s and haPD1IgG-NotI-a. The gene was inserted into plasmid pG8R320 (FIG. 5B) cut with KpnI/NotI to generate plasmid pG8R382. The regulated delayed lysis plasmid has a pUC on and can express ompAΩplz4 in Salmonella to display the synthesized OmpAΩPLZ4 on the CCTS cell surface to target bladder cancer and after lysis of the CCTS cell in tumor cells employs the P_{cmv} promoter to express the haPD1-IgG to prevent inactivation of T cells.

To construct plasmid pG8R383 (FIG. 18B), we used plasmid pCMV3-haPD1-IgG (Sinobiological, Project number BWH2-P) as a template to amplify the gene encoding haPD1-IgG using primers and haPD11gG-C-P2A-a. Then with plasmid pCS2-NXE+mem-KillerRed (Addgene plasmid #45761) as a template, the gene encoding KillerRed mem was amplified using primers P2A-KillerRed-Mem-s and KillerRed-NotIXhoI-a and then extended by PCR using primers P2A-gs and KillerRed-NotIXhoI-a. The two fragments were then inserted into plasmid pG8R320 (FIG. 5B) cut with KpnI/NotI to generate plasmid pG8R383. The lysis plasmid carries genes encoding haPD1-IgG and KillerRed mem separated by the P2A peptide under the control of the P_{cmv} promoter to enable synthesis of haPD1-IgG and KillerRed mem in tumor cells invaded by the CCTS strain. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express synthesis of haPD1-IgG to prevent inactivation of T cells and KillerRed mem to kill the tumor cells.

To construct plasmid pG8R384 (FIG. 18C), we used plasmid pG8R343 (FIG. 10A) as a template to amplify the gene encoding KillerRed mem using primers KillerRed-Mem-KpnI-s and KillerRed-C-P2A-a2. And then with plasmid pCMV3-haPD1-IgG (Sinobiological, Project number BWH2-P) as a template, the gene encoding haPD1-IgG was amplified with primers P2A-haPD11 gG-s and haPD11 gG-NotI-a. The two fragments were then inserted into plasmid pG8R320 (FIG. 5B) cut with KpnI/NotI to generate plasmid pG8R384. The lysis plasmid carries genes encoding KillerRed mem and haPD1-IgG separated by P2A peptide under the control of a P_{cmv} promoter to enable synthesis of KillerRed mem and haPD1-IgG. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express KillerRed mem to kill the tumor cell and haPD1-IgG to prevent the inactivation of T cells. Note that the only difference between pG8R343 and pG8R344 is the order of the sequences encoding the synthesis of haPD1-IgG and KillerRed.

### Example 16. Insertion of the Nucleotide Sequence Encoding the Single-Chain Fragment Variable (scFv) Targeting HER2 into the Third Exposed Loop of the S. Typhimurium ompA Gene to Cause CCTS Strains to Target Bladder, Prostate and Breast Tumors

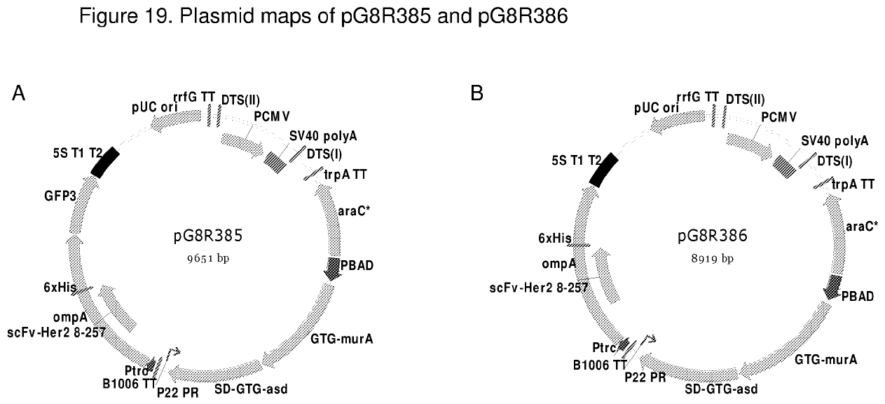

Human epidermal growth factor receptor 2 (HER2) is overexpressed in bladder, gastric, prostate and breast cancers (239-242, 245). Single-chain fragment variables (scFv, ˜25 kDa) penetrate tumors better than large IgG (246) and have faster clearance rates from the circulation to provide greater efficacy (247, 248). The scFv targets Her2 to bind to ErB2+ cells to potentiate delivery of exogenous DNA and siRNA into ErB2+ cells (249-251). We thus generated regulated delayed lysis plasmids pG8R385 (FIG. 19A) and pG8R386 (FIG. 19B) that have a pUC on to synthesize scFv targeting Her2 on the Salmonella cell surface to target attaching to and invading bladder, gastric, prostate and breast cancers and after lysis of the CCTS cell in cancer cells employs the P_{cmv} promoter to drive expression of desired gene sequences important for tumor therapy.

To construct plasmid pG8R385 (FIG. 19A), we used plasmid pG8R320 (FIG. 5B) as a template to amplify a 536 bp fragment using primers P_{trc}-BcII-s and OmpA-Her2-a and a 694 bp fragment using primers OmpA-Her2-s and ompAGFP-BstBI-a. And then with plasmid pACgp67B-Her2 (Addgene Plasmid #10794) as a template (251), the gene encoding Her2 scFv was amplified with primers Her2-s and Her2His-a. With plasmid pYA4090 (FIG. 2C) as a template, the gene encoding GFP was amplified with primer GFP-s and GFP-BstBI-a. The 4 fragments were inserted into plasmid pG8R320 cut with BcII/BstBI to generate plasmid pG8R385. The regulated delayed lysis plasmid has a pUC on and can express ompAfΩHer2 scFv and gfp in Salmonella to display the synthesized OmpAΩHer2 scFv on the CCTS cell surface and GFP in the cytosol and after lysis of the CCTS cell in a cancer cell employs the P_{cmv} promoter to express an inserted gene sequence of importance for cancer cell/tumor therapy.

To construct plasmid pG8R386 (FIG. 19B), the plasmid pG8R385 was cut with BstBI to remove the gene encoding GFP. The 8.9 kb fragment was self-ligated to generate plasmid pG8R386. The regulated delayed lysis plasmid has a pUC on and can express ompAΩHer2 scFv in Salmonella to display the synthesized OmpAΩHer2 scFv on the cell surface and after lysis of the CCTS cell employs the P_{cmv} promoter to express an inserted gene sequence of importance for cancer cell/tumor therapy.

### Example 17. Construction of Universal Vaccine Vectors to Enable Expression of Both Bacterial and Eukaryotic Genes by Insertion of Selected Nucleotide Sequences after the P_{trc} or P_{trc} Bla SSopt Promoter and the P_{CMV} Promoter, Respectively

The regulated delayed lysis plasmid pG8R320 (FIG. 5B) has a pUC on and can express the bacterial gene ompAΩplz4 under the control of the P_{trc} promoter in Salmonella to display the synthesized OmpAΩPLZ4 on the cell surface and after lysis of the cell employs the P_{cmv} promoter to express an inserted gene in eukaryotic cells. It is mainly used with Salmonella strains possessing the ΔompA mutation and mainly targeting bladder cancer cells with PLZ4 peptide. This mutation may not be required in all situations. The targeting to bladder cancer cells limits its usage for other cancer cells. Thus, a universal vector without the OmpAΩPLZ4 can be used for other purposes, such as targeting other cancer types or as a dual antigen delivery system. We generated plasmids pG8R388 (FIG. 19A) and pG8R389 (FIG. 19B) as two universal vectors to enable their use to express and deliver proteins of both bacterial and eukaryotic origins.

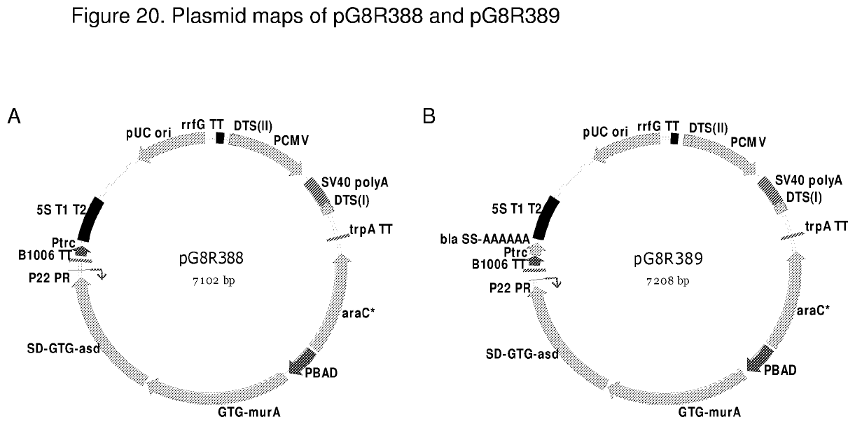

To construct pG8R388 (FIG. 20A), we used plasmid pYA3342 (FIG. 1A) as a template to amplify a 145 bp fragment containing the P_{trc} promoter using primers Ptrc-BcII-s and 4545Ptrc-a. The fragment was inserted into plasmid pG8R320 (FIG. 5B) cut with BcII/BstBI to generate plasmid pG8R388. The regulated delayed lysis plasmid has a pUC on and can express bacterial genes in the Salmonella cytosol and after invasion into a eukaryotic cell and lysis can employ the P_{cmv} promoter to express the eukaryotic gene in animal cells.

To construct pG8R389 (FIG. 20B), we used plasmid pG8R114 (FIG. 2B) as a template to amplify a 237 bp fragment containing the P_{trc} promoter with an optimized bla secretion signal (SS) (68) using primers Ptrc-BcII-s and 4545PtrcBlaAAA-a. The fragment was inserted into plasmid pG8R320 (FIG. 5B) cut with BcII/BstBI to generate plasmid pG8R389. The regulated delayed lysis plasmid has a pUC on and can express bacterial gene fused with bla SSopt in Salmonella and secrete the synthesized protein into the periplasm and invasion into a eukaryotic cell and lysis can employ the P_{cmv} promoter to express the eukaryotic gene in animal cells.

### Example 18. Construction of Dual Plasmids to Cause CCTS Strains to Target Bladder Tumor Cells with LHRH Peptide or HER2 scFv and then Express a Gene Sequence Encoding KillerRed to Potentiate Tumor Cell Killing

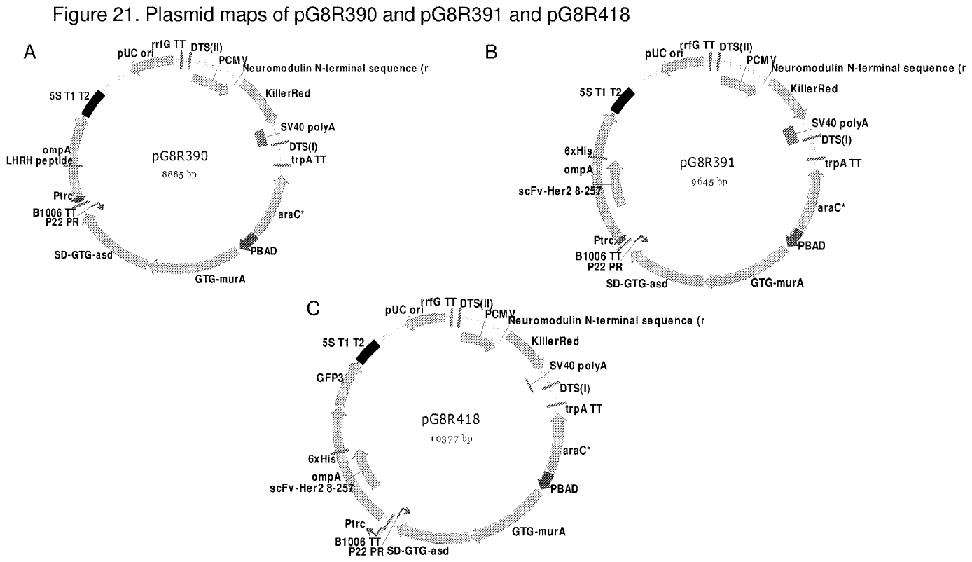

To validate the function of the LHRH peptide and HER2 scFv to target bladder tumor cells, we generated plasmids pG8R390 (FIG. 21A), pG8R391 (FIG. 21B) and pG8R418 (FIG. 21C) with KillerRed mem to potentiate tumor cell killing. To construct pG8R390 (FIG. 20A), the gene encoding KillerRed mem was cut from pG8R327 (FIG. 8C) with KpnI/Not and cloned into plasmid pG8R381 (FIG. 17B) cut with KpnI/NotI to generate plasmid pG8R390. The regulated delayed lysis plasmid has a pUC on and can express ompAΩlhrh in Salmonella to display the synthesized OmpAΩLHRH on the cell surface and after lysis of the CCTS cell in situ employs the P_{cmv} promoter to express the gene encoding KillerRed mem to potentiate tumor cell killing.

To construct pG8R391 (FIG. 20B), the gene encoding KillerRed mem was cut from pG8R327 (FIG. 8C) with KpnI/Not and cloned into plasmid pG8R386 (FIG. 19B) cut with KpnI/NotI to generate plasmid pG8R391. The regulated delayed lysis plasmid has a pUC on and can express ompAΩher2 scFv in Salmonella to display the synthesized OmpAΩHer2 scFv on the CCTS cell surface and after lysis of the CCTS cell after invading into a cancer cell employs the P_{cmv} promoter to express the gene encoding KillerRed mem to potentiate tumor cell killing.

To construct pG8R391 (FIG. 21C), the gene encoding KillerRed mem was cut from pG8R327 (FIG. 8C) with KpnI/Not and cloned into plasmid pG8R385 (FIG. 19A) cut with KpnI/NotI to generate plasmid pG8R418. The regulated delayed lysis plasmid has a pUC on and can express ompAΩher2 scFv in Salmonella to display the synthesized OmpAΩHer2 scFv on the CCTS cell surface and after lysis of the CCTS cell after invading into a cancer cell employs the P_{cmv} promoter to express the gene encoding KillerRed mem to potentiate tumor cell killing.

### Example 19. Construction of Recombinant Plasmid CCTS Strains with Potential to Attach to and Invade into Bladder Tumor Cells to Deliver Desired Cargo to Directly and/or Indirectly Reduce Tumor Survival

All of the candidate CCTS strains listed in Table 4 possess the ΔompA11 mutation which can be substituted by a chromosomal ompAΩplz4 fusion allele and deletion of the asdA gene to enable establishment of a balanced-lethal vector-host strain after introduction of any of the recombinant plasmid vectors displayed in FIGS. 3 to 5 and 7 to 21, all of which encode synthesis of a receptor ligand facilitating specific targeting to bladder tumor cells. Most strains have the ΔP_{murA25}::TT araC P_{araBAD} murA mutation with a ΔasdA mutation to enable the display of the regulated delayed lysis in vivo phenotype that is desirable for delivery of cargoes synthesized by the CCTS strain and also for the delivery of plasmids serving as DNA vaccines to enable synthesis of a desired gene product by tumor cells. The inclusion of the relA mutation facilitates the completeness of lysis by uncoupling the dependance of growth on continued protein synthesis. Most CCTS strains in Table 4 display a means to cause a regulated delayed attenuation in vivo by cessation in the synthesis of the LPS outer core or the LPS 0-antigen. This phenotype also enhances the efficiency of CCTS strains to attach to and invade eukaryotic cells and in this case better display the modified outer membrane protein OmpAΩPLZ4 that is the means for targeting specific attachment to bladder tumor cells. These CCTS strains also have a means to display a regulated delayed synthesis of P_{trc} regulated gene insertions by the araC P_{araBAD} regulated expression of the lacl gene, an attribute that enhances the efficiency and frequency of in vivo colonization of the CCTS strains in target tissues. The presence of the Δ(wza-wcaM)-8 mutation facilitates complete lysis of strains with the regulated delayed lysis phenotype, enhances levels of plasmid encoded protein synthesis and precludes synthesis of exopolysaccharides that contribute to biofilm formation. Most strains have a ΔrecF mutation to reduce inter- and intra-plasmid recombination to enhance construct stability and a ΔendA mutation to eliminate the endonuclease that could degrade the plasmid vector upon lysis of the CCTS cell. Also present, is a ΔsifA mutation that enables the CCTS strain after invasion into a cell to escape the Salmonella containing vesicle (SCV) or endosome. This is important for release of DNA vaccines by CCTS cells since the DNA vaccine must be free to be directed to the tumor cell nucleus to enable transcription of the inserted gene sequence under the control of the plasmid encoded P_{CMV} or P_{EF1α} promoter. In this regard, the DNA vaccine components of all the plasmids containing sequence from pYA4545 (FIG. 1C) contain multiple sequences that direct the plasmid DNA to the cell nucleus (45). Since Salmonella infection into host cells induces pyroptosis that acts to destroy the nuclear organization and function, it is important that CCTS cells delivering DNA vaccines possess mutations such as the ΔsseL and ΔtlpA mutations to delay onset of pyroptosis and thus enhance expression of DNA vaccine encoded genes. Thus, many of the strains listed in Table 4 have such mutations and these mutations can be added to other strains using the suicide vectors listed in Table 2. Some of the strains listed in Table 4 have deletion mutations in the pagL, pagP, lpxR, eptA and arnT genes that alter the structure and activities of the LPS lipid A. These mutations may or may not contribute to the efficacy of CCTS constructs by altering the degree of inflammation in interacting with TLR4. Further modification of these activities can be accomplished by inclusion of the ΔpagP81::Pₗₚₚ lpxE and/or the ΔlpxR93::Pₗₚₚ lpxF deletion-insertion mutations that cause expression of codon-optimized Francisella tularensis genes to delete the 1′ of 4′ phosphates from lipid A to render it non-toxic but retain ability to bind to and activate TLR4. Since many CCTS gene activities are regulated by the sugars arabinose and rhamnose that must be supplied during in vitro cultivation but are absent in animal tissues, the timing of shut off of sugar-regulated gene expression after CCTS cell entry into an animal (human) host can be modulated by whether the sugars supplied during in vitro growth and retained in the cells are or are not quickly metabolized. Thus the mutations ΔaraBAD65::TT and ΔrhaBADSR515 are sometimes added to CCTS strains to delay shut off of the sugar regulated genes for several cell divisions. Additional refinements of strains can be achieved by inclusion of mutations such as ΔP_{tolR67}::::TT araC P_{araBAD} tolR that acts to increase production and release of outer membrane vesicles and other mutations that alter display or non-display of flagellar and fimbrial appendages or component parts to alter recruitment of innate immunity. These activities are well described in WO 2020/096994 A1 and WO 2021/222696 A1.

### Example 20. Evaluation of CCTS Constructs for Ability to Attach to and Invade Bladder Tumor Cells

CCTS strains with pG8R341 specifying synthesis of GFP by the CCTS strain and pG8R342 in which the EGFP activity must be synthesized by the bladder tumor cell after invasion by the CCTS strain. Based on the discussion in Example 19, we will be comparing constructs in χ12417 (ΔP_{murA25}::TT araC P_{BAD} murA ΔwaaL46 Δpmi-2426 ΔasdA27::TT araC P_{BAD} c2 ΔpagL64::TT rhaRS P_{rhaBAD} waaL Δ(wza-wcaM)-8 ΔrelA197::araC P_{BAD} lacl TT ΔrecF126 ΔsifA26 ΔompA11) that was used for our initial work and the much improved strains χ12735 (ΔP_{murA25}::TT araC P_{araBAD} murA ΔasdA33 ΔwaaL46 ΔpagL38::TT rhaRS P_{rhaBAD}2 waaL2 Δ(wza-wcaM)-8 ΔrelA1123 ΔrecF126 ΔsifA26 ΔendA2113 ΔsseL116 ΔtlpA181 ΔrhaBADSR515 ΔaraBAD65::TT ΔompA11) and χ12736 (ΔP_{murA25}::TT araC P_{araBAD} murA ΔasdA33 ΔwaaL46 ΔpagL64::TT rhaRS P_{rhaBAD}1 waaL1 Δ(wza-wcaM)-8 ΔrelA1123 ΔrecF126 ΔsifA26 ΔendA2113 ΔsseL116 ΔtlpA181 ΔrhaBADSR515 ΔaraBAD65::TT ΔompA11) (see Table 4). Strains will be grown in LB broth with 0.1% arabinose and with and without 0.1% rhamnose and evaluated for ability to attach to and invade bladder tumor cells as described in Example 1 and FIG. 6. It is expected based on the information provided in forgoing Examples that strains grown without rhamnose to prevent synthesis of the LPS 0-antigen will be most proficient in attaching to bladder tumor cells. It is also expected that CCTS strains χ12735 and χ12736 will be most proficient in inducing synthesis of EGFP.

### Example 21. Exemplary Sequences

Sequences and SEQ ID NOs related to embodiments described herein are provided infra before the references.

### Example 22. Evaluation of the Effect of 0-Antigen on the CCTS Constructs for Ability to Attach to and Invade Bladder Tumor Cells

Clinical trials showed only 3 out of 25 patients had Salmonella colonization at the tumor sites after intravenous injection (10), indicating that targeting efficiency of Salmonella should be increased. The OmpAΩPLZ4 fusion enables Salmonella displaying a bladder cancer targeting peptide on the surface of Salmonella to target bladder cancer cells. Lipopolysaccharide (LPS) is the structure that covers the Salmonella surface. Modifications of LPS in Salmonella can potentially affect Salmonella tumor targeting. LPS comprises Kdo-lipid A, inner core, outer core and O-antigen side chain (252). Kdo-lipid A is essential to the survival of Salmonella. The LPS mutations, ΔwaaL46, ΔwaaG42, and ΔwaaC41, which enable Salmonella to display defects in the synthesis of O-antigen, outer core and inner core, respectively, were introduced into Salmonella strains. It should be noted that waaC mutant defective in synthesis of the inner core are unable to synthesize and assemble the outer core and O-antigen whereas mutants unable to synthesize the outer LPS core also unable to display the O-antigen. These mutations were introduced into χ12614 to yield strain χ12812, χ12813, and χ12814, respectively (Table 5). A plasmid pG8R341 carrying multiple copies of ompAΩplz4 was introduced into these strains.

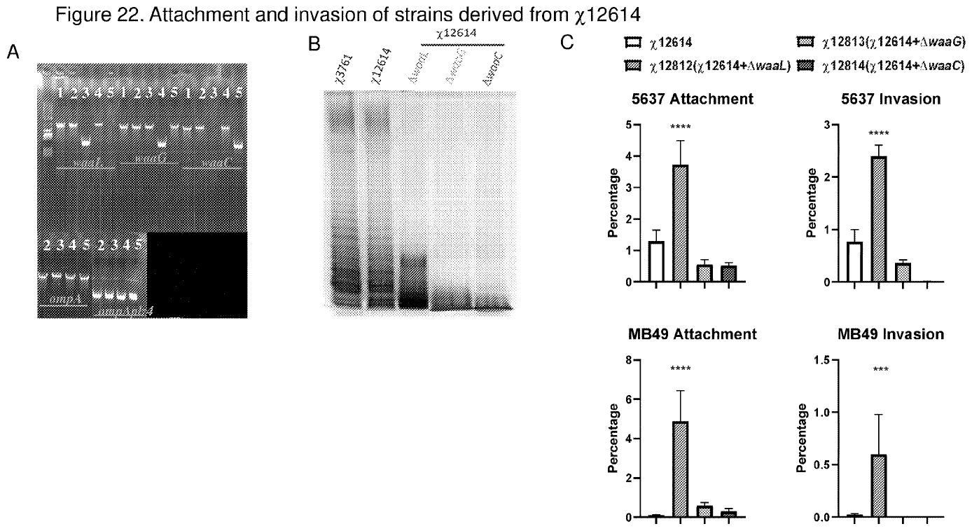

PCR with correspondent primers proved that strains derived from χ12614 have the correct expected genotype and the LPS gel proved that each strain has the right LPS phenotype human bladder cancer cell 5637 and mouse bladder cancer cells MB49 and BBN967 were performed in 24-well culture plates as described previously (152). The χ12614 lineage strains were grown in LB media until OD600 reached 0.85-0.9. The bacteria were collected and resuspended in DMEM media with 10% fetal bovine serum. A MOI 10:1 was used to infect cells for 1 hour. After infection, half of the monolayers were washed with PBS and lysed with PBS containing 0.1% sodium deoxycholate to assess the total number of attached bacteria. The other half of cells was incubated for 1 h with DMEM media containing 100 μg/ml gentamicin to eliminate extracellular bacteria. Monolayers were then lysed with PBS containing 0.1% sodium deoxycholate to assess the total number of internalized bacteria. As shown in FIG. 22C, strain χ12812 with ΔwaaL46 mutation displays the highest abilities in attachment and invasion compared to the other strains abilities to attach to and invade both human and mouse bladder cells.

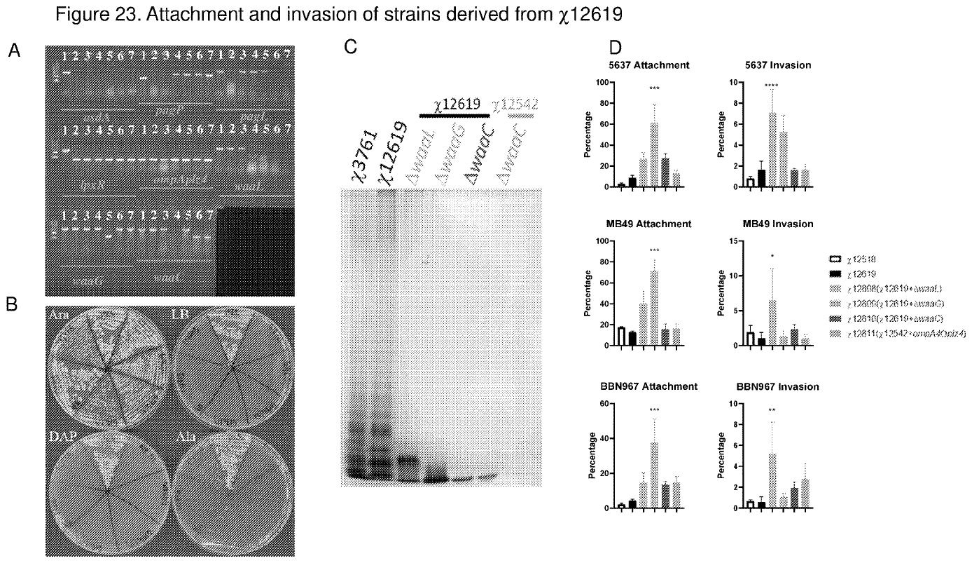

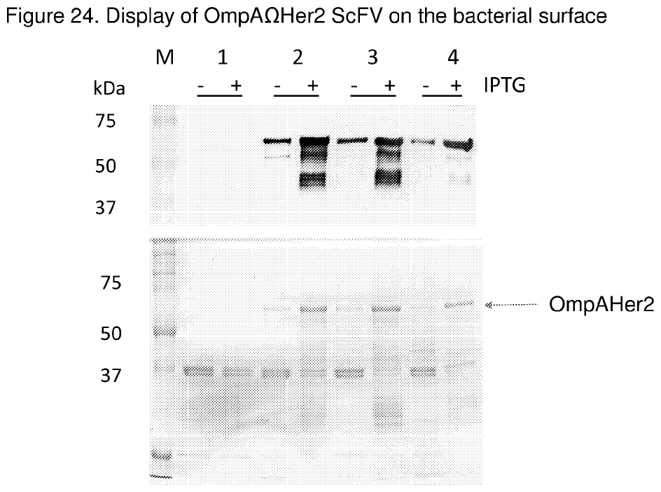

The LPS mutations, ΔwaaL46, ΔwaaG42, and ΔwaaC41 were also introduced into strain χ12619 to generate χ12808, χ12809, and χ12810, respectively. The ompAΩplz4 mutation (Table 2) was also introduced into strain χ12542 (Table 5) to yield strain χ12811 which has the identical genotypes as strain χ12810. These strains only have one copy of ompAΩplz4 in the chromosome. PCR with correspondent primers proved that strains derived from χ12619 have the correct expected genotype and the LPS gel proved that each strain had the right LPS phenotype (FIGS. 23 A and B). All the strains grew on LB with arabinose plates, but not on LB, LB with DAP and LB with alanine plates (FIG. 23C). The evaluation of the abilities of Salmonella cells to attach to and invade into human bladder cancer cell 5637 and mouse bladder cancer cells MB49 and BBN967 were performed in 24-well culture plates as described previously (152). The χ12619 lineage strains were grown in LB media with 0.1% arabinose until OD600 reaches 0.85-0.9. The bacteria were collected and resuspended in DMEM media with 10% fetal bovine serum. A MOI of 10:1 was used to infect cells for 1 hour. After infection, half of the monolayers were washed with PBS and lysed with PBS containing 0.1% sodium deoxycholate to assess the total number of attached bacteria. The other half of the monolayers was incubated for 1 h with DMEM media containing 100 μg/ml gentamicin to eliminate extracellular bacteria. Monolayers were then lysed with PBS containing 0.1% sodium deoxycholate to assess the total number of internalized bacteria. As shown in FIG. 23C, strain χ12809 with ΔwaaG46 mutation displayed the highest abilities in attachment, however, strain χ12808 with ΔwaaL46 displays highest abilities of invasion to both human and mouse bladder cells.

### Example 24. Display of OmpAΩHer2 ScFV on the Bacterial Surface Enables S. Typhimurium Cells to Preferentially Attach to Tumor Cells with Higher Her2 Production Levels

To evaluate the surface display of OmpAΩHer2 ScFV, strain χ12417 was transformed with plasmids pG8R385, pG8R418 and pG8R391 that carry the ompAΩher2 ScFV gene. The strains were grown in LB with 0.1% arabinose. 1 mM IPTG was added to induce the production of OmpAΩHer2 ScFV for 4 hours. The Salmonella outer member proteins (SOMPs) were prepared as described previously (62). The OmpAΩHer2 in strain χ12417 carrying any of the above plasmids can be detected in SOMPs portion of the SDS PAGE gel by Coomassie blue staining and western blot using anti-His6 antibody as an expected band around 65.1 kDa, but not the SOMPs from strain χ12417 (with no plasmid) (FIG. 24).

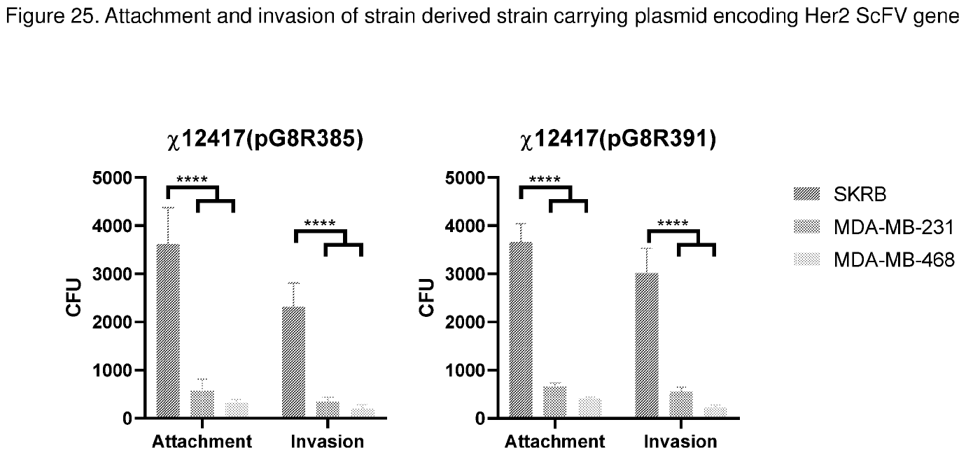

Different cell lines have different HER2 expression status (253). Her2 overexpression (SKBR-3, ATCC® HTB30) and low expression cell lines (MDA-MB-231(ATCC® CRM-HTB-26) and MDA-MB-468 (ATCC® HTB-132)) were used to detect the attachment and invasion of the χ12417(pG8R385) and χ12417(pG8R391) strains (FIG. 25). Overnight cultures of χ12417(pG8R385) and χ12417(pG8R391) were diluted into LB broth with 0.1% arabinose and grown until OD600 reached 0.9. The bacteria were washed once with PBS and then used to infect SKBR-3, MDA-MB-431 and MDA-MB-468 breast cancer cells at an MOI 10:1 for 1 hour. After infection, half of the monolayers were washed with PBS and lysed with PBS containing 0.1% sodium deoxycholate to assess the total number of attached bacteria. The other half of the monolayers were treated for 1 h with DMEM media containing 100 μg/ml gentamicin to eliminate extracellular bacteria. Monolayers were then lysed with PBS containing 0.1% sodium deoxycholate to assess the total number of internalized bacteria. The strain χ12417(pG8R385) and χ12417(pG8R391) producing OmpAΩPLZ4 attached to and invaded to the highest levels the Her2 overexpression cell line SKBR-3, but not to the Her2 low expression cell lines MDA-MB-431 and MDA-MB-468.

### Example 25. KillerRed Kills HEK293T Cells

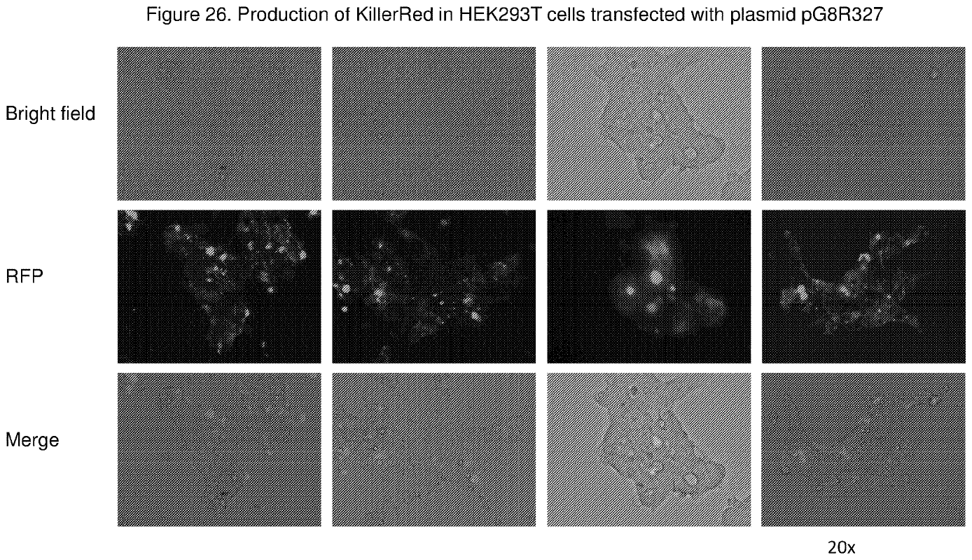

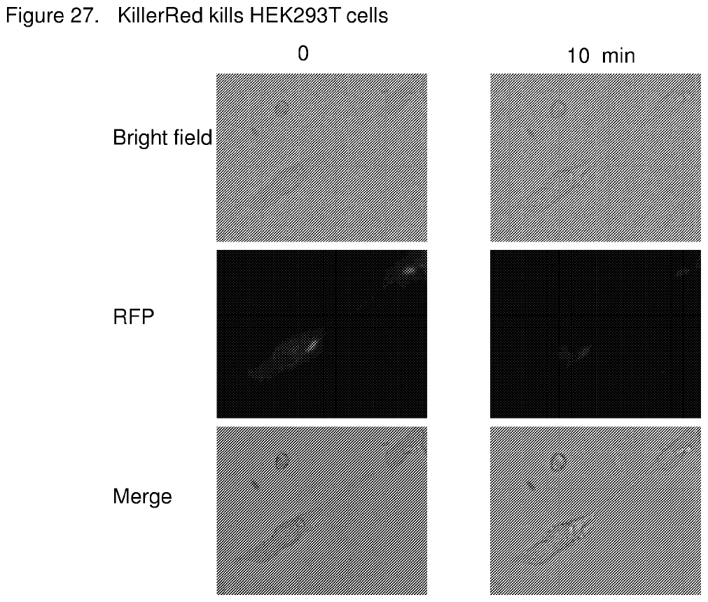

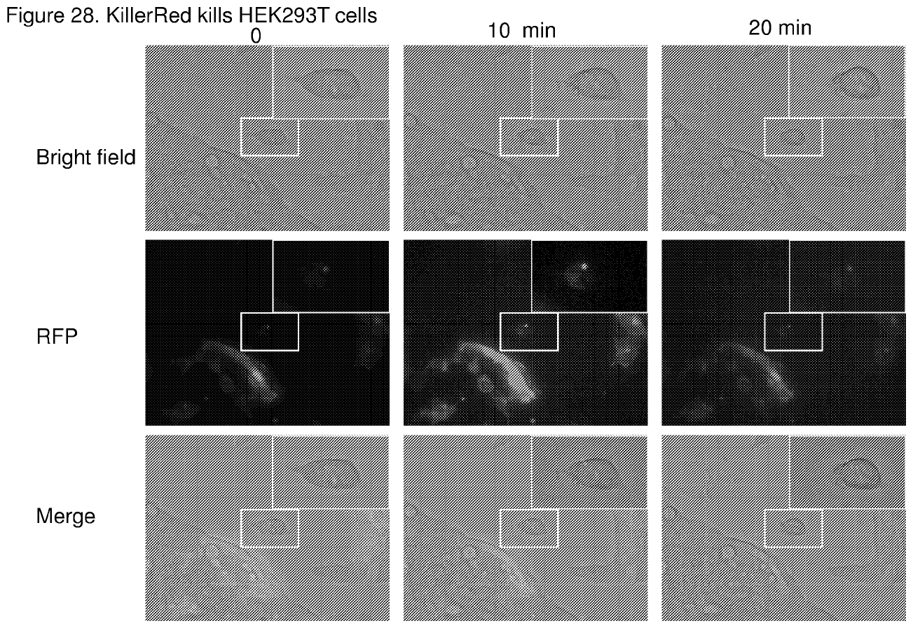

HEK293T cells were transfected with plasmid pG8R327 (FIG. 8C). The fluorescence was observed using EVOS Fl with RFP channel (Thermofisher). The RFP channel in EVOS Fl has 531/40 nm excitation and 593/40 nm emission. KillerRed can absorb 540-580 nm wavelength light and emit an longer 610 nm red light with maximum fluorescence excitation/emission at 585/610 nm. Although the RFP channel is not the optimal wavelength for KillerRed, strong red fluorescence signals were observed in HEK293T cells transfected with plasmid pG8R327 (FIG. 26). Before RFP channel excitation, the cells showed spindle shapes with smooth surfaces. After excitation for 10 min, membrane blebs appear on the surface of the cells (FIG. 27). The phenomena was also observed in other samples. Further excitation for an additional 10 min led to the cell morphology changing to round instead of spindle shaped (FIG. 28). This observation is at lower level but consistent with previous reports (170, 173, 175).

### Example 26. Evaluation of the CCTS Strains with Targeting Peptide In Vivo

The safety of CCTS strains uses oral and intravenous routes. Groups of 5 mice are inoculated with CCTS strains at varying doses ranging from 104 to 109 CFU. The mice are closely monitored for one month to see if any disease symptoms are observed.

Studies are conducted using multiple subcutaneous syngeneic tumor models to evaluate the distribution of CCTS strains based on the specific targeting peptide utilized. To establish the subcutaneous syngeneic tumor, approximately 1-5×10⁶ tumor cells are injected into the right flank of 6-8 week old mice. When tumor sizes reach approximately 100 mm³, the mice are administrated with the CCTS strains intravenously and orally using the highest safe dose determined from the previous experiment. At 24, 48, 72 and 96 hours, the spleen, liver, heart, lung, kidney, and tumors is harvested, weighted, homogenized and plated on LB agar with supplements. Fluorescence detection is performed on the tumors and organs to identify the presence of CCTS strains carry plasmids with the KillerRed or EGFP genes.

To test the target ability of the CCTS strain displayed PLZ4 peptide, BBN963 or MB49 murine bladder cancer cells are injected into the right flank of C57BL/6 mice. Mice are treated with CCTS strains with plasmids carrying the ompA3Ωplz4 gene and monitored as above. To test the target ability of LHRH peptide, high LHRH receptor expression cell lines, such as A2780 human ovarian cancer cells (1-5×10⁶) (235, 241, 243) or human breast cancer cells MCF-7 (ATCC® HTB-22) (254), MDA-MB-231 (ATCC® HTB-26), HCC1806 (ATCC® CRL-2335) are used to generate xenographs in athymic nu/nu mice. Mice are treated with a CCTS strain with a plasmid specifying the LHRH peptide and monitored as above. To test the target ability of Her2 ScFV, Her2 overexpression (SKBR-3, ATCC® HTB30) and low expression cell lines (MDA-MB-231(ATCC® CRM-HTB-26) and MDA-MB-468 (ATCC® HTB-132)), respectively, are used to generate xenographs in athymic nu/nu mice. Mice are treated with a CCTS strain with a plasmid specifying Her2 SCFV and monitored as above.

Over 60% of bladder tumors have little immune cell infiltration inside tumors (255). CXCL11 is a cytokine that can attract CD8 cytotoxic T cells (256, 257). The abilities of CCTS strains delivering plasmids pG8R319 (pBR ori, ompAΩplz4), pG8R320 (pUC ori, ompA3Ωplz4), pG8R322 (pBR ori, ompA3Ωplz4, cxcl11) and pG8R326 (pUC ori, ompA3Ωplz4, cxcl11) are compared to convert an immune “cold” to immune “hot” tumor. When tumors in C57BL/6 mice reach around 100 mm³, mice are fed with PBS, CCTS(pG8R319), CCTS(pG8R320), CCTS(pG8R322), or CCTS(pG8R326). Tumors will be harvested 24, 48, 72 or 96 hours later. Flow cytometry is used to compare the amount of CD8+ cells in different groups at these time points.

To further evaluate the abilities of CCTS strains to destroy tumors, C57BL6 mice carrying subcutaneous bladder tumors are used. When tumors reach the size of around 100 mm³, groups of 5 tumor-bearing mice are treated with a safe dose of CCTS strains with difference cargos, including CXCL11, haPD1, haPD1-IgG. The treatment could be once or multiple times. Mice are monitored for weight, tumor growth and survival. If tumors regress, mice are maintained and monitored to see if regrowth of tumors does or does not occur. Mice are euthanized once tumor sizes reach 1,500 mm³ or at the humane endpoint. Tumors are excised at the endpoint and either frozen directly in liquid nitrogen for storage or fixed in 10% formalin for histology or immunohistochemistry. In another experiment, tumors are collected before humane endpoint and split into 3 portions: formalin-fixation for immunohistochemical staining, fresh frozen for RNA and DNA extraction and deep sequencing; and single cell suspension for single cell sequencing.

Based on the characterizations of all the plasmid constructs in the previous Examples, we expect to observe specific tumor cell targeting in ectopic tumors and the expression of the encoded payloads to exhibit the desired effects.

### Example 27. Evaluation of the CCTS Strains with KillerRed In Vivo

KillerRed has been used in Photodynamic therapy (PDT) (258) (172). Mice carrying subcutaneous tumors are used. When tumors reach the size of around 100 mm³, tumor-bearing mice are treated with a safe dose of CCTS strains specifying synthesis of KillerRed. Fluorescence imaging of tumors are acquired daily in vivo using an IVIS-Spectrum (PerkinElmer, USA) with excitation wavelength of 570 nm and emission wavelength of 620 nm.

A suitable wavelength is used for PDT. When tumors reach the size of around 100 mm³, tumor-bearing mice are treated with a safe dose of CCTS strains delivering KillerRed. Tumors are either treated with a continuous wave or pulsed laser without causing excessive temperature effect on the skin surface. A validation parameter is described by Shirmanova et. al (259). The PDT is carried at 593 nm, 150 mW/cm², 270 J/cm² for the continuous laser wave daily for 7 days, or at 584 nm, 225 mW/cm², 337 J/cm² for the pulsed laser on the days 6, 7, and 8 of tumor growth. Skin surface temperature is monitored using an Infrared thermograph. After the treatment, randomly selected tumors in treated and untreated groups is collected and split into 3 portions: formalin-fixation for immunohistochemical staining, fresh frozen for RNA and DNA extraction and deep sequencing; and single cell suspension for single cell sequencing. During these studies, tumors are monitored with the size measured with a caliper twice a week until the mice reach humane endpoint.

### Example 28. Universal Vaccine Vectors to Enable Expression of Both Bacterial and Eukaryotic Genes by Insertion of Selected Nucleotide Sequences after the P_{trc} or P_{trc} Bla SSopt Promoter and the P_{CMV} Promoter, Respectively

A universal vaccine vector is a single vector that enables expression of genes both in prokaryotic and eukaryotic cells, even though it only specifies expression of gene only either in prokaryotic or eukaryotic cells. The regulated delayed lysis plasmids pG8R388 (FIG. 19A) and pG8R389 (FIG. 19B) are examples of two universal vectors to enable expression and delivery of proteins of both bacterial and eukaryotic origins. Plasmid pG8R388 has a pUC on and can express bacterial genes in the cytosol or on the surface of Salmonella and after invasion into a eukaryotic cell and lysis can employ the P_{cmv} promoter to express the eukaryotic gene(s) in animal cells. Plasmid pG8R389 has a pUC on and can express a bacterial gene fused with bla SSopt in Salmonella and secrete the synthesized protein into the periplasm and upon invasion into a eukaryotic cell and lysis can employ the P_{cmv} promoter to express the eukaryotic gene in animal cells. The prokaryotic promoter is not limited to P_{trc} and could be any promoter that functions in prokaryotic cells. Other prokaryotic promoters can be used. Multiple prokaryotic promoters can be used to drive the expression of multiple genes. Other secretion signals can be used to replace the bla SSopt. The eukaryotic promoter is not limited to P_{cmv} or P_{EF1α} and could be any promoter that functions in eukaryotic cells. Other eukaryotic promoters can be used. Multiple eukaryotic promoters can also be used to drive the expression of multiple genes.

Under the control of a prokaryotic promoter, a single gene can be expressed and multiple genes can also be expressed as an operon or protein fusion with or without suitable linkers. These linkers could be flexible, rigid, cleavable or dipeptide linkers. Some examples are listed by Chen et.al (260-262). An unexhaustive list includes (GGGS)n, (GGGGS)n, (G)n, (EAAAK)n, (XP)n, GCT KESGSVSSEQLAQFRSLD, EGKSSGSGSESKST, and GSAGSAAGSGEF. FIG. 17A illustrates plasmid pG8R380, in which an operon fusion links ompAΩlhrh and gfp. FIG. 19A and FIG. 21C illustrates plasmids pG8R385 and pG8R418, in which an operon fusion links ompAΩher2 SCFV and gfp.

Under the control of a eukaryotic promoter, a single gene can be expressed and multiple genes can also be expressed separated by one or multiple 2A cleavage peptides. The 2A peptide could be P2A, E2A, T2A, F2A or other 2A-like sequences and thus form bi-, tri-, and quad-, penta- or multiple cistronic vectors (263-265). Plasmid pG8R343 (FIG. 10), pG8R344 (FIG. 10), pG8R383 (FIG. 18), pG8R384 (FIG. 18) are examples of bicistrion vectors with the P2A peptide. One or multiple IRES sequences could be used to generate multicistronic constructions for simultaneously expression of multiple genes (177, 179, 266-269). Multiple gene can be linked by a linker described above. FIG. 14 and FIGS. 15B and 15C illustrate examples in which HAC PD1 is linked to CXCL11 through a linker. FIG. 16B-D illustrate examples in which HAC PD1 is linked to EGFP through a 5A linker PPVAT (SEQ ID NO: 155).

Exemplary Sequences
Sequence of pG8R314 (SEQ ID NO: 134 and 149)
Red highlighted: ompA sequence (SEQ ID NO:1 and 140 (DNA) and SEQ ID NO: 2 (amino acid))
Green highlighted: PLZ4 peptide (SEQ ID NO:3 and 141 (DNA) and SEQ ID NO:4 (amino acid))
Cyan highlighted: Linker (SEQ ID NO:5 (DNA) and SEQ ID NO:6 (amino acid))
Yellow highlighted: asd sequence (SEQ ID NO:7 and 143 (DNA) and SEQ ID NO:8 (amino acid))
GGC TCG

| | |
|---|---|
| 1 | GGA TCT TCC GGA AGA CCT TCC ATT CTG AAA TGA GCT GTT GAC AAT TAA TCA TCC GGC TCG |
| | CCT AGA AGG CCT TCT GGA AGG TAA GAC TTT ACT CGA CAA CTG TTA ATT AGT AGG CCG AGC |
| | M K K |
| 61 | 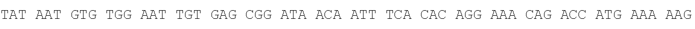 |
| | 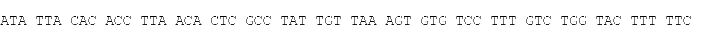 |
| | T A I A I A V A L A G F A T V A Q A A P |
| 121 | 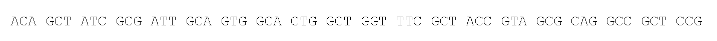 |
| | 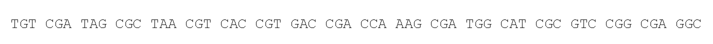 |
| | K D N T W Y A G A K L G W S Q Y H D T G |
| 181 | 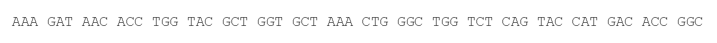 |
| | 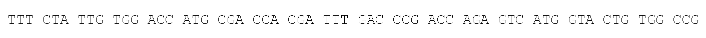 |
| | F I H N D G P T H E N Q L G A G A F G G |
| 241 | 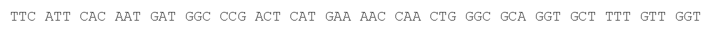 |
| | 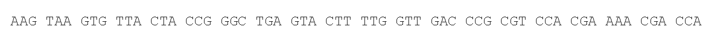 |
| | Y Q V N P Y V G F E M G Y D W L G R M P |
| 301 | 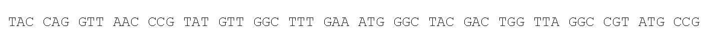 |
| | 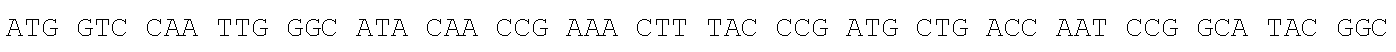 |
| | Y K G D N I N G A Y K A Q G V Q L T A K |
| 361 | 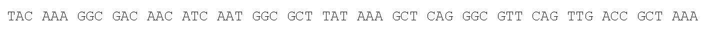 |
| | 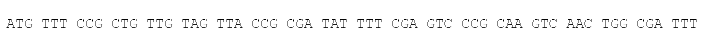 |
| | L G Y P I T D D L D V Y T R L G G M V W |
| 421 | 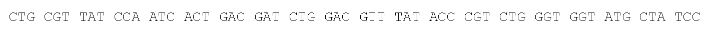 |
| | 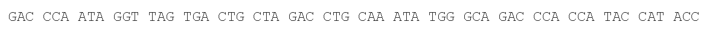 |
| | R A D T K S N V P G A C Q D G R M G F C |
| 481 | 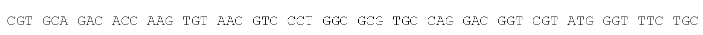 |
| | 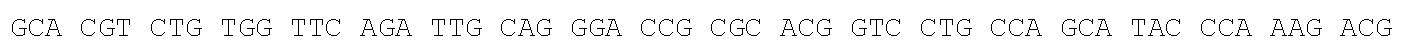 |
| | G G P S T K D H D T G V S P V F A G G I |
| 541 | 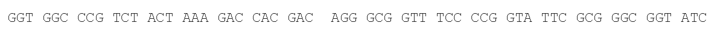 |
| | 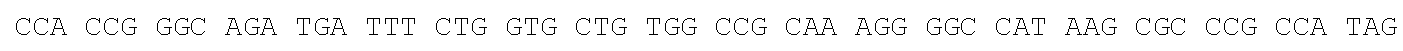 |
| | E Y A I T P E I A T R L E Y Q W T N N I |
| 601 | 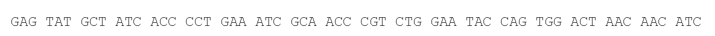 |
| | 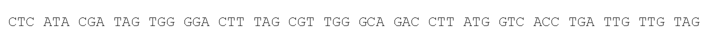 |
| | G D A N T I G T R P D N G L L S V G V S |
| 661 | 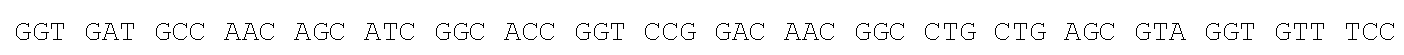 |
| | 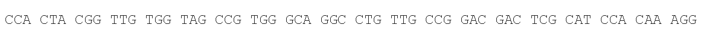 |
| | Y R F G Q Q E A A P V V A P A P A P A P |
| 721 | 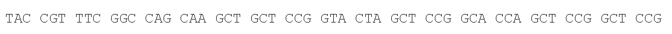 |
| | 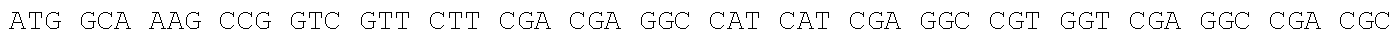 |
| | E V Q T K H F T L K S D V L F N F N K S |
| 781 | 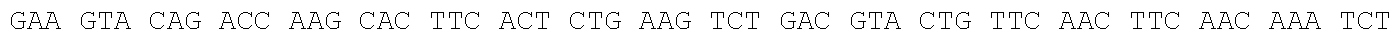 |
| | 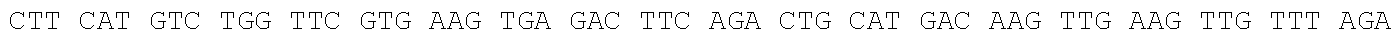 |
| | T L K P E G Q Q A L D Q L Y S Q L S N L |
| 841 | 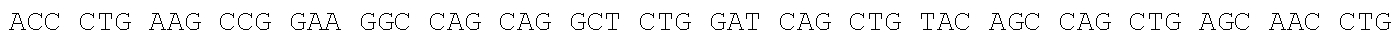 |
| | 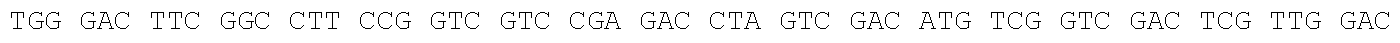 |
| | D P K D G S V V V L G F T D R I G S D A |
| 901 | 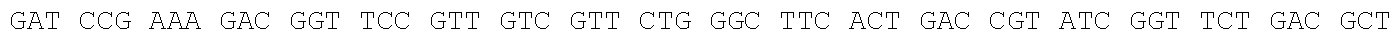 |
| | 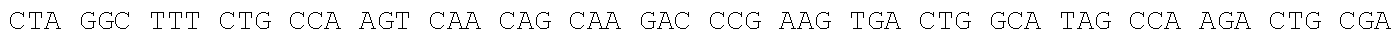 |
| | Y N Q G L S E K R A Q S V V D Y L I S K |
| 961 | 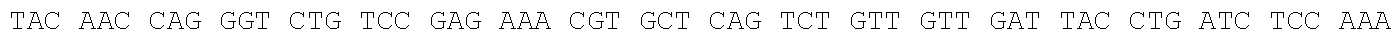 |
| | 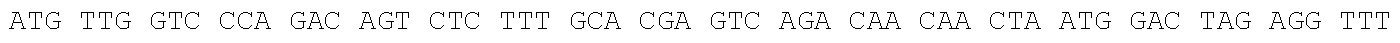 |
| | G I P S D K I S A R G M G E S N P V T G |
| 1021 | 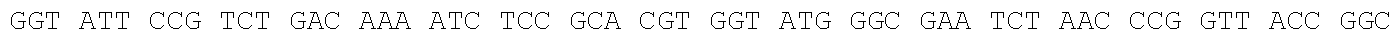 |
| | 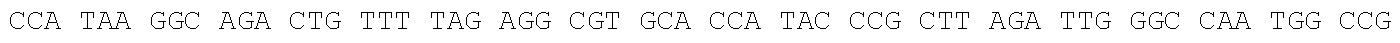 |
| | N T C D N V K P R A A L I D C L A P D R |
| 1081 | 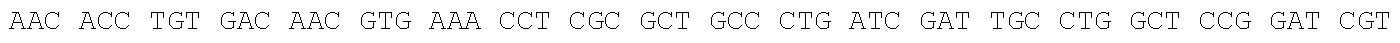 |
| | 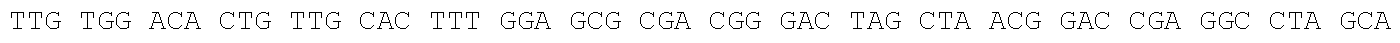 |
| | R V E I E V K G V K D V V T Q P Q A * |
| 1141 | 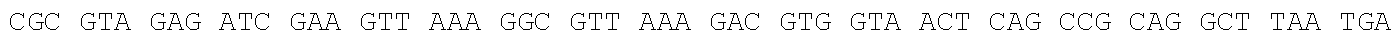 |
| | 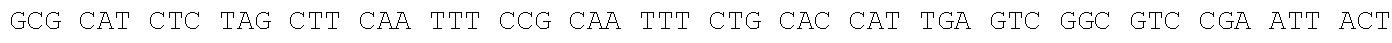 |
| 1201 | GCT CAA GCT TGG CTG TTT TGG CGG ATG AGA CAA GAT TTT CAG CCT GAT ACA GAT TAA ATC |
| | CGA GTT CGA ACC GAC AAA ACC GCC TAC TCT CTT CTA AAA GTC GGA CTA TGT CTA ATT TAG |
| 1261 | AGA ACG CAG AAG CGG TCT GAT AAA ACA GAA TTT GCC TGG CGG CAG TAG CGC GGT GGT CCC |
| | TCT TGC GTC TTC GCC AGA CTA TTT TGT CTT AAA CGG ACC GCC GTC ATC GCG CCA CCA GGG |
| 1321 | ACC TGA CCC CAT GCC GAA CTC AGA AGT GAA ACG CCG TAG CGC CGA TGG TAG TGT GGG GTC |
| | TGG ACT GGG GTA CGG CTT GAG TCT TCA CTT TGC GGC ATC GCG GCT ACC ATC ACA CCC CAG |
| 1381 | TCC CCA TGC GAG AGT AGG GAA CTG CCA GGC ATC AAA TAA AAC GAA AGG CTC AGT CGA AAG |
| | AGG GGT ACG CTC TCA TCC CTT GAC GGT CCG TAG TTT ATT TTG CTT TCC GAG TCA GCT TCC |
| 1441 | ACT GGG CCT TTC GTT TTA TCT GTT GTT TGT CGG TGA ACG CTC TCC TGA GTA GGA CAA ATC |
| | TGA CCC GGA AAG CAA AAT AGA CAA CAA ACA GCC ACT TGC GAG AGG ACT CAT CCT GTT TAG |
| 1501 | CGC CGG GAG CGG ATT TGA ACG TTG CGA AGC AAC GGC CCG GAG GGT GGC GGG CAG GAC GCC |
| | GCG GCC CTC GCC TAA ACT TGC AAC GCT TCG TTG CCG GGC CTC CCA CCG CCC GTC CTG CGG |
| 1561 | CGC CAT AAA CTG CCA GGC ATC AAA TTA AGC AGA AGG CCA TCC TGA CGG ATG GCC TTT TTG |
| | GCG GTA TTT GAC GGT CCG TAG TTT AAT TCG TCT TCC GGT AGG ACT GCC TAC CGG AAA AAC |
| 1621 | CGT TTC TAC AAA CTC TTT TGT TTA TTT TTC TAA ATA CAT TCA AAT ATG TAT CCG CTC ATG |
| | GCA AAG ATG TTT GAG AAA ACA AAT AAA AAG ATT TAT GTA AGT TTA TAC ATA GGC GAG TAC |
| 1681 | AGA CAA TAA CCC TGA TAA ATG CTT CAA TAA TGG AAG ATC TTC CAA CAT CAC AGG TAA ACA |
| | TCT GTT ATT GGG ACT ATT TAC GAA GTT ATT ACC TTC TAG AAG GTT GTA GTG TCC ATT TGT |
| 1741 | GAA ACG TCG GGT CGA TCG GGA AAT TCT TTC CCG GAC GGC GCG GGG TTG GGC AAG CCG CAG |
| | CTT TGC AGC CCA GCT AGC CCT TTA AGA AAG GGC CTG CCG CGC CCC AAC CCG TTC GGC GTC |
| 1801 | GCG CGT CAG TGC TTT TAG CGG GTG TCG GGG CGC AGC CAT GAC CCA GTC ACG TAG CGA TAG |
| | CGC GCA GTC ACG AAA ATC GCC CAC AGC CCC GCG TCG GTA CTG GGT CAG TGC ATC GCT ATC |
| 1861 | CGG AGT GTA TAC TGG CTT AAC TAT GCG GCA TCA GAG CAG ATT GTA CTG AGA GTG CAC CAT |
| | GCC TCA CAT ATG ACC GAA TTG ATA CGC CGT AGT CTC GTC TAA CAT GAC TCT CAC GTG GTA |
| 1921 | ATG CGG TGT GAA ATA CCG CAC AGA TGC GTA AGG AGA AAA TAC CGC ATC AGG CGC TCT TCC |
| | TAC GCC ACA CTT TAT GGC GTG TCT ACG CAT TCC TCT TTT ATG GCG TAG TCC GCG AGA AGG |
| 1981 | GCT TCC TCG CTC ACT GAC TCG GTG CGC TCG GTC GTT CGG CTG CGG CGA GCG GTA TCA GCT |
| | CGA AGG AGC GAG TGA CTG AGC GAC GCG AGC CAG CAA GCC GAC GCC GCT CGC CAT AGT CGA |
| 2041 | CAC TCA AAG GCG GTA ATA CGG TTA TCC ACA GAA TCA GGG GAT AAC GCA GGA AAG AAC ATG |
| | GTG AGT TTC CGC CAT TAT GCC AAT AGG TGT CTT AGT CCC CTA TTG CGT CCT TTC TTG TAC |
| 2101 | TGA GCA AAA GGC CAG CAA AAG GCC AGG AAC CGT AAA AAG GCC GCG TTG CTG GCG TTT TTC |
| | ACT CGT TTT CCG GTC GTT TTC CGG TCC TTG GCA TTT TTC CGG CGC AAC GAC CGC AAA AAG |
| 2161 | CAT AGG CTC CGC CCC CCT GAC GAG CAT CAC AAA AAT CGA CGC TCA AGT CAG AGG TGG CGA |
| | GTA TCC GAG GCG GGG GGA CTG CTC GTA GTG TTT TTA GCT GCG AGT TCA GTC TCC ACC GCT |
| 2221 | AAC CCG ACA GGA CTA TAA AGA TAC CAG GCG TTT CCC CCT GGA AGC TCC CTC GTG CGC TCT |
| | TTG GGC TGT CCT GAT ATT TCT ATG GTC CGC AAA GGG GGA CCT TCG AGG GAG CAC GCG AGA |
| 2281 | CCT GTT CCG ACC CTG CCG CTT ACC GGA TAC CTG TCC GCC TTT CTC CCT TCG GGA AGC GTG |
| | GGA CAA GGC TGG GAC GGC GAA TGG CCT ATG GAC AGG CGG AAA GAG GGA AGC CCT TCG CAC |
| 2341 | GCG CTT TCT CAT AGC TCA CGC TGT AGG TAT CTC AGT TCG GTG TAG GTC GTT CGC TCC AAG |
| | CGC GAA AGA GTA TCG AGT GCG ACA TCC ATA GAG TCA AGC CAC ATC CAG CAA GCG AGG TTC |
| 2401 | CTG GGC TGT GTG CAC GAA CCC CCC GTT CAG CCC GAC CGC TGC GCC TTA TCC GGT AAC TAT |
| | GAC CCG ACA CAC GTG CTT GGG GGG CAA GTC GGG CTG GCG ACG CGG AAT AGG CCA TTG ATA |
| 2461 | CGT CTT GAG TCC AAC CCG GTA AGA CAC GAC TTA TCG CCA CTG GCA GCA GCC ACT GGT AAC |
| | GCA GAA CTC AGG TTG GGC CAT TCT GTG CTG AAT AGC GGT GAC CGT CGT CGG TGA CCA TTG |
| 2521 | AGG ATT AGC AGA GCG AGG TAT GTA GGC GGT GCT ACA GAG TTC TTG AAG TGG TGG CCT AAC |
| | TCC TAA TCG TCT CGC TCC ATA CAT CCG CCA CGA TGT CTC AAG AAC TTC ACC ACC GGA TTG |
| 2581 | TAC GGC TAC ACT AGA AGG ACA GTA TTT GGT ATC TGC GCT CTG CTG AAG CCA GTT ACC TTC |
| | ATG CCG ATG TGA TCT TCC TGT CAT AAA CCA TAG ACG CGA GAC GAC TTC GGT CAA TGG AAG |
| 2641 | GGA AAA AGA GTT GGT AGC TCT TGA TCC GGC AAA CAA ACC ACC GCT GGT AGC GGT GGT TTT |
| | CCT TTT TCT CAA CCA TCG AGA ACT AGG CCG TTT GTT TGG TGG CGA CCA TCG CCA CCA AAA |
| 2701 | TTT GTT TGC AAG CAG CAG ATT ACG CGC AGA AAA AAA GGA TCT CAA GAA GAT CCT TTG ATC |
| | AAA CAA ACG TTC GTC GTC TAA TGC GCG TCT TTT TTT CCT AGA GTT CTT CTA GGA AAC TAG |
| 2761 | TTT TCT ACG GGG TCT GAC GCT CAG TGG AAC GAA AAC TCA CGT TAA GGG ATT TTG GTC ATG |
| | AAA AGA TGC CCC AGA CTG CGA GTC ACC TTG CTT TTG AGT GCA ATT CCC TAA AAC CAG TAC |
| 2821 | AGA TTA TCA AAA AGG ATC TTC ACC TAG ATC CTT TTA AAT TAA AAA TGA AGT TTT AAA TCA |
| | TCT AAT AGT TTT TCC TAG AAG TGG ATC TAG GAA AAT TTA ATT TTT ACT TCA AAA TTT AGT |
| 2881 | ATC TAA AGT ATA TAT GAG TAA ACT TGG TCT GAC AGT CTA GAC TAG GCC AAC TGG CGC AGC |
| | TAG ATT TCA TAT ATA CTC ATT TGA ACC AGA CTG TCA GAT CTG ATC CGG TTG ACC GCG TCG |
| | * A L Q R L M |
| 2941 | ATT CGA CGC AGC GGC TCG GCG GCG CCC CAT AAC AAC TGG TCG CCT ACG GTA AAC GCC GAC |
| | TAA GCT GCG TCG CCG AGC CGC CGC GGG GTA TTG TTG ACC AGC GGA TGC CAT TTG CGG CTG |
| | .. R R L P E A A G W L L Q D G V T F A S L |
| 3001 | AAG AAC TCT GGC CCC ATG TTC AGC TTA CGC AGA CGA CCA ACC GGC GTA GTC AAC GTG CCG |
| | TTC TTG AGA CCG GGG TAC AAG TCG AAT GCG TCT GCT GGT TGG CCG CAT CAG TTG CAC GGC |
| | .. F E P G M N L K R L R G V P T T L T G T |
| 3061 | GTC ACC GCC GCC GGG GTT AAT TCG CGC ATA GTG ATA TCA CGA TCG TTC GGC ACC ACT TTC |
| | CAG TGG CGG CGG CCC CAA TTA AGC GCG TAT CAC TAT AGT GCT AGC AAG CCG TGG TGA AAG |
| | .. V A A P T L E R M T I D R D N P V V K A |
| 3121 | GCC CAC GGA TTA TGT GCC GCC AGC AGT TCT TCC ACC GTC GGA ATG GAT ACC TCT TTT TTC |
| | CGG GTG CCT AAT ACA CGG CGG TCG TCA AGA AGG TGG CAG CCT TAC CTA TGG AGA AAA AAG |
| | .. W P N H A A L L E E V T P I S V E K K L |
| 3181 | AGC TTG ATG GTG AAC GCC TGG CTG TGA CAG CGC AGC GCG CCG ACG CGC ACA CAC AAA CCA |
| | TCG AAC TAC CAC TTG CGG ACC GAC ACT GTC GCG TCG CGC GGC TGC GCG TGT GTG TTT GGT |
| | .. K I T F A Q S H C R L A G V R V C L G D |
| 3241 | TCA ACC GGA ATC ACA GAG GCA GTA TTG AGA ATC TTG TTG GTT TCC GCC TGG CCT TTC CAC |
| | AGT TGG CCT TAG TGT CTC CGT CAT AAC TCT TAG AAC AAC CAA AGG CGG ACC GGA AAG GTG |
| | .. V P I V S A T N L I K N T E A Q G K W E |
| 3301 | TCT TCG CGG CTC TGG CCG TTA TCG AGC TGT TTG TCG ATC CAG GGG ATC AGG CTT CCC GCC |
| | AGA AGC GCC GAG ACC GGC AAT AGC TCG ACA AAC AGC TAG GTC CCC TAG TCC GAA GGG CGG |
| | .. E R S Q G N D L Q K D I W P I L S G A L |
| 3361 | AGC GGT ACG CCA AAG TTA TCA ACC GGC AGC TCG CCG CTG CGG GTC AAT GCC GTA ACT TTG |
| | TCG CCA TGC GGT TTC AAT AGT TGG CCG TCG AGC GGC GAC GCC CAG TTA CGG CAT TGA AAC |
| | .. P V G F N D V P L E G S R T L A T V K R |
| 3421 | CGT TCA ATA TCA AGA ATT GCG GAA GAC GGC GTC GCC AGT TCA TCG GCG ACA TGG CCA TAC |
| | GCA AGT TAT AGT TCT TAA CGC CTT CTG CCG CAG CGG TCA AGT AGC CGC TGT ACC GGT ATG |
| | .. E I D L I A S S P T A L E D A V H G Y L |
| 3481 | AAC TGA CCC ATC TGG GTT AAC AGC TCG CGC ATA TGG CGC GCG CCG CCG CCG GAG GCG GCC |
| | TTG ACT GGG TAG ACC CAA TTG TCG AGC GCG TAT ACC GCG CGC GGC GGC GGC CTC CGC CGG |
| | .. Q G M Q T L L E R M H R A G G G S A A Q |
| 3541 | TGA TAG GTC GCG ACG GAT ACC CAG TCA ACG AGA TTA TGG GCA AAG AGA CCG CCC AGC GAC |
| | ACT ATC CAG CGC TGC CTA TGG GTC AGT TGC TCT AAT ACC CGT TTC TCT GGC GGG TCG CTG |
| | .. Y T A V S V W D V L N H A F L G G L S M |
| 3601 | ATC AAC ATC AGG CTA ACG GTA CAG TTA CCG CCC ACA AAG GTC TTC ACG CCA TTG TTC AGG |
| | TAG TTG TAG TCC GAT TGC CAT GTC AAT GGC GGG TGT TTC CAG AAG TGC GGT AAC AAG TCC |
| | .. L M L S V T C N G G V F T K V G N N L G |
| 3661 | CCG TCG GTA ATC ACG TCC TGG TTG ACC GGG TCG AGA ATA ATA ATG GCA TCA TCT TTC ATG |
| | GGC AGC CAT TAG TGC AGG ACC AAC TGG CCC AGC TCT TAT TAT TAC CGT AGT AGA AAG TAC |
| | .. D T I V D Q N V P D L I I I A D D K M R |
| 3721 | CGC AGC GTA GAA GCC GCA TCA ATC CAG TAA CCC TGC CAT CCG CTT TCG CGC AGC TTT GGA |
| | GCG TCG CAT CTT CGG CGT AGT TAG GTC ATT GGG ACG GTA GGC GAA AGC GCG TCG AAA CCT |
| | .. L T S A A D I W Y G Q W G S E R L K P Y |
| 3781 | TAA ATT TCG TTG GTA TAA TCG CCG CCC TGG CAG GTC ACG ATG ATA TCG AGC GCT TTT AGC |
| | ATT TAA AGC AAC CAT ATT AGC GGC GGG ACC GTC CAG TGC TAC TAT AGC TCG CGA AAA TCG |
| | .. I E N T Y D G G Q C T V I I D L A K L A |
| 3841 | GCA TCC AGA TCA AAA GCG TCC TGT AGC GTG CCG GTG GAG GTG TCG CCG AAG GTG GGC GCC |
| | CGT AGG TCT AGT TTT CGC AGG ACA TCG CAC GGC CAC CTC CAC AGC GGC TTC CAC CCG CGG |
| | .. D L D F A D Q L T G T S T D G F T P A A |
| 3901 | GCC TGT CCA AAC TGG GAG GTA GAA AAG AAA ACA GGG CGA ATA GCG TCG AAA TCG CGC TCC |
| | CGG ACA GGT TTG ACC CTC CAT CTT TTC TTT TGT CCC GCT TAT CGC AGC TTT AGC GCG AGG |
| | .. Q G F Q S T S F F V P R I A D F D R E E |
| 3961 | TCT ACC ATG CGT TGC ATG AGA ACA GAG CCG ACC ATT CCG CGC CAG CCG ATA AAA CCA ACA |
| | AGA TGG TAC GCA ACG TAC TCT TGT CTC GGC TGG TAA GGC GCG GTC GGC TAT TTT GGT TGT |
| | .. V M R Q M L V S G V M G R W G I F G V N |
| 4021 | TTT TTC ATA GCG TTT TTT TCC TGC AAA GAG ATG TGC |
| | AAA AAG TAT CGC AAA AAA AGG ACG TTT CTC TAC ACG |
| | .. K M |

Sequence of pG8R320 (SEQ ID NO: 135 and 150)
Red highlighted: ompA sequence (SEQ ID NO: 1 and 140 (DNA) and SEQ ID NO: 2 (amino acid))
Green highlighted: PLZ4 peptide (SEQ ID NO: 3 and 141 (DNA) and SEQ ID NO: 4 (amino acid))
Cyan highlighted: Linker (SEQ ID NO: 5 (DNA) and SEQ ID NO: 6 (amino acid))
Yellow highlighted: asd sequence (SEQ ID NO: 142 and 145 (DNA) and SEQ ID NO: 144 (amino acid))
Grey highlighted: murA sequence (SEQ ID NO: 9 and 146 (DNA) and SEQ ID NO: 10 (amino acid))
Pink highlighted: araC sequence (SEQ ID NO: 11 and 147 (DNA) and SEQ ID NO: 12 (amino acid))

| | |
|---|---|
| 1 | GAC TCT TCG CGA TGT ACG GGC CAG ATA TAC GCG TTA ACT GCA GTC TAG ATT ATG CGA AAG |
| | CTG AGA AGC GCT ACA TGC CCG GTC TAT ATG CGC AAT TGA CGT CAG ATC TAA TAC GCT TTC |
| 61 | GCC ATC CTG ACG GAT GGC CTT TTT GTT TAA ACG GAT CCG CGA CAT TGA TTA TTG ACT AGT |
| | CGG TAG GAC TGC CTA CCG GAA AAA CAA ATT TGC CTA GGC GCT GTA ACT AAT AAC TGA TCA |
| 121 | TAT TAA TAG TAA TCA ATT ACG GGG TCA TTA GGG GAC TTT CCG GGG ACT TTC CTC CCC ACG |
| | ATA ATT ATC ATT AGT TAA TGC CCC AGT AAT CCC CTG AAA GGC CCC TGA AAG GAG GGG TGC |
| 181 | CGG GGG ACT TTC CGC CAC GGG CGG GGA CTT TCC GGG GAC TTT CCG TTC ATA GCC CAT ATA |
| | GCC CCC TGA AAG GCG GTG CCC GCC CCT GAA AGG CCC CTG AAA GGC AAG TAT CGG GTA TAT |
| 241 | TGG AGT TCC GCG TTA CAT AAC TTA CGG TAA ATG GCC CGC CTG GCT GAC CGC CCA ACG ACC |
| | ACC TCA AGG CGC AAT GTA TTG AAT GCC ATT TAC CGG GCG GAC CGA CTG GCG GGT TGC TGG |
| 301 | CCC GCC CAT TGA CGT CAA TAA TGA CGT ATG TTC CCA TAG TAA CGC CAA TAG GGA CTT TCC |
| | GGG CGG GTA ACT GCA GTT ATT ACT GCA TAC AAG GGT ATC ATT GCG GTT ATC CCT GAA AGG |
| 361 | ATT GAC GTC AAT GGG TGG ACT ATT TAC GGT AAA CTG CCC ACT TGG CAG TAC ATC AAG TGT |
| | TAA CTG CAG TTA CCC ACC TGA TAA ATG CCA TTT GAC GGG TGA ACC GTC ATG TAG TTC ACA |
| 421 | ATC ATA TGC CAA GTA CGC CCC CTA TTG ACG TCA ATG ACG GTA AAT GGC CCG CCT GGC ATT |
| | TAG TAT ACG GTT CAT GCG GGG GAT AAC TGC AGT TAC TGC CAT TTA CCG GGC GGA CCG TAA |
| 481 | ATG CCC AGT ACA TGA CCT TAT GGG ACT TTC CTA CTT GGC AGT ACA TCT ACG TAT TAG TCA |
| | TAC GGG TCA TGT ACT GGA ATA CCC TGA AAG GAT ACA TCA ATG GGC GTG GAT AGC GGT TTG |
| 541 | TCG CTA TTA CCA TGG TGA TGC GGT TTT GGC AGT ACA TCA ATG GGC GTG GAT AGC GGT TTG |
| | AGC GAT AAT GGT ACC ACT ACG CCA AAA CCG TCA TGT AGT TAC CCG CAC CTA TCG CCA AAC |
| 601 | ACT CAC GGG GAT TTC CAA GTC TCC ACC CCA TTG ACG TCA ATG GGA GTT TGT TTT GGC ACC |
| | TGA GTG CCC CTA AAG GTT CAG AGG TGG GGT AAC TGC AGT TAC CCT CAA ACA AAA CCG TGG |
| 661 | AAA ATC AAC GGG ACT TTC CAA AAT GTC GTA ACA ACT CCG CCC CAT TGA CGC AAA TGG GCG |
| | TTT TAG TTG CCC TGA AAG GTT TTA CAG CAT TGT TGA GGC GGG GTA ACT GCG TTT ACC CGC |
| 721 | GTA GGC GTG TAC GGT GGG AGG TCT ATA TAA GCA GAG CTC TCT GGC TAA CTA GAG AAC CCA |
| | CAT CCG CAC ATG CCA CCC TCC AGA TAT ATT CGT CTC GAG AGA CCG ATT GAT CTC TTG GGT |
| 781 | CTG CTT ACT GGC TTA TCG AAA TTA ATA CGA CTC ACT ATA GGG AGA CCC AAG CTG GCT AGC |
| | GAC GAA TGA CCG AAT AGC TTT AAT TAT GCT GAG TGA TAT CCC TCT GGG TTC GAC CGA TCG |
| 841 | GTT TAA ACT TAA GCT TGG TAC CGA GCT CGG ATC CAC TAG TCC AGT GTG GTG GAA TTC TGC |
| | CAA ATT TGA ATT CGA ACC ATG GCT CGA GCC TAG GTG ATC AGG TCA CAC CAC CTT AAG ACG |
| 901 | AGA TAT CCA GCA CAG TGG CGG CCG CTC GAG AAT GCT TCG AGC AGA CAT GAT AAG ATA CAT |
| | TCT ATA GGT CGT GTC ACC GCC GGC GAG CTC TTA CGA AGC TCG TCT GTA CTA TTC TAT GTA |
| 961 | TGA ATA GGT CGT GTC ACC GCC GGC GAG CTC TTA CGA AGC TCG TCT GTA CTA TTC TAT GTA |
| | ACT ACT CAA ACC TGT TTG GTG TTG ATC TTA CGT CAC TTT TTT TAC GAA ATA AAC ACT TTA |
| 1021 | TTG TGA TGC TAT TGC TTT ATT TGT AAC CAT TAT AAG CTG CAA TAA ACA AGT TAA CAA CAA |
| | AAC ACT ACG ATA ACG AAA TAA ACA TTG GTA ATA TTC GAC GTT ATT TGT TCA ATT GTT GTT |
| 1081 | CAA TTG CAT TCA TTT TAT GTT TCA GGT TCA GGG GGA GAT GTG GGA GGT TTT TTA AAG CAA |
| | GTT AAC GTA AGT AAA ATA CAA AGT CCA AGT CCC CCT CTA CAC CCT CCA AAA AAT TTC GTT |
| 1141 | GTA AAA CCT CTA CAA ATG TGG TAA AAT CCG ATA AGG ATC GAT CCG GGG CAT GCA ACC AGC |
| | CAT TTT GGA GAT GTT TAC ACC ATT TTA GGC TAT TCC TAG CTA GGC CCC GTA CGT TGG TCG |
| 1201 | TGT GGA ATG TGT GTC AGT TAG GGT GTG GAA AGT CCC CAG GCT CCC CAG CAG GCA GAA GTA |
| | ACA CCT TAC ACA CAG TCA ATC CCA CAC CTT TCA GGG GTC CGA GGG GTC GTC CGT CTT CAT |
| 1261 | TGC AAA GCA TGT GGG GAT GCG GTG GGC TCT ATG GCT TCT ACT GGG CGG TTT TAT GGA CAG |
| | ACG TTT CGT ACA CCC CTA CGC CAC CCG AGA TAC CGA AGA TGA CCC GCC AAA ATA CCT GTC |
| 1321 | CAA GCG AAC CGG AAT TGC CAG CTG GGG CGC CCT CTG GTA AGG TTG GGA AGC CCT GCA AAG |
| | GTT CGC TTG GCC TTA ACG GTC GAC CCC GCG GGA GAC CAT TCC AAC CCT TCG GGA CGT TTC |
| 1381 | TAA ACT GGA TGG CTT TCT CGC CGC CAA GGA TCT GTC GAC CCC TAG ATT TCA GTG CAA TTT |
| | ATT TGA CCT ACC GAA AGA GCG GCG GTT CCT AGA CAG CTG GGG ATC TAA AGT CAC GTT AAA |
| 1441 | ATC TCT TCA AAT GTA GCA CCT GAA GTC AGC CCC ATA CGA TAT AAG TTG TTG GAA GAT CTA |
| | TAG AGA AGT TTA CAT CGT GGA CTT CAG TCG GGG TAT GCT ATA TTC AAC AAC CTT CTA GAT |
| 1501 | GCC CGC CTA ATG AGC GGG CTT TTT TTT AAT TCG CAA TTC CCC GAT GCA TAA TGT GCC TGT |
| | CGG GCG GAT TAC TCG CCC GAA AAA AAA TTA AGC GTT AAG GGG CTA CGT ATT ACA CGG ACA |
| 1561 | CAA ATG GAC GAA GCA GGG ATT CTG CAA ACC CTA TGC TAC TCC GTC AAG CCG TCA ATT GTC |
| | GTT TAC CTG CTT CGT CCC TAA GAC GTT TGG GAT ACG ATG AGG CAG TTC GGC AGT TAA CAG |
| 1621 | 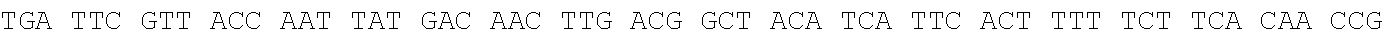 |
| | 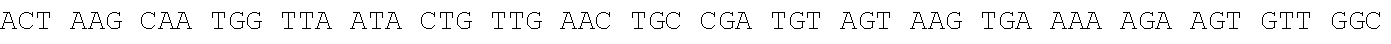 |
| | S L K V A V D N V K E E C G A |
| 1681 | 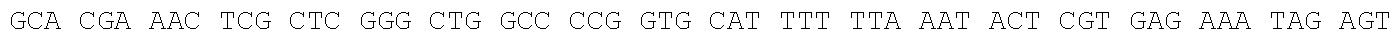 |
| | 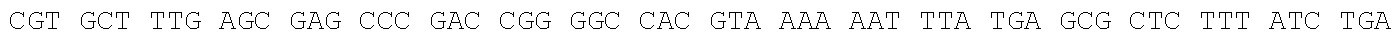 |
| | .. R F E S P S A G T C K K F V R S F Y L Q |
| 1741 | 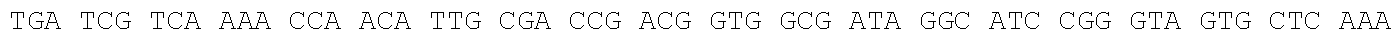 |
| | 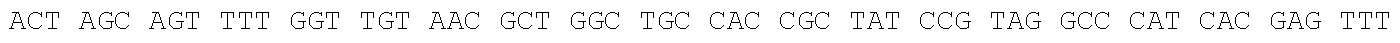 |
| | .. D D F G V N R G V T A I P M R T T S L L |
| 1801 | 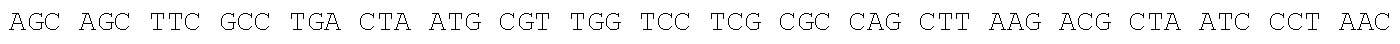 |
| | 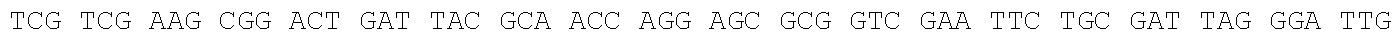 |
| | .. L K A Q S I R Q D E R W S L V S I G L Q |
| 1861 | 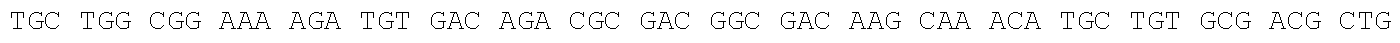 |
| | 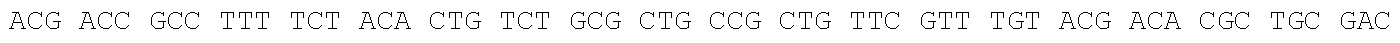 |
| | .. Q R F L H S L R S P S L C V H Q A V S A |
| 1921 | 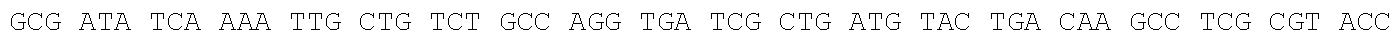 |
| | 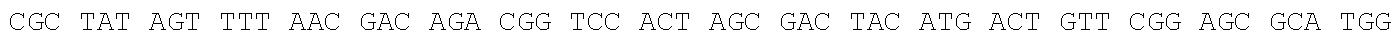 |
| | .. I D F N S D A L H D S I Y Q C A E R V R |
| 1981 | 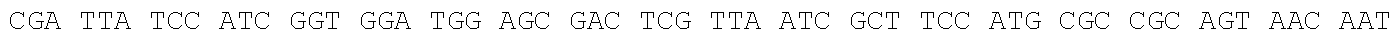 |
| | 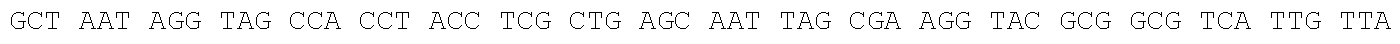 |
| | .. N D M P P H L S E N I A E M R R L L L Q |
| 2041 | 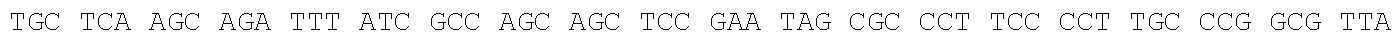 |
| | 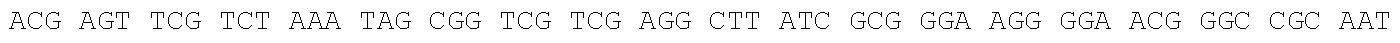 |
| | .. E L L N I A L L E S Y R G E G Q G A N I |
| 2101 | 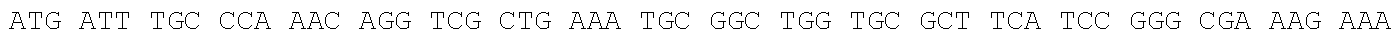 |
| | 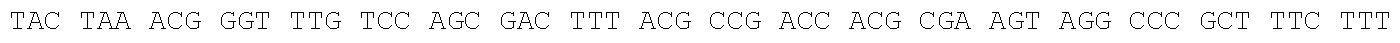 |
| | .. I Q G F L D S F H P Q H A E D P R F F G |
| 2161 | 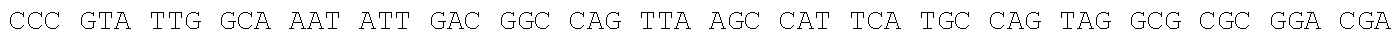 |
| | 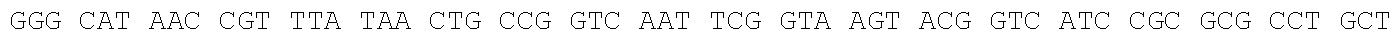 |
| | .. T N A F I S P W N L W E H W Y A R P R F |
| 2221 | 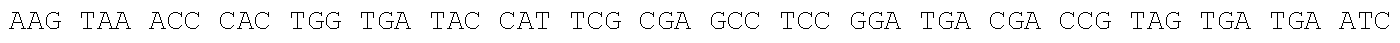 |
| | 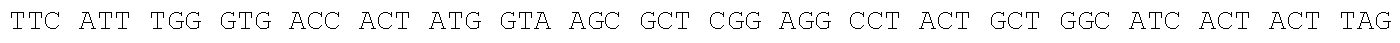 |
| | .. Y V W Q H Y W E R A E P H R G Y H H I E |
| 2281 | 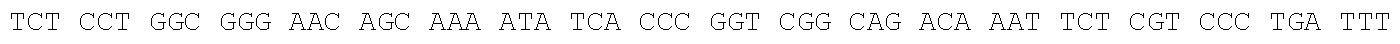 |
| | 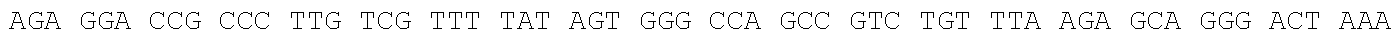 |
| | .. G P P F L L I D G P R C V F E R G Q N K |
| 2341 | 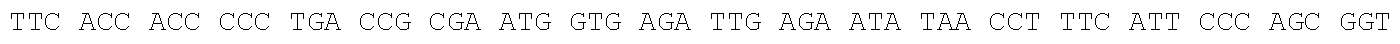 |
| | 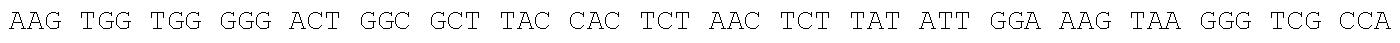 |
| | .. V V G Q G R I T L N L I Y G K M G L P R |
| 2401 | 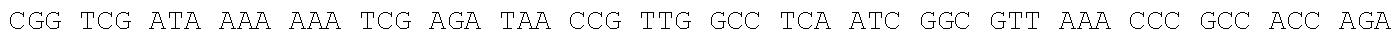 |
| | 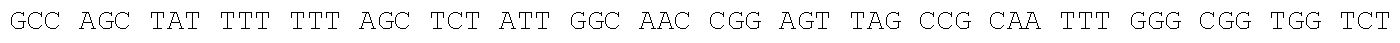 |
| | .. D I F F D L Y G N A E I P T L G A V L H |
| 2461 | 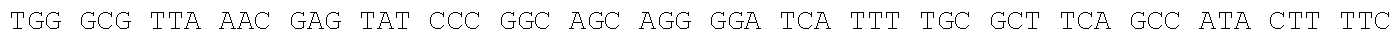 |
| | 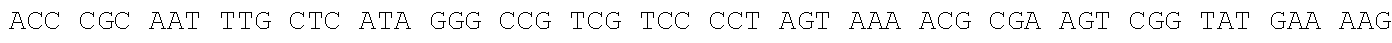 |
| | .. A N F S Y G P L L P D N Q A E A M |
| 2521 | ATA CTC CCA CCA TTC AGA GAA GAA ACC AAT TGT CCA TAT TGC ATC AGA CAT TGC CGT CAC |
| | TAT GAG GGT GGT AAG TCT CTT CTT TGG TTA ACA GGT ATA ACG TAG TCT GTA ACG GCA GTG |
| 2581 | TGC GTC TTT TAC TGG CTC TTC TCG CTA ACC CAA CCG GTA ACC CCG CTT ATT AAA AGC ATT |
| | ACG CAG AAA ATG ACC GAG AAG AGC GAT TGG GTT GGC CAT TGG GGC GAA TAA TTT TCG TAA |
| 2641 | CTG TAA CAA AGC GGG ACC AAA GCC ATG ACA AAA ACG CGT AAC AAA AGT GTC TAT AAT CAC |
| | GAC ATT GTT TCG CCC TGG TTT CGG TAC TGT TTT TGC GCA TTG TTT TCA CAG ATA TTA GTG |
| 2701 | GGC AGA AAA GTC CAC ATT GAT TAT TTG CAC GGC GTC ACA CTT TGC TAT GCC ATA GCA TTT |
| | CCG TCT TTT CAG GTG TAA CTA ATA AAC GTG CCG CAG TGT GAA ACG ATA CGG TAT CGT AAA |
| 2761 | TTA TCC ATA AGA TTA GCG GAT CCT ACC TGA CGC TTT TTA TCG CAA CTC TCT ACT GTT TCT |
| | AAT AGG TAT TCT AAT CGC CTA GGA TGG ACT GCG AAA AAT AGC GTT GAG AGA TGA CAA AGA |
| | M D K F R V |
| 2821 | 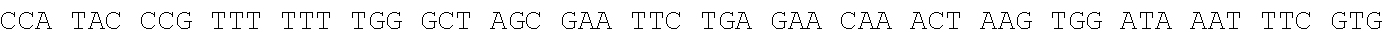 |
| | 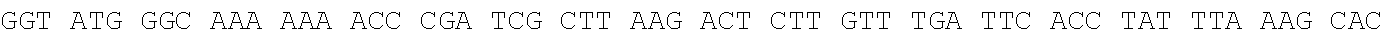 |
| | . Q G P T K L Q G E V T I S G A K N A A L |
| 2881 | 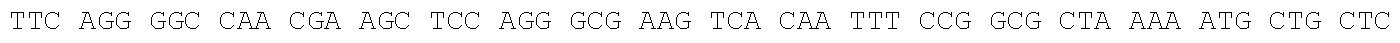 |
| | 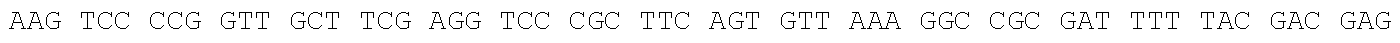 |
| | . P I L F A A L L A E E P V E I Q N V P K |
| 2941 | 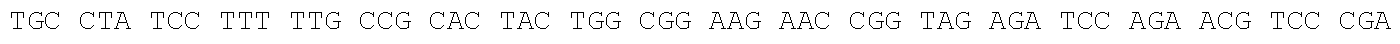 |
| | 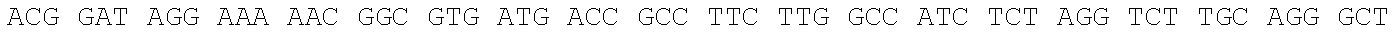 |
| | . L K D V D T S M K L L S Q L G A K V E R |
| 3001 | 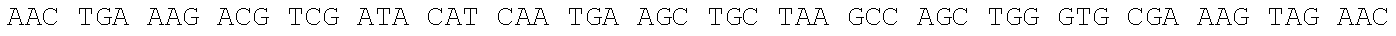 |
| | 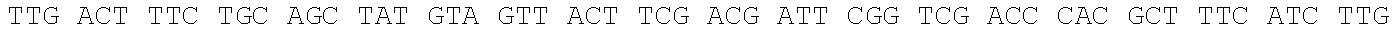 |
| | . N G S V H I D A R D V N V F C A P Y D L |
| 3061 | 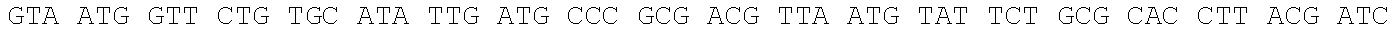 |
| | 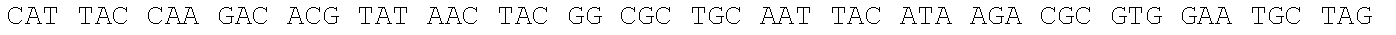 |
| | . V K T M R A S I W A L G P L V A R F G Q |
| 3121 | 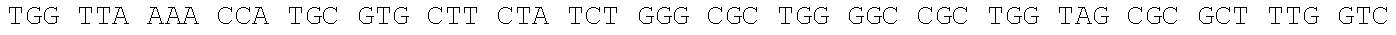 |
| | 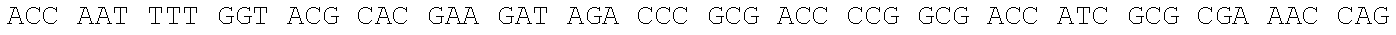 |
| | . G Q V S L P G G C T I G A R P V D L H I |
| 3181 | 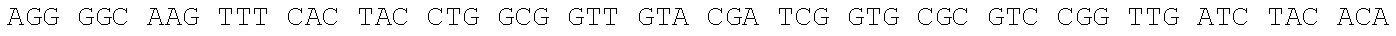 |
| | 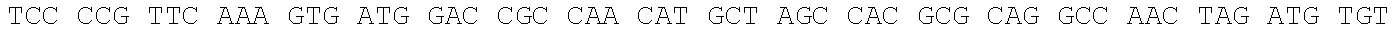 |
| | . S G L E Q L G A T I K L E E G Y V K A S |
| 3241 | 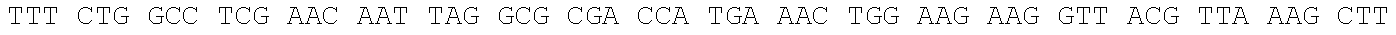 |
| | 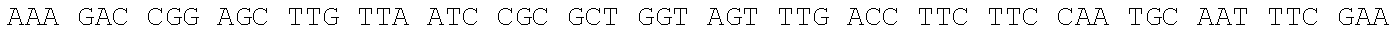 |
| | . V D G R L K G A H I V M D K V S V G A T |
| 3301 | 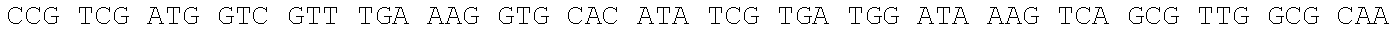 |
| | 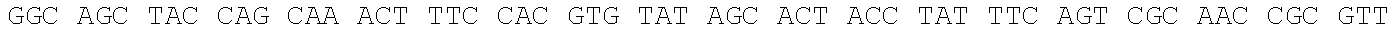 |
| | . V T I M C A A T L A E G T T I I E N A A |
| 3361 | 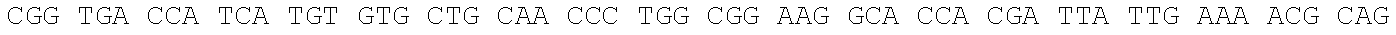 |
| | 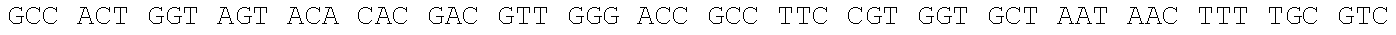 |
| | . R E P E I V D T A N F L I T L G A K I S |
| 3421 | 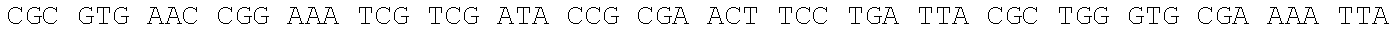 |
| | 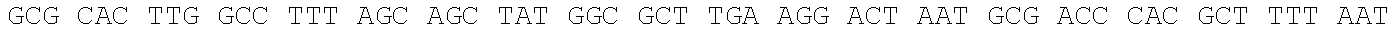 |
| | . G Q G T D R I V I E G V E R L G G G V Y |
| 3481 | 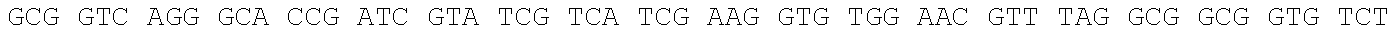 |
| | 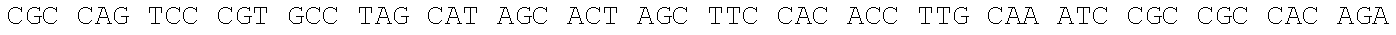 |
| | . R V L P D R I E T G T F L V A A A I S R |
| 3541 | 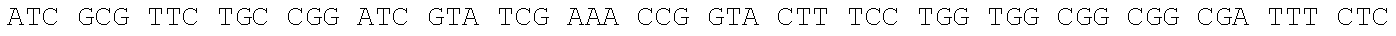 |
| | 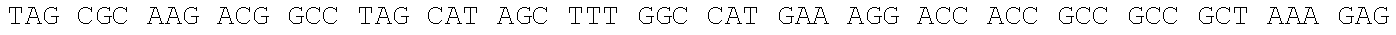 |
| | . G K I I C R N A Q P D T L D A V L A K L |
| 3601 | 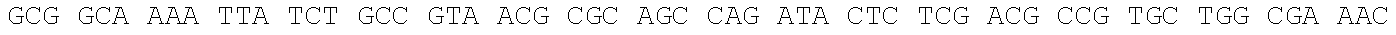 |
| | 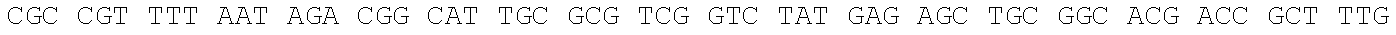 |
| | . R D A G A D I E V G E D W I S L D M H G |
| 3661 | 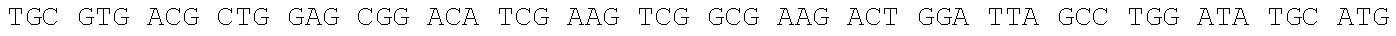 |
| | 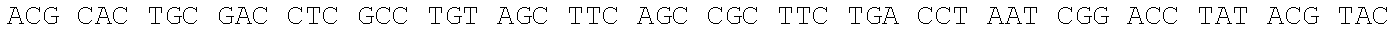 |
| | . K R P K A V N V R T A P H P A F P T D M |
| 3721 | 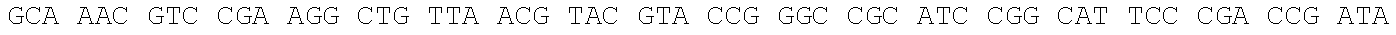 |
| | 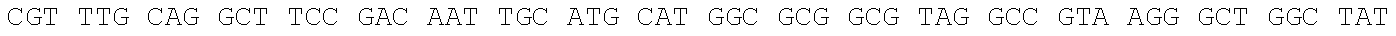 |
| | . Q A Q F T L L N L V A E G T G F I T E T |
| 3781 | 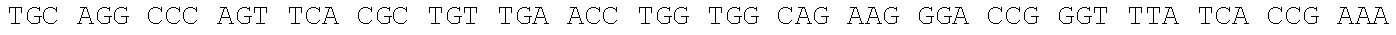 |
| | 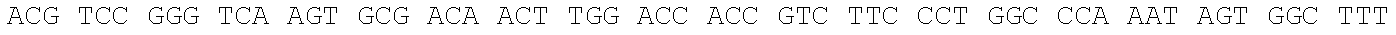 |
| | . V F E N R F M H V P E L S R M G A H A E |
| 3841 | 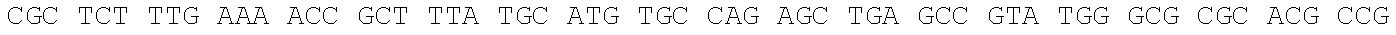 |
| | 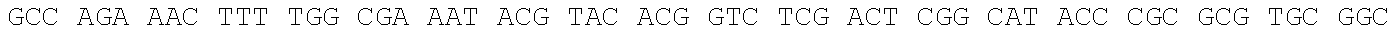 |
| | . I E S N T V I C H G V E K L S G A Q V M |
| 3901 | 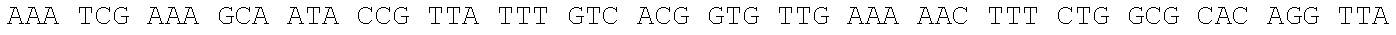 |
| | 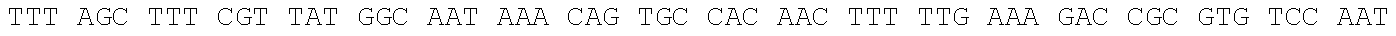 |
| | . A T D L R A S A S L V L A G C I A E G T |
| 3961 | 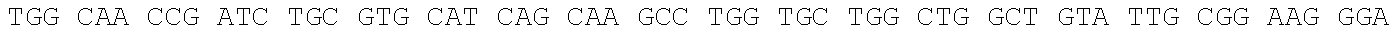 |
| | 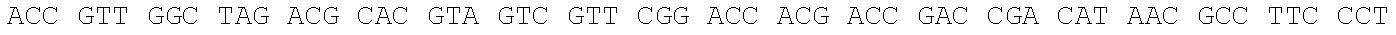 |
| | . T V V D R I Y H I D R G Y E R I E D K L |
| 4021 | 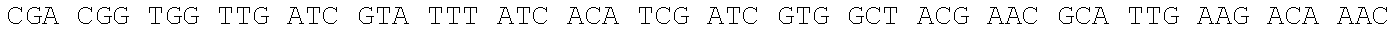 |
| | 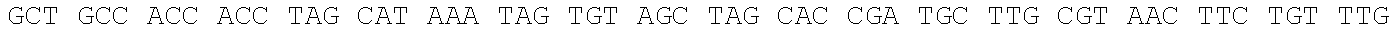 |
| | . R A L G A N I E R V K G E |
| 4081 | 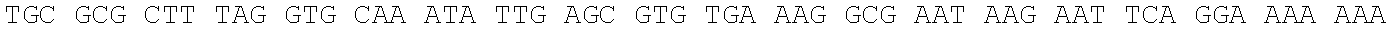 |
| | 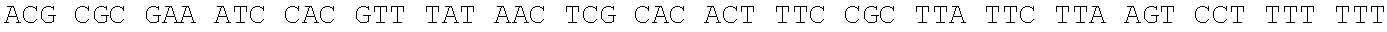 |
| | M K N V G F I G W R G M V G S V L M Q |
| 4141 | CGC TGT GAA AAA TGT TGG TTT TAT CGG CTG GCG CGG AAT GGT CGG CTC TGT TCT CAT GCA |
| | GCG ACA CTT TTT ACA ACC AAA ATA GCC GAC CGC GCC TTA CCA GCC GAG ACA AGA GTA CGT |
| | . R M V E E R D F D A I R P V F F S T S Q |
| 4201 | ACG CAT GGT AGA GGA GCG CGA TTT CGA CGC TAT TCG CCC TGT TTT CTT TTC TAC CTC CCA |
| | TGC GTA CCA TCT CCT CGC GCT AAA GCT GCG ATA AGC GGG ACA AAA GAA AAG ATG GAG GGT |
| | . F G Q A A P T F G D T S T G T L Q D A F |
| 4261 | GTT TGG ACA GGC GGC GCC CAC CTT CGG CGA CAC CTC CAC CGG CAC GCT ACA GGA CGC TTT |
| | CAA ACC TGT CCG CCG CGG GTG GAA GCC GCT GTG GAG GTG GCC GTG CGA TGT CCT GCG AAA |
| | . D L D A L K A L D I I V T C Q G G D Y T |
| 4321 | TGA TCT GGA TGC GCT AAA AGC GCT CGA TAT CAT CGT GAC CTG CCA GGG CGG CGA TTA TAC |
| | ACT AGA CCT ACG CGA TTT TCG CGA GCT ATA GTA GCA CTG GAC GGT CCC GCC GCT AAT ATG |
| | . N E I Y P K L R E S G W Q G Y W I D A A |
| 4381 | CAA CGA AAT TTA TCC AAA GCT GCG CGA AAG CGG ATG GCA GGG TTA CTG GAT TGA TGC GGC |
| | GTT GCT TTA AAT AGG TTT CGA CGC GCT TTC GCC TAC CGT CCC AAT GAC CTA ACT ACG CCG |
| | . S T L R M K D D A I I I L D P V N Q D V |
| 4441 | TTC TAC GCT GCG CAT GAA AGA TGA TGC CAT TAT TAT TCT CGA CCC GGT CAA CCA GGA CGT |
| | AAG ATG CGA CGC GTA CTT TCT ACT ACG GTA ATA ATA AGA GCT GGG CCA GTT GGT CCT GCA |
| | . I T D G L N N G V K T F V G G N C T V S |
| 4501 | GAT TAC CGA CGG CCT GAA CAA TGG CGT GAA GAC CTT TGT GGG CGG TAA CTG TAC CGT TAG |
| | CTA ATG GCT GCC GGA CTT GTT ACC GCA CTT CTG GAA ACA CCC GCC ATT GAC ATG GCA ATC |
| | . L M L M S L G G L F A H N L V D W V S V |
| 4561 | CCT GAT GTT GAT GTC GCT GGG CGG TCT CTT TGC CCA TAA TCT CGT TGA CTG GGT ATC CGT |
| | GGA CTA CAA CTA CAG CGA CCC GCC AGA GAA ACG GGT ATT AGA GCA ACT GAC CCA TAG GCA |
| | . A T Y Q A A S G G G A R H M R E L L T Q |
| 4621 | CGC GAC CTA TCA GGC CGC CTC CGG CGG CGG CGC GCG CCA TAT GCG CGA GCT GTT AAC CCA |
| | GCG CTG GAT AGT CCG GCG GAG GCC GCC GCC GCG CGC GGT ATA CGC GCT CGA CAA TTG GGT |
| | . M G Q L Y G H V A D E L A T P S S A I L |
| 4681 | GAT GGG TCA GTT GTA TGG CCA TGT CGC CGA TGA ACT GGC GAC GCC GTC TTC CGC AAT TCT |
| | CTA CCC AGT CAA CAT ACC GGT ACA GCG GCT ACT TGA CCG CTG CGG CAG AAG GCG TTA AGA |
| | . D I E R K V T A L T R S G E L P V D N F |
| 4741 | TGA TAT TGA ACG CAA AGT TAC GGC ATT GAC CCG CAG CGG CGA GCT GCC GGT TGA TAA CTT |
| | ACT ATA ACT TGC GTT TCA ATG CCG TAA CTG GGC GTC GCC GCT CGA CGG CCA ACT ATT GAA |
| | . G V P L A G S L I P W I D K Q L D N G Q |
| 4801 | TGG CGT ACC GCT GGC GGG AAG CCT GAT CCC CTG GAT CGA CAA ACA GCT CGA TAA CGG CCA |
| | ACC GCA TGG CGA CCG CCC TTC GGA CTA GGG GAC CTA GCT GTT TGT CGA GCT ATT GCC GGT |
| | . S R E E W K G Q A E T N K I L N T A S V |
| 4861 | GAG CCG CGA AGA GTG GAA AGG CCA GGC GGA AAC CAA CAA GAT TCT CAA TAC TGC CTC TGT |
| | CTC GGC GCT TCT CAC CTT TCC GGT CCG CCT TTG GTT GTT CTA AGA GTT ATG ACG GAG ACA |
| | . I P V D G L C V R V G A L R C H S Q A F |
| 4921 | GAT TCC GGT TGA TGG TTT GTG TGT GCG CGT CGG CGC GCT GCG CTG TCA CAG CCA GGC GTT |
| | CTA AGG CCA ACT ACC AAA CAC ACA CGC GCA GCC GCG CGA CGC GAC AGT GTC GGT CCG CAA |
| | . T I K L K K E V S I P T V E E L L A A H |
| 4981 | CAC CAT CAA GCT GAA AAA AGA GGT ATC CAT TCC GAC GGT GGA AGA ACT GCT GGC GGC ACA |
| | GTG GTA GTT CGA CTT TTT TCT CCA TAG GTA AGG CTG CCA CCT TCT TGA CGA CCG CCG TGT |
| | . N P W A K V V P N D R D I T M R E L T P |
| 5041 | TAA TCC GTG GGC GAA AGT GGT GCC GAA CGA TCG TGA TAT CAC TAT GCG CGA ATT AAC CCC |
| | ATT AGG CAC CCG CTT TCA CCA CGG CTT GCT AGC ACT ATA GTG ATA CGC GCT TAA TTG GGG |
| | . A A V T G T L T T P V G R L R K L N M G |
| 5101 | GGC GGC GGT GAC CGG CAC GTT GAC TAC GCC GGT TGG TCG TCT GCG TAA GCT GAA CAT GGG |
| | CCG CCG CCA CTG GCC GTG CAA CTG ATG CGG CCA ACC AGC AGA CGC ATT CGA CTT GTA CCC |
| | . P E F L S A F T V G D Q L L W G A A E P |
| 5161 | GCC AGA GTT CTT GTC GGC GTT TAC CGT AGG CGA CCA GTT GTT ATG GGG CGC CGC CGA GCC |
| | CGG TCT CAA GAA CAG CCG CAA ATG GCA TCC GCT GGT CAA CAA TAC CCC GCG GCG GCT CGG |
| | . L R R M L R Q L A |
| 5221 | GCT GCG TCG AAT GCT GCG CCA GTT GGC GTA GTC TAG CTG CAC GAT ACC GTC GAC TTG TAC |
| | CGA CGC AGC TTA CGA CGC GGT CAA CCG CAT CAG ATC GAC GTG CTA TGG CAG CTG AAC ATG |
| 5281 | ATA GAC TCG CTC CGA AAT TAA AGA ACA CTT AAA TTA TCT ACT AAA GGA ATC TTT AGT CAA |
| | TAT CTG AGC GAG GCT TTA ATT TCT TGT GAA TTT AAT AGA TGA TTT CCT TAG AAA TCA GTT |
| 5341 | GTT TAT TTA AGA TGA CTT AAC TAT GAA TAC ACA ATT GAT GGG TGA GCG TAG GAA AAA AAA |
| | CAA ATA AAT TCT ACT GAA TTG ATA CTT ATG TGT TAA CTA CCC ACT CGC ATC CTT TTT TTT |
| 5401 | ACC CCG CCC CTG ACA GGG CGG GGT TTT TTT TGA TCA TTC TGA AAT GAG CTG TTG ACA ATT |
| | TGG GGC GGG GAC TGT CCC GCC CCA AAA AAA ACT AGT AAG ACT TTA CTC GAC AAC TGT TAA |
| 5461 | AAT CAT CCG GCT CGT ATA ATG TGT GGA ATT GTG AGC GGA TAA CAA TTT CAC ACA GGA AAC |
| | TTA GTA GGC CGA GCA TAT TAC ACA CCT TAA CAC TCG CCT ATT GTT AAA GTG TGT CCT TTG |
| | M K K T A I A I A V A L A G F A T V A |
| 5521 | 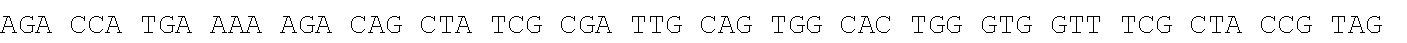 |
| | 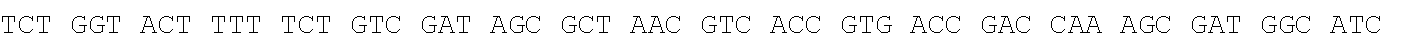 |
| | . Q A A P K D N T W Y A G A K L G W S Q Y |
| 5581 | 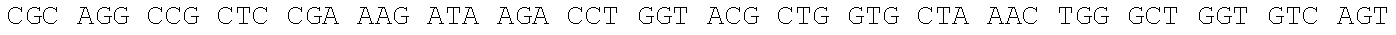 |
| | 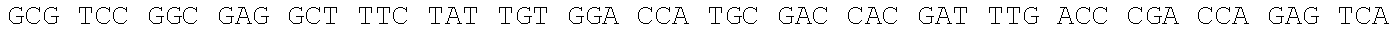 |
| | . H D T G F I H N D G P T H E N Q L G A G |
| 5641 | 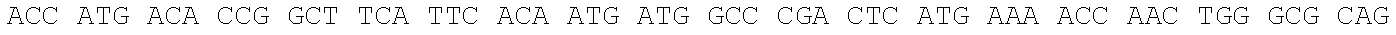 |
| | 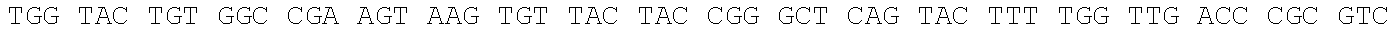 |
| | . A F G G Y Q V N P Y V G F E M G Y D W L |
| 5701 | 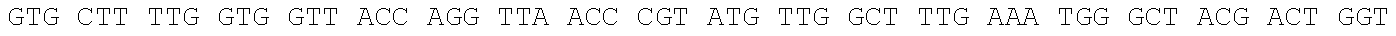 |
| | 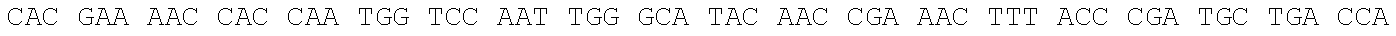 |
| | . G R M P Y K G D N I N G A Y K A Q G V Q |
| 5761 | 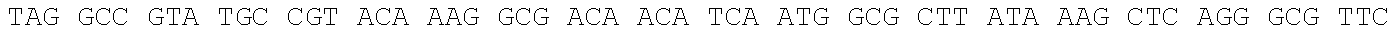 |
| | 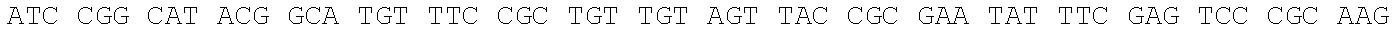 |
| | . L T A K L G Y P I T D D L D V Y T R L G |
| 5821 | 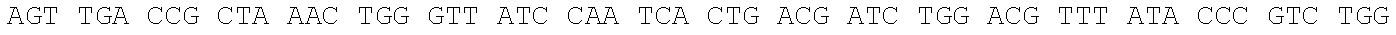 |
| | 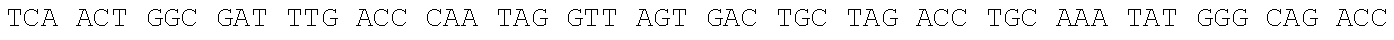 |
| | . G M V W R A D T K S N V F G A C Q D G R |
| 5881 | 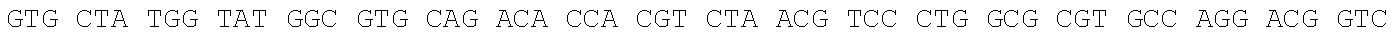 |
| | 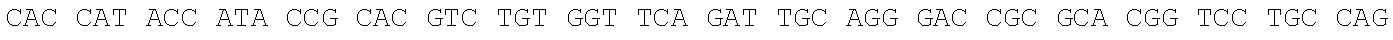 |
| | . M G F C G G P S T K D H D T G V S P V F |
| 5941 | 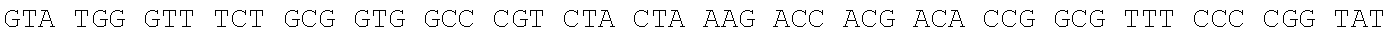 |
| | 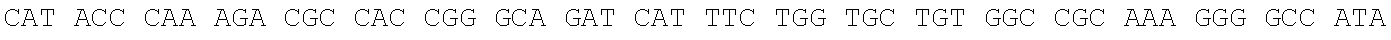 |
| | . A G G I E Y A I T P E I A T R L E Y Q W |
| 6001 | 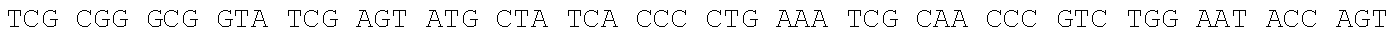 |
| | 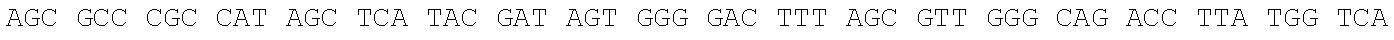 |
| | . T N N I G D A N T I G T R P D N G L L S |
| 6061 | 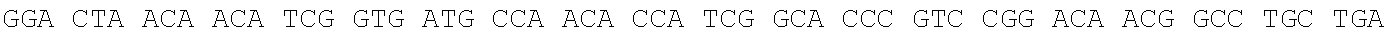 |
| | 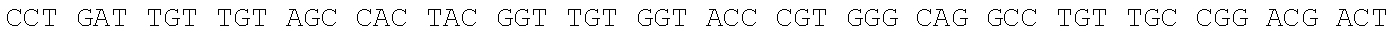 |
| | . V G V S Y R F G Q Q E A A P V V A P A P |
| 6121 | 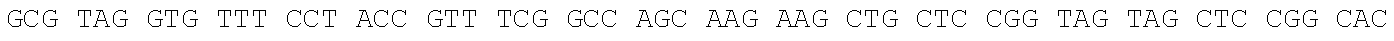 |
| | 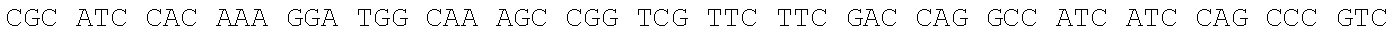 |
| | . A P A P E V Q T K H F T L K S D V L F N |
| 6181 | 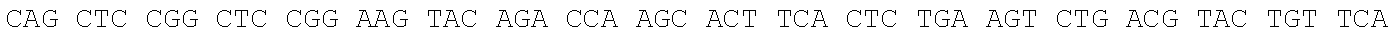 |
| | 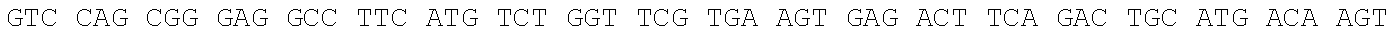 |
| | . F N K S T L K P E G Q Q A L D Q L Y S Q |
| 6241 | 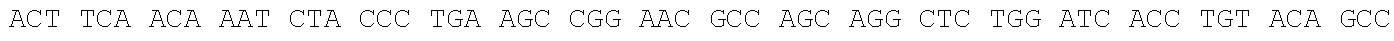 |
| | 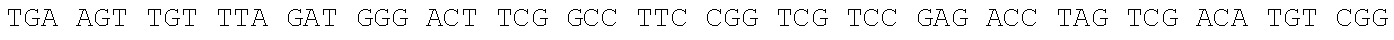 |
| | . L S N L D P K D G S V V V L G F T D R I |
| 6301 | 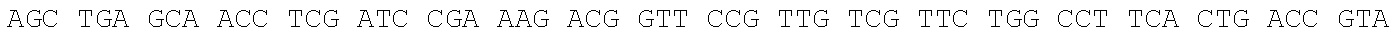 |
| | 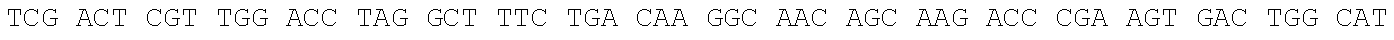 |
| | . G S D A Y N Q G L S E K R A Q S V V D Y |
| 6361 | 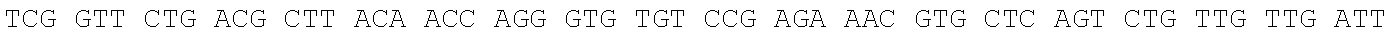 |
| | 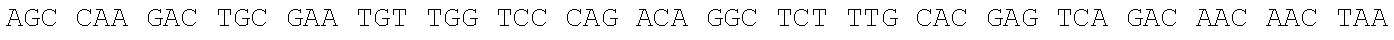 |
| | . L I S K G I P S D K I S A R G M G E S N |
| 6421 | 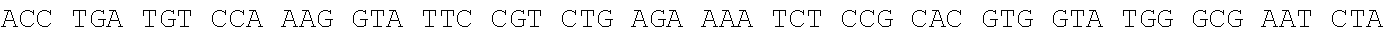 |
| | 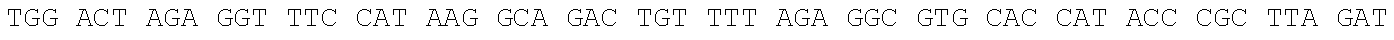 |
| | . P V T G N T C D N V K P R A A L I D C L |
| 6481 | 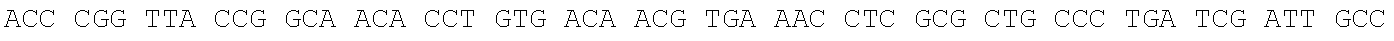 |
| | 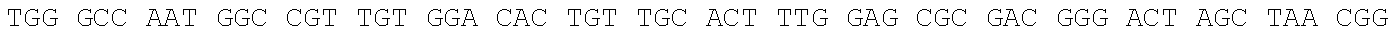 |
| | . A P D R R V E I E V K G V K D V V T Q P |
| 6541 | 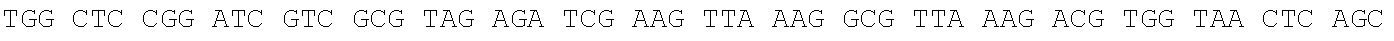 |
| | 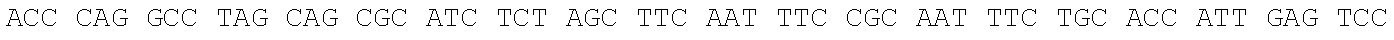 |
| | . Q A * |
| 6601 | 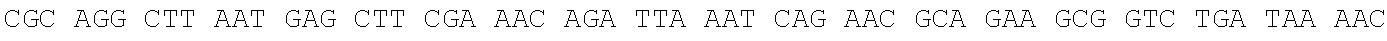 |
| | 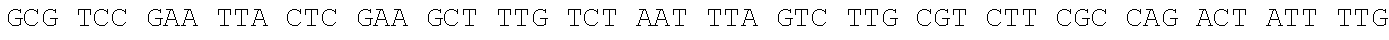 |
| 6661 | AGT TTG CCT GGC GGC AGT AGC GCG GTG GTC CCA CCT GAC CCC ATG CCG AAC TCA GAA GTG |
| | TCA AAC GGA CCG CCG TCA TCG CGC CAC CAG GGT GGA CTG GGG TAC GGC TTG AGT CTT CAC |
| 6721 | AAA CGC CGT AGC GCC GAT GGT AGT GTG GGG TCT CCC CAT GCG AGA GTA GGG AAC TGC CAG |
| | TTT GCG GCA TCG CGG CTA CCA TCA CAC CCC AGA GGG GTA CGC TCT CAT CCC TTG ACG GTC |
| 6781 | GCA TCA AAT AAA ACG AAA GGC TCA GTC GAA AGA CTG GGC CTT TCG TTT TAT CTG TTG TTT |
| | CGT AGT TTA TTT TGC TTT CCG AGT CAG CTT TCT GAC CCG GAA AGC AAA ATA GAC AAC AAA |
| 6841 | GTC GGT GAA CGC TCT CCT GAG TAG GAC AAA TCC GCC GGG AGC GGA TTT GAA CGT TGC GAA |
| | CAG CCA CTT GCG AGA GGA CTC ATC CTG TTT AGG CGG CCC TCG CCT AAA CTT GCA ACG CTT |
| 6901 | GCA ACG GCC CGG AGG GTG GCG GGC AGG ACG CCC GCC ATA AAC TGC CAG GCA TCA AAT TAA |
| | CGT TGC CGG GCC TCC CAC CGC CCG TCC TGC GGG CGG TAT TTG ACG GTC CGT AGT TTA ATT |
| 6961 | GCA GAA GGC CAT CCT GAC GGA TGG CCT TTT TGC GTT TCT ACA AAC TCT TTT TGT TTA TTT |
| | CGT CTT CCG GTA GGA CTG CCT ACC GGA AAA ACG CAA AGA TGT TTG AGA AAA ACA AAT AAA |
| 7021 | TTC TAA ATA CAT TCA AAT ATG TAT CCG CTC ATG AGA CAA TAA CCC TGA TAA ATG CTT CAA |
| | AAG ATT TAT GTA AGT TTA TAC ATA GGC GAG TAC TCT GTT ATT GGG ACT ATT TAC GAA GTT |
| 7081 | TAA TGG AAG ATC TTC CAA CAT CAC AGG TAA ACA GAA ACG TCG GGT CGA TCG GGA AAT TCT |
| | ATT ACC TTC TAG AAG GTT GTA GTG TCC ATT TGT CTT TGC AGC CCA GCT AGC CCT TTA AGA |
| 7141 | TTC CCG GAC GGC GCG GGG TTG GGC AAG CCG CAG GCG CGT CAG TGC TTT TAG CGG GTG TCG |
| | AAG GGC CTG CCG CGC CCC AAC CCG TTC GGC GTC CGC GCA GTC ACG AAA ATC GCC CAC AGC |
| 7201 | GGG CAG CCC TGA ACC AGT CAC GGG ATC GAT CTG TGC GGT ATT TCA CAC CGC ATA CAG GTG |
| | CCC GTC GGG ACT TGG TCA GTG CCC TAG CTA GAC ACG CCA TAA AGT GTG GCG TAT GTC CAC |
| 7261 | GCA CTT TTC GGG GAA ATG TGC GCG GAA CCC CTA TTT GTT TAT TTT TCT AAA TAC ATT CAA |
| | CGT GAA AAG CCC CTT TAC ACG CGC CTT GGG GAT AAA CAA ATA AAA AGA TTT ATG TAA GTT |
| 7321 | ATA TGT ATC CGC TCA TGA GAC AAT AAC CCT GAT AAA TGC TTC AAT AAT AGC ACG TGC TAA |
| | TAT ACA TAG GCG AGT ACT CTG TTA TTG GGA CTA TTT ACG AAG TTA TTA TCG TGC ACG ATT |
| 7381 | AAC TTC ATT TTT AAT TTA AAA GGA TCT AGG TGA AGA TCC TTT TTG ATA ATC TCA TGA CCA |
| | TTG AAG TAA AAA TTA AAT TTT CCT AGA TCC ACT TCT AGG AAA AAC TAT TAG AGT ACT GGT |
| 7441 | AAA TCC CTT AAC GTG AGT TTT CGT TCC ACT GAG CGT CAG ACC CCG TAG AAA AGA TCA AAG |
| | TTT AGG GAA TTG CAC TCA AAA GCA AGG TGA CTC GCA GTC TGG GGC ATC TTT TCT AGT TTC |
| 7501 | GAT CTT CTT GAG ATC CTT TTT TTC TGC GCG TAA TCT GCT GCT TGC AAA CAA AAA AAC CAC |
| | CTA GAA GAA CTC TAG GAA AAA AAG ACG CGC ATT AGA CGA CGA ACG TTT GTT TTT TTG GTG |
| 7561 | CGC TAC CAG CGG TGG TTT GTT TGC CGG ATC AAG AGC TAC CAA CTC TTT TTC CGA AGG TAA |
| | GCG ATG GTC GCC ACC AAA CAA ACG GCC TAG TTC TCG ATG GTT GAG AAA AAG GCT TCC ATT |
| 7621 | CTG GCT TCA GCA GAG CGC AGA TAC CAA ATA CTG TCC TTC TAG TGT AGC CGT AGT TAG GCC |
| | GAC CGA AGT CGT CTC GCG TCT ATG GTT TAT GAC AGG AAG ATC ACA TCG GCA TCA ATC CGG |
| 7681 | ACC ACT TCA AGA ACT CTG TAG CAC CGC CTA CAT ACC TCG CTC TGC TAA TCC TGT TAC CAG |
| | TGG TGA AGT TCT TGA GAC ATC GTG GCG GAT GTA TGG AGC GAG ACG ATT AGG ACA ATG GTC |
| 7741 | TGG CTG CTG CCA GTG GCG ATA AGT CGT GTC TTA CCG GGT TGG ACT CAA GAC GAT AGT TAC |
| | ACC GAC GAC GGT CAC CGC TAT TCA GCA CAG AAT GGC CCA ACC TGA GTT CTG CTA TCA ATG |
| 7801 | CGG ATA AGG CGC AGC GGT CGG GCT GAA CGG GGG GTT CGT GCA CAC AGC CCA GCT TGG AGC |
| | GCC TAT TCC GCG TCG CCA GCC CGA CTT GCC CCC CAA GCA CGT GTG TCG GGT CGA ACC TCG |
| 7861 | GAA CGA CCT ACA CCG AAC TGA GAT ACC TAC AGC GTG AGC TAT GAG AAA GCG CCA CGC TTC |
| | CTT GCT GGA TGT GGC TTG ACT CTA TGG ATG TCG CAC TCG ATA CTC TTT CGC GGT GCG AAG |
| 7921 | CCG AAG GGA GAA AGG CGG ACA GGT ATC CGG TAA GCG GCA GGG TCG GAA CAG GAG AGC GCA |
| | GGC TTC CCT CTT TCC GCC TGT CCA TAG GCC ATT CGC CGT CCC AGC CTT GTC CTC TCG CGT |
| 7981 | CGA GGG AGC TTC CAG GGG GAA ACG CCT GGT ATC TTT ATA GTC CTG TCG GGT TTC GCC ACC |
| | GCT CCC TCG AAG GTC CCC CTT TGC GGA CCA TAG AAA TAT CAG GAC AGC CCA AAG CGG TGG |
| 8041 | TCT GAC TTG AGC GTC GAT TTT TGT GAT GCT CGT CAG GGG GGC GGA GCC TAT GGA AAA ACG |
| | AGA CTG AAC TCG CAG CTA AAA ACA CTA CGA GCA GTC CCC CCG CCT CGG ATA CCT TTT TGC |
| 8101 | CCA GCA ACG CGG CCT TTT TAC GGT TCC TGG GCT TTT GCT GGC CTT TTG CTC ACA TGT TCT |
| | GGT CGT TGC GCC GGA AAA ATG CCA AGG ACC CGA AAA CGA CCG GAA AAC GAG TGT ACA AGA |

Human CXCL11 (SEQ ID NO: 13 (DNA) and SEQ ID NO: 14 (amino acid))

| | |
|---|---|
| | M S V K G M A I A L A V I L C A T V V Q |
| 1 | ATG AGT GTG AAG GGC ATG GCT ATA GCC TTG GCT GTG ATA TTG TGT GCT ACA GTT GTT CAA |
| | G F P M F K R G R C L C I G P G V K A V |
| 61 | GGC TTC CCC ATG TTC AAA AGA GGA CGC TGT CTT TGC ATA GGC CCT GGG GTA AAA GCA GTG |
| | K V A D I E K A S I M Y P S N N C D K I |
| 121 | AAA GTG GCA GAT ATT GAG AAA GCC TCC ATA ATG TAC CCA AGT AAC AAC TGT GAC AAA ATA |
| | E V I I T L K E N K G Q R C L N P K S K |
| 181 | GAA GTG ATT ATT ACC CTG AAA GAA AAT AAA GGA CAA CGA TGC CTA AAT CCC AAA TCG AAG |
| | Q A R L I I K K V E R K N F * |
| 241 | CAA GCA AGG CTT ATA ATC AAA AAA GTT GAA AGA AAG AAT TTT TAA |

Mouse CXCL11(SEQ ID NO: 15 (DNA) AND SEQ ID NO: 16 (amino acis))

| | |
|---|---|
| | M N R K V T A I A L A A I I W A T A A Q |
| 1 | ATG AAC AGG AAG GTC ACA GCC ATA GCC CTG GCT GCG ATC ATC TGG GCC ACA GCT GCT CAA |
| | G F L M F K Q G R C L C I G P G M K A V |
| 61 | GGC TTC CTT ATG TTC AAA CAG GGG CGC TGT CTT TGC ATC GGC CCC GGG ATG AAA GCC GTC |
| | K M A E I E K A S V I Y P S N G C D K V |
| 121 | AAA ATG GCA GAG ATC GAG AAA GCT TCT GTA ATT TAC CCG AGT AAC GGC TGC GAC AAA GTT |
| | E V I V T M K A H K R Q R C L D P R S K |
| 181 | GAA GTG ATT GTT ACT ATG AAG GCT CAT AAA CGA CAA AGG TGC CTG GAC CCC AGA TCC AAG |
| | Q A R L I M Q A I E K K N F L R R Q N M |
| 241 | CAA GCT CGC CTC ATA ATG CAG GCA ATA GAA AAA AAG AAT TTT TTA AGG CGT CAA AAC ATG |
| | * |
| 301 | TAA |

KillerRed-memo (SEQ ID NO: 17 (DNA) and SEQ ID NO: 18 (amino acid))

| | |
|---|---|
| | M L C C M R R T K Q V E K N D E D Q K I |
| 1 | ATG CTG TGC TGT ATG AGA AGA ACC AAA CAG GTT GAA AAG AAT GAT GAG GAC CAA AAG ATC |
| | S E G G P A L F Q S D M T F K I F I D G |
| 61 | TCC GAG GGC GGC CCC GCC CTG TTC CAG AGC GAC ATG ACC TTC AAA ATC TTC ATC GAC GGC |
| | E V N G Q K F T I V A D G S S K F P H G |
| 121 | GAG GTG AAC GGC CAG AAG TTC ACC ATC GTG GCC GAC GGC AGC AGC AAG TTC CCC CAC GGC |
| | D F N V H A V C E T G K L P M S W K P I |
| 181 | GAC TTC AAC GTG CAC GCC GTG TGC GAG ACC GGC AAG CTG CCC ATG AGC TGG AAG CCC ATC |
| | C H L I Q Y G E P F F A R Y P D G I S H |
| 241 | TGC CAC CTG ATC CAG TAC GGC GAG CCC TTC TTC GCC CGC TAC CCC GAC GGC ATC AGC CAT |
| | F A Q E C F P E G L S I D R T V R F E N |
| 301 | TTC GCC CAG GAG TGC TTC CCC GAG GGC CTG AGC ATC GAC CGC ACC GTG CGC TTC GAG AAC |
| | D G T M T S H H T Y E L D D T C V V S R |
| 361 | GAC GGC ACC ATG ACC AGC CAC CAC ACC TAC GAG CTG GAC GAC ACC TGC GTG GTG AGC CGC |
| | I T V N C D G F Q P D G P I M R D Q L V |
| 421 | ATC ACC GTG AAC TGC GAC GGC TTC CAG CCC GAC GGC CCC ATC ATG CGC GAC CAG CTG GTG |
| | D I L P N E T H M F P H G P N A V R Q L |
| 481 | GAC ATC CTG CCC AAC GAG ACC CAC ATG TTC CCC CAC GGC CCC AAC GCC GTG CGC CAG CTG |
| | A F I G F T T A D G G L M M G H F D S K |
| 541 | GCC TTC ATC GGC TTC ACC ACC GCC GAC GGC GGC CTG ATG ATG GGC CAC TTC GAC AGC AAG |
| | M T F N G S R A I E I P G P H F V T I I |
| 601 | ATG ACC TTC AAC GGC AGC CGC GCC ATC GAG ATC CCC GGC CCA CAC TTC GTG ACC ATC ATC |
| | T K Q M R D T S D K R D H V C Q R E V A |
| 661 | ACC AAG CAG ATG AGG GAC ACC AGC GAC AAG CGC GAC CAC GTG TGC CAG CGC GAG GTG GCC |
| | Y A H S V P R I T S A I G S D E D * |
| 721 | TAC GCC CAC AGC GTG CCC CGC ATC ACC AGC GCC ATC GGT AGC GAC GAG GAT TAA |

KillerRed-mito (SEQ ID NO: 19 (DNA) and SEQ ID NO: 20 (amino acid))

| | |
|---|---|
| | M S V L T P L L L R G L T G S A R R L P |
| 1 | ATG TCC GTC CTG ACG CCG CTG CTG CTG CGG GGC TTG ACA GGC TCG GCC CGG CGG CTC CCA |
| | V P R A K I H S L G D L S V L T P L L L |
| 61 | GTG CCG CGC GCC AAG ATC CAT TCG TTG GGG GAT CTG TCC GTC CTG ACG CCG CTG CTG CTG |
| | R G L T G S A R R L P V P R A K I H S L |
| 121 | CGG GGC TTG ACA GGC TCG GCC CGG CGG CTC CCA GTG CCG CGC GCC AAG ATC CAT TCG TTG |
| | G D P P V A T M G S E G G P A L F Q S D |
| 181 | GGG GAT CCA CCG GTC GCC ACC ATG GGT TCA GAG GGC GGC CCC GCC CTG TTC CAG AGC GAC |
| | M T F K I F I D G E V N G Q K F T I V A |
| 241 | ATG ACC TTC AAA ATC TTC ATC GAC GGC GAG GTG AAC GGC CAG AAG TTC ACC ATC GTG GCC |
| | D G S S K F P H G D F N V H A V C E T G |
| 301 | GAC GGC AGC AGC AAG TTC CCC CAC GGC GAC TTC AAC GTG CAC GCC GTG TGC GAG ACC GGC |
| | K L P M S W K P I C H L I Q Y G E P F F |
| 361 | AAG CTG CCC ATG AGC TGG AAG CCC ATC TGC CAC CTG ATC CAG TAC GGC GAG CCC TTC TTC |
| | A R Y P D G I S H F A Q E C F P E G L S |
| 421 | GCC CGC TAC CCC GAC GGC ATC AGC CAT TTC GCC CAG GAG TGC TTC CCC GAG GGC CTG AGC |
| | I D R T V R F E N D G T M T S H H T Y E |
| 481 | ATC GAC CGC ACC GTG CGC TTC GAG AAC GAC GGC ACC ATG ACC AGC CAC CAC ACC TAC GAG |
| | L D D T C V V S R I T V N C D G F Q P D |
| 541 | CTG GAC GAC ACC TGC GTG GTG AGC CGC ATC ACC GTG AAC TGC GAC GGC TTC CAG CCC GAC |
| | G P I M R D Q L V D I L P N E T H M F P |
| 601 | GGC CCC ATC ATG CGC GAC CAG CTG GTG GAC ATC CTG CCC AAC GAG ACC CAC ATG TTC CCC |
| | H G P N A V R Q L A F I G F T T A D G G |
| 661 | CAC GGC CCC AAC GCC GTG CGC CAG CTG GCC TTC ATC GGC TTC ACC ACC GCC GAC GGC GGC |
| | L M M G H F D S K M T F N G S R A I E I |
| 721 | CTG ATG ATG GGC CAC TTC GAC AGC AAG ATG ACC TTC AAC GGC AGC CGC GCC ATC GAG ATC |
| | P G P H F V T I I T K Q M R D T S D K R |
| 781 | CCC GGC CCA CAC TTC GTG ACC ATC ATC ACC AAG CAG ATG AGG GAC ACC AGC GAC AAG CGC |
| | D H V C Q R E V A Y A H S V P R I T S A |
| 841 | GAC CAC GTG TGC CAG CGC GAG GTG GCC TAC GCC CAC AGC GTG CCC CGC ATC ACC AGC GCC |
| | I G S D E D * |
| 901 | ATC GGT AGC GAC GAG GAT TAA |

HLAB in pG8R345 (SEQ ID NO: 21 (DNA) and SEQ ID NO: 22(amino acid))

| | |
|---|---|
| | M L V M A P R T V L L L L S A A L A L T |
| 1 | ATG CTG GTC ATG GCG CCC CGA ACC GTC CTC CTG CTG CTC TCG GCG GCC CTG GCC CTG ACC |
| | E T W A G S G L A V L A V |
| 61 | GAG ACC TGG GCC GGC TCC GGT ACC GGG CCC GGG CCT AGG GGC CTG GCT GTC CTG GCA GTT |
| | V V I G A V V A A V M C R R K S S G G K |
| 121 | GTG GTC ATC GGA GCT GTG GTC GCT GCT GTG ATG TGT AGG AGG AAG AGT TCA GGT GGA AAA |
| | G G S Y S Q A A C S D S A Q G S D V S L |
| 181 | GGA GGG AGC TAC TCT CAG GCT GCG TGC AGC GAC AGT GCC CAG GGC TCT GAT GTG TCT CTC |
| | T A * |
| 241 | ACA GCT TGA |

OmpA-LHRH peptide (SEQ ID NO: 136) and DNA (SEQ ID NO: 151) in pG8R380
and pG8R 381
Green highlighted: LHRH peptide (SEQ ID NO: 23 (DNA) and SEQ ID NO: 24 (amino acid))

| | |
|---|---|
| | M K K T A I A I A V A L A G F A T V A Q |
| 1 | ATG AAA AAG ACA GCT ATC GCG ATT GCA GTG GCA CTG GCT GGT TTC GCT ACC GTA GCG CAG |
| | A A P K D N T W Y A G A K L G W S Q Y H |
| 61 | GCC GCT CCG AAA GAT AAC ACC TGG TAC GCT GGT GCT AAA CTG GGC TGG TCT CAG TAC CAT |
| | D T G F I H N D G P T H E N Q L G A G A |
| 121 | GAC ACC GGC TTC ATT CAC AAT GAT GGC CCG ACT CAT GAA AAC CAA CTG GGC GCA GGT GCT |
| | F G G Y Q V N P Y V G F E M G Y D W L G |
| 181 | TTT GGT GGT TAC CAG GTT AAC CCG TAT GTT GGC TTT GAA ATG GGC TAC GAC TGG TTA GGC |
| | R M P Y K G D N I N G A Y K A Q G V Q L |
| 241 | CGT ATG CCG TAC AAA GGC GAC AAC ATC AAT GGC GCT TAT AAA GCT CAG GGC GTT CAG TTG |
| | T A K L G Y P I T D D L D V Y T R L G G |
| 301 | ACC GCT AAA CTG GGT TAT CCA ATC ACT GAC GAT CTG GAC GTT TAT ACC CGT CTG GGT GGT |
| | M V W R A D T K S N V P G A C E H W S Y |
| 361 | 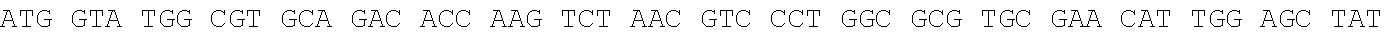 |
| | G L R P G C G G P S T K D H D T G V S P |
| 421 | 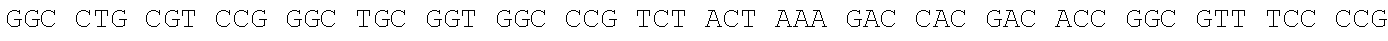 |
| | V F A G G I E Y A I T P E I A T R L E Y |
| 481 | GTA TTC GCG GGC GGT ATC GAG TAT GCT ATC ACC CCT GAA ATC GCA ACC CGT CTG GAA TAC |
| | Q W T N N I G D A N T I G T R P D N G L |
| 541 | CAG TGG ACT AAC AAC ATC GGT GAT GCC AAC ACC ATC GGC ACC CGT CCG GAC AAC GGC CTG |
| | L S V G V S Y R F G Q Q E A A P V V A P |
| 601 | CTG AGC GTA GGT GTT TCC TAC CGT TTC GGC CAG CAA GAA GCT GCT CCG GTA GTA GCT CCG |
| | A P A P A P E V Q T K H F T L K S D V L |
| 661 | GCA CCA GCT CCG GCT CCG GAA GTA CAG ACC AAG CAC TTC ACT CTG AAG TCT GAC GTA CTG |
| | F N F N K S T L K P E G Q Q A L D Q L Y |
| 721 | TTC AAC TTC AAC AAA TCT ACC CTG AAG CCG GAA GGC CAG CAG GCT CTG GAT CAG CTG TAC |
| | S Q L S N L D P K D G S V V V L G F T D |
| 781 | AGC CAG CTG AGC AAC CTG GAT CCG AAA GAC GGT TCC GTT GTC GTT CTG GGC TTC ACT GAC |
| | R I G S D A Y N Q G L S E K R A Q S V V |
| 841 | CGT ATC GGT TCT GAC GCT TAC AAC CAG GGT CTG TCC GAG AAA CGT GCT CAG TCT GTT GTT |
| | D Y L I S K G I P S D K I S A R G M G E |
| 901 | GAT TAC CTG ATC TCC AAA GGT ATT CCG TCT GAC AAA ATC TCC GCA CGT GGT ATG GGC GAA |
| | S N P V T G N T C D N V K P R A A L I D |
| 961 | TCT AAC CCG GTT ACC GGC AAC ACC TGT GAC AAC GTG AAA CCT CGC GCT GCC CTG ATC GAT |
| | C L A P D R R V E I E V K G V K D V V T |
| 1021 | TGC CTG GCT CCG GAT CGT CGC GTA GAG ATC GAA GTT AAA GGC GTT AAA GAC GTG GTA ACT |
| | Q P Q A * |
| 1081 | CAG CCG CAG GCT TAA |

LHRH peptide (SEQ ID NO: 25)
EHWSYGLRPG
or
Glu-His-Trp-Ser-tYR-Gly-Leu-Arg-Pro-Gly
KillerRed memo-P2A-HumanCXCL11 (SEQ ID NO: 52 and SEQ ID NO: 156)
Red highlighted: Neuromodulin N-terminal sequence (mem)(SEQ ID NO: 26 (DNA) and SEQ ID NO: 27
(amino acid))
Cyan highlighted: KillerRed sequence (SEQ ID NO: 28 (DNA) and SEQ ID NO: 29 (amino acid))
Yellow highlighted: GSG P2A sequence (SEQ ID NO: 30 (DNA) and SEQ ID NO: 31 (amino acid))
Pink highlighted: Human CXCL11 sequence (SEQ ID NO: 32 (DNA) and SEQ ID NO: 33 (amino acid))

| | |
|---|---|
| | M L C C M R R T K Q V E K N D E D Q K I |
| 1 | 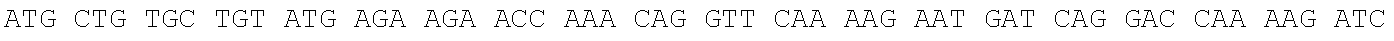 |
| | S E G G P A L F Q S D M T F K I F I D G |
| 61 | 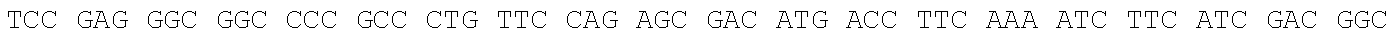 |
| | E V N G Q K F T I V A D G S S K F P H G |
| 121 | 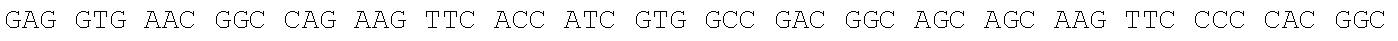 |
| | D F N V H A V C E T G K L P M S W K P I |
| 181 | 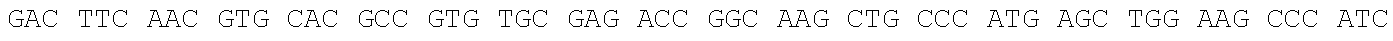 |
| | C H L I Q Y G E P F F A R Y P D G I S H |
| 241 | 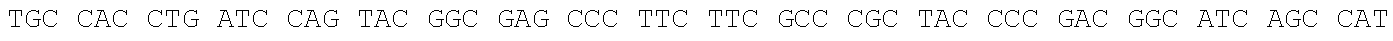 |
| | F A Q E C F P E G L S I D R T V R F E N |
| 301 | 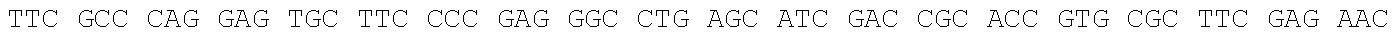 |
| | D G T M T S H H T Y E L D D T C V V S R |
| 361 | 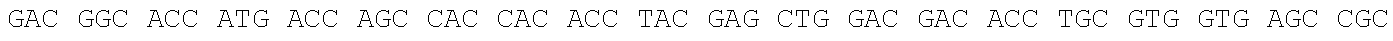 |
| | I T V N C D G F Q P D G P I M R D Q L V |
| 421 | 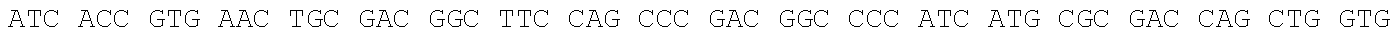 |
| | D I L P N E T H M F P H G P N A V R Q L |
| 481 | 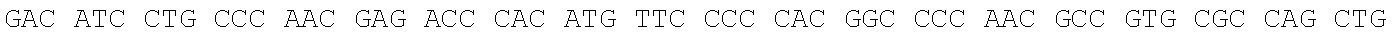 |
| | A F I G F T T A D G G L M M G H F D S K |
| 541 | 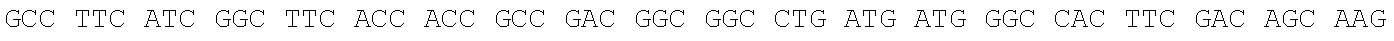 |
| | M T F N G S R A I E I P G P H F V T I I |
| 601 | 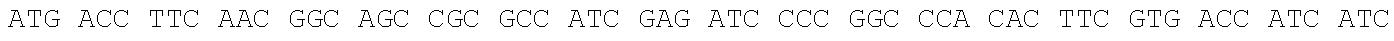 |
| | T K Q M R D T S D K R D H V C Q R E V A |
| 661 | 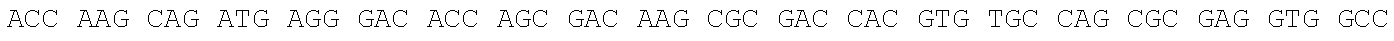 |
| | Y A H S V P R I T S A I G S D E D G S G |
| 721 | 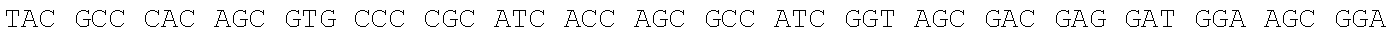 |
| | A T N F S L L K Q A G D V E E N P G P M |
| 781 | 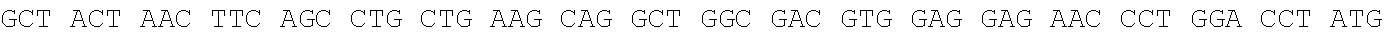 |
| | S V K G M A I A L A V I L C A T V V Q G |
| 841 | 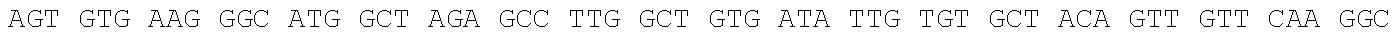 |
| | F P M F K R G R C L C I G P G V K A V K |
| 901 | 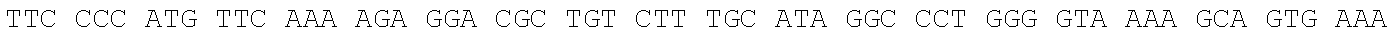 |
| | V A D I E K A S I M Y P S N N C D K I E |
| 961 | 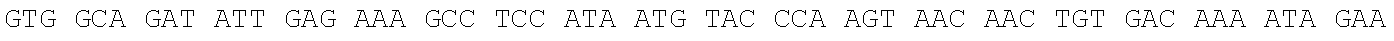 |
| | V I I T L K E N K G Q R C L N P K S K Q |
| 1021 | 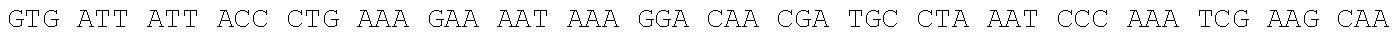 |
| | A R L I I K K V E R K N F * |
| 1081 | 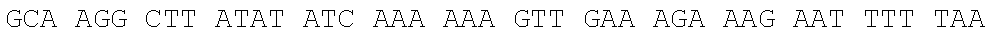 |

KillerRed memo-P2A-Mouse CXCL11 (SEQ ID NO: 53 and SEQ ID NO: 157)
Red highlighted: Neuromodulin N-terminal sequence (mem)(SEQ ID NO: 34 (DNA) and SEQ ID NO: 35(amino acid))
Cyan highlighted: KillerRed sequence (SEQ ID NO: 36 (DNA) and SEQ ID NO: 37 (amino acid))
Yellow highlighted: GSG P2A sequence (SEQ ID NO: 38 (DNA) and SEQ ID NO: 39 (amino acid))
Pink highlighted: Mouse CXCL11 sequence (SEQ ID NO: 40 (DNA) and SEQ ID NO: 41 (amino acid))

| | |
|---|---|
| | M L C C M R R T K Q V E K N D E D Q K I |
| 1 | 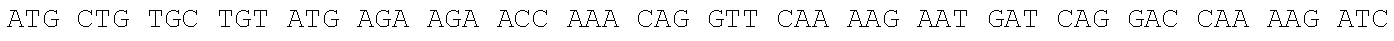 |
| | S E G G P A L F Q S D M T F K I F I D G |
| 61 | 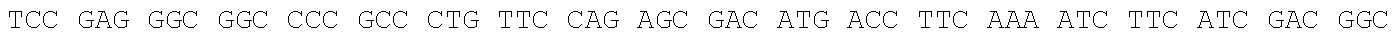 |
| | E V N G Q K F T I V A D G S S K F P H G |
| 121 | 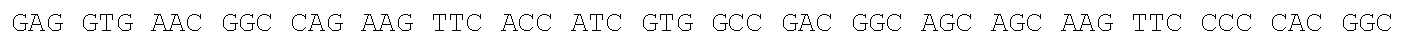 |
| | D F N V H A V C E T G K L P M S W K P I |
| 181 | 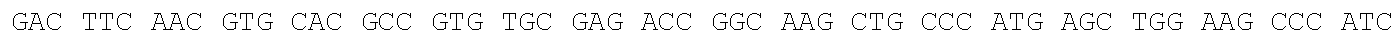 |
| | C H L I Q Y G E P F F A R Y P D G I S H |
| 241 | 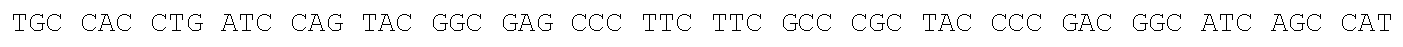 |
| | F A Q E C F P E G L S I D R T V R F E N |
| 301 | 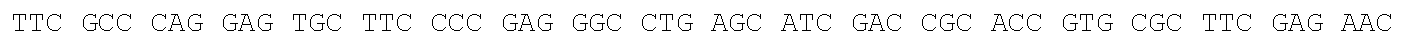 |
| | D G T M T S H H T Y E L D D T C V V S R |
| 361 | 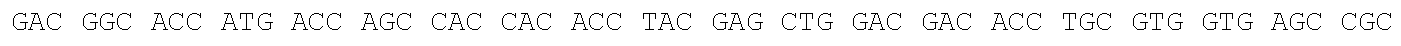 |
| | I T V N C D G F Q P D G P I M R D Q L V |
| 421 | 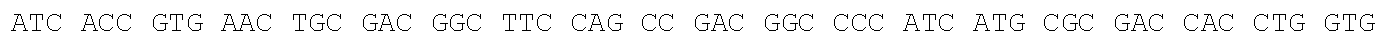 |
| | D I L P N E T H M F P H G P N A V R Q L |
| 481 | 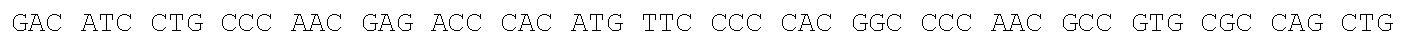 |
| | A F I G F T T A D G G L M M G H F D S K |
| 541 | 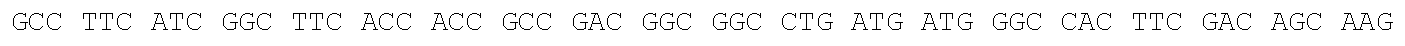 |
| | M T F N G S R A I E I P G P H F V T I I |
| 601 | 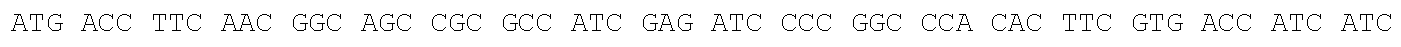 |
| | T K Q M R D T S D K R D H V C Q R E V A |
| 661 | 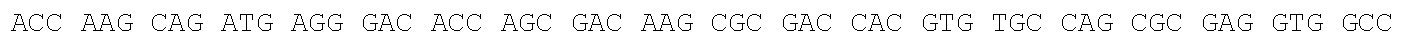 |
| | Y A H S V P R I T S A I G S D E D G S G |
| 721 | 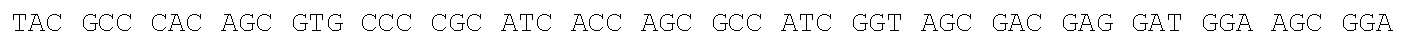 |
| | A T N F S L L K Q A G D V E E N P G P M |
| 781 | 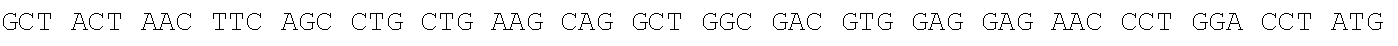 |
| | S V K G M A I A L A V I L C A T V V Q G |
| 841 | 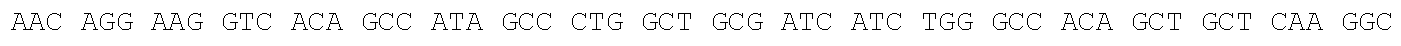 |
| | F P M F K R G R C L C I G P G V K A V K |
| 901 | 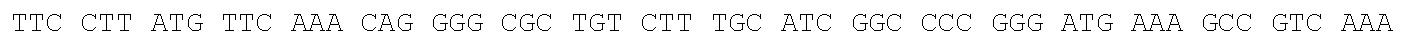 |
| | V A D I E K A S I M Y P S N N C D K I E |
| 961 | 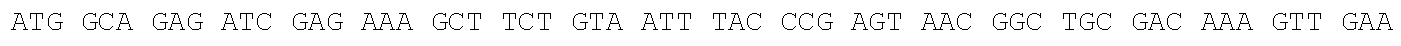 |
| | V I I T L K E N K G Q R C L N P K S K Q |
| 1021 | 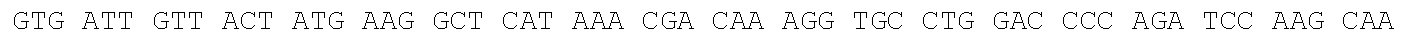 |
| | A R L I I K K V E R K N F L R R Q N M * |
| 1081 | 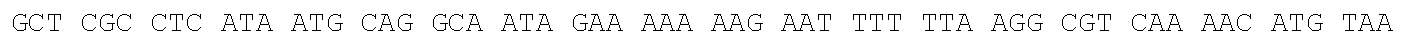 |

GSG P2A sequence (SEQ ID NO: 42 (DNA) and (SEQ ID NO: 43 (amino acid))

| | |
|---|---|
| | G S G A T N F S L L K Q A G D V E E N P |
| 1 | GGA AGC GGA GCT ACT AAC TTC AGC CTG CTG AAG CAG GCT GGC GAC GTG GAG GAG AAC CCT |
| | G P |
| 61 | GGA CCT |

Her2 ScFV sequence (SEQ ID NO: 137 and SEQ ID NO: 158)
Red highlighted: ompA sequence (mem)(SEQ ID NO: 44 (DNA) and SEQ ID NO: 45 (amino acid))
Green highlighted: Her2 ScFV sequence (SEQ ID NO: 46 (DNA) and SEQ ID NO: 47 (amino acid))

| | |
|---|---|
| | M K K T A I A I A V A L A G F A T V A Q |
| 1 | 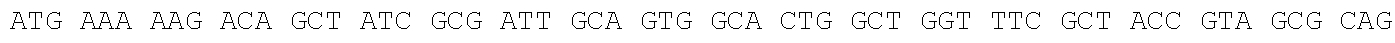 |
| | A A P K D N T W Y A G A K L G W S Q Y H |
| 61 | 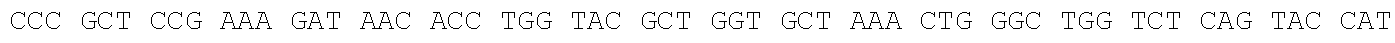 |
| | D T G F I H N D G P T H E N Q L G A G A |
| 121 | 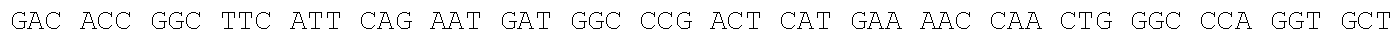 |
| | F G G Y Q V N P Y V G F E M G Y D W L G |
| 181 | 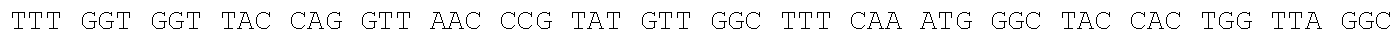 |
| | R M P Y K G D N I N G A Y K A Q G V Q L |
| 241 | 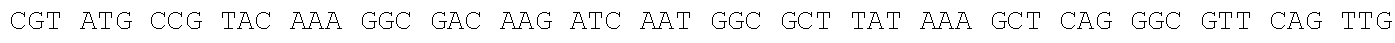 |
| | T A K L G Y P I T D D L D V Y T R L G G |
| 301 | 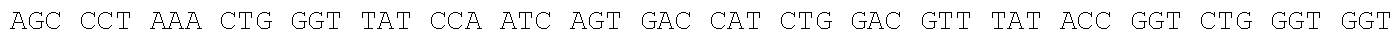 |
| | M V W R A D T K S N V P G Q S G A E V K |
| 361 | 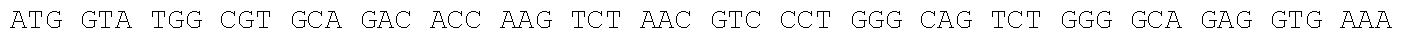 |
| | K P G E S L K I S C K G S G Y S F T S Y |
| 421 | 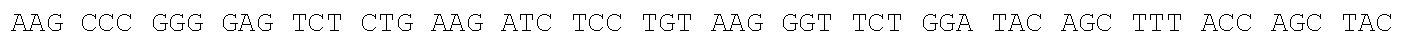 |
| | W I A W V R Q M P G K G L E Y M G L I Y |
| 481 | 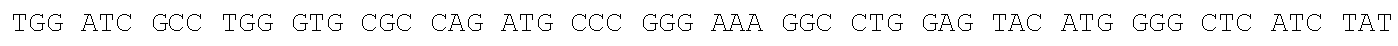 |
| | P G D S D T K Y S P S F Q G Q V T I S V |
| 541 | 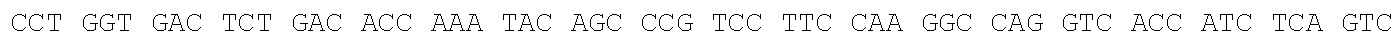 |
| | D K S V S T A Y L Q W S S L K P S D S A |
| 601 | 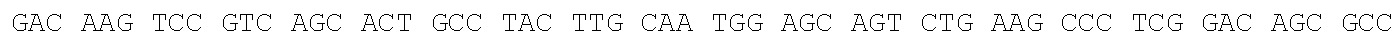 |
| | V Y F C A R H D V G Y C S S S N C A K W |
| 661 | 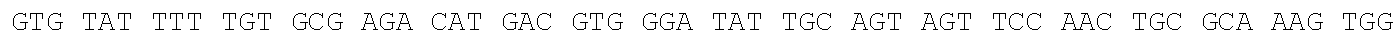 |
| | P E Y F Q H W G Q G T L V T V S S G G G |
| 721 | 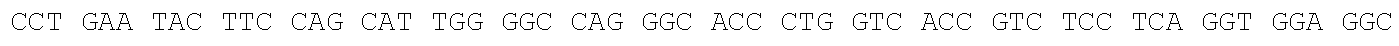 |
| | G S G G G G S G G G G S Q S V L T Q P P |
| 781 | 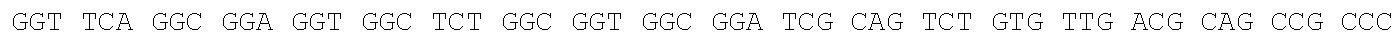 |
| | S V S A A P G Q K V T I S C S G S S S N |
| 841 | 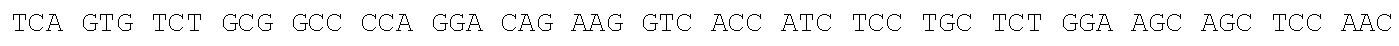 |
| | I G N N Y V S W Y Q Q L P G T A P K L L |
| 901 | 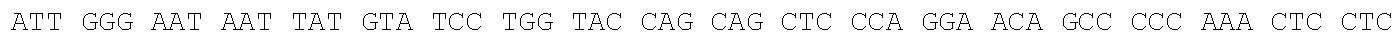 |
| | I Y D H T N R P A G V P D R F S G S K S |
| 961 | 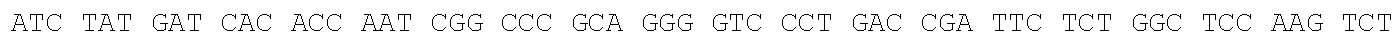 |
| | G T S A S L A I S G F R S E D E A D Y Y |
| 1021 | 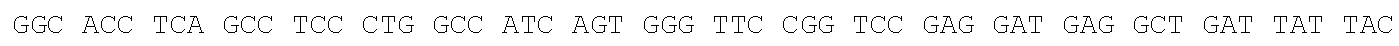 |
| | C A S W D Y T L S G W V F G G G T K L T |
| 1081 | 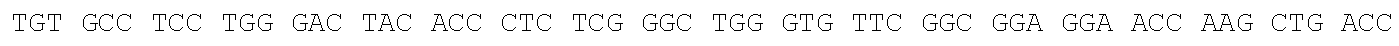 |
| | V L G A A A G G G G S H H H H H H G P S |
| 1141 | 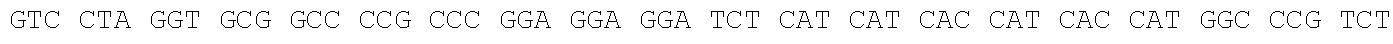 |
| | T K D H D T G V S P V F A G G I E Y A I |
| 1201 | 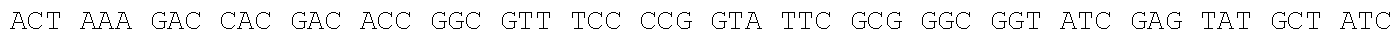 |
| | T P E I A T R L E Y Q W T N N I G D A N |
| 1261 | 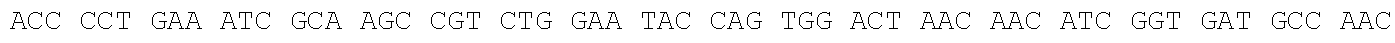 |
| | T I G T R P D N G L L S V G V S Y R F G |
| 1321 | 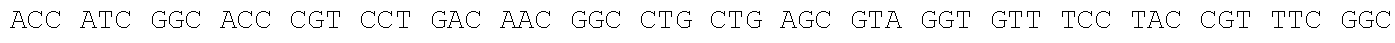 |
| | Q Q E A A P V V A P A P A P A P E V Q T |
| 1381 | 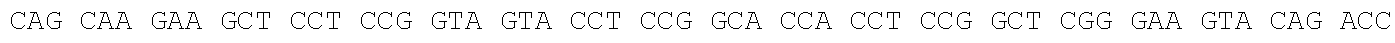 |
| | K H F T L K S D V L F N F N K S T L K P |
| 1441 | 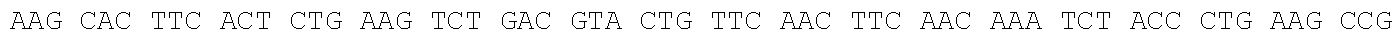 |
| | E G Q Q A L D Q L Y S Q L S N L D P K D |
| 1501 | 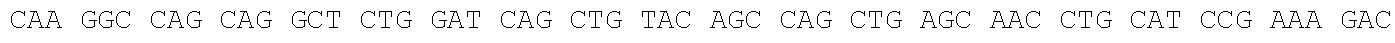 |
| | G S V V V L G F T D R I G S D A Y N Q G |
| 1561 | 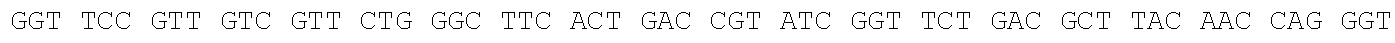 |
| | L S E K R A Q S V V D Y L I S K G I P S |
| 1621 | 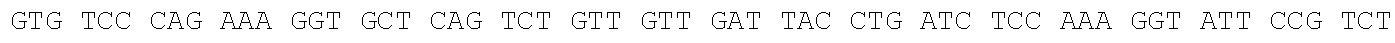 |
| | D K I S A R G M G E S N P V T G N T C D |
| 1681 | 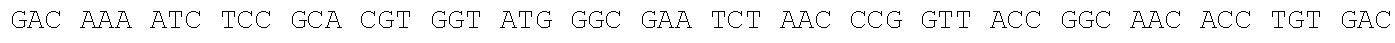 |
| | N V K P R A A L I D C L A P D R R V E I |
| 1741 | 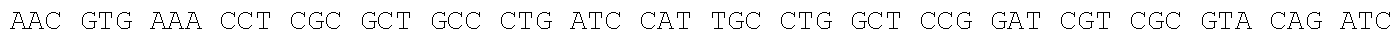 |
| | E V K G V K D V V T Q P Q A * |
| 1801 | 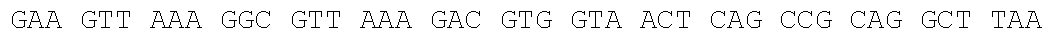 |

HAC-PD1 sequence (SEQ ID NO: 48(DNA) and SEQ ID NO: 49(amino acid))

| | |
|---|---|
| | M D S P D R P W N P P T F S P A L L V V |
| 1 | ATG GAT TCC CCA GAT AGA CCA TGG AAC CCA CCA ACT TTC TCC CCA GCT TTG TTG GTC GTC |
| | T E G D N A T F T C S F S N T S E S F H |
| 61 | ACT GAA GGT GAT AAC GCT ACT TTC ACT TGT TCC TTC TCC AAC ACT TCC GAA TCC TTC CAT |
| | V V W H R E S P S G Q T D T L A A F P E |
| 121 | GTT GTT TGG CAT CGT GAA TCC CCA TCC GGT CAA ACT GAT ACA TTG GCT GCT TTC CCA GAA |
| | D R S Q P G Q D A R F R V T Q L P N G R |
| 181 | GAT AGA TCC CAA CCA GGT CAA GAT GCT AGA TTC AGA GTT ACT CAA TTG CCA AAC GGT AGA |
| | D F H M S V V R A R R N D S G T Y V C G |
| 241 | GAT TTC CAC ATG TCC GTC GTC AGA GCT AGA AGA AAC GAT TCC GGT ACT TAT GTT TGT GGT |
| | V I S L A P K I Q I K E S L R A E L R V |
| 301 | GTT ATT TCC CTT GCT CCA AAG ATT CAA ATT AAG GAA TCC TTG AGA GCT GAA TTG AGA GTC |
| | T E R |
| 361 | ACT GAA AGA |

haPD1-IgG sequence (SEQ ID NO: 50 (DNA) and SEQ ID NO: 51(amino acid))

| | |
|---|---|
| | M G W S C I I L F L V A T A T G V H S F |
| 1 | ATG GGC TGG TCC TGT ATC ATC CTG TTC CTG GTG GCT ACA GCC ACA GGA GTG CAT AGT TTC |
| | L D S P D R P W N P P T F S P A L L V V |
| 61 | TTA GAC TCC CCA GAC AGG CCC TGG AAC CCC CCC ACC TTC TCC CCA GCC CTG CTC GTG GTG |
| | T E G D N A T F T C S F S N T S E S F H |
| 121 | ACC GAA GGG GAC AAC GCC ACC TTC ACC TGC AGC TTC TCC AAC ACA TCG GAG AGC TTC CAT |
| | V I W H R E S P S G Q T D T L A A F P E |
| 181 | GTA ATC TGG CAC CGC GAG AGC CCC AGC GGC CAG ACG GAC ACG CTG GCC GCC TTC CCC GAG |
| | D R S Q P G Q D C R F R V T Q L P N G R |
| 241 | GAC CGC AGC CAG CCC GGC CAG GAC TGC CGC TTC CGT GTC ACA CAA CTG CCC AAC GGG CGT |
| | D F H M S V V R A R R N D S G T Y V C G |
| 301 | GAC TTC CAC ATG AGC GTG GTC AGG GCC CGG CGC AAT GAC AGC GGC ACC TAC GTC TGT GGG |
| | V I S L A P K I Q I K E S L R A E L R V |
| 361 | GTC ATC TCC CTG GCC CCC AAG ATC CAG ATC AAA GAG AGC CTG CGG GCA GAG CTC AGG GTG |
| | T E R R A E V P T A H P S P S P R P A G |
| 421 | ACA GAG AGA AGG GCA GAA GTG CCC ACA GCC CAC CCC AGC CCC TCA CCC AGG CCA GCC GGC |
| | Q F Q T L E P R G P T I K P C P P C K C |
| 481 | CAG TTC CAA ACC CTG GAG CCC AGA GGG CCC ACA ATC AAG CCC TGT CCT CCA TGC AAA TGC |
| | P A P N L L G G P S V F I F P P K I K D |
| 541 | CCA GCA CCT AAC CTC TTG GGT GGA CCA TCC GTC TTC ATC TTC CCT CCA AAG ATC AAG GAT |
| | V L M I S L S P I V T C V V V D V S E D |
| 601 | GTA CTC ATG ATC TCC CTG AGC CCC ATA GTC ACA TGT GTG GTG GTG GAT GTG AGC GAG GAT |
| | D P D V Q I S W F V N N V E V H T A Q T |
| 661 | GAC CCA GAT GTC CAG ATC AGC TGG TTT GTG AAC AAC GTG GAA GTA CAC ACA GCT CAG ACA |
| | Q T H R E D Y N S T L R V V S A L P I Q |
| 721 | CAA ACC CAT AGA GAG GAT TAC AAC AGT ACT CTC CGG GTG GTC AGT GCC CTC CCC ATC CAG |
| | H Q D W M S G K E F K C K V N N K D L P |
| 781 | CAC CAG GAC TGG ATG AGT GGC AAG GAG TTC AAA TGC AAG GTC AAC AAC AAA GAC CTC CCA |
| | A P I E R T I S K P K G S V R A P Q V Y |
| 841 | GCG CCC ATC GAG AGA ACC ATC TCA AAA CCC AAA GGG TCA GTA AGA GCT CCA CAG GTA TAT |
| | V L P P P E E E M T K K Q V T L T C M V |
| 901 | GTC TTG CCT CCA CCA GAA GAA GAG ATG ACT AAG AAA CAG GTC ACT CTG ACC TGC ATG GTC |
| | T D F M P E D I Y V E W T N N G K T E L |
| 961 | ACA GAC TTC ATG CCT GAA GAC ATT TAC GTG GAG TGG ACC AAC AAC GGG AAA ACA GAG CTA |
| | N Y K N T E P V L D S D G S Y F M Y S K |
| 1021 | AAC TAC AAG AAC ACT GAA CCA GTC CTG GAC TCT GAT GGT TCT TAC TTC ATG TAC AGC AAG |
| | L R V E K K N W V E R N S Y S C S V V H |
| 1081 | CTG AGA GTG GAA AAG AAG AAC TGG GTG GAA AGA AAT AGC TAC TCC TGT TCA GTG GTC CAC |
| | E G L H N H H T T K S F S R T P G K * |
| 1141 | GAG GGT CTG CAC AAT CAC CAC ACG ACT AAG AGC TTC TCC CGG ACT CCA GGT AAA TAA |

Sequence of pG8R388 (SEQ ID NO: 138 and 152)
Yellow highlighted: asd sequence (SEQ ID NO: 142 and 145 (DNA) and SEQ ID NO:144 (amino acid))
Grey highlighted: murA sequence (SEQ ID NO: 9 and 146 (DNA) and SEQ ID NO: 10 (amino acid))
Pink highlighted: araC sequence (SEQ ID NO: 11 and 147 (DNA) and SEQ ID NO: 12 (amino acid))

| | |
|---|---|
| 1 | GAC TCT TCG CGA TGT ACG GGC CAG ATA TAC GCG TTA ACT GCA GTC TAG ATT ATG CGA AAG |
| | CTG AGA AGC GCT ACA TGC CCG GTC TAT ATG CGC AAT TGA CGT CAG ATC TAA TAC GCT TTC |
| 61 | GCC ATC CTG ACG GAT GGC CTT TTT GTT TAA ACG GAT CCG CGA CAT TGA TTA TTG ACT AGT |
| | CGG TAG GAC TGC CTA CCG GAA AAA CAA ATT TGC CTA GGC GCT GTA ACT AAT AAC TGA TCA |
| 121 | TAT TAA TAG TAA TCA ATT ACG GGG TCA TTA GGG GAC TTT CCG GGG ACT TTC CTC CCC ACG |
| | ATA ATT ATC ATT AGT TAA TGC CCC AGT AAT CCC CTG AAA GGC CCC TGA AAG GAG GGG TGC |
| 181 | CGG GGG ACT TTC CGC CAC GGG CGG GGA CTT TCC GGG GAC TTT CCG TTC ATA GCC CAT ATA |
| | GCC CCC TGA AAG GCG GTG CCC GCC CCT GAA AGG CCC CTG AAA GGC AAG TAT CGG GTA TAT |
| 241 | TGG AGT TCC GCG TTA CAT AAC TTA CGG TAA ATG GCC CGC CTG GCT GAC CGC CCA ACG ACC |
| | ACC TCA AGG CGC AAT GTA TTG AAT GCC ATT TAC CGG GCG GAC CGA CTG GCG GGT TGC TGG |
| 301 | CCC GCC CAT TGA CGT CAA TAA TGA CGT ATG TTC CCA TAG TAA CGC CAA TAG GGA CTT TCC |
| | GGG CGG GTA ACT GCA GTT ATT ACT GCA TAC AAG GGT ATC ATT GCG GTT ATC CCT GAA AGG |
| 361 | ATT GAC GTC AAT GGG TGG ACT ATT TAC GGT AAA CTG CCC ACT TGG CAG TAC ATC AAG TGT |
| | TAA CTG CAG TTA CCC ACC TGA TAA ATG CCA TTT GAC GGG TGA ACC GTC ATG TAG TTC ACA |
| 421 | ATC ATA TGC CAA GTA CGC CCC CTA TTG ACG TCA ATG ACG GTA AAT GGC CCG CCT GGC ATT |
| | TAG TAT ACG GTT CAT GCG GGG GAT AAC TGC AGT TAC TGC CAT TTA CCG GGC GGA CCG TAA |
| 481 | ATG CCC AGT ACA TGA CCT TAT GGG ACT TTC CTA CTT GGC AGT ACA TCT ACG TAT TAG TCA |
| | TAC GGG TCA TGT ACT GGA ATA CCC TGA AAG GAT GAA CCG TCA TGT AGA TGC ATA ATC AGT |
| 541 | TCG CTA TTA CCA TGG TGA TGC GGT TTT GGC AGT ACA TCA ATG GGC GTG GAT AGC GGT TTG |
| | AGC GAT AAT GGT ACC ACT ACG CCA AAA CCG TCA TGT AGT TAC CCG CAC CTA TCG CCA AAC |
| 601 | ACT CAC GGG GAT TTC CAA GTC TCC ACC CCA TTG ACG TCA ATG GGA GTT TGT TTT GGC ACC |
| | TGA GTG CCC CTA AAG GTT CAG AGG TGG GGT AAC TGC AGT TAC CCT CAA ACA AAA CCG TGG |
| 661 | AAA ATC AAC GGG ACT TTC CAA AAT GTC GTA ACA ACT CCG CCC CAT TGA CGC AAA TGG GCG |
| | TTT TAG TTG CCC TGA AAG GTT TTA CAG CAT TGT TGA GGC GGG GTA ACT GCG TTT ACC CGC |
| 721 | GTA GGC GTG TAC GGT GGG AGG TCT ATA TAA GCA GAG CTC TCT GGC TAA CTA GAG AAC CCA |
| | CAT CCG CAC ATG CCA CCC TCC AGA TAT ATT CGT CTC GAG AGA CCG ATT GAT CTC TTG GGT |
| 781 | CTG CTT ACT GGC TTA TCG AAA TTA ATA CGA CTC ACT ATA GGG AGA CCC AAG CTG GCT AGC |
| | GAC GAA TGA CCG AAT AGC TTT AAT TAT GCT GAG TGA TAT CCC TCT GGG TTC GAC CGA TCG |
| 841 | GTT TAA ACT TAA GCT TGG TAC CGA GCT CGG ATC CAC TAG TCC AGT GTG GTG GAA TTC TGC |
| | CAA ATT TGA ATT CGA ACC ATG GCT CGA GCC TAG GTG ATC AGG TCA CAC CAC CTT AAG ACG |
| 901 | AGA TAT CCA GCA CAG TGG CGG CCG CTC GAG AAT GCT TCG AGC AGA CAT GAT AAG ATA CAT |
| | TCT ATA GGT CGT GTC ACC GCC GGC GAG CTC TTA CGA AGC TCG TCT GTA CTA TTC TAT GTA |
| 961 | TGA TGA GTT TGG ACA AAC CAC AAC TAG AAT GCA GTG AAA AAA ATG CTT TAT TTG TGA AAT |
| | ACT ACT CAA ACC TGT TTG GTG TTG ATC TTA CGT CAC TTT TTT TAC GAA ATA AAC ACT TTA |
| 1021 | TTG TGA TGC TAT TGC TTT ATT TGT AAC CAT TAT AAG CTG CAA TAA ACA AGT TAA CAA CAA |
| | AAC ACT ACG ATA ACG AAA TAA ACA TTG GTA ATA TTC GAC GTT ATT TGT TCA ATT GTT GTT |
| 1081 | CAA TTG CAT TCA TTT TAT GTT TCA GGT TCA GGG GGA GAT GTG GGA GGT TTT TTA AAG CAA |
| | GTT AAC GTA AGT AAA ATA CAA AGT CCA AGT CCC CCT CTA CAC CCT CCA AAA AAT TTC GTT |
| 1141 | GTA AAA CCT CTA CAA ATG TGG TAA AAT CCG ATA AGG ATC GAT CCG GGG CAT GCA ACC AGC |
| | CAT TTT GGA GAT GTT TAC ACC ATT TTA GGC TAT TCC TAG CTA GGC CCC GTA CGT TGG TCG |
| 1201 | TGT GGA ATG TGT GTC AGT TAG GGT GTG GAA AGT CCC CAG CGA GGG GTC GTC CGT CTT CAT |
| | ACA CCT TAC ACA CAG TCA ATC CCA CAC CTT TCA GGG GTC CGA GGG GTC GTC CGT CTT CAT |
| 1261 | TGC AAA GCA TGT GGG GAT GCG GTG GGC TCT ATG GCT TCT ACT GGG CGG TTT TAT GGA CAG |
| | ACG TTT CGT ACA CCC CTA CGC CAC CCG AGA TAC CGA AGA TGA CCC GCC AAA ATA CCT GTC |
| 1321 | CAA GCG AAC CGG AAT TGC CAG CTG GGG CGC CCT CTG GTA AGG TTG GGA AGC CCT GCA AAG |
| | GTT CGC TTG GCC TTA ACG GTC GAC CCC GCG GGA GAC CAT TCC AAC CCT TCG GGA CGT TTC |
| 1381 | TAA ACT GGA TGG CTT TCT CGC CGC CAA GGA TCT GTC GAC CCC TAG ATT TCA GTG CAA TTT |
| | ATT TGA CCT ACC GAA AGA GCG GCG GTT CCT AGA CAG CTG GGG ATC TAA AGT CAC GTT AAA |
| 1441 | ATC TCT TCA AAT GTA GCA CCT GAA GTC AGC CCC ATA CGA TAT AAG TTG TTG GAA GAT CTA |
| | TAG AGA AGT TTA CAT CGT GGA CTT CAG TCG GGG TAT GCT ATA TTC AAC AAC CTT CTA GAT |
| 1501 | GCC CGC CTA ATG AGC GGG CTT TTT TTT AAT TCG CAA TTC CCC GAT GCA TAA TGT GCC TGT |
| | CGG GCG GAT TAC TCG CCC GAA AAA AAA TTA AGC GTT AAG GGG CTA CGT ATT ACA CGG ACA |
| 1561 | CAA ATG GAC GAA GCA GGG ATT CTG CAA ACC CTA TGC TAC TCC GTC AAG CCG TCA ATT GTC |
| | GTT TAC CTG CTT CGT CCC TAA GAC GTT TGG GAT ACG ATG AGG CAG TTC GGC AGT TAA CAG |
| 1621 | 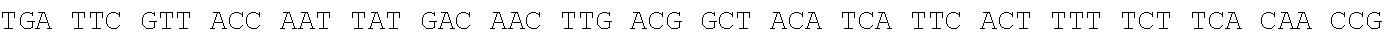 |
| | 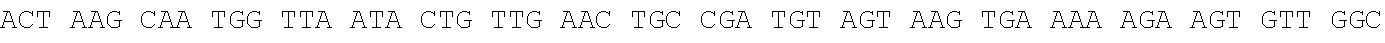 |
| | S L K V A V D N V K E E C G A |
| 1681 | 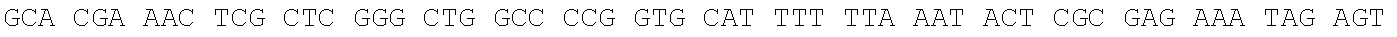 |
| | 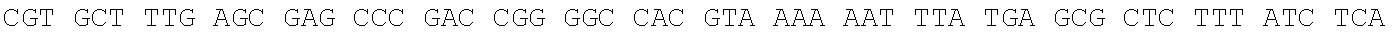 |
| | .. R F E S P S A G T C K K F V R S F Y L Q |
| 1741 | 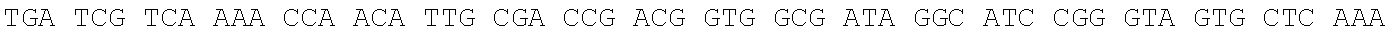 |
| | 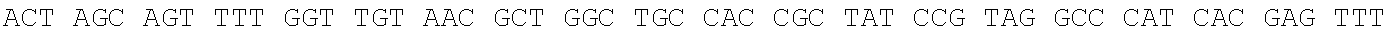 |
| | .. D D F G V N R G V T A I P M R T T S L L |
| 1801 | 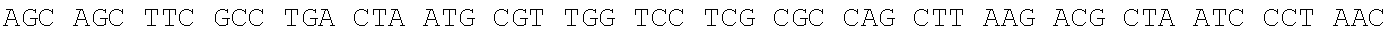 |
| | 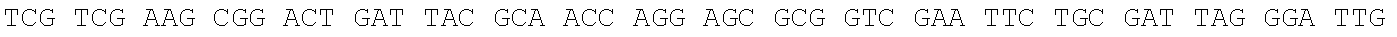 |
| | .. L K A Q S I R Q D E R W S L V S I G L Q |
| 1861 | 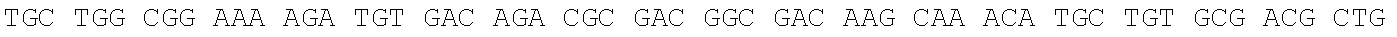 |
| | 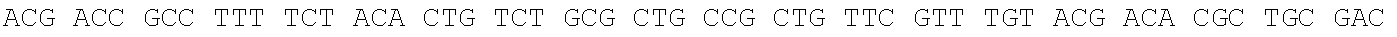 |
| | .. Q R F L H S L R S P S L C V H Q A V S A |
| 1921 | 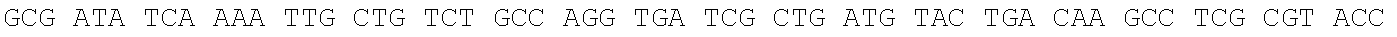 |
| | 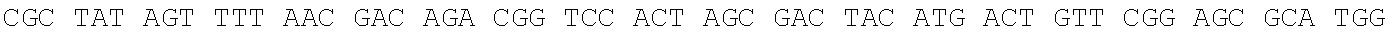 |
| | .. I D F N S D A L H D S I Y Q C A E R V R |
| 1981 | 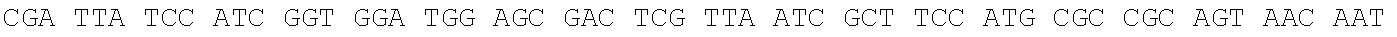 |
| | 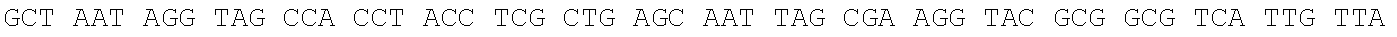 |
| | .. N D M P P H L S E N I A E M R R L L L Q |
| 2041 | 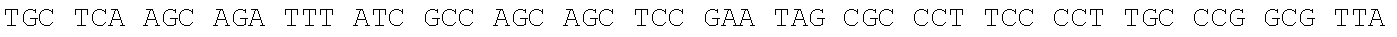 |
| | 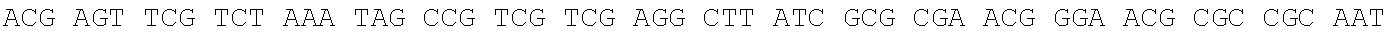 |
| | .. E L L N I A L L E S Y R G E G Q G A N I |
| 2101 | 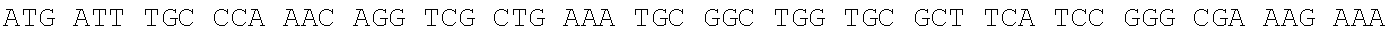 |
| | 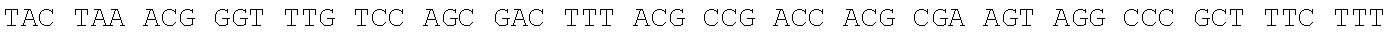 |
| | .. I Q G F L D S F H P Q H A E D P R F F G |
| 2161 | 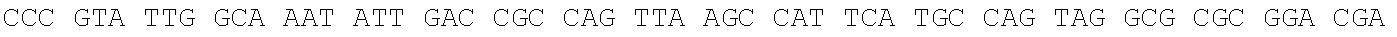 |
| | 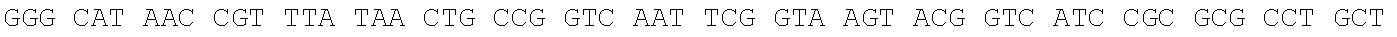 |
| | .. T N A F I S P W N L W E H W Y A R P R F |
| 2221 | 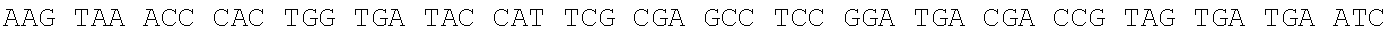 |
| | 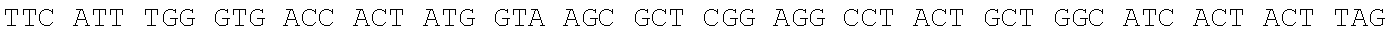 |
| | .. Y V W Q H Y W E R A E P H R G Y H H I E |
| 2281 | 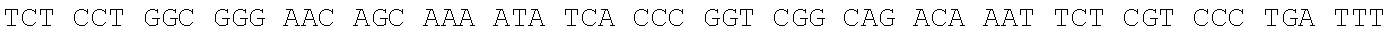 |
| | 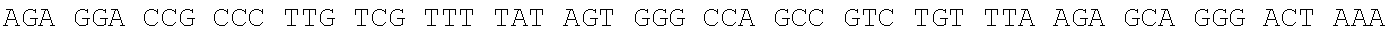 |
| | .. G P P F L L I D G P R C V F E R G Q N K |
| 2341 | 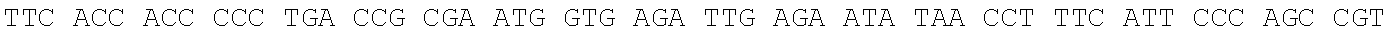 |
| | 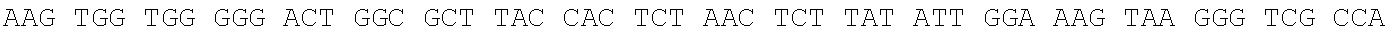 |
| | .. V V G Q G R I T L N L I Y G K M G L P R |
| 2401 | 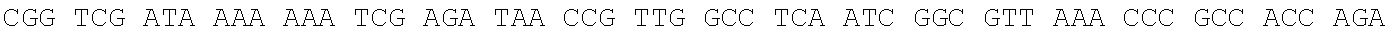 |
| | 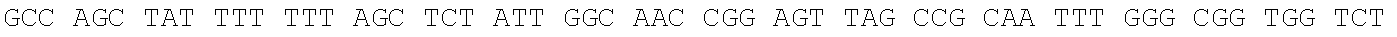 |
| | .. D I F F D L Y G N A E I P T L G A V L H |
| 2461 | 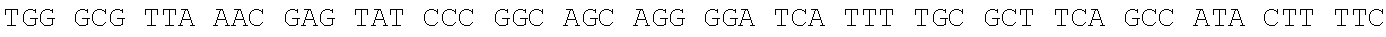 |
| | 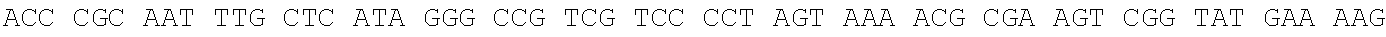 |
| | .. A N F S Y G P L L P D N Q A E A M |
| 2521 | ATA CTC CCA CCA TTC AGA GAA GAA ACC AAT TGT CCA TAT TGC ATC AGA CAT TGC CGT CAC |
| | TAT GAG GGT GGT AAG TCT CTT CTT TGG TTA ACA GGT ATA ACG TAG TCT GTA ACG GCA GTG |
| 2581 | TGC GTC TTT TAC TGG CTC TTC TCG CTA ACC CAA CCG GTA ACC CCG CTT ATT AAA AGC ATT |
| | ACG CAG AAA ATG ACC GAG AAG AGC GAT TGG GTT GGC CAT TGG GGC GAA TAA TTT TCG TAA |
| 2641 | CTG TAA CAA AGC GGG ACC AAA GCC ATG ACA AAA ACG CGT AAC AAA AGT GTC TAT AAT CAC |
| | GAC ATT GTT TCG CCC TGG TTT CGG TAC TGT TTT TGC GCA TTG TTT TCA CAG ATA TTA GTG |
| 2701 | GGC AGA AAA GTC CAC ATT GAT TAT TTG CAC GGC GTC ACA CTT TGC TAT GCC ATA GCA TTT |
| | CCG TCT TTT CAG GTG TAA CTA ATA AAC GTG CCG CAG TGT GAA ACG ATA CGG TAT CGT AAA |
| 2761 | TTA TCC ATA AGA TTA GCG GAT CCT ACC TGA CGC TTT TTA TCG CAA CTC TCT ACT GTT TCT |
| | AAT AGG TAT TCT AAT CGC CTA GGA TGG ACT GCG AAA AAT AGC GTT GAG AGA TGA CAA AGA |
| | M D K F R V |
| 2821 | 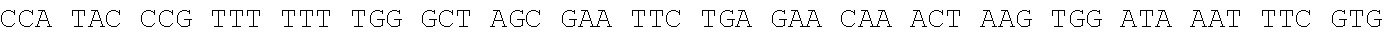 |
| | 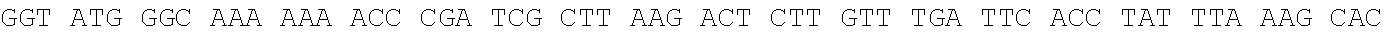 |
| | . Q G P T K L Q G E V T I S G A K N A A L |
| 2881 | 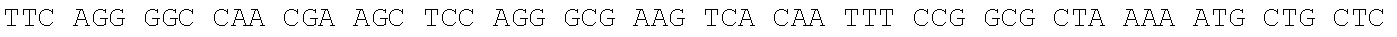 |
| | 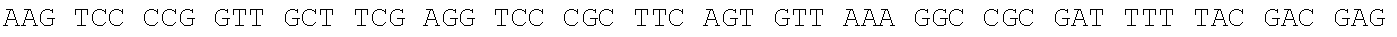 |
| | . P I L F A A L L A E E P V E I Q N V P K |
| 2941 | 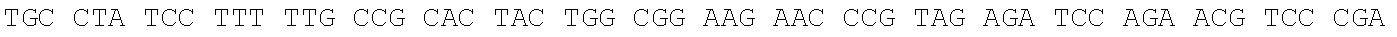 |
| | 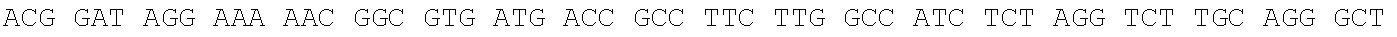 |
| | . L K D V D T S M K L L S Q L G A K V E R |
| 3001 | 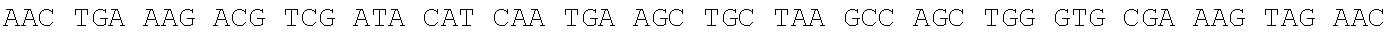 |
| | 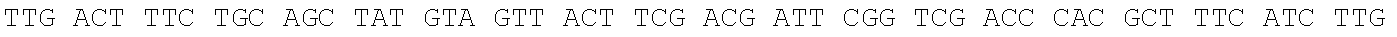 |
| | . N G S V H I D A R D V N V F C A P Y D L |
| 3061 | 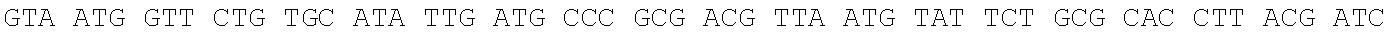 |
| | 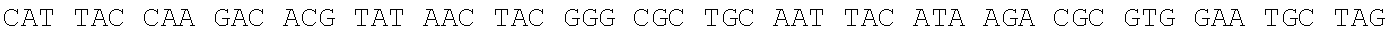 |
| | . V K T M R A S I W A L G P L V A R F G Q |
| 3121 | 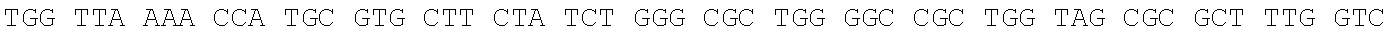 |
| | 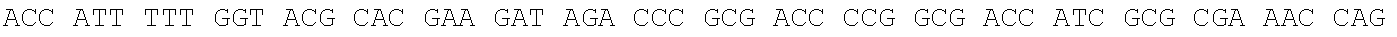 |
| | . G Q V S L P G G C T I G A R P V D L H I |
| 3181 | 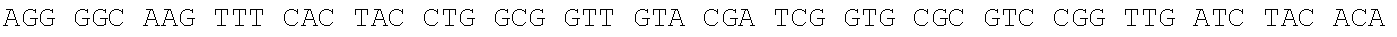 |
| | 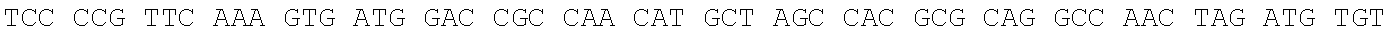 |
| | . S G L E Q L G A T I K L E E G Y V K A S |
| 3241 | 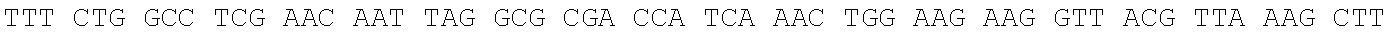 |
| | 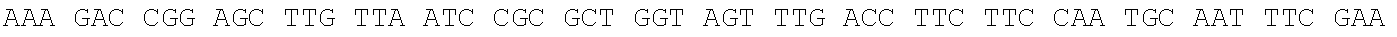 |
| | . V D G R L K G A H I V M D K V S V G A T |
| 3301 | 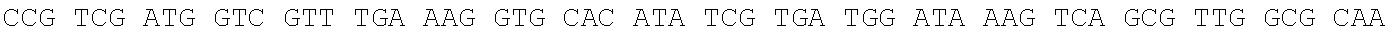 |
| | 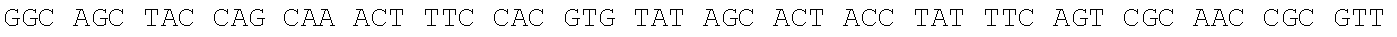 |
| | . V T I M C A A T L A E G T T I I E N A A |
| 3361 | 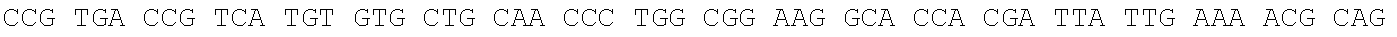 |
| | 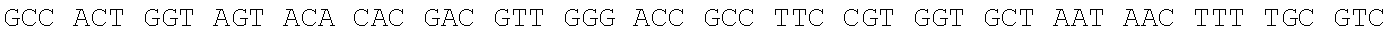 |
| | . R E P E I V D T A N F L I T L G A K I S |
| 3421 | 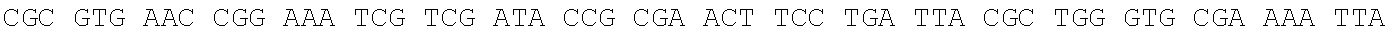 |
| | 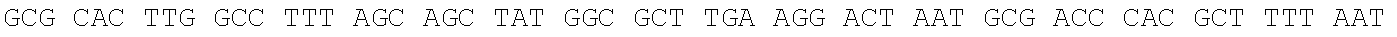 |
| | . G Q G T D R I V I E G V E R L G G G V Y |
| 3481 | 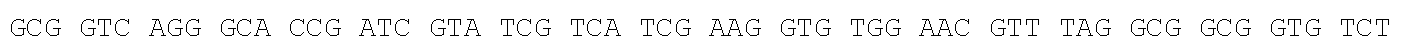 |
| | 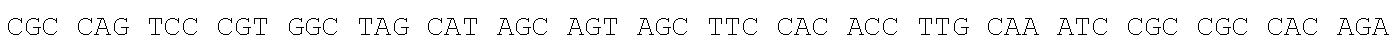 |
| | . R V L P D R I E T G T F L V A A A I S R |
| 3541 | 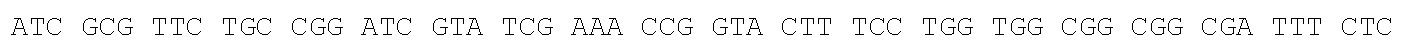 |
| | 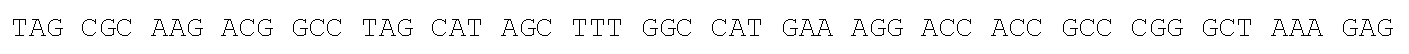 |
| | . G K I I C R N A Q P D T L D A V L A K L |
| 3601 | 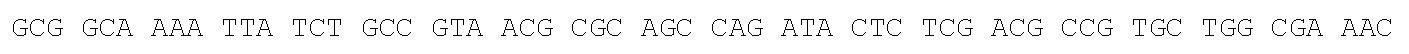 |
| | 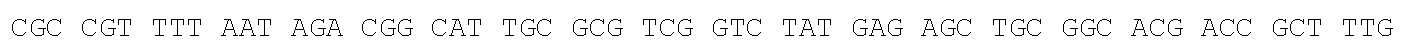 |
| | . R D A G A D I E V G E D W I S L D M H G |
| 3661 | 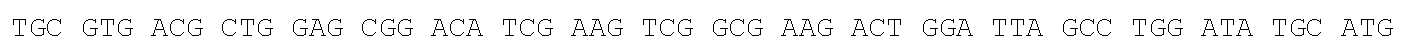 |
| | 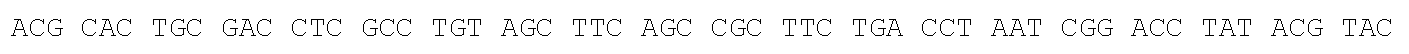 |
| | . K R P K A V N V R T A P H P A F P T D M |
| 3721 | 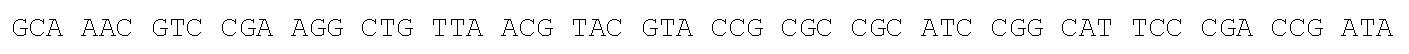 |
| | 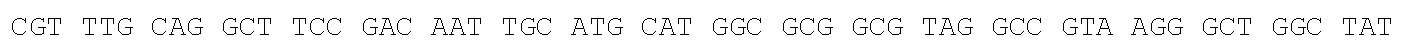 |
| | . Q A Q F T L L N L V A E G T G F I T E T |
| 3781 | 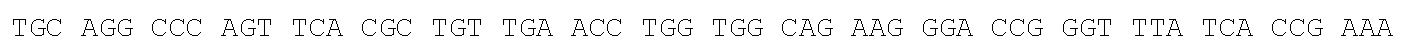 |
| | 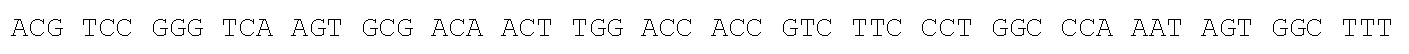 |
| | . V F E N R F M H V P E L S R M G A H A E |
| 3841 | 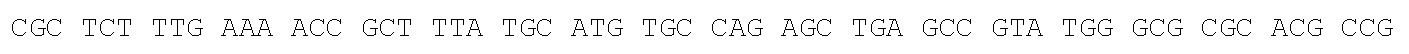 |
| | 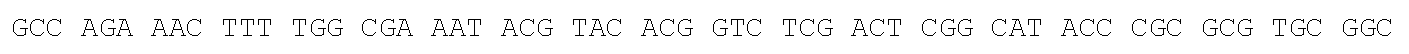 |
| | . I E S N T V I C H G V E K L S G A Q V M |
| 3901 | 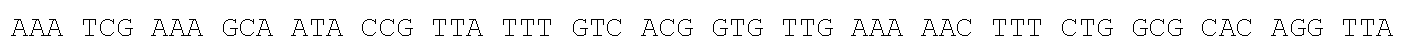 |
| | 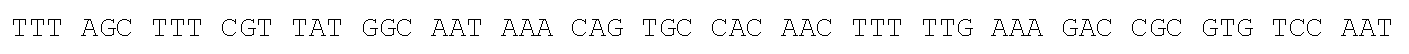 |
| | . A T D L R A S A S L V L A G C I A E G T |
| 3961 | 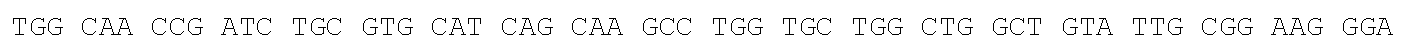 |
| | 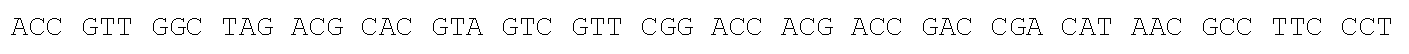 |
| | . T V V D R I Y H I D R G Y E R I E D K L |
| 4021 | 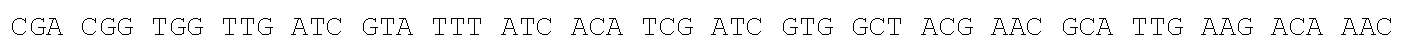 |
| | 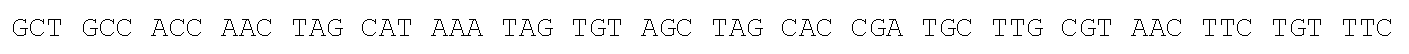 |
| | . R A L G A N I E R V K G E |
| 4081 | 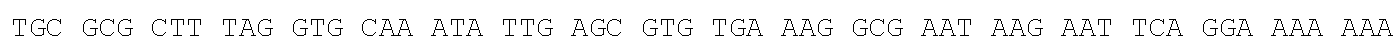 |
| | 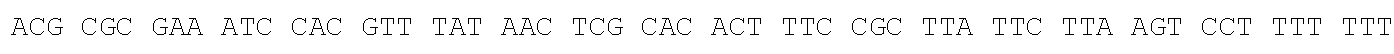 |
| | M K N V G F I G W R G M V G S V L M Q |
| 4141 | CGC TGT GAA AAA TGT TGG TTT TAT CGG CTG GCG CGG AAT GGT CGG CTC TGT TCT CAT GCA |
| | GCG ACA CTT TTT ACA ACC AAA ATA GCC GAC CGC GCC TTA CCA GCC GAG ACA AGA GTA CGT |
| | . R M V E E R D F D A I R P V F F S T S Q |
| 4201 | ACG CAT GGT AGA GGA GCG CGA TTT CGA CGC TAT TCG CCC TGT TTT CTT TTC TAC CTC CCA |
| | TGC GTA CCA TCT CCT CGC GCT AAA GCT GCG ATA AGC GGG ACA AAA GAA AAG ATG GAG GGT |
| | . F G Q A A P T F G D T S T G T L Q D A F |
| 4261 | GTT TGG ACA GGC GGC GCC CAC CTT CGG CGA CAC CTC CAC CGG CAC GCT ACA GGA CGC TTT |
| | CAA ACC TGT CCG CCG CGG GTG GAA GCC GCT GTG GAG GTG GCC GTG CGA TGT CCT GCG AAA |
| | . D L D A L K A L D I I V T C Q G G D Y T |
| 4321 | TGA TCT GGA TGC GCT AAA AGC GCT CGA TAT CAT CGT GAC CTG CCA GGG CGG CGA TTA TAC |
| | ACT AGA CCT ACG CGA TTT TCG CGA GCT ATA GTA GCA CTG GAC GGT CCC GCC GCT AAT ATG |
| | . N E I Y P K L R E S G W Q G Y W I D A A |
| 4381 | CAA CGA AAT TTA TCC AAA GCT GCG CGA AAG CGG ATG GCA GGG TTA CTG GAT TGA TGC GGC |
| | GTT GCT TTA AAT AGG TTT CGA CGC GCT TTC GCC TAC CGT CCC AAT GAC CTA ACT ACG CCG |
| | . S T L R M K D D A I I I L D P V N Q D V |
| 4441 | TTC TAC GCT GCG CAT GAA AGA TGA TGC CAT TAT TAT TCT CGA CCC GGT CAA CCA GGA CGT |
| | AAG ATG CGA CGC GTA CTT TCT ACT ACG GTA ATA ATA AGA GCT GGG CCA GTT GGT CCT GCA |
| | . I T D G L N N G V K T F V G G N C T V S |
| 4501 | GAT TAC CGA CGG CCT GAA CAA TGG CGT GAA GAC CTT TGT GGG CGG TAA CTG TAC CGT TAG |
| | CTA ATG GCT GCC GGA CTT GTT ACC GCA CTT CTG GAA ACA CCC GCC ATT GAC ATG GCA ATC |
| | . L M L M S L G G L F A H N L V D W V S V |
| 4561 | CCT GAT GTT GAT GTC GCT GGG CGG TCT CTT TGC CCA TAA TCT CGT TGA CTG GGT ATC CGT |
| | GGA CTA CAA CTA CAG CGA CCC GCC AGA GAA ACG GGT ATT AGA GCA ACT GAC CCA TAG GCA |
| | . A T Y Q A A S G G G A R H M R E L L T Q |
| 4621 | CGC GAC CTA TCA GGC CGC CTC CGG CGG CGG CGC GCG CCA TAT GCG CGA GCT GTT AAC CCA |
| | GCG CTG GAT AGT CCG GCG GAG GCC GCC GCC GCG CGC GGT ATA CGC GCT CGA CAA TTG GGT |
| | . M G Q L Y G H V A D E L A T P S S A I L |
| 4681 | GAT GGG TCA GTT GTA TGG CCA TGT CGC CGA TGA ACT GGC GAC GCC GTC TTC CGC AAT TCT |
| | CTA CCC AGT CAA CAT ACC GGT ACA GCG GCT ACT TGA CCG CTG CGG CAG AAG GCG TTA AGA |
| | . D I E R K V T A L T R S G E L P V D N F |
| 4741 | TGA TAT TGA ACG CAA AGT TAC GGC ATT GAC CCG CAG CGG CGA GCT GCC GGT TGA TAA CTT |
| | ACT ATA ACT TGC GTT TCA ATG CCG TAA CTG GGC GTC GCC GCT CGA CGG CCA ACT ATT GAA |
| | . G V P L A G S L I P W I D K Q L D N G Q |
| 4801 | TGG CGT ACC GCT GGC GGG AAG CCT GAT CCC CTG GAT CGA CAA ACA GCT CGA TAA CGG CCA |
| | ACC GCA TGG CGA CCG CCC TTC GGA CTA GGG GAC CTA GCT GTT TGT CGA GCT ATT GCC GGT |
| | . S R E E W K G Q A E T N K I L N T A S V |
| 4861 | GAG CCG CGA AGA GTG GAA AGG CCA GGC GGA AAC CAA CAA GAT TCT CAA TAC TGC CTC TGT |
| | CTC GGC GCT TCT CAC CTT TCC GGT CCG CCT TTG GTT GTT CTA AGA GTT ATG ACG GAG ACA |
| | . I P V D G L C V R V G A L R C H S Q A F |
| 4921 | GAT TCC GGT TGA TGG TTT GTG TGT GCG CGT CGG CGC GCT GCG CTG TCA CAG CCA GGC GTT |
| | CTA AGG CCA ACT ACC AAA CAC ACA CGC GCA GCC GCG CGA CGC GAC AGT GTC GGT CCG CAA |
| | . T I K L K K E V S I P T V E E L L A A H |
| 4981 | CAC CAT CAA GCT GAA AAA AGA GGT ATC CAT TCC GAC GGT GGA AGA ACT GCT GGC GGC ACA |
| | GTG GTA GTT CGA CTT TTT TCT CCA TAG GTA AGG CTG CCA CCT TCT TGA CGA CCG CCG TGT |
| | . N P W A K V V P N D R D I T M R E L T P |
| 5041 | TAA TCC GTG GGC GAA AGT GGT GCC GAA CGA TCG TGA TAT CAC TAT GCG CGA ATT AAC CCC |
| | ATT AGG CAC CCG CTT TCA CCA CGG CTT GCT AGC ACT ATA GTG ATA CGC GCT TAA TTG GGG |
| | . A A V T G T L T T P V G R L R K L N M G |
| 5101 | GGC GGC GGT GAC CGG CAC GTT GAC TAC GCC GGT TGG TCG TCT GCG TAA GCT GAA CAT GGG |
| | CCG CCG CCA CTG GCC GTG CAA CTG ATG CGG CCA ACC AGC AGA CGC ATT CGA CTT GTA CCC |
| | . P E F L S A F T V G D Q L L W G A A E P |
| 5161 | GCC AGA GTT CTT GTC GGC GTT TAC CGT AGG CGA CCA GTT GTT ATG GGG CGC CGC CGA GCC |
| | CGG TCT CAA GAA CAG CCG CAA ATG GCA TCC GCT GGT CAA CAA TAC CCC GCG GCG GCT CGG |
| | . L R R M L R Q L A |
| 5221 | GCT GCG TCG AAT GCT GCG CCA GTT GGC GTA GTC TAG CTG CAC GAT ACC GTC GAC TTG TAC |
| | CGA CGC AGC TTA CGA CGC GGT CAA CCG CAT CAG ATC GAC GTG CTA TGG CAG CTG AAC ATG |
| 5281 | ATA GAC TCG CTC CGA AAT TAA AGA ACA CTT AAA TTA TCT ACT AAA GGA ATC TTT AGT CAA |
| | TAT CTG AGC GAG GCT TTA ATT TCT TGT GAA TTT AAT AGA TGA TTT CCT TAG AAA TCA GTT |
| 5341 | GTT TAT TTA AGA TGA CTT AAC TAT GAA TAC ACA ATT GAT GGG TGA GCG TAG GAA AAA AAA |
| | CAA ATA AAT TCT ACT GAA TTG ATA CTT ATG TGT TAA CTA CCC ACT CGC ATC CTT TTT TTT |
| 5401 | ACC CCG CCC CTG ACA GGG CGG GGT TTT TTT TGA TCA TTC TGA AAT GAG CTG TTG ACA ATT |
| | TGG GGC GGG GAC TGT CCC GCC CCA AAA AAA ACT AGT AAG ACT TTA CTC GAC AAC TGT TAA |
| 5461 | AAT CAT CCG GCT CGT ATA ATG TGT GGA ATT GTG AGC GGA TAA CAA TTT CAC ACA GGA AAC |
| | TTA GTA GGC CGA GCA TAT TAC ACA CCT TAA CAC TCG CCT ATT GTT AAA GTG TGT CCT TTG |
| 5521 | AGA CCC TAG GAG TAC TCC GCG GCC CGG GCC CCT GCA GGT TCG AAA CAG ATT AAA TCA GAA |
| | TCT GGG ATC CTC ATG AGG CGC CGG GCC CGG GGA CGT CCA AGC TTT GTC TAA TTT AGT CTT |
| 5581 | CGC AGA AGC GGT CTG ATA AAA CAG AAT TTG CCT GGC GGC AGT AGC GCG GTG GTC CCA CCT |
| | GCG TCT TCG CCA GAC TAT TTT GTC TTA AAC GGA CCG CCG TCA TCG CGC CAC CAG GGT GGA |
| 5641 | GAC CCC ATG CCG AAC TCA GAA GTG AAA CGC CGT AGC GCC GAT GGT AGT GTG GGG TCT CCC |
| | CTG GGG TAC GGC TTG AGT CTT CAC TTT GCG GCA TCG CGG CTA CCA TCA CAC CCC AGA GGG |
| 5701 | CAT GCG AGA GTA GGG AAC TGC CAG GCA TCA AAT AAA ACG AAA GGC TCA GTC GAA AGA CTG |
| | GTA CGC TCT CAT CCC TTG ACG GTC CGT AGT TTA TTT TGC TTT CCG AGT CAG CTT TCT GAC |
| 5761 | GGC CTT TCG TTT TAT CTG TTG TTT GTC GGT GAA CGC TCT CCT GAG TAG GAC AAA TCC GCC |
| | CCG GAA AGC AAA ATA GAC AAC AAA CAG CCA CTT GCG AGA GGA CTC ATC CTG TTT AGG CGG |
| 5821 | GGG AGC GGA TTT GAA CGT TGC GAA GCA ACG GCC CGG AGG GTG GCG GGC AGG ACG CCC GCC |
| | CCC TCG CCT AAA CTT GCA ACG CTT CGT TGC CGG GCC TCC CAC CGC CCG TCC TGC GGG CGG |
| 5881 | ATA AAC TGC CAG GCA TCA AAT TAA GCA GAA GGC CAT CCT GAC GGA TGG CCT TTT TGC GTT |
| | TAT TTG ACG GTC CGT AGT TTA ATT CGT CTT CCG GTA GGA CTG CCT ACC GGA AAA ACG CAA |
| 5941 | TCT ACA AAC TCT TTT TGT TTA TTT TTC TAA ATA CAT TCA AAT ATG TAT CCG CTC ATG AGA |
| | AGA TGT TTG AGA AAA ACA AAT AAA AAG ATT TAT GTA AGT TTA TAC ATA GGC GAG TAC TCT |
| 6001 | CAA TAA CCC TGA TAA ATG CTT CAA TAA TGG AAG ATC TTC CAA CAT CAC AGG TAA ACA GAA |
| | GTT ATT GGG ACT ATT TAC GAA GTT ATT ACC TTC TAG AAG GTT GTA GTG TCC ATT TGT CTT |
| 6061 | ACG TCG GGT CGA TCG GGA AAT TCT TTC CCG GAC GGC GCG GGG TTG GGC AAG CCG CAG GCG |
| | TGC AGC CCA GCT AGC CCT TTA AGA AAG GGC CTG CCG CGC CCC AAC CCG TTC GGC GTC CGC |
| 6121 | CGT CAG TGC TTT TAG CGG GTG TCG GGG CAG CCC TGA ACC AGT CAC GGG ATC GAT CTG TGC |
| | GCA GTC ACG AAA ATC GCC CAC AGC CCC GTC GGG ACT TGG TCA GTG CCC TAG CTA GAC ACG |
| 6181 | GGT ATT TCA CAC CGC ATA CAG GTG GCA CTT TTC GGG GAA ATG TGC GCG GAA CCC CTA TTT |
| | CCA TAA AGT GTG GCG TAT GTC CAC CGT GAA AAG CCC CTT TAC ACG CGC CTT GGG GAT AAA |
| 6241 | GTT TAT TTT TCT AAA TAC ATT CAA ATA TGT ATC CGC TCA TGA GAC AAT AAC CCT GAT AAA |
| | CAA ATA AAA AGA TTT ATG TAA GTT TAT ACA TAG GCG AGT ACT CTG TTA TTG GGA CTA TTT |
| 6301 | TGC TTC AAT AAT AGC ACG TGC TAA AAC TTC ATT TTT AAT TTA AAA GGA TCT AGG TGA AGA |
| | ACG AAG TTA TTA TCG TGC ACG ATT TTG AAG TAA AAA TTA AAT TTT CCT AGA TCC ACT TCT |
| 6361 | TCC TTT TTG ATA ATC TCA TGA CCA AAA TCC CTT AAC GTG AGT TTT CGT TCC ACT GAG CGT |
| | AGG AAA AAC TAT TAG AGT ACT GGT TTT AGG GAA TTG CAC TCA AAA GCA AGG TGA CTC GCA |
| 6421 | CAG ACC CCG TAG AAA AGA TCA AAG GAT CTT CTT GAG ATC CTT TTT TTC TGC GCG TAA TCT |
| | GTC TGG GGC ATC TTT TCT AGT TTC CTA GAA GAA CTC TAG GAA AAA AAG ACG CGC ATT AGA |
| 6481 | GCT GCT TGC AAA CAA AAA AAC CAC CGC TAC CAG CGG TGG TTT GTT TGC CGG ATC AAG AGC |
| | CGA CGA ACG TTT GTT TTT TTG GTG GCG ATG GTC GCC ACC AAA CAA ACG GCC TAG TTC TCG |
| 6541 | TAC CAA CTC TTT TTC CGA AGG TAA CTG GCT TCA GCA GAG CGC AGA TAC CAA ATA CTG TCC |
| | AAG ATC ACA TCG GCA TCA ATC CGG TGG TGA AGT TCT TGA GAC ATC GTG GCG GAT GTA TGG |
| 6601 | TTC TAG TGT AGC CGT AGT TAG GCC ACC ACT TCA AGA ACT CTG TAG CAC CGC CTA CAT ACC |
| | AAG ATC ACA TCG GCA TCA ATC CGG TGG TGA AGT TCT TGA GAC ATC GTG GCG GAT GTA TGG |
| 6661 | TCG CTC TGC TAA TCC TGT TAC CAG TGG CTG CTG CCA GTG GCG ATA AGT CGT GTC TTA CCG |
| | AGC GAG ACG ATT AGG ACA ATG GTC ACC GAC GAC GGT CAC CGC TAT TCA GCA CAG AAT GGC |
| 6721 | GGT TGG ACT CAA GAC GAT AGT TAC CGG ATA AGG CGC AGC GGT CGG GCT GAA CGG GGG GTT |
| | CCA ACC TGA GTT CTG CTA TCA ATG GCC TAT TCC GCG TCG CCA GCC CGA CTT GCC CCC CAA |
| 6781 | CGT GCA CAC AGC CCA GCT TGG AGC GAA CGA CCT ACA CCG AAC TGA GAT ACC TAC AGC GTG |
| | GCA CGT GTG TCG GGT CGA ACC TCG CTT GCT GGA TGT GGC TTG ACT CTA TGG ATG TCG CAC |
| 6841 | AGC TAT GAG AAA GCG CCA CGC TTC CCG AAG GGA GAA AGG CGG ACA GGT ATC CGG TAA GCG |
| | TCG ATA CTC TTT CGC GGT GCG AAG GGC TTC CCT CTT TCC GCC TGT CCA TAG GCC ATT CGC |
| 6901 | GCA GGG TCG GAA CAG GAG AGC GCA CGA GGG AGC TTC CAG GGG GAA ACG CCT GGT ATC TTT |
| | CGT CCC AGC CTT GTC CTC TCG CGT GCT CCC TCG AAG GTC CCC CTT TGC GGA CCA TAG AAA |
| 6961 | ATA GTC CTG TCG GGT TTC GCC ACC TCT GAC TTG AGC GTC GAT TTT TGT GAT GCT CGT CAG |
| | TAT CAG GAC AGC CCA AAG CGG TGG AGA CTG AAC TCG CAG CTA AAA ACA CTA CGA GCA GTC |
| 7021 | GGG GGC GGA GCC TAT GGA AAA ACG CCA GCA ACG CGG CCT TTT TAC GGT TCC TGG GCT TTT |
| | CCC CCG CCT CGG ATA CCT TTT TGC GGT CGT TGC GCC GGA AAA ATG CCA AGG ACC CGA AAA |
| 7081 | GCT GGC CTT TTG CTC ACA TGT TCT |
| | CGA CCG GAA AAC GAG TGT ACA AGA |

Sequence of pG8R389 (SEQ ID NO: 139 and 153)
Red highlight: optimal bla sequence (SEQ ID NO: 127 (DNA) and SEQ ID NO: 128 (amino acid))
Yellow highlighted: asd sequence (SEQ ID NO: 142 and 145(DNA) and SEQ ID NO: 144 (amino acid))
Grey highlighted: murA sequence (SEQ ID NO: 9 and 146 (DNA) and SEQ ID NO: 10 (amino acid))
Pink highlighted: araC sequence (SEQ ID NO: 11 and 147 (DNA) and SEQ ID NO: 12 (amino acid))

| | |
|---|---|
| 1 | GAC TCT TCG CGA TGT ACG GGC CAG ATA TAC GCG TTA ACT GCA GTC TAG ATT ATG CGA AAG |
| | CTG AGA AGC GCT ACA TGC CCG GTC TAT ATG CGC AAT TGA CGT CAG ATC TAA TAC GCT TTC |
| 61 | GCC ATC CTG ACG GAT GGC CTT TTT GTT TAA ACG GAT CCG CGA CAT TGA TTA TTG ACT AGT |
| | CGG TAG GAC TGC CTA CCG GAA AAA CAA ATT TGC CTA GGC GCT GTA ACT AAT AAC TGA TCA |
| 121 | TAT TAA TAG TAA TCA ATT ACG GGG TCA TTA GGG GAC TTT CCG GGG ACT TTC CTC CCC ACG |
| | ATA ATT ATC ATT AGT TAA TGC CCC AGT AAT CCC CTG AAA GGC CCC TGA AAG GAG GGG TGC |
| 181 | CGG GGG ACT TTC CGC CAC GGG CGG GGA CTT TCC GGG GAC TTT CCG TTC ATA GCC CAT ATA |
| | GCC CCC TGA AAG GCG GTG CCC GCC CCT GAA AGG CCC CTG AAA GGC AAG TAT CGG GTA TAT |
| 241 | TGG AGT TCC GCG TTA CAT AAC TTA CGG TAA ATG GCC CGC CTG GCT GAC CGC CCA ACG ACC |
| | ACC TCA AGG CGC AAT GTA TTG AAT GCC ATT TAC CGG GCG GAC CGA CTG GCG GGT TGC TGG |
| 301 | CCC GCC CAT TGA CGT CAA TAA TGA CGT ATG TTC CCA TAG TAA CGC CAA TAG GGA CTT TCC |
| | GGG CGG GTA ACT GCA GTT ATT ACT GCA TAC AAG GGT ATC ATT GCG GTT ATC CCT GAA AGG |
| 361 | ATT GAC GTC AAT GGG TGG ACT ATT TAC GGT AAA CTG CCC ACT TGG CAG TAC ATC AAG TGT |
| | TAA CTG CAG TTA CCC ACC TGA TAA ATG CCA TTT GAC GGG TGA ACC GTC ATG TAG TTC ACA |
| 421 | ATC ATA TGC CAA GTA CGC CCC CTA TTG ACG TCA ATG ACG GTA AAT GGC CCG CCT GGC ATT |
| | TAG TAT ACG GTT CAT GCG GGG GAT AAC TGC AGT TAC TGC CAT TTA CCG GGC GGA CCG TAA |
| 481 | ATG CCC AGT ACA TGA CCT TAT GGG ACT TTC CTA CTT GGC AGT ACA TCT ACG TAT TAG TCA |
| | TAC GGG TCA TGT ACT GGA ATA CCC TGA AAG GAT GAA CCG TCA TGT AGA TGC ATA ATC AGT |
| 541 | TCG CTA TTA CCA TGG TGA TGC GGT TTT GGC AGT ACA TCA ATG GGC GTG GAT AGC GGT TTG |
| | AGC GAT AAT GGT ACC ACT ACG CCA AAA CCG TCA TGT AGT TAC CCG CAC CTA TCG CCA AAC |
| 601 | ACT CAC GGG GAT TTC CAA GTC TCC ACC CCA TTG ACG TCA ATG GGA GTT TGT TTT GGC ACC |
| | TGA GTG CCC CTA AAG GTT CAG AGG TGG GGT AAC TGC AGT TAC CCT CAA ACA AAA CCG TGG |
| 661 | AAA ATC AAC GGG ACT TTC CAA AAT GTC GTA ACA ACT CCG CCC CAT TGA CGC AAA TGG GCG |
| | TTT TAG TTG CCC TGA AAG GTT TTA CAG CAT TGT TGA GGC GGG GTA ACT GCG TTT ACC CGC |
| 721 | GTA GGC GTG TAC GGT GGG AGG TCT ATA TAA GCA GAG CTC TCT GGC TAA CTA GAG AAC CCA |
| | CAT CCG CAC ATG CCA CCC TCC AGA TAT ATT CGT CTC GAG AGA CCG ATT GAT CTC TTG GGT |
| 781 | CTG CTT ACT GGC TTA TCG AAA TTA ATA CGA CTC ACT ATA GGG AGA CCC AAG CTG GCT AGC |
| | GAC GAA TGA CCG AAT AGC TTT AAT TAT GCT GAG TGA TAT CCC TCT GGG TTC GAC CGA TCG |
| 841 | GTT TAA ACT TAA GCT TGG TAC CGA GCT CGG ATC CAC TAG TCC AGT GTG GTG GAA TTC TGC |
| | CAA ATT TGA ATT CGA ACC ATG GCT CGA GCC TAG GTG ATC AGG TCA CAC CAC CTT AAG ACG |
| 901 | AGA TAT CCA GCA CAG TGG CGG CCG CTC GAG AAT GCT TCG AGC AGA CAT GAT AAG ATA CAT |
| | TCT ATA GGT CGT GTC ACC GCC GGC GAG CTC TTA CGA AGC TCG TCT GTA CTA TTC TAT GTA |
| 961 | TGA TGA GTT TGG ACA AAC CAC AAC TAG AAT GCA GTG AAA AAA ATG CTT TAT TTG TGA AAT |
| | ACT ACT CAA ACC TGT TTG GTG TTG ATC TTA CGT CAC TTT TTT TAC GAA ATA AAC ACT TTA |
| 1021 | TTG TGA TGC TAT TGC TTT ATT TGT AAC CAT TAT AAG CTG CAA TAA ACA AGT TAA CAA CAA |
| | AAC ACT ACG ATA ACG AAA TAA ACA TTG GTA ATA TTC GAC GTT ATT TGT TCA ATT GTT GTT |
| 1081 | CAA TTG CAT TCA TTT TAT GTT TCA GGT TCA GGG GGA GAT GTG GGA GGT TTT TTA AAG CAA |
| | GTT AAC GTA AGT AAA ATA CAA AGT CCA AGT CCC CCT CTA CAC CCT CCA AAA AAT TTC GTT |
| 1141 | GTA AAA CCT CTA CAA ATG TGG TAA AAT CCG ATA AGG ATC GAT CCG GGG CAT GCA ACC AGC |
| | CAT TTT GGA GAT GTT TAC ACC ATT TTA GGC TAT TCC TAG CTA GGC CCC GTA CGT TGG TCG |
| 1201 | TGT GGA ATG TGT GTC AGT TAG GGT GTG GAA AGT CCC CAG CGA GGG GTC GTC CGT CTT CAT |
| | ACA CCT TAC ACA CAG TCA ATC CCA CAC CTT TCA GGG GTC CGA GGG GTC GTC CGT CTT CAT |
| 1261 | TGC AAA GCA TGT GGG GAT GCG GTG GGC TCT ATG GCT TCT ACT GGG CGG TTT TAT GGA CAG |
| | ACG TTT CGT ACA CCC CTA CGC CAC CCG AGA TAC CGA AGA TGA CCC GCC AAA ATA CCT GTC |
| 1321 | CAA GCG AAC CGG AAT TGC CAG CTG GGG CGC CCT CTG GTA AGG TTG GGA AGC CCT GCA AAG |
| | GTT CGC TTG GCC TTA ACG GTC GAC CCC GCG GGA GAC CAT TCC AAC CCT TCG GGA CGT TTC |
| 1381 | TAA ACT GGA TGG CTT TCT CGC CGC CAA GGA TCT GTC GAC CCC TAG ATT TCA GTG CAA TTT |
| | ATT TGA CCT ACC GAA AGA GCG GCG GTT CCT AGA CAG CTG GGG ATC TAA AGT CAC GTT AAA |
| 1441 | ATC TCT TCA AAT GTA GCA CCT GAA GTC AGC CCC ATA CGA TAT AAG TTG TTG GAA GAT CTA |
| | TAG AGA AGT TTA CAT CGT GGA CTT CAG TCG GGG TAT GCT ATA TTC AAC AAC CTT CTA GAT |
| 1501 | GCC CGC CTA ATG AGC GGG CTT TTT TTT AAT TCG CAA TTC CCC GAT GCA TAA TGT GCC TGT |
| | CGG GCG GAT TAC TCG CCC GAA AAA AAA TTA AGC GTT AAG GGG CTA CGT ATT ACA CGG ACA |
| 1561 | CAA ATG GAC GAA GCA GGG ATT CTG CAA ACC CTA TGC TAC TCC GTC AAG CCG TCA ATT GTC |
| | GTT TAC CTG CTT CGT CCC TAA GAC GTT TGG GAT ACG ATG AGG CAG TTC GGC AGT TAA CAG |
| 1621 | 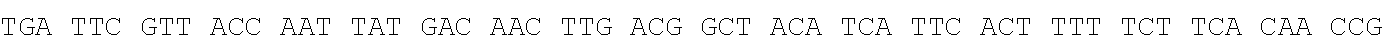 |
| | 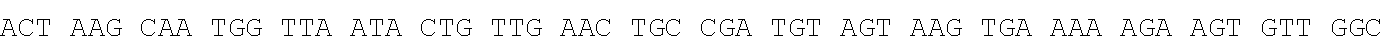 |
| | S L K V A V D N V K E E C G A |
| 1681 | 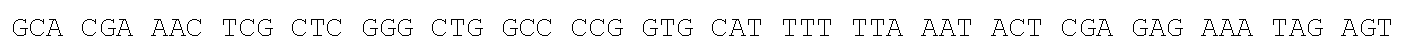 |
| | 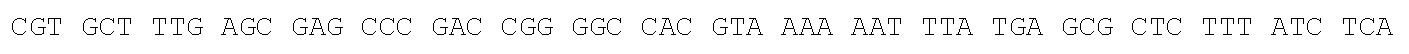 |
| | .. R F E S P S A G T C K K F V R S F Y L Q |
| 1741 | 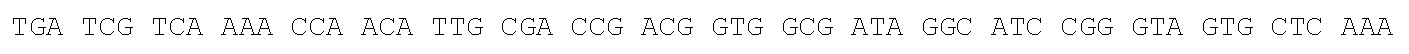 |
| | 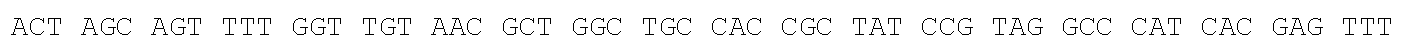 |
| | .. D D F G V N R G V T A I P M R T T S L L |
| 1801 | 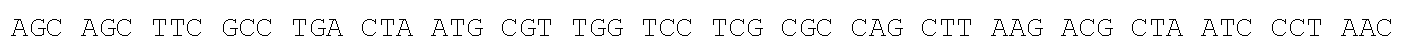 |
| | 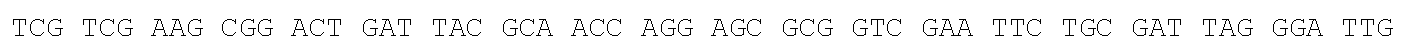 |
| | .. L K A Q S I R Q D E R W S L V S I G L Q |
| 1861 | 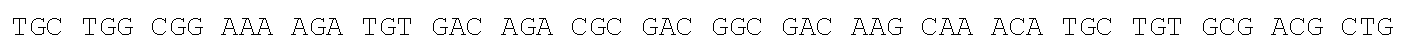 |
| | 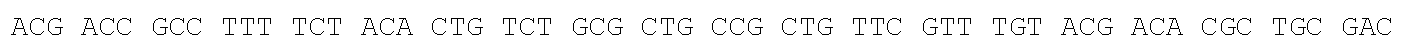 |
| | .. Q R F L H S L R S P S L C V H Q A V S A |
| 1921 | 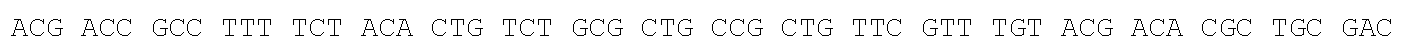 |
| | 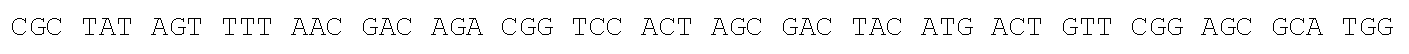 |
| | .. I D F N S D A L H D S I Y Q C A E R V R |
| 1981 | 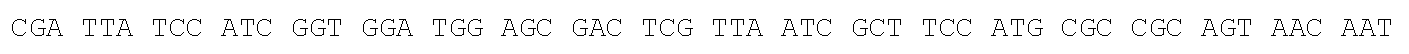 |
| | 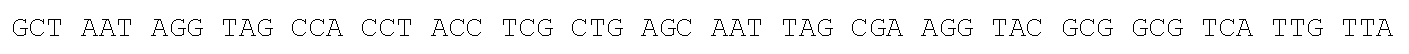 |
| | .. N D M P P H L S E N I A E M R R L L L Q |
| 2041 | 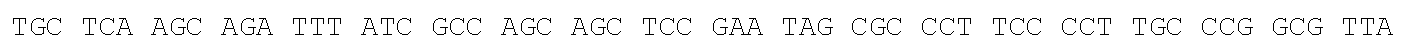 |
| | 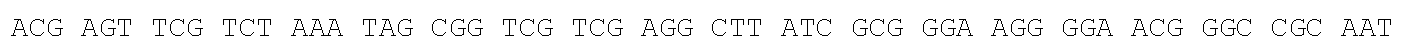 |
| | .. E L L N I A L L E S Y R G E G Q G A N I |
| 2101 | 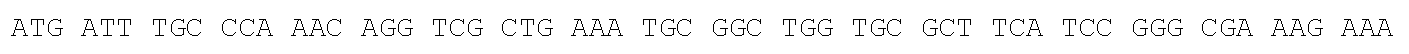 |
| | 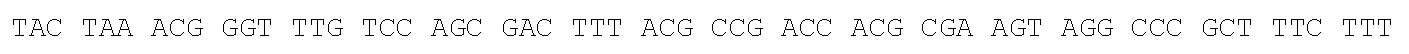 |
| | .. I Q G F L D S F H P Q H A E D P R F F G |
| 2161 | 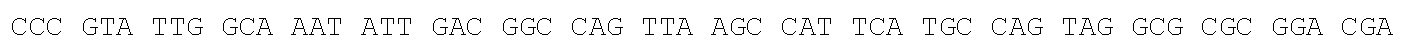 |
| | 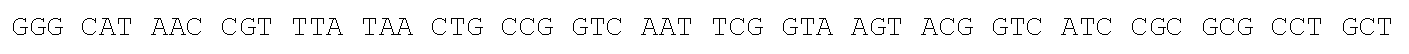 |
| | .. T N A F I S P W N L W E H W Y A R P R F |
| 2221 | 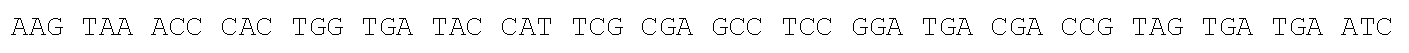 |
| | 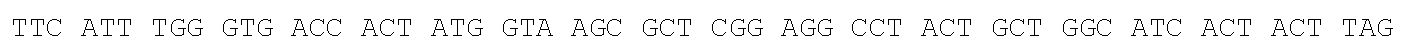 |
| | .. Y V W Q H Y W E R A E P H R G Y H H I E |
| 2281 | 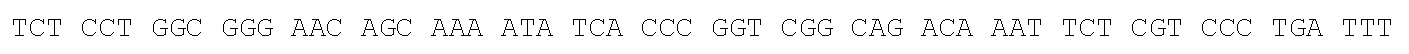 |
| | 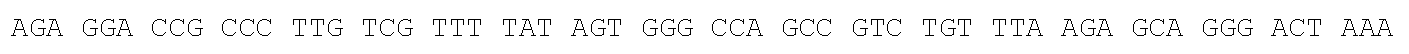 |
| | .. G P P F L L I D G P R C V F E R G Q N K |
| 2341 | 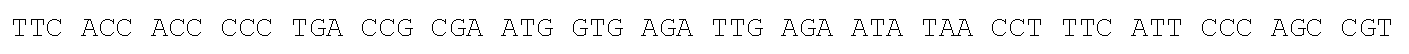 |
| | 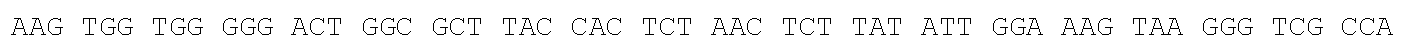 |
| | .. V V G Q G R I T L N L I Y G K M G L P R |
| 2401 | 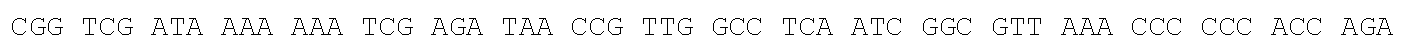 |
| | 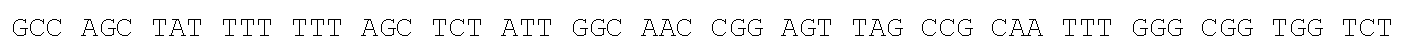 |
| | .. D I F F D L Y G N A E I P T L G A V L H |
| 2461 | 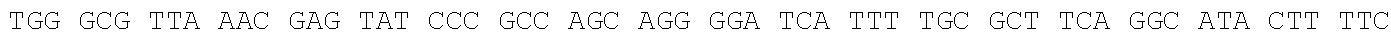 |
| | 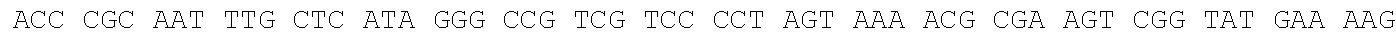 |
| | .. A N F S Y G P L L P D N Q A E A M |
| 2521 | ATA CTC CCA CCA TTC AGA GAA GAA ACC AAT TGT CCA TAT TGC ATC AGA CAT TGC CGT CAC |
| | TAT GAG GGT GGT AAG TCT CTT CTT TGG TTA ACA GGT ATA ACG TAG TCT GTA ACG GCA GTG |
| 2581 | TGC GTC TTT TAC TGG CTC TTC TCG CTA ACC CAA CCG GTA ACC CCG CTT ATT AAA AGC ATT |
| | ACG CAG AAA ATG ACC GAG AAG AGC GAT TGG GTT GGC CAT TGG GGC GAA TAA TTT TCG TAA |
| 2641 | CTG TAA CAA AGC GGG ACC AAA GCC ATG ACA AAA ACG CGT AAC AAA AGT GTC TAT AAT CAC |
| | GAC ATT GTT TCG CCC TGG TTT CGG TAC TGT TTT TGC GCA TTG TTT TCA CAG ATA TTA GTG |
| 2701 | GGC AGA AAA GTC CAC ATT GAT TAT TTG CAC GGC GTC ACA CTT TGC TAT GCC ATA GCA TTT |
| | CCG TCT TTT CAG GTG TAA CTA ATA AAC GTG CCG CAG TGT GAA ACG ATA CGG TAT CGT AAA |
| 2761 | TTA TCC ATA AGA TTA GCG GAT CCT ACC TGA CGC TTT TTA TCG CAA CTC TCT ACT GTT TCT |
| | AAT AGG TAT TCT AAT CGC CTA GGA TGG ACT GCG AAA AAT AGC GTT GAG AGA TGA CAA AGA |
| | M D K F R V |
| 2821 | 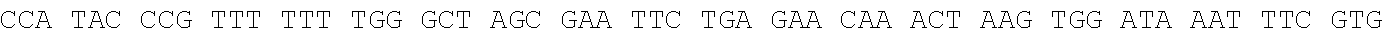 |
| | 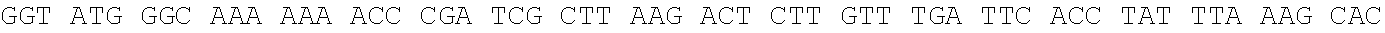 |
| | . Q G P T K L Q G E V T I S G A K N A A L |
| 2881 | 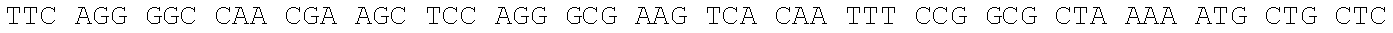 |
| | 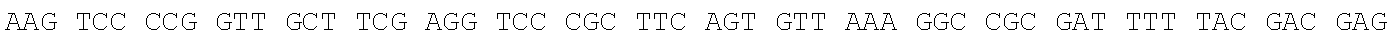 |
| | . P I L F A A L L A E E P V E I Q N V P K |
| 2941 | 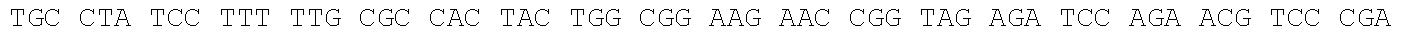 |
| | 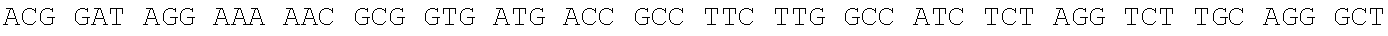 |
| | . L K D V D T S M K L L S Q L G A K V E R |
| 3001 | 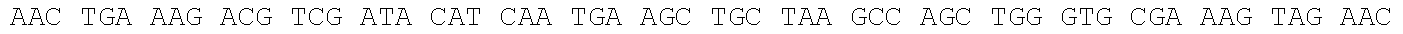 |
| | 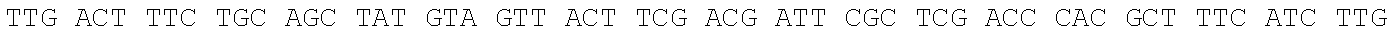 |
| | . N G S V H I D A R D V N V F C A P Y D L |
| 3061 | 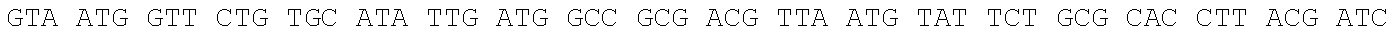 |
| | 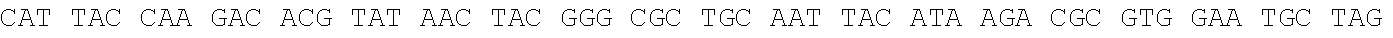 |
| | . V K T M R A S I W A L G P L V A R F G Q |
| 3121 | 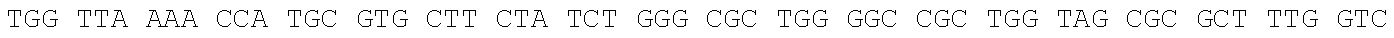 |
| | 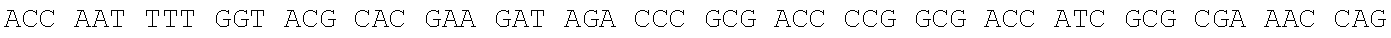 |
| | . G Q V S L P G G C T I G A R P V D L H I |
| 3181 | 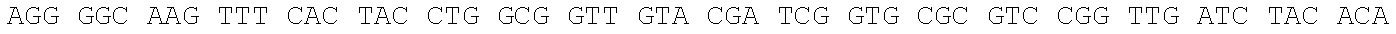 |
| | 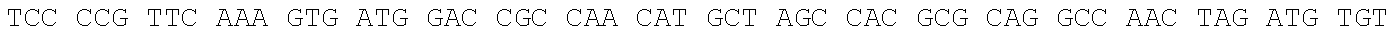 |
| | . S G L E Q L G A T I K L E E G Y V K A S |
| 3241 | 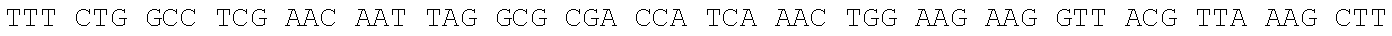 |
| | 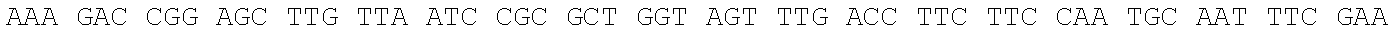 |
| | . V D G R L K G A H I V M D K V S V G A T |
| 3301 | 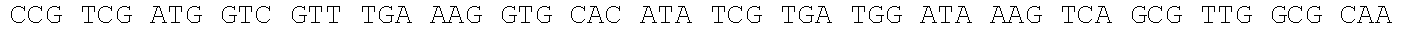 |
| | 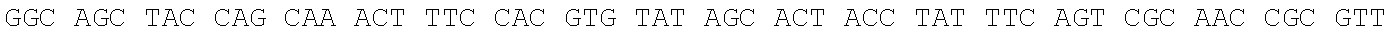 |
| | . V T I M C A A T L A E G T T I I E N A A |
| 3361 | 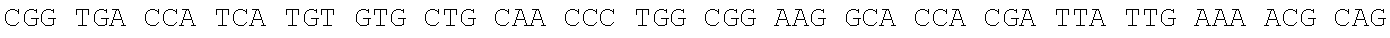 |
| | 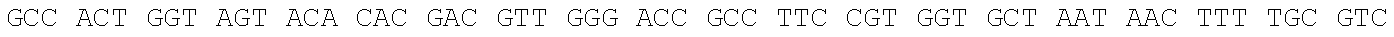 |
| | . R E P E I V D T A N F L I T L G A K I S |
| 3421 | 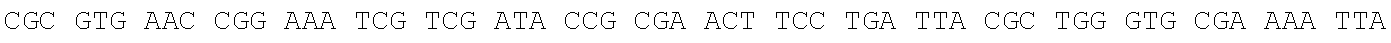 |
| | 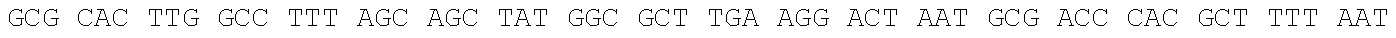 |
| | . G Q G T D R I V I E G V E R L G G G V Y |
| 3481 | 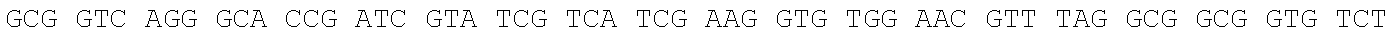 |
| | 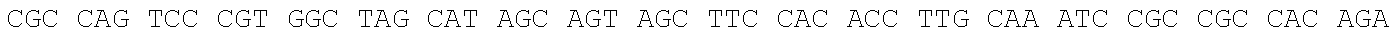 |
| | . R V L P D R I E T G T F L V A A A I S R |
| 3541 | 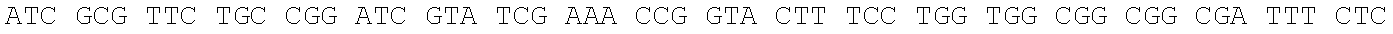 |
| | 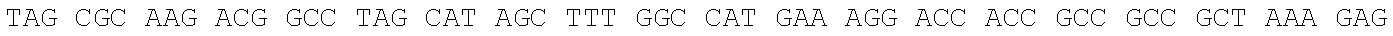 |
| | . G K I I C R N A Q P D T L D A V L A K L |
| 3601 | 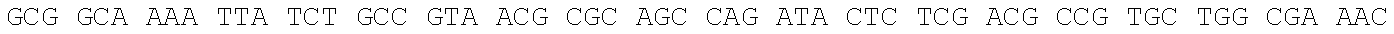 |
| | 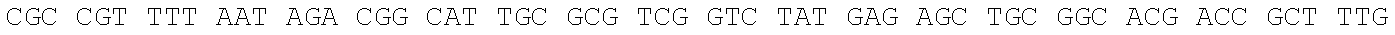 |
| | . R D A G A D I E V G E D W I S L D M H G |
| 3661 | 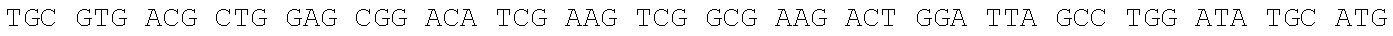 |
| | 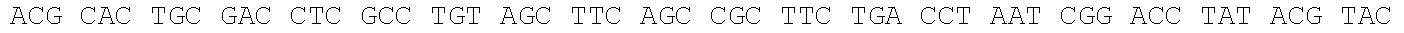 |
| | . K R P K A V N V R T A P H P A F P T D M |
| 3721 | 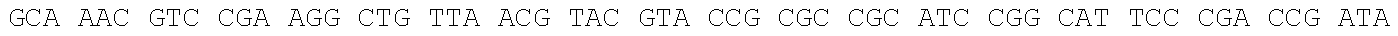 |
| | 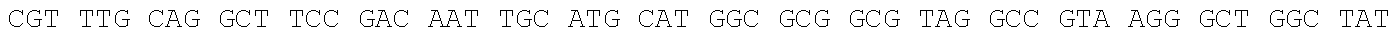 |
| | . Q A Q F T L L N L V A E G T G F I T E T |
| 3781 | 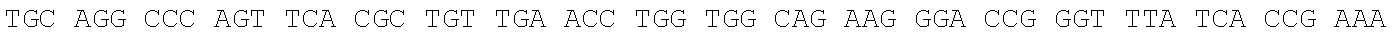 |
| | 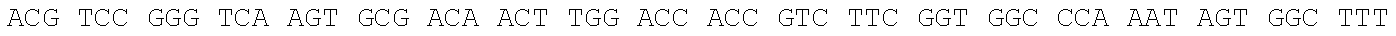 |
| | . V F E N R F M H V P E L S R M G A H A E |
| 3841 | 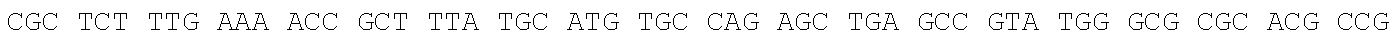 |
| | 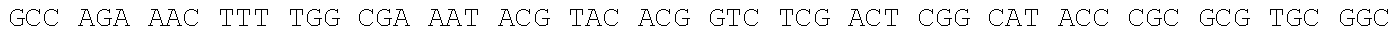 |
| | . I E S N T V I C H G V E K L S G A Q V M |
| 3901 | 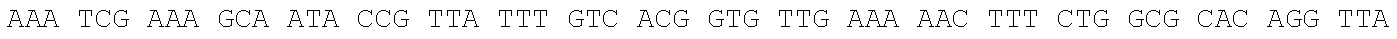 |
| | 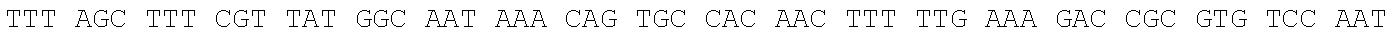 |
| | . A T D L R A S A S L V L A G C I A E G T |
| 3961 | 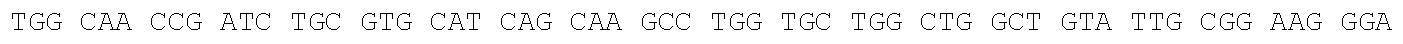 |
| | 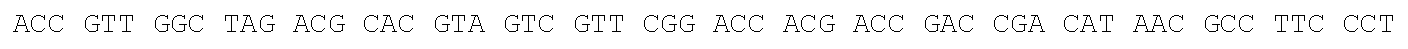 |
| | . T V V D R I Y H I D R G Y E R I E D K L |
| 4021 | 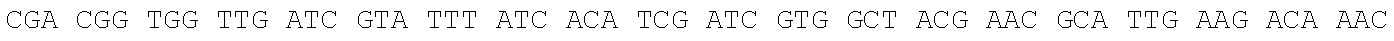 |
| | 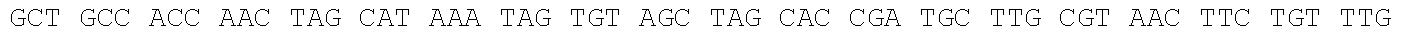 |
| | . R A L G A N I E R V K G E |
| 4081 | 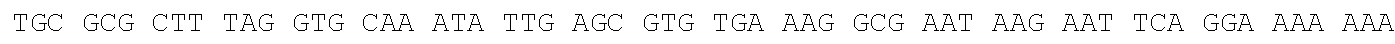 |
| | 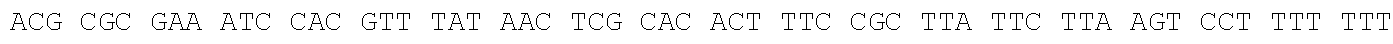 |
| | M K N V G F I G W R G M V G S V L M Q |
| 4141 | CGC TGT GAA AAA TGT TGG TTT TAT CGG CTG GCG CGG AAT GGT CGG CTC TGT TCT CAT GCA |
| | GCG ACA CTT TTT ACA ACC AAA ATA GCC GAC CGC GCC TTA CCA GCC GAG ACA AGA GTA CGT |
| | . R M V E E R D F D A I R P V F F S T S Q |
| 4201 | ACG CAT GGT AGA GGA GCG CGA TTT CGA CGC TAT TCG CCC TGT TTT CTT TTC TAC CTC CCA |
| | TGC GTA CCA TCT CCT CGC GCT AAA GCT GCG ATA AGC GGG ACA AAA GAA AAG ATG GAG GGT |
| | . F G Q A A P T F G D T S T G T L Q D A F |
| 4261 | GTT TGG ACA GGC GGC GCC CAC CTT CGG CGA CAC CTC CAC CGG CAC GCT ACA GGA CGC TTT |
| | CAA ACC TGT CCG CCG CGG GTG GAA GCC GCT GTG GAG GTG GCC GTG CGA TGT CCT GCG AAA |
| | . D L D A L K A L D I I V T C Q G G D Y T |
| 4321 | TGA TCT GGA TGC GCT AAA AGC GCT CGA TAT CAT CGT GAC CTG CCA GGG CGG CGA TTA TAC |
| | ACT AGA CCT ACG CGA TTT TCG CGA GCT ATA GTA GCA CTG GAC GGT CCC GCC GCT AAT ATG |
| | . N E I Y P K L R E S G W Q G Y W I D A A |
| 4381 | CAA CGA AAT TTA TCC AAA GCT GCG CGA AAG CGG ATG GCA GGG TTA CTG GAT TGA TGC GGC |
| | GTT GCT TTA AAT AGG TTT CGA CGC GCT TTC GCC TAC CGT CCC AAT GAC CTA ACT ACG CCG |
| | . S T L R M K D D A I I I L D P V N Q D V |
| 4441 | TTC TAC GCT GCG CAT GAA AGA TGA TGC CAT TAT TAT TCT CGA CCC GGT CAA CCA GGA CGT |
| | AAG ATG CGA CGC GTA CTT TCT ACT ACG GTA ATA ATA AGA GCT GGG CCA GTT GGT CCT GCA |
| | . I T D G L N N G V K T F V G G N C T V S |
| 4501 | GAT TAC CGA CGG CCT GAA CAA TGG CGT GAA GAC CTT TGT GGG CGG TAA CTG TAC CGT TAG |
| | CTA ATG GCT GCC GGA CTT GTT ACC GCA CTT CTG GAA ACA CCC GCC ATT GAC ATG GCA ATC |
| | . L M L M S L G G L F A H N L V D W V S V |
| 4561 | CCT GAT GTT GAT GTC GCT GGG CGG TCT CTT TGC CCA TAA TCT CGT TGA CTG GGT ATC CGT |
| | GGA CTA CAA CTA CAG CGA CCC GCC AGA GAA ACG GGT ATT AGA GCA ACT GAC CCA TAG GCA |
| | . A T Y Q A A S G G G A R H M R E L L T Q |
| 4621 | CGC GAC CTA TCA GGC CGC CTC CGG CGG CGG CGC GCG CCA TAT GCG CGA GCT GTT AAC CCA |
| | GCG CTG GAT AGT CCG GCG GAG GCC GCC GCC GCG CGC GGT ATA CGC GCT CGA CAA TTG GGT |
| | . M G Q L Y G H V A D E L A T P S S A I L |
| 4681 | GAT GGG TCA GTT GTA TGG CCA TGT CGC CGA TGA ACT GGC GAC GCC GTC TTC CGC AAT TCT |
| | CTA CCC AGT CAA CAT ACC GGT ACA GCG GCT ACT TGA CCG CTG CGG CAG AAG GCG TTA AGA |
| | . D I E R K V T A L T R S G E L P V D N F |
| 4741 | TGA TAT TGA ACG CAA AGT TAC GGC ATT GAC CCG CAG CGG CGA GCT GCC GGT TGA TAA CTT |
| | ACT ATA ACT TGC GTT TCA ATG CCG TAA CTG GGC GTC GCC GCT CGA CGG CCA ACT ATT GAA |
| | . G V P L A G S L I P W I D K Q L D N G Q |
| 4801 | TGG CGT ACC GCT GGC GGG AAG CCT GAT CCC CTG GAT CGA CAA ACA GCT CGA TAA CGG CCA |
| | ACC GCA TGG CGA CCG CCC TTC GGA CTA GGG GAC CTA GCT GTT TGT CGA GCT ATT GCC GGT |
| | . S R E E W K G Q A E T N K I L N T A S V |
| 4861 | GAG CCG CGA AGA GTG GAA AGG CCA GGC GGA AAC CAA CAA GAT TCT CAA TAC TGC CTC TGT |
| | CTC GGC GCT TCT CAC CTT TCC GGT CCG CCT TTG GTT GTT CTA AGA GTT ATG ACG GAG ACA |
| | . I P V D G L C V R V G A L R C H S Q A F |
| 4921 | GAT TCC GGT TGA TGG TTT GTG TGT GCG CGT CGG CGC GCT GCG CTG TCA CAG CCA GGC GTT |
| | CTA AGG CCA ACT ACC AAA CAC ACA CGC GCA GCC GCG CGA CGC GAC AGT GTC GGT CCG CAA |
| | . T I K L K K E V S I P T V E E L L A A H |
| 4981 | CAC CAT CAA GCT GAA AAA AGA GGT ATC CAT TCC GAC GGT GGA AGA ACT GCT GGC GGC ACA |
| | GTG GTA GTT CGA CTT TTT TCT CCA TAG GTA AGG CTG CCA CCT TCT TGA CGA CCG CCG TGT |
| | . N P W A K V V P N D R D I T M R E L T P |
| 5041 | TAA TCC GTG GGC GAA AGT GGT GCC GAA CGA TCG TGA TAT CAC TAT GCG CGA ATT AAC CCC |
| | ATT AGG CAC CCG CTT TCA CCA CGG CTT GCT AGC ACT ATA GTG ATA CGC GCT TAA TTG GGG |
| | . A A V T G T L T T P V G R L R K L N M G |
| 5101 | GGC GGC GGT GAC CGG CAC GTT GAC TAC GCC GGT TGG TCG TCT GCG TAA GCT GAA CAT GGG |
| | CCG CCG CCA CTG GCC GTG CAA CTG ATG CGG CCA ACC AGC AGA CGC ATT CGA CTT GTA CCC |
| | . P E F L S A F T V G D Q L L W G A A E P |
| 5161 | GCC AGA GTT CTT GTC GGC GTT TAC CGT AGG CGA CCA GTT GTT ATG GGG CGC CGC CGA GCC |
| | CGG TCT CAA GAA CAG CCG CAA ATG GCA TCC GCT GGT CAA CAA TAC CCC GCG GCG GCT CGG |
| | . L R R M L R Q L A |
| 5221 | GCT GCG TCG AAT GCT GCG CCA GTT GGC GTA GTC TAG CTG CAC GAT ACC GTC GAC TTG TAC |
| | CGA CGC AGC TTA CGA CGC GGT CAA CCG CAT CAG ATC GAC GTG CTA TGG CAG CTG AAC ATG |
| 5281 | ATA GAC TCG CTC CGA AAT TAA AGA ACA CTT AAA TTA TCT ACT AAA GGA ATC TTT AGT CAA |
| | TAT CTG AGC GAG GCT TTA ATT TCT TGT GAA TTT AAT AGA TGA TTT CCT TAG AAA TCA GTT |
| 5341 | GTT TAT TTA AGA TGA CTT AAC TAT GAA TAC ACA ATT GAT GGG TGA GCG TAG GAA AAA AAA |
| | CAA ATA AAT TCT ACT GAA TTG ATA CTT ATG TGT TAA CTA CCC ACT CGC ATC CTT TTT TTT |
| 5401 | ACC CCG CCC CTG ACA GGG CGG GGT TTT TTT TGA TCA TTC TGA AAT GAG CTG TTG ACA ATT |
| | TGG GGC GGG GAC TGT CCC GCC CCA AAA AAA ACT AGT AAG ACT TTA CTC GAC AAC TGT TAA |
| 5461 | AAT CAT CCG GCT CGT ATA ATG TGT GGA ATT GTG AGC GGA TAA CAA TTT CAC ACA GGA AAC |
| | TTA GTA GGC CGA GCA TAT TAC ACA CCT TAA CAC TCG CCT ATT GTT AAA GTG TGT CCT TTG |
| | M K K Q H F R V A L I P F F A A F C L |
| 5521 | 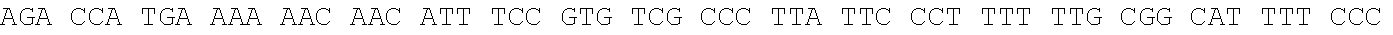 |
| | 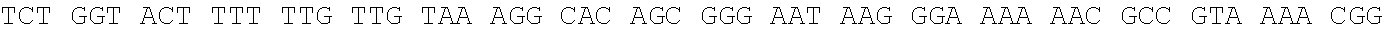 |
| | . P V F A H P E T L V K V K D A E |
| 5581 | 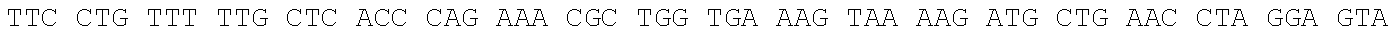 |
| | 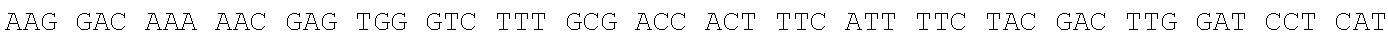 |
| 5641 | CTC CGC GGC CCG GGC CCC TGC AGG TTC GAA ACA GAT TAA ATC AGA ACG CAG AAG CGG TCT |
| | GAG GCG CCG GGC CCG GGG ACG TCC AAG CTT TGT CTA ATT TAG TCT TGC GTC TTC GCC AGA |
| 5701 | GAT AAA ACA GAA TTT GCC TGG CGG CAG TAG CGC GGT GGT CCC ACC TGA CCC CAT GCC GAA |
| | CTA TTT TGT CTT AAA CGG ACC GCC GTC ATC GCG CCA CCA GGG TGG ACT GGG GTA CGG CTT |
| 5761 | CTC AGA AGT GAA ACG CCG TAG CGC CGA TGG TAG TGT GGG GTC TCC CCA TGC GAG AGT AGG |
| | GAG TCT TCA CTT TGC GGC ATC GCG GCT ACC ATC ACA CCC CAG AGG GGT ACG CTC TCA TCC |
| 5821 | GAA CTG CCA GGC ATC AAA TAA AAC GAA AGG CTC AGT CGA AAG ACT GGG CCT TTC GTT TTA |
| | CTT GAC GGT CCG TAG TTT ATT TTG CTT TCC GAG TCA GCT TTC TGA CCC GGA AAG CAA AAT |
| 5881 | TCT GTT GTT TGT CGG TGA ACG CTC TCC TGA GTA GGA CAA ATC CGC CGG GAG CGG ATT TGA |
| | AGA CAA CAA ACA GCC ACT TGC GAG AGG ACT CAT CCT GTT TAG GCG GCC CTC GCC TAA ACT |
| 5941 | ACG TTG CGA AGC AAC GGC CCG GAG GGT GGC GGG CAG GAC GCC CGC CAT AAA CTG CCA GGC |
| | TGC AAC GCT TCG TTG CCG GGC CTC CCA CCG CCC GTC CTG CGG GCG GTA TTT GAC GGT CCG |
| 6001 | ATC AAA TTA AGC AGA AGG CCA TCC TGA CGG ATG GCC TTT TTG CGT TTC TAC AAA CTC TTT |
| | TAG TTT AAT TCG TCT TCC GGT AGG ACT GCC TAC CGG AAA AAC GCA AAG ATG TTT GAG AAA |
| 6061 | TTG TTT ATT TTT CTA AAT ACA TTC AAA TAT GTA TCC GCT CAT GAG ACA ATA ACC CTG ATA |
| | AAC AAA TAA AAA GAT TTA TGT AAG TTT ATA CAT AGG CGA GTA CTC TGT TAT TGG GAC TAT |
| 6121 | AAT GCT TCA ATA ATG GAA GAT CTT CCA ACA TCA CAG GTA AAC AGA AAC GTC GGG TCG ATC |
| | TTA CGA AGT TAT TAC CTT CTA GAA GGT TGT AGT GTC CAT TTG TCT TTG CAG CCC AGC TAG |
| 6181 | GGG AAA TTC TTT CCC GGA CGG CGC GGG GTT GGG CAA GCC GCA GGC GCG TCA GTG CTT TTA |
| | CCC TTT AAG AAA GGG CCT GCC GCG CCC CAA CCC GTT CGG CGT CCG CGC AGT CAC GAA AAT |
| 6241 | GCG GGT GTC GGG GCA GCC CTG AAC CAG TCA CGG GAT CGA TCT GTG CGG TAT TTC ACA CCG |
| | CGC CCA CAG CCC CGT CGG GAC TTG GTC AGT GCC CTA GCT AGA CAC GCC ATA AAG TGT GGC |
| 6301 | CAT ACA GGT GGC ACT TTT CGG GGA AAT GTG CGC GGA ACC CCT ATT TGT TTA TTT TTC TAA |
| | GTA TGT CCA CCG TGA AAA GCC CCT TTA CAC GCG CCT TGG GGA TAA ACA AAT AAA AAG ATT |
| 6361 | ATA CAT TCA AAT ATG TAT CCG CTC ATG AGA CAA TAA CCC TGA TAA ATG CTT CAA TAA TAG |
| | TAT GTA AGT TTA TAC ATA GGC GAG TAC TCT GTT ATT GGG ACT ATT TAC GAA GTT ATT ATC |
| 6421 | CAC GTG CTA AAA CTT CAT TTT TAA TTT AAA AGG ATC TAG GTG AAG ATC CTT TTT GAT AAT |
| | GTG CAC GAT TTT GAA GTA AAA ATT AAA TTT TCC TAG ATC CAC TTC TAG GAA AAA CTA TTA |
| 6481 | CTC ATG ACC AAA ATC CCT TAA CGT GAG TTT TCG TTC CAC TGA GCG TCA GAC CCC GTA GAA |
| | GAG TAC TGG TTT TAG GGA ATT GCA CTC AAA AGC AAG GTG ACT CGC AGT CTG GGG CAT CTT |
| 6541 | AAG ATC AAA GGA TCT TCT TGA GAT CCT TTT TTT CTG CGC GTA ATC TGC TGC TTG CAA ACA |
| | TTC TAG TTT CCT AGA AGA ACT CTA GGA AAA AAA GAC GCG CAT TAG ACG ACG AAC GTT TGT |
| 6601 | AAA AAA CCA CCG CTA CCA GCG GTG GTT TGT TTG CCG GAT CAA GAG CTA CCA ACT CTT TTT |
| | TTT TTT GGT GGC GAT GGT CGC CAC CAA ACA AAC GGC CTA GTT CTC GAT GGT TGA GAA AAA |
| 6661 | CCG AAG GTA ACT GGC TTC AGC AGA GCG CAG ATA CCA AAT ACT GTC CTT CTA GTG TAG CCG |
| | GGC TTC CAT TGA CCG AAG TCG TCT CGC GTC TAT GGT TTA TGA CAG GAA GAT CAC ATC GGC |
| 6721 | TAG TTA GGC CAC CAC TTC AAG AAC TCT GTA GCA CCG CCT ACA TAC CTC GCT CTG CTA ATC |
| | ATC AAT CCG GTG GTG AAG TTC TTG AGA CAT CGT GGC GGA TGT ATG GAG CGA GAC GAT TAG |
| 6781 | CTG TTA CCA GTG GCT GCT GCC AGT GGC GAT AAG TCG TGT CTT ACC GGG TTG GAC TCA AGA |
| | GAC AAT GGT CAC CGA CGA CGG TCA CCG CTA TTC AGC ACA GAA TGG CCC AAC CTG AGT TCT |
| 6841 | CGA TAG TTA CCG GAT AAG GCG CAG CGG TCG GGC TGA ACG GGG GGT TCG TGC ACA CAG CCC |
| | GCT ATC AAT GGC CTA TTC CGC GTC GCC AGC CCG ACT TGC CCC CCA AGC ACG TGT GTC GGG |
| 6901 | AGC TTG GAG CGA ACG ACC TAC ACC GAA CTG AGA TAC CTA CAG CGT GAG CTA TGA GAA AGC |
| | TCG AAC CTC GCT TGC TGG ATG TGG CTT GAC TCT ATG GAT GTC GCA CTC GAT ACT CTT TCG |
| 6961 | GCC ACG CTT CCC GAA GGG AGA AAG GCG GAC AGG TAT CCG GTA AGC GGC AGG GTC GGA ACA |
| | CGG TGC GAA GGG CTT CCC TCT TTC CGC CTG TCC ATA GGC CAT TCG CCG TCC CAG CCT TGT |
| 7021 | GGA GAG CGC ACG AGG GAG CTT CCA GGG GGA AAC GCC TGG TAT CTT TAT AGT CCT GTC GGG |
| | CCT CTC GCG TGC TCC CTC GAA GGT CCC CCT TTG CGG ACC ATA GAA ATA TCA GGA CAG CCC |
| 7081 | TTT CGC CAC CTC TGA CTT GAG CGT CGA TTT TTG TGA TGC TCG TCA GGG GGG CGG AGC CTA |
| | AAA GCG GTG GAG ACT GAA CTC GCA GCT AAA AAC ACT ACG AGC AGT CCC CCC GCC TCG GAT |
| 7141 | TGG AAA AAC GCC AGC AAC GCG GCC TTT TTA CGG TTC CTG GGC TTT TGC TGG CCT TTT GCT |
| | ACC TTT TTG CGG TCG TTG CGC CGG AAA AAT GCC AAG GAC CCG AAA ACG ACC GGA AAA CGA |
| 7201 | CAC ATG TTC T |
| | GTG TAC AAG A |

GSG T2A sequence (derived from thoseassigna virus 2A) (SEQ ID NO: 129
(amin acid))
(GSG) EGRGSLL TCGDVEENPGP
GSG E2A sequence (derived from equine rhinitis A virus) (SEQ ID NO: 130
(amin acid))
(GSG) QCTNYALLKLAGDVESNPGP
GSG F2A sequence (derived from foot-and-mouth disease virus) (SEQ ID NO: 131
(amin acid))
(GSG) VKQTLNFDLLKLAGDVESNPGP
Linker sequence in pG8R362-pG8R365 (SEQ ID NO: 132 (DNA)) (SEQ ID NO: 133
(amino acid))

| | |
|---|---|
| | G G S G G G S E G G G S E G G G S E G G |
| 1 | GGC GGC TCC GGC GGC GGC AGT GAG GGC GGT GGA AGT GAA GGC GGA GGA AGC GAG GGA GGA |
| | G S E G G G S E G G G S G G G S |
| 61 | GGC TCC GAA GGA GGC GCG TCT GAG GGT GGA GGC AGC GGC GGC GGA AGC |

### REFERENCES

1. Centers for Disease Control and Prevention NCfHS. 2021. Underlying Cause of Death 1999-2020 on CDC WONDER Online Database, released in 2021. Data are from the Multiple Cause of Death Files, 1999-2020, as compiled from data provided by the 57 vital statistics jurisdictions through the Vital Statistics Cooperative Program.
2. Anonymous. William Coley. https://en.wikipedia.org/wiki/William_Coley. Accessed August 28.
3. Coley W B. 1891. II. Contribution to the Knowledge of Sarcoma. Ann Surg 14:199-220.
4. Malmgren R A, Flanigan C C. 1955. Localization of the vegetative form of Clostridium tetani in mouse tumors following intravenous spore administration. Cancer Res 15:473-478.
5. Forbes N S. 2010. Engineering the perfect (bacterial) cancer therapy. Nat Rev Cancer 10:785-794.
6. Kohwi Y, Imai K, Tamura Z, Hashimoto Y. 1978. Antitumor effect of Bifidobacterium infantis in mice. Gan 69:613-618.
7. Yu Y A, Shabahang S, Timiryasova T M, Zhang Q, Beltz R, Gentschev I, Goebel W, Szalay A A. 2004. Visualization of tumors and metastases in live animals with bacteria and vaccinia virus encoding light-emitting proteins. Nat Biotechnol 22:313-320.
8. Pan Z K, Weiskirch L M, Paterson Y. 1999. Regression of established B16F10 melanoma with a recombinant Listeria monocytogenes vaccine. Cancer Res 59:5264-5269.
9. Wang C Z, Kazmierczak R A, Eisenstark A. 2016. Strains, Mechanism, and Perspective: Salmonella-Based Cancer Therapy. Int J Microbiol 2016:5678702.
10. Toso J F, Gill V J, Hwu P, Marincola F M, Restifo N P, Schwartzentruber D J, Sherry R M, Topalian S L, Yang J C, Stock F, Freezer L J, Morton K E, Seipp C, Haworth L, Mavroukakis S, White D, MacDonald S, Mao J, Sznol M, Rosenberg S A. 2002. Phase I study of the intravenous administration of attenuated Salmonella typhimurium to patients with metastatic melanoma. J Clin Oncol 20:142-152.
11. Heimann D M, Rosenberg S A. 2003. Continuous intravenous administration of live genetically modified Salmonella typhimurium in patients with metastatic melanoma. J Immunother 26:179-180.
12. Thamm D H, Kurzman I D, King I, Li Z, Sznol M, Dubielzig R R, Vail D M, MacEwen E G. 2005. Systemic administration of an attenuated, tumor-targeting Salmonella typhimurium to dogs with spontaneous neoplasia: phase I evaluation. Clin Cancer Res 11:4827-4834.
13. Nemunaitis J, Cunningham C, Senzer N, Kuhn J, Cramm J, Litz C, Cavagnolo R, Cahill A, Clairmont C, Sznol M. 2003. Pilot trial of genetically modified, attenuated Salmonella expressing the E. coli cytosine deaminase gene in refractory cancer patients. Cancer Gene Ther 10:737-744.
14. Luo Y, Kong Q, Yang J, Mitra A, Golden G, Wanda S Y, Roland K L, Jensen R V, Ernst P B, Curtiss R, III. 2012. Comparative genome analysis of the high pathogenicity Salmonella Typhimurium strain UK-1. PLoS One 7:e40645.
15. Sanapala S, Mosca L, Wang S, Curtiss R, III. 2018. Comparative evaluation of Salmonella Typhimurium vaccines derived from UK-1 and 14028S: Importance of inherent virulence. PLoS One 13:e0203526.
16. Feigner S, Kocijancic D, Frahm M, Curtiss R, III, Erhardt M, Weiss S. 2016. Optimizing Salmonella enterica serovar Typhimurium for bacteria-mediated tumor therapy. Gut Microbes 7:171-177.
17. Broadway K M, Modise T, Jensen R V, Scharf B E. 2014. Complete genome sequence of Salmonella enterica serovar Typhimurium VNP20009, a strain engineered for tumor targeting. J Biotechnol 192 Pt A:177-178.
18. Broadway K M, Denson E A, Jensen R V, Scharf B E. 2015. Rescuing chemotaxis of the anticancer agent Salmonella enterica serovar Typhimurium VNP20009. J Biotechnol 211:117-120.
19. Kong Q, Six D A, Liu Q, Gu L, Roland K L, Raetz C R, Curtiss R, III. 2011. Palmitoylation state impacts induction of innate and acquired immunity by the Salmonella enterica serovar Typhimurium msbB mutant. Infect Immun 79:5027-5038.
20. Kong Q, Six D A, Liu Q, Gu L, Wang S, Alamuri P, Raetz C R, Curtiss R, III. 2012. Phosphate groups of lipid A are essential for Salmonella enterica serovar Typhimurium virulence and affect innate and adaptive immunity. Infect Immun 80:3215-3224.
21. Kong Q, Six D A, Roland K L, Liu Q, Gu L, Reynolds C M, Wang X, Raetz C R, Curtiss R, III. 2011. Salmonella synthesizing 1-dephosphorylated lipopolysaccharide exhibits low endotoxic activity while retaining its immunogenicity. J Immunol 187:412-423.
22. Kong Q, Yang J, Liu Q, Alamuri P, Roland K L, Curtiss R, III. 2011. Effect of deletion of genes involved in lipopolysaccharide core and O-antigen synthesis on virulence and immunogenicity of Salmonella enterica serovar Typhimurium. Infect Immun 79:4227-4239.
23. Hoffman R M. 2011. Tumor-seeking Salmonella amino acid auxotrophs. Curr Opin Biotechnol 22:917-923.
24. Zhao M, Yang M, Li X M, Jiang P, Baranov E, Li S, Xu M, Penman S, Hoffman R M. 2005. Tumor-targeting bacterial therapy with amino acid auxotrophs of GFP-expressing Salmonella typhimurium. Proc Natl Acad Sci USA 102:755-760.
25. Zhao M, Yang M, Ma H, Li X, Tan X, Li S, Yang Z, Hoffman R M. 2006. Targeted therapy with a Salmonella typhimurium leucine-arginine auxotroph cures orthotopic human breast tumors in nude mice. Cancer Res 66:7647-7652.
26. Nagakura C, Hayashi K, Zhao M, Yamauchi K, Yamamoto N, Tsuchiya H, Tomita K, Bouvet M, Hoffman R M. 2009. Efficacy of a genetically-modified Salmonella typhimurium in an orthotopic human pancreatic cancer in nude mice. Anticancer Res 29:1873-1878.
27. Schmitz-Winnenthal F H, Hohmann N, Niethammer A G, Friedrich T, Lubenau H, Springer M, Breiner K M, Mikus G, Weitz J, Ulrich A, Buechler M W, Pianka F, Klaiber U, Diener M, Leowardi C, Schimmack S, Sisic L, Keller A V, Koc R, Springfeld C, Knebel P, Schmidt T, Ge Y, Bucur M, Stamova S, Podola L, Haefeli W E, Grenacher L, Beckhove P. 2015. Anti-angiogenic activity of VXM01, an oral T-cell vaccine against VEGF receptor 2, in patients with advanced pancreatic cancer: A randomized, placebo-controlled, phase 1 trial. Oncoimmunology 4:el001217.
28. Gniadek T J, Augustin L, Schottel J, Leonard A, Saltzman D, Greeno E, Batist G. 2020. A Phase I, Dose Escalation, Single Dose Trial of Oral Attenuated Salmonella typhimurium Containing Human IL-2 in Patients With Metastatic Gastrointestinal Cancers. J Immunother 43:217-221.
29. Selmic L E, Burton J H, Thamm D H, Withrow S J, Lana S E. 2014. Comparison of carboplatin and doxorubicin-based chemotherapy protocols in 470 dogs after amputation for treatment of appendicular osteosarcoma. J Vet Intern Med 28:554-563.
30. Fritz S E, Henson M S, Greengard E, Winter A L, Stuebner K M, Yoon U, Wilk V L, Borgatti A, Augustin L B, Modiano J F, Saltzman D A. 2016. A phase I clinical study to evaluate safety of orally administered, genetically engineered Salmonella enterica serovar Typhimurium for canine osteosarcoma. Vet Med Sci 2:179-190.
31. Ganai S, Arenas R B, Forbes N S. 2009. Tumour-targeted delivery of TRAIL using Salmonella typhimurium enhances breast cancer survival in mice. Br J Cancer 101:1683-1691.
32. Loeffler M, Le'Negrate G, Krajewska M, Reed J C. 2008. Inhibition of tumor growth using Salmonella expressing Fas ligand. J Natl Cancer Inst 100:1113-1116.
33. Ryan R M, Green J, Williams P J, Tazzyman S, Hunt S, Harmey J H, Kehoe S C, Lewis C E. 2009. Bacterial delivery of a novel cytolysin to hypoxic areas of solid tumors. Gene Ther 16:329-339.
34. Nguyen V H, Kim H S, Ha J M, Hong Y, Choy H E, Min J J. 2010. Genetically engineered Salmonella typhimurium as an imageable therapeutic probe for cancer. Cancer Res 70:18-23.
35. Saltzman D A, Heise C P, Hasz D E, Zebede M, Kelly S M, Curtiss R, III, Leonard A S, Anderson P M. 1996. Attenuated Salmonella typhimurium containing interleukin-2 decreases MC-38 hepatic metastases: a novel anti-tumor agent. Cancer Biother Radiopharm 11:145-153.
36. Sorenson B S, Banton K L, Frykman N L, Leonard A S, Saltzman D A. 2008. Attenuated Salmonella typhimurium with interleukin 2 gene prevents the establishment of pulmonary metastases in a model of osteosarcoma. J Pediatr Surg 43:1153-1158.
37. al-Ramadi B K, Fernandez-Cabezudo M J, E I-Hasasna H, A I-Salam S, Bashir G, Chouaib S. 2009. Potent anti-tumor activity of systemically-administered IL2-expressing Salmonella correlates with decreased angiogenesis and enhanced tumor apoptosis. Clin Immunol 130:89-97.
38. Nagaraj S, Pisarev V, Kinarsky L, Sherman S, Muro-Cacho C, Altieri D C, Gabrilovich D I. 2007. Dendritic cell-based full-length survivin vaccine in treatment of experimental tumors. J Immunother 30:169-179.
39. Bertino P, Panigada M, Soprana E, Bianchi V, Bertilaccio S, Sanvito F, Rose A H, Yang H, Gaudino G, Hoffmann P R, Siccardi A, Carbone M. 2013. Fowlpox-based survivin vaccination for malignant mesothelioma therapy. Int J Cancer 133:612-623.
40. Srivastava A K, Sharma R K, Yolcu E S, Ulker V, MacLeod K, Dinc G, Shirwan H. 2012. Prime-boost vaccination with SA-4-1 BBL costimulatory molecule and survivin eradicates lung carcinoma in CD8+T and N K cell dependent manner. PLoS One 7:e48463.
41. Ishizaki H, Manuel E R, Song G Y, Srivastava T, Sun S, Diamond D J, Ellenhorn J D. 2011. Modified vaccinia Ankara expressing survivin combined with gemcitabine generates specific antitumor effects in a murine pancreatic carcinoma model. Cancer Immunol Immunother 60:99-109.
42. Curtiss R, III. 2015. Antigen Delivery System I I: Development of Live Attenuated Bacterial Vectors p 1233-1269. In Mestecky J, Strober W, Russell M W, Kelsall B L, Cheroutre H, Lambrecht B N (ed), Mucosal Immunology (Fourth Edition) doi: http://dx.doi.org/10.1016/B978-0-12-415847-4.00064-1. Academic Press, Boston.
43. Curtiss R, III. 2005. Antigen Delivery Systems I I: Development of Live Recombinant Attenuated Bacterial Antigen and DNA Vaccine Delivery Vector Vaccines p 1009-1037. In Mestecky J, Lamm M E, McGhee J R, Bienenstock J, Mayer L, Strober W (ed), Mucosal Immunology (Third Edition) doi: http://dx.doi.org/10.1016/B978-012491543-5/50060-7. Academic Press, Burlington.
44. Galen J E, Curtiss R, III. 2014. The delicate balance in genetically engineering live vaccines. Vaccine 32:4376-4385.
45. Kong W, Brovold M, Koeneman B A, Clark-Curtiss J, Curtiss R, III. 2012. Turning self-destructing Salmonella into a universal DNA vaccine delivery platform. Proc Natl Acad Sci USA 109:19414-19419.
46. Curtiss R, III, Kelly S M, Tinge S A, Tacket C O, Levine M M, Srinivasan J, Koopman M. 1994. Recombinant Salmonella vectors in vaccine development. Dev Biol Stand 82:23-33.
47. Curtiss R, III, Xin W, Li Y, Kong W, Wanda S Y, Gunn B, Wang S. 2010. New technologies in using recombinant attenuated Salmonella vaccine vectors. Crit Rev Immunol 30:255-270.
48. Kong W, Clark-Curtiss J, Curtiss R, III. 2013. Utilizing Salmonella for antigen delivery: the aims and benefits of bacterial delivered vaccination. Expert Rev Vaccines 12:345-347.
49. Wang S, Kong Q, Curtiss R, III. 2013. New technologies in developing recombinant attenuated Salmonella vaccine vectors. Microb Pathog 58:17-28.
50. Curtiss R, III., Wanda S Y, Gunn B M, Zhang X, Tinge S A, Ananthnarayan V, Mo H, Wang S, Kong W. 2009. Salmonella enterica serovar Typhimurium strains with regulated delayed attenuation in vivo. Infect Immun 77:1071-1082.
51. Li Y, Wang S, Scarpellini G, Gunn B, Xin W, Wanda S Y, Roland K L, Curtiss R, III. 2009. Evaluation of new generation Salmonella enterica serovar Typhimurium vaccines with regulated delayed attenuation to induce immune responses against PspA. Proc Natl Acad Sci USA 106:593-598.
52. Juarez-Rodriguez M D, Yang J, Kader R, Alamuri P, Curtiss R, III, Clark-Curtiss J E. 2012. Live attenuated Salmonella vaccines displaying regulated delayed lysis and delayed antigen synthesis to confer protection against Mycobacterium tuberculosis. Infect Immun 80:815-831.
53. Wang S, Li Y, Scarpellini G, Kong W, Shi H, Baek C H, Gunn B, Wanda S Y, Roland K L, Zhang X, Senechal-Willis P, Curtiss R, III. 2010. Salmonella vaccine vectors displaying delayed antigen synthesis in vivo to enhance immunogenicity. Infect Immun 78:3969-3980.
54. Wang S, Li Y, Shi H, Sun W, Roland K L, Curtiss R, III. 2011. Comparison of a regulated delayed antigen synthesis system with in vivo-inducible promoters for antigen delivery by live attenuated Salmonella vaccines. Infect Immun 79:937-949.
55. Xin W, Wanda S Y, Li Y, Wang S, Mo H, Curtiss R, III. 2008. Analysis of type II secretion of recombinant pneumococcal PspA and PspC in a Salmonella enterica serovar Typhimurium vaccine with regulated delayed antigen synthesis. Infect Immun 76:3241-3254.
56. Shi H, Wang S, Curtiss R, III. 2013. Evaluation of regulated delayed attenuation strategies for Salmonella enterica serovar Typhi vaccine vectors in neonatal and infant mice. Clin Vaccine Immunol 20:931-944.
57. Shi H, Wang S, Roland K L, Gunn B M, Curtiss R, III. 2010. Immunogenicity of a live recombinant Salmonella enterica serovar Typhimurium vaccine expressing pspA in neonates and infant mice born from naive and immunized mothers. Clin Vaccine Immunol 17:363-371.
58. Ehretsmann C P, Carpousis A J, Krisch H M. 1992. Specificity of Escherichia coli endoribonuclease RNase E: in vivo and in vitro analysis of mutants in a bacteriophage T4 mRNA processing site. Genes Dev 6:149-159.
59. McDowall K J, Kaberdin V R, Wu S W, Cohen S N, Lin-Chao S. 1995. Site-specific RNase E cleavage of oligonucleotides and inhibition by stem-loops. Nature 374:287-290.
60. Lin-Chao S, Wong T T, McDowall K J, Cohen S N. 1994. Effects of nucleotide sequence on the specificity of rne-dependent and RNase E-mediated cleavages of RNA I encoded by the pBR322 plasmid. J Biol Chem 269:10797-10803.
61. Kang H Y, Curtiss R, III. 2003. Immune responses dependent on antigen location in recombinant attenuated Salmonella typhimurium vaccines following oral immunization. FEMS Immunol Med Microbiol 37:99-104.
62. Kang H Y, Srinivasan J, Curtiss R, III. 2002. Immune responses to recombinant pneumococcal PspA antigen delivered by live attenuated Salmonella enterica serovar Typhimurium vaccine. Infect Immun 70:1739-1749.
63. Konjufca V, Jenkins M, Wang S, Juarez-Rodriguez M D, Curtiss R, III. 2008. Immunogenicity of recombinant attenuated Salmonella enterica serovar Typhimurium vaccine strains carrying a gene that encodes Eimeria tenella antigen SO7. Infect Immun 76:5745-5753.
64. Muralinath M, Kuehn M J, Roland K L, Curtiss R, III. 2011. Immunization with Salmonella enterica serovar Typhimurium-derived outer membrane vesicles delivering the pneumococcal protein PspA confers protection against challenge with Streptococcus pneumoniae. Infect Immun 79:887-894.
65. Kong W, Wanda S Y, Zhang X, Bollen W, Tinge S A, Roland K L, Curtiss R, III. 2008. Regulated programmed lysis of recombinant Salmonella in host tissues to release protective antigens and confer biological containment. Proc Natl Acad Sci USA 105:9361-9366.
66. Ashraf S, Kong W, Wang S, Yang J, Curtiss R, III. 2011. Protective cellular responses elicited by vaccination with influenza nucleoprotein delivered by a live recombinant attenuated Salmonella vaccine. Vaccine 29:3990-4002.
67. Ameiss K, Ashraf S, Kong W, Pekosz A, Wu W H, Milich D, Billaud J N, Curtiss R, III. 2010. Delivery of woodchuck hepatitis virus-like particle presented influenza M2e by recombinant attenuated Salmonella displaying a delayed lysis phenotype. Vaccine 28:6704-6713.
68. Jiang Y, Mo H, Willingham C, Wang S, Park J Y, Kong W, Roland K L, Curtiss R, III. 2015. Protection Against Necrotic Enteritis in Broiler Chickens by Regulated Delayed Lysis Salmonella Vaccines. Avian Dis 59:475-485.
69. Germanier R, Furer E. 1971. Immunity in experimental salmonellosis. I I. Basis for the avirulence and protective capacity of gal E mutants of Salmonella typhimurium. Infect Immun 4:663-673.
70. Collins L V, Attridge S, Hackett J. 1991. Mutations at rfc or pmi attenuate Salmonella typhimurium virulence for mice. Infect Immun 59:1079-1085.
71. Frey S E, Lottenbach K R, Hill H, Blevins T P, Yu Y, Zhang Y, Brenneman K E, Kelly-Aehle S M, McDonald C, Jansen A, Curtiss R, III. 2013. A Phase I, dose-escalation trial in adults of three recombinant attenuated Salmonella Typhi vaccine vectors producing Streptococcus pneumoniae surface protein antigen PspA. Vaccine 31:4874-4880.
72. Gunn B M, Wanda S Y, Burshell D, Wang C, Curtiss R, III. 2010. Construction of recombinant attenuated Salmonella enterica serovar Typhimurium vaccine vector strains for safety in newborn and infant mice. Clin Vaccine Immunol 17:354-362.
73. Wang S, Hofacre C L, Wanda S Y, Zhou J, Callum R A, Nordgren B, Curtiss R, III. 2022. A triple-sugar regulated Salmonella vaccine protects against Clostridium perfringens-induced necrotic enteritis in broiler chickens. Poult Sci 101:101592.
74. Bentley R. 1990. The shikimate pathway—a metabolic tree with many branches. Crit Rev Biochem Mol Biol 25:307-384.
75. Galan J E, Nakayama K, Curtiss R, III. 1990. Cloning and characterization of the asd gene of Salmonella typhimurium: use in stable maintenance of recombinant plasmids in Salmonella vaccine strains. Gene 94:29-35.
76. Black S, Wright N G. 1955. Aspartic β-semialdehyde dehydrogenase and aspartic β-semialdehyde. J Biol Chem 213:39-50.
77. Nakayama K, Kelly S M, Curtiss R, III 1988. Construction of an Asd⁺ expression-cloning Vector: stable maintenance and high level expression of cloned genes in a Salmonella vaccine strain. Nat Biotech 6:693-697.
78. Xin W, Wanda S Y, Zhang X, Santander J, Scarpellini G, Ellis K, Alamuri P, Curtiss R, III. 2012. The Asd⁺-DadB⁺ dual-plasmid system offers a novel means to deliver multiple protective antigens by a recombinant attenuated Salmonella vaccine. Infect Immun 80:3621-3633.
79. Curtiss R, III. Apr. 15, 2003 2012. Regulated attenuation of live vaccines to enhance cross-protective immunogenicity. U.S. Pat. No. 8,133,493.
80. Lin H C, Lei S P, Studnicka G, Wilcox G. 1985. The araBAD operon of Salmonella typhimurium LT2. III. Nucleotide sequence of araD and its flanking regions, and primary structure of its product, L-ribulose-5-phosphate 4-epimerase. Gene 34:129-134.
81. Lin H C, Lei S P, Wilcox G. 1985. The araBAD operon of Salmonella typhimurium LT2. I I. Nucleotide sequence of araA and primary structure of its product, L-arabinose isomerase. Gene 34:123-128.
82. Lin H C, Lei S P, Wilcox G. 1985. The araBAD operon of Salmonella typhimurium LT2. I. Nucleotide sequence of araB and primary structure of its product, ribulokinase. Gene 34:111-122.
83. Lee J H, Nishitani J, Wilcox G. 1984. Genetic characterization of Salmonella typhimurium LT2 ara mutations. J Bacteriol 158:344-346.
84. Horwitz A H, Heffernan L, Morandi C, Lee J H, Timko J, Wilcox G. 1981. DNA sequence of the araBAD-araC controlling region in Salmonella typhimurium LT2. Gene 14:309-319.
85. Moralejo P, Egan S M, Hidalgo E, Aguilar J. 1993. Sequencing and characterization of a gene cluster encoding the enzymes for L-rhamnose metabolism in Escherichia coli. J Bacteriol 175:5585-5594.
86. Egan S M, Schleif R F. 1993. A regulatory cascade in the induction of rhaBAD. J Mol Biol 234:87-98.
87. Lee H, Hsu F F, Turk J, Groisman E A. 2004. The PmrA-regulated pmrC gene mediates phosphoethanolamine modification of lipid A and polymyxin resistance in Salmonella enterica. J Bacteriol 186:4124-4133.
88. Trent M, Raetz C. 2002. Cloning of EptA, the lipid A phosphoethanolamine transferase associated with polymyxin resistance. J Endotoxin Res 8:159.
89. Trent M S, Ribeiro A A, Lin S, Cotter R J, Raetz C R. 2001. An inner membrane enzyme in Salmonella and Escherichia coli that transfers 4-amino-4-deoxy-L-arabinose to lipid A: induction on polymyxin-resistant mutants and role of a novel lipid-linked donor. J Biol Chem 276:43122-43131.
90. Iino T, Komeda Y, Kutsukake K, Macnab R M, Matsumura P, Parkinson J S, Simon M I, Yamaguchi S. 1988. New unified nomenclature for the flagellar genes of Escherichia coli and Salmonella typhimurium. Microbiol Rev 52:533-535.
91. Iino T. 1977. Genetics of structure and function of bacterial flagella. Annu Rev Genet 11:161-182.
92. Iino T. 1969. Genetics and chemistry of bacterial flagella. Bacteriol Rev 33:454-475.
93. Liu Q, Liu Q, Yi J, Liang K, Hu B, Zhang X, Curtiss R, III, Kong Q. 2016. Outer membrane vesicles from flagellin-deficient Salmonella enterica serovar Typhimurium induce cross-reactive immunity and provide cross-protection against heterologous Salmonella challenge. Sci Rep 6:34776.
94. Zieg J, Simon M. 1980. Analysis of the nucleotide sequence of an invertible controlling element. Proc Natl Acad Sci USA 77:4196-4200.
95. Silverman M, Simon M. 1980. Phase variation: genetic analysis of switching mutants. Cell 19:845-854.
96. Silverman M, Zieg J, Simon M. 1979. Flagellar-phase variation: isolation of the rh1 gene. J Bacteriol 137:517-523.
97. Kutsukake K, Iino T. 1980. Inversions of specific DNA segments in flagellar phase variation of Salmonella and inversion systems of bacteriophages P1 and Mu. Proc Natl Acad Sci USA 77:7338-7341.
98. Bonifield H R, Hughes K T. 2003. Flagellar phase variation in Salmonella enterica is mediated by a posttranscriptional control mechanism. J Bacteriol 185:3567-3574.
99. Kutsukake K, Iino T. 1980. A trans-acting factor mediates inversion of a specific DNA segment in flagellar phase variation of Salmonella. Nature 284:479-481.
100. Yamamoto S, Kutsukake K. 2006. FIjA-mediated posttranscriptional control of phase 1 flagellin expression in flagellar phase variation of Salmonella enterica serovar Typhimurium. J Bacteriol 188:958-967.
101. Romling U, Bian Z, Hammar M, Sierralta W D, Normark S. 1998. Curli fibers are highly conserved between Salmonella typhimurium and Escherichia coli with respect to operon structure and regulation. J Bacteriol 180:722-731.
102. Laniewski P, Baek C H, Roland K L, Curtiss R, III. 2017. Analysis of Spleen-Induced Fimbria Production in Recombinant Attenuated Salmonella enterica Serovar Typhimurium Vaccine Strains. MBio 8:e01189-01117.
103. Lockman H A, Curtiss R, III. 1992. Isolation and characterization of conditional adherent and non-type 1 fimbriated Salmonella typhimurium mutants. Mol Microbiol 6:933-945.
104. Thankavel K, Shah A H, Cohen M S, Ikeda T, Lorenz R G, Curtiss R, III, Abraham S N. 1999. Molecular basis for the enterocyte tropism exhibited by Salmonella typhimurium type 1 fimbriae. J Biol Chem 274:5797-5809.
105. Freudl R, Cole S T. 1983. Cloning and molecular characterization of the ompA gene from Salmonella typhimurium. Eur J Biochem 134:497-502.
106. Norris F A, Wilson M P, Wallis T S, Galyov E E, Majerus P W. 1998. SopB, a protein required for virulence of Salmonella dublin, is an inositol phosphate phosphatase. Proc Natl Acad Sci USA 95:14057-14059.
107. Reis B P, Zhang S, Tsolis R M, Baumler A J, Adams L G, Santos R L. 2003. The attenuated sopB mutant of Salmonella enterica serovar Typhimurium has the same tissue distribution and host chemokine response as the wild type in bovine Peyer's patches. Vet Microbiol 97:269-277.
108. Zhang S, Santos R L, Tsolis R M, Stender S, Hardt W D, Baumler A J, Adams L G. 2002. The Salmonella enterica serotype typhimurium effector proteins SipA, SopA, SopB, SopD, and SopE2 act in concert to induce diarrhea in calves. Infect Immun 70:3843-3855.
109. Goncharoff P, Nichols B P. 1988. Evolution of aminobenzoate synthases: nucleotide sequences of Salmonella typhimurium and Klebsiella aerogenes pabB. Mol Biol Evol 5:531-548.
110. Kaplan J B, Merkel W K, Nichols B P. 1985. Evolution of glutamine amidotransferase genes. Nucleotide sequences of the pabA genes from Salmonella typhimurium, Klebsiella aerogenes and Serratia marcescens. J Mol Biol 183:327-340.
111. Schnaitman C A, Klena J D. 1993. Genetics of lipopolysaccharide biosynthesis in enteric bacteria. Microbiol Rev 57:655-682.
112. Kadrmas J L, Raetz C R. 1998. Enzymatic synthesis of lipopolysaccharide in Escherichia coli. Purification and properties of heptosyltransferase i. J Biol Chem 273:2799-2807.
113. Klena J D, Ashford R S, 2nd, Schnaitman C A. 1992. Role of Escherichia coli K-12 rfa genes and the rfp gene of Shige//a dysenteriae 1 in generation of lipopolysaccharide core heterogeneity and attachment of O antigen. J Bacteriol 174:7297-7307.
114. Heinrichs D E, Yethon J A, Whitfield C. 1998. Molecular basis for structural diversity in the core regions of the lipopolysaccharides of Escherichia coli and Salmonella enterica. Mol Microbiol 30:221-232.
115. Stevenson G, Andrianopoulos K, Hobbs M, Reeves P R. 1996. Organization of the Escherichia coli K-12 gene cluster responsible for production of the extracellular polysaccharide colanic acid. J Bacteriol 178:4885-4893.
116. Whitfield C. 2006. Biosynthesis and assembly of capsular polysaccharides in Escherichia coli. Annu Rev Biochem 75:39-68.
117. Pizarro-Cerda J, Tedin K. 2004. The bacterial signal molecule, ppGpp, regulates Salmonella virulence gene expression. Mol Microbiol 52:1827-1844.
118. Torok I, Kari C. 1980. Accumulation of ppGpp in a relA mutant of Escherichia coli during amino acid starvation. J Biol Chem 255:3838-3840.
119. Curtiss R, III, Wang S, Wanda S-Y, Kong W. Feb. 10, 2016 2018. Regulated expression of antigen and/or regulated attenuation to enhance vaccine immunogenicity and/or safety patent U S 9, 885, 051 B2.
120. Ahmer B M, Tran M, Heffron F. 1999. The virulence plasmid of Salmonella typhimurium is self-transmissible. J Bacteriol 181:1364-1368.
121. Na H S, Kim H J, Lee H C, Hong Y, Rhee J H, Choy H E. 2006. Immune response induced by Salmonella typhimurium defective in ppGpp synthesis. Vaccine 24:2027-2034.
122. Rudd K E, Bochner B R, Cashel M, Roth J R. 1985. Mutations in the spoT gene of Salmonella typhimurium: effects on his operon expression. J Bacteriol 163:534-542.
123. Cohen A, Laban A. 1983. Plasmidic recombination in Escherichia coli K-12: the role of recF gene function. Mol Gen Genet 189:471-474.
124. Kolodner R, Fishel R A, Howard M. 1985. Genetic recombination of bacterial plasmid DNA: effect of RecF pathway mutations on plasmid recombination in Escherichia coli. J Bacteriol 163:1060-1066.
125. Zhang X, Wanda S Y, Brenneman K, Kong W, Roland K, Curtiss R, III. 2011. Improving Salmonella vector with rec mutation to stabilize the DNA cargoes. BMC Microbiol 11:31.
126. Beuz6n C R, Meresse S, Unsworth K E, Ruiz-Albert J, Garvis S, Waterman S R, Ryder T A, Boucrot E, Holden D W. 2000. Salmonella maintains the integrity of its intracellular vacuole through the action of SifA. EMBO J 19:3235-3249.
127. Ohlson M B, Huang Z, Alto N M, Blanc M P, Dixon J E, Chai J, Miller S I. 2008. Structure and function of Salmonella SifA indicate that its interactions with SKIP, SseJ, and RhoA family GTPases induce endosomal tubulation. Cell Host Microbe 4:434-446.
128. Saltzman D A, Katsanis E, Heise C P, Hasz D E, Kelly S M, Curtiss R, III, Leonard A S, Anderson P M. 1997. Patterns of hepatic and splenic colonization by an attenuated strain of Salmonella typhimurium containing the gene for human interleukin-2: a novel anti-tumor agent. Cancer Biother Radiopharm 12:37-45.
129. Saltzman D A, Katsanis E, Heise C P, Hasz D E, Vigdorovich V, Kelly S M, Curtiss R, III, Leonard A S, Anderson P M. 1997. Antitumor mechanisms of attenuated Salmonella typhimurium containing the gene for human interleukin-2: a novel antitumor agent? J Pediatr Surg 32:301-306.
130. Schodel F, Kelly S M, Peterson D L, Milich D R, Curtiss R, III. 1994. Hybrid hepatitis B virus core-pre-S proteins synthesized in avirulent Salmonella typhimurium and Salmonella typhi for oral vaccination. Infect Immun 62:1669-1676.
131. Augustin L B, Milbauer L, Hastings S E, Leonard A S, Saltzman D A, Schottel J L. 2021. Salmonella enterica Typhimurium engineered for nontoxic systemic colonization of autochthonous tumors. J Drug Target 29:294-299.
132. Curtiss R, III., Porter S B, Munson M, Tinge S A, Hassan J O, Gentry-Weeks C, Kelly S M. 1991. Nonrecombinant and recombinant avirulent Salmonella live vaccines for poultry, p 169-198. In Blankenship L C, Bailey J H S, Cox N A, Stern N J, Meinersmann R J (ed), Colonization control of human bacterial enteropathogens in poultry. Academic Press New York
133. Zhang X, Kelly S M, Bollen W S, Curtiss R, III. 1997. Characterization and immunogenicity of Salmonella typhimurium SL1344 and UK-1 Acrp and Acdt deletion mutants. Infect Immun 65:5381-5387.
134. Sanapala S, Mosca L, Wang S, Curtiss R, III 2018. Comparative evaluation of Salmonella vaccines derived from UK-1 and 14028S submitted. 135. Bertani G. 1951. Studies on lysogenesis. I. The mode of phage liberation by lysogenic Escherichia coli. J Bacteriol 62:293-300.
136. Sambrook J, Russell D W. 2001. Molecular cloning: a laboratory manual, 3rd ed. Cold Spring Harbor Laboratory Press, Cold Spring Harbor, N.Y.
137. Schmieger H. 1972. Phage P22-mutants with increased or decreased transduction abilities. Mol Gen Genet 119:75-88.
138. Schmieger H, Backhaus H. 1976. Altered cotransduction frequencies exhibited by H T-mutants of Salmonella-phage P22. Mol Gen Genet 143:307-309.
139. Kang H Y, Dozois C M, Tinge S A, Lee T H, Curtiss R, III. 2002. Transduction-mediated transfer of unmarked deletion and point mutations through use of counterselectable suicide vectors. J Bacteriol 184:307-312.
140. Edwards R A, Keller L H, Schifferli D M. 1998. Improved allelic exchange vectors and their use to analyze 987P fimbria gene expression. Gene 207:149-157.
141. Quandt J, Hynes M F. 1993. Versatile suicide vectors which allow direct selection for gene replacement in gram-negative bacteria. Gene 127:15-21.
142. Roland K, Curtiss R, III., Sizemore D. 1999. Construction and evaluation of a Δcya Δcrp Salmonella typhimurium strain expressing avian pathogenic Escherichia coli 078 LPS as a vaccine to prevent airsacculitis in chickens. Avian Dis 43:429-441.
143. Shi H, Santander J, Brenneman K E, Wanda S Y, Wang S, Senechal P, Sun W, Roland K L, Curtiss R, III. 2010. Live recombinant Salmonella Typhi vaccines constructed to investigate the role of rpoS in eliciting immunity to a heterologous antigen. PLoS One 5:e11142.
144. Wilmes-Riesenberg M R, Bearson B, Foster J W, Curtiss R, III. 1996. Role of the acid tolerance response in virulence of Salmonella typhimurium. Infect Immun 64:1085-1092.
145. Murray G L, Attridge S R, Morona R. 2005. Inducible serum resistance in Salmonella typhimurium is dependent on wzz_{fepE}-regulated very long O antigen chains. Microbes Infect 7:1296-1304.
146. Hitchcock P J, Brown T M. 1983. Morphological heterogeneity among Salmonella lipopolysaccharide chemotypes in silver-stained polyacrylamide gels. J Bacteriol 154:269-277.
147. Lin T Y, Zhang H, Luo J, Li Y, Gao T, Lara P N, Jr., de Vere White R, Lam K S, Pan C X. 2012. Multifunctional targeting micelle nanocarriers with both imaging and therapeutic potential for bladder cancer. Int J Nanomedicine 7:2793-2804.
148. Lin T Y, Li Y P, Zhang H, Luo J, Goodwin N, Gao T, White Rde V, Lam K S, Pan C X. 2013. Tumor-targeting multifunctional micelles for imaging and chemotherapy of advanced bladder cancer. Nanomedicine (Lond) 8:1239-1251.
149. Li Y, Lin T Y, Luo Y, Liu Q, Xiao W, Guo W, Lac D, Zhang H, Feng C, Wachsmann-Hogiu S, Walton J H, Cherry S R, Rowland D J, Kukis D, Pan C, Lam K S. 2014. A smart and versatile theranostic nanomedicine platform based on nanoporphyrin. Nat Commun 5:4712.
150. Lin T Y, Li Y, Liu Q, Chen J L, Zhang H, Lac D, Zhang H, Ferrara K W, Wachsmann-Hogiu S, Li T, Airhart S, deVere White R, Lam K S, Pan C X. 2016. Novel theranostic nanoporphyrins for photodynamic diagnosis and trimodal therapy for bladder cancer. Biomaterials 104:339-351.
151. Zhang H, Aina O H, Lam K S, de Vere White R, Evans C, Henderson P, Lara P N, Wang X, Bassuk J A, Pan C X. 2012. Identification of a bladder cancer-specific ligand using a combinatorial chemistry approach. Urol Oncol 30:635-645.
152. Galan J E, Curtiss R, III. 1989. Cloning and molecular characterization of genes whose products allow Salmonella typhimurium to penetrate tissue culture cells. Proc Natl Acad Sci USA 86:6383-6387.
153. Schneider C A, Rasband W S, Eliceiri K W. 2012. NIH Image to ImageJ: 25 years of image analysis. Nat Methods 9:671-675.
154. Curtiss R, III., Wang S. 2021. Induction of protective immunity against antigens. U S patent U.S. Pat. No. 10,988,729.
155. Joseph M, Enting D. 2019. Immune Responses in Bladder Cancer-Role of Immune Cell Populations, Prognostic Factors and Therapeutic Implications. Front Oncol 9:1270.
156. Cole K E, Strick C A, Paradis T J, Ogborne K T, Loetscher M, Gladue R P, Lin W, Boyd J G, Moser B, Wood D E, Sahagan B G, Neote K. 1998. Interferon-inducible T cell alpha chemoattractant (I-TAC): a novel non-ELR CXC chemokine with potent activity on activated T cells through selective high affinity binding to CXCR3. J Exp Med 187:2009-2021.
157. Naumann U, Cameroni E, Pruenster M, Mahabaleshwar H, Raz E, Zerwes H G, Rot A, Thelen M. 2010. CXCR7 functions as a scavenger for CXCL12 and CXCL11. PLoS One 5:e9175.
158. Colvin R A, Campanella G S, Sun J, Luster A D. 2004. Intracellular domains of CXCR3 that mediate CXCL9, CXCL10, and CXCL11 function. J Biol Chem 279:30219-30227.
159. Karin N. 2020. CXCR3 Ligands in Cancer and Autoimmunity, Chemoattraction of Effector T Cells, and Beyond. Front Immunol 11:976.
160. Gao Q, Zhang Y. 2021. CXCL11 Signaling in the Tumor Microenvironment. Adv Exp Med Biol 1302:41-50.
161. Van Raemdonck K, Van den Steen P E, Liekens S, Van Damme J, Struyf S. 2015. CXCR3 ligands in disease and therapy. Cytokine Growth Factor Rev 26:311-327.
162. Billottet C, Quemener C, Bikfalvi A. 2013. CXCR3, a double-edged sword in tumor progression and angiogenesis. Biochim Biophys Acta 1836:287-295.
163. Biber K, Dijkstra I, Trebst C, De Groot C J, Ransohoff R M, Boddeke H W. 2002. Functional expression of CXCR3 in cultured mouse and human astrocytes and microglia. Neuroscience 112:487-497.
164. Sanchez-Martin L, Sanchez-Mateos P, Cabanas C. 2013. CXCR7 impact on CXCL12 biology and disease. Trends Mol Med 19:12-22.
165. Tokunaga R, Zhang W, Naseem M, Puccini A, Berger M D, Soni S, McSkane M, Baba H, Lenz H J. 2018. CXCL9, CXCL10, CXCL11/CXCR3 axis for immune activation—A target for novel cancer therapy. Cancer Treat Rev 63:40-47.
166. Cao Y, Jiao N, Sun T, Ma Y, Zhang X, Chen H, Hong J, Zhang Y. 2021. CXCL11 Correlates With Antitumor Immunity and an Improved Prognosis in Colon Cancer. Front Cell Dev Biol 9:646252.
167. Moon E K, Wang L S, Bekdache K, Lynn R C, Lo A, Thorne S H, Albelda S M. 2018. Intra-tumoral delivery of CXCL11 via a vaccinia virus, but not by modified T cells, enhances the efficacy of adoptive T cell therapy and vaccines. Oncoimmunology 7:e1395997.
168. Gao Q, Wang S, Chen X, Cheng S, Zhang Z, Li F, Huang L, Yang Y, Zhou B, Yue D, Wang D, Cao L, Maimela N R, Zhang B, Yu J, Wang L, Zhang Y. 2019. Cancer-cell-secreted CXCL11 promoted CD8(+) T cells infiltration through docetaxel-induced-release of HMGB1 in NSCLC. J Immunother Cancer 7:42.
169. Wang P, Yang X, Xu W, Li K, Chu Y, Xiong S. 2010. Integrating individual functional moieties of CXCL10 and CXCL11 into a novel chimeric chemokine leads to synergistic antitumor effects: a strategy for chemokine-based multi-target-directed cancer therapy. Cancer Immunol Immunother 59:1715-1726.
170. Bulina M E, Chudakov D M, Britanova O V, Yanushevich Y G, Staroverov D B, Chepurnykh T V, Merzlyak E M, Shkrob M A, Lukyanov S, Lukyanov K A. 2006. A genetically encoded photosensitizer. Nat Biotechnol 24:95-99.
171. Yuan M, Liu C, Li J, Ma W, Yu X, Zhang P, Ji Y. 2019. The effects of photodynamic therapy on leukemia cells mediated by KillerRed, a genetically encoded fluorescent protein photosensitizer. BMC Cancer 19:934.
172. Liu J, Wang F, Qin Y, Feng X. 2021. Advances in the Genetically Engineered KillerRed for Photodynamic Therapy Applications. Int J Mol Sci 22:10130.
173. Liao Z X, Li Y C, Lu H M, Sung H W. 2014. A genetically-encoded KillerRed protein as an intrinsically generated photosensitizer for photodynamic therapy. Biomaterials 35:500-508.
174. Carpentier P, Violot S, Blanchoin L, Bourgeois D. 2009. Structural basis for the phototoxicity of the fluorescent protein KillerRed. FEBS Lett 583:2839-2842.
175. Bulina M E, Lukyanov K A, Britanova O V, Onichtchouk D, Lukyanov S, Chudakov D M. 2006. Chromophore-assisted light inactivation (CALI) using the phototoxic fluorescent protein KillerRed. Nat Protoc 1:947-953.
176. Brosius J, Erfle M, Storella J. 1985. Spacing of the −10 and −35 regions in the tac promoter. Effect on its in vivo activity. J Biol Chem 260:3539-3541.
177. Pelletier J, Sonenberg N. 1988. Internal initiation of translation of eukaryotic mRNA directed by a sequence derived from poliovirus RNA. Nature 334:320-325.
178. Kim J H, Lee S R, Li L H, Park H J, Park J H, Lee K Y, Kim M K, Shin B A, Choi S Y. 2011. High cleavage efficiency of a 2A peptide derived from porcine teschovirus-1 in human cell lines, zebrafish and mice. PLoS One 6:e18556.
179. Jang S K, Krausslich H G, Nicklin M J, Duke G M, Palmenberg A C, Wimmer E. 1988. A segment of the 5′ nontranslated region of encephalomyocarditis virus RNA directs internal entry of ribosomes during in vitro translation. J Virol 62:2636-2643.
180. Kreiter S, Selmi A, Diken M, Sebastian M, Osterloh P, Schild H, Huber C, Tureci O, Sahin U. 2008. Increased antigen presentation efficiency by coupling antigens to MHC class I trafficking signals. J Immunol 180:309-318.
181. Hinnebusch A G, Ivanov I P, Sonenberg N. 2016. Translational control by 5′-untranslated regions of eukaryotic mRNAs. Science 352:1413-1416.
182. Sonenberg N. 1994. mRNA translation: influence of the 5′ and 3′ untranslated regions. Curr Opin Genet Dev 4:310-315.
183. Yamashita A, Takeuchi O. 2017. Translational control of mRNAs by 3′-Untranslated region binding proteins. BMB Rep 50:194-200.
184. Holtkamp S, Kreiter S, Selmi A, Simon P, Koslowski M, Huber C, Tureci O, Sahin U. 2006. Modification of antigen-encoding RNA increases stability, translational efficacy, and T-cell stimulatory capacity of dendritic cells. Blood 108:4009-4017.
185. Castle J C, Kreiter S, Diekmann J, Lower M, van de Roemer N, de Graaf J, Selmi A, Diken M, Boegel S, Paret C, Koslowski M, Kuhn A N, Britten C M, Huber C, Tureci O, Sahin U. 2012. Exploiting the mutanome for tumor vaccination. Cancer Res 72:1081-1091.
186. Kreiter S, Vormehr M, van de Roemer N, Diken M, Lower M, Diekmann J, Boegel S, Schrors B, Vascotto F, Castle J C, Tadmor A D, Schoenberger S P, Huber C, Tureci O, Sahin U. 2015. Mutant MHC class II epitopes drive therapeutic immune responses to cancer. Nature 520:692-696.
187. Kranz L M, Diken M, Haas H, Kreiter S, Loquai C, Reuter K C, Meng M, Fritz D, Vascotto F, Hefesha H, Grunwitz C, Vormehr M, Husemann Y, Selmi A, Kuhn A N, Buck J, Derhovanessian E, Rae R, Attig S, Diekmann J, Jabulowsky R A, Heesch S, Hassel J, Langguth P, Grabbe S, Huber C, Tureci O, Sahin U. 2016. Systemic RNA delivery to dendritic cells exploits antiviral defence for cancer immunotherapy. Nature 534:396-401.
188. Ott P A, Hu Z, Keskin D B, Shukla S A, Sun J, Bozym D J, Zhang W, Luoma A, Giobbie-Hurder A, Peter L, Chen C, Olive O, Carter T A, Li S, Lieb D J, Eisenhaure T, Gjini E, Stevens J, Lane W J, Javeri I, Nellaiappan K, Salazar A M, Daley H, Seaman M, Buchbinder El, Yoon C H, Harden M, Lennon N, Gabriel S, Rodig S J, Barouch D H, Aster J C, Getz G, Wucherpfennig K, Neuberg D, Ritz J, Lander E S, Fritsch E F, Hacohen N, Wu C J. 2017. An immunogenic personal neoantigen vaccine for patients with melanoma. Nature 547:217-221.
189. Gubin M M, Artyomov M N, Mardis E R, Schreiber R D. 2015. Tumor neoantigens: building a framework for personalized cancer immunotherapy. J Clin Invest 125:3413-3421.
190. Schumacher T N, Schreiber R D. 2015. Neoantigens in cancer immunotherapy. Science 348:69-74.
191. Ott P A, Hu-Lieskovan S, Chmielowski B, Govindan R, Naing A, Bhardwaj N, Margolin K, Awad M M, Hellmann M D, Lin J J, Friedlander T, Bushway M E, Balogh K N, Sciuto T E, Kohler V, Turnbull S J, Besada R, Curran R R, Trapp B, Scherer J, Poran A, Harjanto D, Barthelme D, Ting Y S, Dong J Z, Ware Y, Huang Y, Huang Z, Wanamaker A, Cleary L D, Moles M A, Manson K, Greshock J, Khondker Z S, Fritsch E, Rooney M S, DeMario M, Gaynor R B, Srinivasan L. 2020. A Phase Ib Trial of Personalized Neoantigen Therapy Plus Anti-PD-1 in Patients with Advanced Melanoma, Non-small Cell Lung Cancer, or Bladder Cancer. Cell 183:347-362 e324.
192. Desrichard A, Snyder A, Chan T A. 2016. Cancer Neoantigens and Applications for Immunotherapy. Clin Cancer Res 22:807-812.
193. Capietto A H, Jhunjhunwala S, Delamarre L. 2017. Characterizing neoantigens for personalized cancer immunotherapy. Curr Opin Immunol 46:58-65.
194. Zhu Y, Liu J. 2021. The Role of Neoantigens in Cancer Immunotherapy. Front Oncol 11:682325.
195. Pearlman A H, Hwang M S, Konig M F, Hsiue E H, Douglass J, DiNapoli S R, Mog B J, Bettegowda C, Pardoll D M, Gabelli S B, Papadopoulos N, Kinzler K W, Vogelstein B, Zhou S. 2021. Targeting public neoantigens for cancer immunotherapy. Nat Cancer 2:487-497.
196. Ma W, Pham B, Li T. 2022. Cancer neoantigens as potential targets for immunotherapy. Clin Exp Metastasis 39:51-60.
197. Castle J C, Uduman M, Pabla S, Stein R B, Buell J S. 2019. Mutation-Derived Neoantigens for Cancer Immunotherapy. Front Immunol 10:1856.
198. Hu Z, Leet D E, Allesoe R L, Oliveira G, Li S, Luoma A M, Liu J, Forman J, Huang T, lorgulescu J B, Holden R, Sarkizova S, Gohil S H, Redd R A, Sun J, Elagina L, Giobbie-Hurder A, Zhang W, Peter L, Ciantra Z, Rodig S, Olive O, Shetty K, Pyrdol J, Uduman M, Lee P C, Bachireddy P, Buchbinder El, Yoon C H, Neuberg D, Pentelute B L, Hacohen N, Livak K J, Shukla S A, Olsen L R, Barouch D H, Wucherpfennig K W, Fritsch E F, Keskin D B, Wu C J, Ott P A. 2021. Personal neoantigen vaccines induce persistent memory T cell responses and epitope spreading in patients with melanoma. Nat Med 27:515-525.
199. Vasconcelos-Nobrega C, Colaco A, Lopes C, Oliveira P A. 2012. Review: BBN as an urothelial carcinogen. In Vivo 26:727-739.
200. Summerhayes I C, Franks L M. 1979. Effects of donor age on neoplastic transformation of adult mouse bladder epithelium in vitro. J Natl Cancer Inst 62:1017-1023.
201. Lattime E C, Gomella L G, McCue P A. 1992. Murine bladder carcinoma cells present antigen to BCG-specific CD4⁺ T-cells. Cancer Res 52:4286-4290.
202. Saito R, Smith C C, Utsumi T, Bixby L M, Kardos J, Wobker S E, Stewart K G, Chai S, Manocha U, Byrd K M, Damrauer J S, Williams S E, Vincent B G, Kim W Y. 2018. Molecular Subtype-Specific Immunocompetent Models of High-Grade Urothelial Carcinoma Reveal Differential Neoantigen Expression and Response to Immunotherapy. Cancer Res 78:3954-3968.
203. Prosch S, Stein J, Staak K, Liebenthal C, Volk H D, Kruger D H. 1996. Inactivation of the very strong HCMV immediate early promoter by DNA CpG methylation in vitro. Biol Chem Hoppe Seyler 377:195-201.
204. Brooks A R, Harkins R N, Wang P, Qian H S, Liu P, Rubanyi G M. 2004. Transcriptional silencing is associated with extensive methylation of the CMV promoter following adenoviral gene delivery to muscle. J Gene Med 6:395-404.
205. Uetsuki T, Naito A, Nagata S, Kaziro Y. 1989. Isolation and characterization of the human chromosomal gene for polypeptide chain elongation factor-1a. J Biol Chem 264:5791-5798.
206. Kim D W, Uetsuki T, Kaziro Y, Yamaguchi N, Sugano S. 1990. Use of the human elongation factor 1α promoter as a versatile and efficient expression system. Gene 91:217-223.
207. Kaufman W L, Kocman I, Agrawal V, Rahn H P, Besser D, Gossen M. 2008. Homogeneity and persistence of transgene expression by omitting antibiotic selection in cell line isolation. Nucleic Acids Res 36:e111.
208. Teschendorf C, Warrington K H, Jr., Siemann D W, Muzyczka N. 2002. Comparison of the EF-1 a and the CMV promoter for engineering stable tumor cell lines using recombinant adeno-associated virus. Anticancer Res 22:3325-3330.
209. Gopalkrishnan R V, Christiansen K A, Goldstein N I, DePinho R A, Fisher P B. 1999. Use of the human EF-1 a promoter for expression can significantly increase success in establishing stable cell lines with consistent expression: a study using the tetracycline-inducible system in human cancer cells. Nucleic Acids Res 27:4775-4782.
210. Byun H M, Suh D, Jeong Y, Wee H S, Kim J M, Kim W K, Ko J J, Kim J S, Lee Y B, Oh Y K. 2005. Plasmid vectors harboring cellular promoters can induce prolonged gene expression in hematopoietic and mesenchymal progenitor cells. Biochem Biophys Res Commun 332:518-523.
211. Wang X, Xu Z, Tian Z, Zhang X, Xu D, Li Q, Zhang J, Wang T. 2017. The EF-1α promoter maintains high-level transgene expression from episomal vectors in transfected CHO-K1 cells. J Cell Mol Med 21:3044-3054.
212. Gill D R, Smyth S E, Goddard C A, Pringle I A, Higgins C F, Colledge W H, Hyde S C. 2001. Increased persistence of lung gene expression using plasmids containing the ubiquitin C or elongation factor 1α promoter. Gene Ther 8:1539-1546.
213. Pardoll D M. 2012. The blockade of immune checkpoints in cancer immunotherapy. Nat Rev Cancer 12:252-264.
214. Brahmer J R, Tykodi S S, Chow L Q, Hwu W J, Topalian S L, Hwu P, Drake C G, Camacho L H, Kauh J, Odunsi K, Pitot H C, Hamid O, Bhatia S, Martins R, Eaton K, Chen S, Salay T M, Alaparthy S, Grosso J F, Korman A J, Parker S M, Agrawal S, Goldberg S M, Pardoll D M, Gupta A, Wigginton J M. 2012. Safety and activity of anti-PD-L1 antibody in patients with advanced cancer. N Engl J Med 366:2455-2465.
215. Topalian S L, Hodi F S, Brahmer J R, Gettinger S N, Smith D C, McDermott D F, Powderly J D, Carvajal R D, Sosman J A, Atkins M B, Leming P D, Spigel D R, Antonia S J, Horn L, Drake C G, Pardoll D M, Chen L, Sharfman W H, Anders R A, Taube J M, McMiller T L, Xu H, Korman A J, Jure-Kunkel M, Agrawal S, McDonald D, Kollia G D, Gupta A, Wigginton J M, Sznol M. 2012. Safety, activity, and immune correlates of anti-PD-1 antibody in cancer. N Engl J Med 366:2443-2454.
216. Hamid O, Robert C, Daud A, Hodi F S, Hwu W J, Kefford R, Wolchok J D, Hersey P, Joseph R W, Weber J S, Dronca R, Gangadhar T C, Patnaik A, Zarour H, Joshua A M, Gergich K, Elassaiss-Schaap J, Algazi A, Mateus C, Boasberg P, Tumeh P C, Chmielowski B, Ebbinghaus S W, Li X N, Kang S P, Ribas A. 2013. Safety and tumor responses with lambrolizumab (anti-PD-1) in melanoma. N Engl J Med 369:134-144.
217. Motzer R J, Rini B I, McDermott D F, Redman B G, Kuzel T M, Harrison M R, Vaishampayan U N, Drabkin H A, George S, Logan T F, Margolin K A, Plimack E R, Lambert A M, Waxman I M, Hammers H J. 2015. Nivolumab for Metastatic Renal Cell Carcinoma: Results of a Randomized Phase I I Trial. J Clin Oncol 33:1430-1437.
218. Rizvi N A, Mazieres J, Planchard D, Stinchcombe T E, Dy G K, Antonia S J, Horn L, Lena H, Minenza E, Mennecier B, Otterson G A, Campos L T, Gandara D R, Levy B P, Nair S G, Zalcman G, Wolf J, Souquet P J, Baldini E, Cappuzzo F, Chouaid C, Dowlati A, Sanborn R, Lopez-Chavez A, Grohe C, Huber R M, Harbison C T, Baudelet C, Lestini B J, Ramalingam S S. 2015. Activity and safety of nivolumab, an anti-PD-1 immune checkpoint inhibitor, for patients with advanced, refractory squamous non-small-cell lung cancer (CheckMate 063): a phase 2, single-arm trial. Lancet Oncol 16:257-265.
219. Tobias J, Steinberger P, Drinic M, Wiedermann U. 2021. Emerging targets for anticancer vaccination: PD-1. ESMO Open 6:100278.
220. Lee C M, Tannock I F. 2010. The distribution of the therapeutic monoclonal antibodies cetuximab and trastuzumab within solid tumors. BMC Cancer 10:255.
221. Sharkey R M, Gold D V, Aninipot R, Vagg R, Ballance C, Newman E S, Ostella F, Hansen H J, Goldenberg D M. 1990. Comparison of tumor targeting in nude mice by murine monoclonal antibodies directed against different human colorectal cancer antigens. Cancer Res 50:828s-834s.
222. Scott A M, Wolchok J D, Old U. 2012. Antibody therapy of cancer. Nat Rev Cancer 12:278-287.
223. Baldo B A. 2022. Immune- and Non-Immune-Mediated Adverse Effects of Monoclonal Antibody Therapy: A Survey of 110 Approved Antibodies. Antibodies (Basel) 11:17.
224. Weiner G J. 2015. Building better monoclonal antibody-based therapeutics. Nat Rev Cancer 15:361-370.
225. Brahmer J R, Drake C G, Wollner I, Powderly J D, Picus J, Sharfman W H, Stankevich E, Pons A, Salay T M, McMiller T L, Gilson M M, Wang C, Selby M, Taube J M, Anders R, Chen L, Korman A J, Pardoll D M, Lowy I, Topalian S L. 2010. Phase I study of single-agent anti-programmed death-1 (MDX-1106) in refractory solid tumors: safety, clinical activity, pharmacodynamics, and immunologic correlates. J Clin Oncol 28:3167-3175.
226. Garcia-Marquez M A, Thelen M, Reinke S, Keller D, Wennhold K, Lehmann J, Veldman J, Borchmann S, Rosenwald A, Sasse S, Diepstra A, Borchmann P, Engert A, Klapper W, von Bergwelt-Baildon M, Brockelmann P J, Schlosser H A. 2022. Reverted exhaustion phenotype of circulating lymphocytes as immune correlate of anti-PD1 first-line treatment in Hodgkin lymphoma. Leukemia 36:760-771.
227. Mor A, Strazza M. 2021. Bridging the Gap: Connecting the Mechanisms of Immune-Related Adverse Events and Autoimmunity Through PD-1. Front Cell Dev Biol 9:790386.
228. Pascolutti R, Sun X, Kao J, Maute R L, Ring A M, Bowman G R, Kruse A C. 2016. Structure and Dynamics of P D-L1 and an Ultra-High-Affinity PD-1 Receptor Mutant. Structure 24:1719-1728.
229. Maute R L, Gordon S R, Mayer A T, McCracken M N, Natarajan A, Ring N G, Kimura R, Tsai J M, Manglik A, Kruse A C, Gambhir S S, Weissman I L, Ring A M. 2015. Engineering high-affinity PD-1 variants for optimized immunotherapy and immuno-PET imaging. Proc Natl Acad Sci USA 112:E6506-6514.
230. Tan S, Liu K, Chai Y, Zhang C W, Gao S, Gao G F, Qi J. 2018. Distinct P D-L1 binding characteristics of therapeutic monoclonal antibody durvalumab. Protein Cell 9:135-139.
231. Li Y, Cong Y, Jia M, He Q, Zhong H, Zhao Y, Li H, Yan M, You J, Liu J, Chen L, Hang H, Wang S. 2021. Targeting IL-21 to tumor-reactive T cells enhances memory T cell responses and anti-PD-1 antibody therapy. Nat Commun 12:951.
232. Sasada R, Onda H, Igarashi K. 1987. The establishment of IL-2 producing cells by genetic engineering. Cell Struct Funct 12:205-217.
233. Sasada R, Marumoto R, Igarashi K. 1988. Secretion of human EGF and IgE in mammalian cells by recombinant DNA techniques; use of a IL-2 leader sequence. Cell Struct Funct 13:129-141.
234. Taniguchi T, Matsui H, Fujita T, Takaoka C, Kashima N, Yoshimoto R, Hamuro J. 1983. Structure and expression of a cloned cDNA for human interleukin-2. Nature 302:305-310.
235. Engel J B, Tinneberg H R, Rick F G, Berkes E, Schally A V. 2016. Targeting of Peptide Cytotoxins to LHRH Receptors For Treatment of Cancer. Curr Drug Targets 17:488-494.
236. Minko T, Dharap S S, Pakunlu R I, Wang Y. 2004. Molecular targeting of drug delivery systems to cancer. Curr Drug Targets 5:389-406.
237. Dharap S S, Qiu B, Williams G C, Sinko P, Stein S, Minko T. 2003. Molecular targeting of drug delivery systems to ovarian cancer by BH3 and LHRH peptides. J Control Release 91:61-73.
238. Dharap S S, Minko T. 2003. Targeted proapoptotic LHRH-BH3 peptide. Pharm Res 20:889-896.
239. Szabo Z, Dezso B, Fodor K, Szegedi K, Flasko T, Szabo E, Olah G, Sipos E, Dobos N, Gardi J, Schally A V, Halmos G. 2021. Expression of Luteinizing Hormone-Releasing Hormone (LHRH) and Type-I LHRH Receptor in Transitional Cell Carcinoma Type of Human Bladder Cancer. Molecules 26:1253.
240. Lae M, Couturier J, Oudard S, Radvanyi F, Beuzeboc P, Vieillefond A. 2010. Assessing HER2 gene amplification as a potential target for therapy in invasive urothelial bladder cancer with a standardized methodology: results in 1005 patients. Ann Oncol 21:815-819.
241. Dharap S S, Wang Y, Chandna P, Khandare J J, Qiu B, Gunaseelan S, Sinko P J, Stein S, Farmanfarmaian A, Minko T. 2005. Tumor-specific targeting of an anticancer drug delivery system by LHRH peptide. Proc Natl Acad Sci USA 102:12962-12967.
242. Iqbal N, Iqbal N. 2014. Human Epidermal Growth Factor Receptor 2 (HER2) in Cancers: Overexpression and Therapeutic Implications. Mol Biol Int 2014:852748.
243. Li X, Taratula O, Taratula O, Schumann C, Minko T. 2017. LHRH-Targeted Drug Delivery Systems for Cancer Therapy. Mini Rev Med Chem 17:258-267.
244. Nagy A, Schally A V. 2005. Targeting of cytotoxic luteinizing hormone-releasing hormone analogs to breast, ovarian, endometrial, and prostate cancers. Biol Reprod 73:851-859.
245. Slamon D J, Clark G M, Wong S G, Levin W J, Ullrich A, McGuire W L. 1987. Human breast cancer: correlation of relapse and survival with amplification of the HER-2/neu oncogene. Science 235:177-182.
246. Yokota T, Milenic D E, Whitlow M, Schlom J. 1992. Rapid tumor penetration of a single-chain Fv and comparison with other immunoglobulin forms. Cancer Res 52:3402-3408.
247. Adams G P, McCartney J E, Tai M S, Oppermann H, Huston J S, Stafford W F, 3rd, Bookman M A, Fand I, Houston L L, Weiner L M. 1993. Highly specific in vivo tumor targeting by monovalent and divalent forms of 741F8 anti-c-erbB-2 single-chain Fv. Cancer Res 53:4026-4034.
248. Milenic D E, Yokota T, Filpula D R, Finkelman M A, Dodd S W, Wood J F, Whitlow M, Snoy P, Schlom J. 1991. Construction, binding properties, metabolism, and tumor targeting of a single-chain Fv derived from the pancarcinoma monoclonal antibody CC49. Cancer Res 51:6363-6371.
249. Li X, Stuckert P, Bosch I, Marks J D, Marasco W A. 2001. Single-chain antibody-mediated gene delivery into ErbB2-positive human breast cancer cells. Cancer Gene Ther 8:555-565.
250. Schier R, McCall A, Adams G P, Marshall K W, Merritt H, Yim M, Crawford R S, Weiner L M, Marks C, Marks J D. 1996. Isolation of picomolar affinity anti-c-erbB-2 single-chain Fv by molecular evolution of the complementarity determining regions in the center of the antibody binding site. J Mol Biol 263:551-567.
251. Song E, Zhu P, Lee S K, Chowdhury D, Kussman S, Dykxhoorn D M, Feng Y, Palliser D, Weiner D B, Shankar P, Marasco W A, Lieberman J. 2005. Antibody mediated in vivo delivery of small interfering RNAs via cell-surface receptors. Nat Biotechnol 23:709-717.
252. Raetz C R, Whitfield C. 2002. Lipopolysaccharide endotoxins. Annu Rev Biochem 71:635-700.
253. Subik K, Lee J F, Baxter L, Strzepek T, Costello D, Crowley P, Xing L, Hung M C, Bonfiglio T, Hicks D G, Tang P. 2010. The Expression Patterns of E R, P R, HER2, CK5/6, EGFR, Ki-67 and A R by Immunohistochemical Analysis in Breast Cancer Cell Lines. Breast Cancer (Auckl) 4:35-41.
254. Blankenstein M A, Henkelman M S, Klijn J G. 1985. Direct inhibitory effect of a luteinizing hormone-releasing hormone agonist on MCF-7 human breast cancer cells. Eur J Cancer Clin Oncol 21:1493-1499.
255. Meng J, Lu X, Zhou Y, Zhang M, Ge Q, Zhou J, Hao Z, Gao S, Yan F, Liang C. 2021. Tumor immune microenvironment-based classifications of bladder cancer for enhancing the response rate of immunotherapy. Mol Ther Oncolytics 20:410-421.
256. Berencsi K, Meropol N J, Hoffman J P, Sigurdson E, Giles L, Rani P, Somasundaram R, Zhang T, Kalabis J, Caputo L, Furth E, Swoboda R, Marincola F, Herlyn D. 2007. Colon carcinoma cells induce CXCL11-dependent migration of CXCR3-expressing cytotoxic T lymphocytes in organotypic culture. Cancer Immunol Immunother 56:359-370.
257. Nazari A, Ahmadi Z, Hassanshahi G, Abbasifard M, Taghipour Z, Falahati-Pour S K, Khorramdelazad H. 2020. Effective Treatments for Bladder Cancer Affecting CXCL9/CXCL10/CXCL11/CXCR3 Axis: A Review. Oman Med J 35:e103.
258. Yan L, Kanada M, Zhang J, Okazaki S, Terakawa S. 2015. Photodynamic Treatment of Tumor with Bacteria Expressing KillerRed. PLoS One 10:e0131518.
259. Shirmanova M, Yuzhakova D, Snopova L, Perelman G, Serebrovskaya E, Lukyanov K, Turchin I, Subochev P, Lukyanov S, Kamensky V, Zagaynova E. 2015. Towards PDT with Genetically Encoded Photosensitizer KillerRed: A Comparison of Continuous and Pulsed Laser Regimens in an Animal Tumor Model. PLoS One 10:e0144617.
260. Chen X, Zaro J L, Shen W C. 2013. Fusion protein linkers: property, design and functionality. Adv Drug Deliv Rev 65:1357-1369.
261. Reddy Chichili V P, Kumar V, Sivaraman J. 2013. Linkers in the structural biology of protein-protein interactions. Protein Sci 22:153-167.
262. Patel D K, Menon D V, Patel D H, Dave G. 2022. Linkers: A synergistic way for the synthesis of chimeric proteins. Protein Expr Purif 191:106012.
263. Liu Z, Chen O, Wall J B J, Zheng M, Zhou Y, Wang L, Vaseghi H R, Qian L, Liu J. 2017. Systematic comparison of 2A peptides for cloning multi-genes in a polycistronic vector. Sci Rep 7:2193.
264. Luke G A, de Felipe P, Lukashev A, Kallioinen S E, Bruno E A, Ryan M D. 2008. Occurrence, function and evolutionary origins of ‘2A-like’ sequences in virus genomes. J Gen Virol 89:1036-1042.
265. Szymczak-Workman A L, Vignali K M, Vignali D A. 2012. Design and construction of 2A peptide-linked multicistronic vectors. Cold Spring Harb Protoc 2012:199-204.
266. Hellen C U, Sarnow P. 2001. Internal ribosome entry sites in eukaryotic mRNA molecules. Genes Dev 15:1593-1612.
267. Kozak M. 2005. A second look at cellular mRNA sequences said to function as internal ribosome entry sites. Nucleic Acids Res 33:6593-6602.
268. Shaimardanova A A, Kitaeva K V, Abdrakhmanova, I I, Chernov V M, Rutland C S, Rizvanov A A, Chulpanova D S, Solovyeva V V. 2019. Production and Application of Multicistronic Constructs for Various Human Disease Therapies. Pharmaceutics 11.
269. Kaufman R J, Davies M V, Wasley L C, Michnick D. 1991. Improved vectors for stable expression of foreign genes in mammalian cells by use of the untranslated leader sequence from EMC virus. Nucleic Acids Res 19:4485-4490.

1. A genetically modified Salmonella cell (GMSC) engineered to exhibit regulated delayed lysis in vivo, the GMSC comprising a first heterologous nucleic acid that encodes a first gene product that causes the GMSC to be selectively localized to and/or internalized by target cells in vivo and a second heterologous nucleic acid that encodes a second gene product that facilitates killing of the target cells following internalization.

2. The GMSC of claim 1, wherein the first heterologous nucleic acid and the second heterologous nucleic acid are present on a plasmid in a balanced-lethal vector-host.

3. The GMSC of claim 1, wherein the GMSC is of a cancer cell targeting Salmonella (CCTS) strain.

4. The GMSC of claim 1 comprising one or more mutations set forth in Table 1.

5. The GMSC of claim 1 comprising a genotype as set forth in Table 3.

6. The GMSC of claim 1, wherein the first heterologous nucleic acid comprises a nucleic acid sequence encoding OmpA operably linked to a nucleic acid sequence encoding PLZ4.

7. The GMSC of claim 1, wherein the second heterologous nucleic acid comprises a nucleic acid sequence encoding CXCL11 or KillerRed, or both.

8. The GMSC of claim 7, wherein the second heterologous nucleic acid comprises a nucleic acid sequence encoding KillerRed.

9. The GMSC of claim 8, wherein the second heterologous nucleic acid comprises a sequence encoding SEQ ID NO:18 and SEQ ID NO:20; or an amino sequence comprising at least 90% or 95% sequence identity therewith.

10. The GMSC of claim 9, wherein CXCL11 is fused to KillerRed.

11. The GMSC of claim 10, wherein a P2A peptide is situated between CXCL11 and KillerRed.

12. The GMSC of claim 11, wherein the second heterologous nucleic acid comprises a sequence encoding SEQ ID NO: 52 or SEQ ID NO: 53; or an amino sequence comprising at least 90% or 95% sequence identity therewith

13. The GMSC of claim 1, wherein the second heterologous nucleic acid comprises a nucleic acid sequence encoding KillerRed, KillerRed fused to neuromodulin N-terminal sequence, or KillerRed fused to a mitochondrial targeting sequence.

14. The GMSC of claim 13, wherein the second heterologous nucleic acid comprises a sequence encoding SEQ ID NO:18, SEQ ID NO:20, SEQ ID NO: 52 and SEQ ID NO: 53; or an amino sequence comprising at least 90% or 95% sequence identity therewith

15. The GMSC of claim 1, wherein the first heterologous nucleic acid comprises a nucleic acid sequence encoding a LHRH peptide or HER2ScFv, or both.

16. The GMSC of claim 15, wherein the first heterologous nucleic acid comprises a nucleic acid sequence encoding at least one selected from the group consisting of SEQ ID NO: 24, SEQ ID NO:25, and SEQ ID NO: 47; or an amino sequence comprising at least 90% or 95% sequence identity therewith.

17. The GMSC of claim 1, wherein the second heterologous nucleic acid comprises a nucleic acid sequence encoding a HAC-PD1 or HaPD1-IgG, or both.

18. The GMSC of claim 17, wherein the second heterologous nucleic acid comprises a nucleic acid sequence encoding at least one selected from the group consisting of SEQ ID NO: 49, and SEQ ID NO: 51; or an amino sequence comprising at least 90% or 95% sequence identity therewith.

19. The GMSC of claim 1, wherein the second heterologous nucleic acid comprises a nucleic acid sequence encoding an HLA peptide.

20. The GMSC of claim 19, wherein the second heterologous nucleic acid comprises a nucleic acid sequence encoding SEQ ID NO: 21; or an amino sequence comprising at least 90% or 95% sequence identity therewith.

21. The GMSC of claim 6, wherein the first heterologous nucleic acid comprises a nucleic acid sequence encoding SEQ ID NO: 2, or an amino sequence comprising at least 90% or 95% sequence identity therewith.

22. The GMSC of claim 7, wherein the second heterologous nucleic acid sequence encodes SEQ ID NO: 14 or SEQ ID NO: 16, or an amino acid sequence comprising at least 90% or 95% sequence identity therewith.

23. The GMSC of claim 7, wherein the second heterologous nucleic acid sequence encodes SEQ ID NO: 18 or SEQ ID NO: 20, or an amino acid sequence comprising at least 90% or 95% sequence identity therewith.

24. (canceled)

25. A genetically modified Salmonella cell (GMSC) engineered to exhibit regulated delayed lysis in vivo, the GMSC comprising a first heterologous nucleic acid that encodes a first gene product that causes the GMSC to be selectively localized to and/or internalized by target cells in vivo and a second heterologous nucleic acid that encodes a second gene product that facilitates killing of the target cells following internalization; wherein expression of the first heterologous nucleic acid is controlled by a bacterial promoter, optionally further comprising a sequence for secretion of the first gene product; and wherein expression of the second heterologous nucleic acid is controlled by a eukaryotic promoter for delivery to the nucleus of the target cells, wherein the bacterial promoter optionally comprises P_{trc}, P_{tac}, P_{lac}, or Pₗₚₚ, wherein the sequence for secretion optionally comprises bla SSopt and wherein the eukaryotic promoter optionally comprises P_{CMV} or P_{EF1α}.

26. A composition comprising a GMSC of claim 1 and a pharmaceutically acceptable carrier.

27. A method for treating cancer comprising administering a therapeutically effective amount of the composition of claim 26 to a subject who has cancer.

28-45. (canceled)


SEQUENCE LISTING (158 sequences)

SEQ ID NO: 1 - DNA, 1053 bp, Synthetic construct
  source 1..1053: mol_type = other DNA
atgaaaaagacagctatcgcgattgcagtggcactggctggtttcgctaccgtagcgcaggccgctccgaaagataacacctggtacgctggtgctaaactgggctggtctcagtaccatgacaccggcttcattcacaatgatggcccgactcatgaaaaccaactgggcgcaggtgcttttggtggttaccaggttaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggcctgctgagcgtaggtgtttcctaccgtttcggccagcaagaagctgctccggtagtagctccggcaccagctccggctccggaagtacagaccaagcacttcactctgaagtctgacgtactgttcaacttcaacaaatctaccctgaagccggaaggccagcaggctctggatcagctgtacagccagctgagcaacctggatccgaaagacggttccgttgtcgttctgggcttcactgaccgtatcggttctgacgcttacaaccagggtctgtccgagaaacgtgctcagtctgttgttgattacctgatctccaaaggtattccgtctgacaaaatctccgcacgtggtatgggcgaatctaacccggttaccggcaacacctgtgacaacgtgaaacctcgcgctgccctgatcgattgcctggctccggatcgtcgcgtagagatcgaagttaaaggcgttaaagacgtggtaactcagccgcaggcttaa

SEQ ID NO: 2 - protein, 350 aa, Synthetic construct
MKKTAIAIAVALAGFATVAQAAPKDNTWYAGAKLGWSQYHDTGFIHNDGPTHENQLGAGAFGGYQVNPYVGFEMGYDWLGRMPYKGDNINGAYKAQGVQLTAKLGYPITDDLDVYTRLGGMVWRADTKSNVPGGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 3 - DNA, 27 bp, Synthetic construct
  source 1..27: mol_type = other DNA
tgccaggacggtcgtatgggtttctgc

SEQ ID NO: 4 - protein, 9 aa, Synthetic construct
CQDGRMGFC

SEQ ID NO: 5-6 - skipped (2 entries)

SEQ ID NO: 7 - DNA, 1107 bp, Synthetic construct
  source 1..1107: mol_type = other DNA
ctaggccaactggcgcagcattcgacgcagcggctcggcggcgccccataacaactggtcgcctacggtaaacgccgacaagaactctggccccatgttcagcttacgcagacgaccaaccggcgtagtcaacgtgccggtcaccgccgccggggttaattcgcgcatagtgatatcacgatcgttcggcaccactttcgcccacggattatgtgccgccagcagttcttccaccgtcggaatggatacctcttttttcagcttgatggtgaacgcctggctgtgacagcgcagcgcgccgacgcgcacacacaaaccatcaaccggaatcacagaggcagtattgagaatcttgttggtttccgcctggcctttccactcttcgcggctctggccgttatcgagctgtttgtcgatccaggggatcaggcttcccgccagcggtacgccaaagttatcaaccggcagctcgccgctgcgggtcaatgccgtaactttgcgttcaatatcaagaattgcggaagacggcgtcgccagttcatcggcgacatggccatacaactgacccatctgggttaacagctcgcgcatatggcgcgcgccgccgccggaggcggcctgataggtcgcgacggatacccagtcaacgagattatgggcaaagagaccgcccagcgacatcaacatcaggctaacggtacagttaccgcccacaaaggtcttcacgccattgttcaggccgtcggtaatcacgtcctggttgaccgggtcgagaataataatggcatcatctttcatgcgcagcgtagaagccgcatcaatccagtaaccctgccatccgctttcgcgcagctttggataaatttcgttggtataatcgccgccctggcaggtcacgatgatatcgagcgcttttagcgcatccagatcaaaagcgtcctgtagcgtgccggtggaggtgtcgccgaaggtgggcgccgcctgtccaaactgggaggtagaaaagaaaacagggcgaatagcgtcgaaatcgcgctcctctaccatgcgttgcatgagaacagagccgaccattccgcgccagccgataaaaccaacatttttcat

SEQ ID NO: 8 - protein, 368 aa, Synthetic construct
ALQRLMRRLPEAAGWLLQDGVTFASLFEPGMNLKRLRGVPTTLTGTVAAPTLERMTIDRDNPVVKAWPNHAALLEEVTPISVEKKLKITFAQSHCRLAGVRVCLGDVPIVSATNLIKNTEAQGKWEERSQGNDLQKDIWPILSGALPVGFNDVPLEGSRTLATVKREIDLIASSPTALEDAVHGYLQGMQTLLERMHRAGGGSAAQYTAVSVWDVLNHAFLGGLSMLMLSVTCNGGVFTKVGNNLGDTIVDQNVPDLIIIADDKMRLTSAADIWYGQWGSERLKPYIENTYDGGQCTVIIDLAKLADLDFADQLTGTSTDGFTPAAQGFQSTSFFVPRIADFDREEVMRQMLVSGVMGRWGIFGVNKM

SEQ ID NO: 9 - DNA, 1257 bp, Synthetic construct
  source 1..1257: mol_type = other DNA
gtggataaatttcgtgttcaggggccaacgaagctccagggcgaagtcacaatttccggcgctaaaaatgctgctctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaa

SEQ ID NO: 10 - protein, 419 aa, Synthetic construct
MDKFRVQGPTKLQGEVTISGAKNAALPILFAALLAEEPVEIQNVPKLKDVDTSMKLLSQLGAKVERNGSVHIDARDVNVFCAPYDLVKTMRASIWALGPLVARFGQGQVSLPGGCTIGARPVDLHISGLEQLGATIKLEEGYVKASVDGRLKGAHIVMDKVSVGATVTIMCAATLAEGTTIIENAAREPEIVDTANFLITLGAKISGQGTDRIVIEGVERLGGGVYRVLPDRIETGTFLVAAAISRGKIICRNAQPDTLDAVLAKLRDAGADIEVGEDWISLDMHGKRPKAVNVRTAPHPAFPTDMQAQFTLLNLVAEGTGFITETVFENRFMHVPELSRMGAHAEIESNTVICHGVEKLSGAQVMATDLRASASLVLAGCIAEGTTVVDRIYHIDRGYERIEDKLRALGANIERVKGE

SEQ ID NO: 11 - DNA, 876 bp, Synthetic construct
  source 1..876: mol_type = other DNA
tgacaacttgacggctacatcattcactttttcttcacaaccggcacgaaactcgctcgggctggccccggtgcattttttaaatactcgcgagaaatagagttgatcgtcaaaaccaacattgcgaccgacggtggcgataggcatccgggtagtgctcaaaagcagcttcgcctgactaatgcgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaacccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccat

SEQ ID NO: 12 - protein, 292 aa, Synthetic construct
SLKVAVDNVKEECGARFESPSAGTCKKFVRSFYLQDDFGVNRGVTAIPMRTTSLLLKAQSIRQDERWSLVSIGLQQRFLHSLRSPSLCVHQAVSAIDFNSDALHDSIYQCAERVRNDMPPHLSENIAEMRRLLLQELLNIALLESYRGEGQGANIIQGFLDSFHPQHAEDPRFFGTNAFISPWNLWEHWYARPRFYVWQHYWERAEPHRGYHHIEGPPFLLIDGPRCVFERGQNKVVGQGRITLNLIYGKMGLPRDIFFDLYGNAEIPTLGAVLHANFSYGPLLPDNQAEAM

SEQ ID NO: 13 - DNA, 285 bp, Homo sapiens
  source 1..285: mol_type = genomic DNA
atgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaa

SEQ ID NO: 14 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 15 - DNA, 303 bp, Mus musculus
  source 1..303: mol_type = genomic DNA
atgaacaggaaggtcacagccatagccctggctgcgatcatctgggccacagctgctcaaggcttccttatgttcaaacaggggcgctgtctttgcatcggccccgggatgaaagccgtcaaaatggcagagatcgagaaagcttctgtaatttacccgagtaacggctgcgacaaagttgaagtgattgttactatgaaggctcataaacgacaaaggtgcctggaccccagatccaagcaagctcgcctcataatgcaggcaatagaaaaaaagaattttttaaggcgtcaaaacatgtaa

SEQ ID NO: 16 - protein, 100 aa, Mus musculus
MNRKVTAIALAAIIWATAAQGFLMFKQGRCLCIGPGMKAVKMAEIEKASVIYPSNGCDKVEVIVTMKAHKRQRCLDPRSKQARLIMQAIEKKNFLRRQNM

SEQ ID NO: 17 - DNA, 774 bp, Synthetic construct
  source 1..774: mol_type = other DNA
atgctgtgctgtatgagaagaaccaaacaggttgaaaagaatgatgaggaccaaaagatctccgagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccgacggcatcagccatttcgcccaggagtgcttccccgagggcctgagcatcgaccgcaccgtgcgcttcgagaacgacggcaccatgaccagccaccacacctacgagctggacgacacctgcgtggtgagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgggccacttcgacagcaagatgaccttcaacggcagccgcgccatcgagatccccggcccacacttcgtgaccatcatcaccaagcagatgagggacaccagcgacaagcgcgaccacgtgtgccagcgcgaggtggcctacgcccacagcgtgccccgcatcaccagcgccatcggtagcgacgaggattaa

SEQ ID NO: 18 - protein, 257 aa, Synthetic construct
MLCCMRRTKQVEKNDEDQKISEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDED

SEQ ID NO: 19 - DNA, 921 bp, Synthetic construct
  source 1..921: mol_type = other DNA
atgtccgtcctgacgccgctgctgctgcggggcttgacaggctcggcccggcggctcccagtgccgcgcgccaagatccattcgttgggggatctgtccgtcctgacgccgctgctgctgcggggcttgacaggctcggcccggcggctcccagtgccgcgcgccaagatccattcgttgggggatccaccggtcgccaccatgggttcagagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccgacggcatcagccatttcgcccaggagtgcttccccgagggcctgagcatcgaccgcaccgtgcgcttcgagaacgacggcaccatgaccagccaccacacctacgagctggacgacacctgcgtggtgagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgggccacttcgacagcaagatgaccttcaacggcagccgcgccatcgagatccccggcccacacttcgtgaccatcatcaccaagcagatgagggacaccagcgacaagcgcgaccacgtgtgccagcgcgaggtggcctacgcccacagcgtgccccgcatcaccagcgccatcggtagcgacgaggattaa

SEQ ID NO: 20 - protein, 306 aa, Synthetic construct
MSVLTPLLLRGLTGSARRLPVPRAKIHSLGDLSVLTPLLLRGLTGSARRLPVPRAKIHSLGDPPVATMGSEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDED

SEQ ID NO: 21 - DNA, 249 bp, Synthetic construct
  source 1..249: mol_type = other DNA
atgctggtcatggcgccccgaaccgtcctcctgctgctctcggcggccctggccctgaccgagacctgggccggctccggtaccgggcccgggcctaggggcctggctgtcctggcagttgtggtcatcggagctgtggtcgctgctgtgatgtgtaggaggaagagttcaggtggaaaaggagggagctactctcaggctgcgtgcagcgacagtgcccagggctctgatgtgtctctcacagcttga

SEQ ID NO: 22 - protein, 75 aa, Synthetic construct
MLVMAPRTVLLLLSAALALTETWAGSGLAVLAVVVIGAVVAAVMCRRKSSGGKGGSYSQAACSDSAQGSDVSLTA

SEQ ID NO: 23 - DNA, 36 bp, Synthetic construct
  source 1..36: mol_type = other DNA
tgcgaacattggagctatggcctgcgtccgggctgc

SEQ ID NO: 24 - protein, 12 aa, Synthetic construct
CEHWSYGLRPGC

SEQ ID NO: 25 - protein, 10 aa, Synthetic construct
EHWSYGLRPG

SEQ ID NO: 26 - DNA, 60 bp, Synthetic construct
  source 1..60: mol_type = other DNA
atgctgtgctgtatgagaagaaccaaacaggttgaaaagaatgatgaggaccaaaagatc

SEQ ID NO: 27 - protein, 20 aa, Synthetic construct
MLCCMRRTKQVEKNDEDQKI

SEQ ID NO: 28 - DNA, 711 bp, Synthetic construct
  source 1..711: mol_type = other DNA
tccgagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccgacggcatcagccatttcgcccaggagtgcttccccgagggcctgagcatcgaccgcaccgtgcgcttcgagaacgacggcaccatgaccagccaccacacctacgagctggacgacacctgcgtggtgagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgggccacttcgacagcaagatgaccttcaacggcagccgcgccatcgagatccccggcccacacttcgtgaccatcatcaccaagcagatgagggacaccagcgacaagcgcgaccacgtgtgccagcgcgaggtggcctacgcccacagcgtgccccgcatcaccagcgccatcggtagcgacgaggat

SEQ ID NO: 29 - protein, 237 aa, Synthetic construct
SEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDED

SEQ ID NO: 30 - DNA, 66 bp, Synthetic construct
  source 1..66: mol_type = other DNA
ggaagcggagctactaacttcagcctgctgaagcaggctggcgacgtggaggagaaccctggacct

SEQ ID NO: 31 - protein, 22 aa, Synthetic construct
GSGATNFSLLKQAGDVEENPGP

SEQ ID NO: 32 - DNA, 282 bp, Homo sapiens
  source 1..282: mol_type = genomic DNA
atgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttt

SEQ ID NO: 33 - protein, 94 aa, Homo sapiens; identical to SEQ ID NO: 14

SEQ ID NO: 34 - DNA, 60 bp, Synthetic construct; identical to SEQ ID NO: 26
  source 1..60: mol_type = other DNA

SEQ ID NO: 35 - protein, 20 aa, Synthetic construct
MLCCMRRTKQVEKNDEDQKI

SEQ ID NO: 36 - DNA, 711 bp, Synthetic construct; identical to SEQ ID NO: 28
  source 1..711: mol_type = other DNA

SEQ ID NO: 37 - protein, 237 aa, Synthetic construct; identical to SEQ ID NO: 29

SEQ ID NO: 38 - DNA, 66 bp, Synthetic construct; identical to SEQ ID NO: 30
  source 1..66: mol_type = other DNA

SEQ ID NO: 39 - protein, 22 aa, Synthetic construct
GSGATNFSLLKQAGDVEENPGP

SEQ ID NO: 40 - DNA, 300 bp, Mus musculus
  source 1..300: mol_type = genomic DNA
atgaacaggaaggtcacagccatagccctggctgcgatcatctgggccacagctgctcaaggcttccttatgttcaaacaggggcgctgtctttgcatcggccccgggatgaaagccgtcaaaatggcagagatcgagaaagcttctgtaatttacccgagtaacggctgcgacaaagttgaagtgattgttactatgaaggctcataaacgacaaaggtgcctggaccccagatccaagcaagctcgcctcataatgcaggcaatagaaaaaaagaattttttaaggcgtcaaaacatg

SEQ ID NO: 41 - protein, 100 aa, Mus musculus; identical to SEQ ID NO: 16

SEQ ID NO: 42 - DNA, 66 bp, Synthetic construct; identical to SEQ ID NO: 30
  source 1..66: mol_type = other DNA

SEQ ID NO: 43 - protein, 22 aa, Synthetic construct
GSGATNFSLLKQAGDVEENPGP

SEQ ID NO: 44 - DNA, 1095 bp, Synthetic construct
  source 1..1095: mol_type = other DNA
atgaaaaagacagctatcgcgattgcagtggcactggctggtttcgctaccgtagcgcaggccgctccgaaagataacacctggtacgctggtgctaaactgggctggtctcagtaccatgacaccggcttcattcacaatgatggcccgactcatgaaaaccaactgggcgcaggtgcttttggtggttaccaggttaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcgcggccgccggcggaggaggatctcatcatcaccatcaccatggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggcctgctgagcgtaggtgtttcctaccgtttcggccagcaagaagctgctccggtagtagctccggcaccagctccggctccggaagtacagaccaagcacttcactctgaagtctgacgtactgttcaacttcaacaaatctaccctgaagccggaaggccagcaggctctggatcagctgtacagccagctgagcaacctggatccgaaagacggttccgttgtcgttctgggcttcactgaccgtatcggttctgacgcttacaaccagggtctgtccgagaaacgtgctcagtctgttgttgattacctgatctccaaaggtattccgtctgacaaaatctccgcacgtggtatgggcgaatctaacccggttaccggcaacacctgtgacaacgtgaaacctcgcgctgccctgatcgattgcctggctccggatcgtcgcgtagagatcgaagttaaaggcgttaaagacgtggtaactcagccgcaggcttaa

SEQ ID NO: 45 - protein, 364 aa, Synthetic construct
MKKTAIAIAVALAGFATVAQAAPKDNTWYAGAKLGWSQYHDTGFIHNDGPTHENQLGAGAFGGYQVNPYVGFEMGYDWLGRMPYKGDNINGAYKAQGVQLTAKLGYPITDDLDVYTRLGGMVWRADTKSNVPGAAAGGGGSHHHHHHGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 46 - DNA, 750 bp, Synthetic construct
  source 1..750: mol_type = other DNA
cagtctggggcagaggtgaaaaagcccggggagtctctgaagatctcctgtaagggttctggatacagctttaccagctactggatcgcctgggtgcgccagatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcagtagttccaactgcgcaaagtggcctgaatacttccagcattggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggaggaaccaagctgaccgtcctaggt

SEQ ID NO: 47 - protein, 250 aa, Synthetic construct
QSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLG

SEQ ID NO: 48 - DNA, 369 bp, Synthetic construct
  source 1..369: mol_type = other DNA
atggattccccagatagaccatggaacccaccaactttctccccagctttgttggtcgtcactgaaggtgataacgctactttcacttgttccttctccaacacttccgaatccttccatgttgtttggcatcgtgaatccccatccggtcaaactgatacattggctgctttcccagaagatagatcccaaccaggtcaagatgctagattcagagttactcaattgccaaacggtagagatttccacatgtccgtcgtcagagctagaagaaacgattccggtacttatgtttgtggtgttatttcccttgctccaaagattcaaattaaggaatccttgagagctgaattgagagtcactgaaaga

SEQ ID NO: 49 - protein, 123 aa, Synthetic construct
MDSPDRPWNPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTER

SEQ ID NO: 50 - DNA, 1197 bp, Synthetic construct
  source 1..1197: mol_type = other DNA
atgggctggtcctgtatcatcctgttcctggtggctacagccacaggagtgcatagtttcttagactccccagacaggccctggaacccccccaccttctccccagccctgctcgtggtgaccgaaggggacaacgccaccttcacctgcagcttctccaacacatcggagagcttccatgtaatctggcaccgcgagagccccagcggccagacggacacgctggccgccttccccgaggaccgcagccagcccggccaggactgccgcttccgtgtcacacaactgcccaacgggcgtgacttccacatgagcgtggtcagggcccggcgcaatgacagcggcacctacgtctgtggggtcatctccctggcccccaagatccagatcaaagagagcctgcgggcagagctcagggtgacagagagaagggcagaagtgcccacagcccaccccagcccctcacccaggccagccggccagttccaaaccctggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccaggtaaataa

SEQ ID NO: 51 - protein, 398 aa, Synthetic construct
MGWSCIILFLVATATGVHSFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 52 - protein, 373 aa, Synthetic construct
MLCCMRRTKQVEKNDEDQKISEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDEDGSGATNFSLLKQAGDVEENPGPMSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 53 - protein, 379 aa, Synthetic construct
MLCCMRRTKQVEKNDEDQKISEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDEDGSGATNFSLLKQAGDVEENPGPMNRKVTAIALAAIIWATAAQGFLMFKQGRCLCIGPGMKAVKMAEIEKASVIYPSNGCDKVEVIVTMKAHKRQRCLDPRSKQARLIMQAIEKKNFLRRQNM

SEQ ID NO: 54 - DNA, 48 bp, Synthetic construct
  source 1..48: mol_type = other DNA
gataacaatttcacacaggaaacagaccatgaaaaagacagctatcgc

SEQ ID NO: 55 - DNA, 46 bp, Synthetic construct
  source 1..46: mol_type = other DNA
gaaacccatacgaccgtcctggcacgcgccagggacgttagacttg

SEQ ID NO: 56 - DNA, 49 bp, Synthetic construct
  source 1..49: mol_type = other DNA
ccaggacggtcgtatgggtttctgcggtggcccgtctactaaagaccac

SEQ ID NO: 57 - DNA, 46 bp, Synthetic construct
  source 1..46: mol_type = other DNA
gccaaaacagccaagcttgagctcattaagcctgcggctgagttac

SEQ ID NO: 58 - DNA, 34 bp, Synthetic construct
  source 1..34: mol_type = other DNA
cccagcagtctagaatgaaaaagacagctatcgc

SEQ ID NO: 59 - DNA, 40 bp, Synthetic construct
  source 1..40: mol_type = other DNA
ctgcaaagagatgtgcggatctctagattatgcgaaaggc

SEQ ID NO: 60 - DNA, 45 bp, Synthetic construct
  source 1..45: mol_type = other DNA
caacagctcatttcagaatggaagaaaaaaaagcccgctcattag

SEQ ID NO: 61 - DNA, 45 bp, Synthetic construct
  source 1..45: mol_type = other DNA
ctaatgagcgggcttttttttcttccattctgaaatgagctgttg

SEQ ID NO: 62 - DNA, 40 bp, Synthetic construct
  source 1..40: mol_type = other DNA
gcctttcgcataatctagagatccgcacatctctttgcag

SEQ ID NO: 63 - DNA, 58 bp, Synthetic construct
  source 1..58: mol_type = other DNA
gtaactcagccgcaggcttaatgagcttcgaaacagattaaatcagaacgcagaagcg

SEQ ID NO: 64 - DNA, 60 bp, Synthetic construct
  source 1..60: mol_type = other DNA
aaaaaaaaccccgccctgtcaggggcggggtttttttttcctacgctcacccatcaattg

SEQ ID NO: 65 - DNA, 59 bp, Synthetic construct
  source 1..59: mol_type = other DNA
gattaattgtcaacagctcatttcagaatgatcaaaaaaaaccccgccctgtcaggggc

SEQ ID NO: 66 - DNA, 52 bp, Synthetic construct
  source 1..52: mol_type = other DNA
cgcccctgacagggcggggttttttttgatcattctgaaatgagctgttgac

SEQ ID NO: 67 - DNA, 52 bp, Synthetic construct
  source 1..52: mol_type = other DNA
cttctgcgttctgatttaatctgtttcgaagctcattaagcctgcggctgag

SEQ ID NO: 68 - DNA, 46 bp, Synthetic construct
  source 1..46: mol_type = other DNA
cgtttaaacttaagcttggtaccgccatgagtgtgaagggcatggc

SEQ ID NO: 69 - DNA, 50 bp, Synthetic construct
  source 1..50: mol_type = other DNA
gtctgctcgaagcattctcgagcggccgcttaaaaattctttctttcaac

SEQ ID NO: 70 - DNA, 47 bp, Synthetic construct
  source 1..47: mol_type = other DNA
gcgtttaaacttaagcttggtaccgccatgaacaggaaggtcacagc

SEQ ID NO: 71 - DNA, 52 bp, Synthetic construct
  source 1..52: mol_type = other DNA
ctcgaagcattctcgagcggccgcttacatgttttgacgccttaaaaaattc

SEQ ID NO: 72 - DNA, 58 bp, Synthetic construct
  source 1..58: mol_type = other DNA
gcgtttaaacttaagcttggtaccgccaccatgctgtgctgtatgagaagaaccaaac

SEQ ID NO: 73 - DNA, 45 bp, Synthetic construct
  source 1..45: mol_type = other DNA
gctcgaagcattctcgagcggccgctttaatcctcgtcgctaccg

SEQ ID NO: 74 - DNA, 52 bp, Synthetic construct
  source 1..52: mol_type = other DNA
gcgtttaaacttaagcttggtaccgccaccatgtccgtcctgacgccgctgc

SEQ ID NO: 75 - DNA, 49 bp, Synthetic construct
  source 1..49: mol_type = other DNA
cgcaggcttaatgagctcaaggaacagtcaatgagtaaaggagaagaac

SEQ ID NO: 76 - DNA, 47 bp, Synthetic construct
  source 1..47: mol_type = other DNA
catccgccaaaacagccaagcttattatttgtatagttcatccatgc

SEQ ID NO: 77 - DNA, 46 bp, Synthetic construct
  source 1..46: mol_type = other DNA
gtttaaacttaagcttggtaccaccaaaatggtgagcaagggcgag

SEQ ID NO: 78 - DNA, 42 bp, Synthetic construct
  source 1..42: mol_type = other DNA
ctgctcgaagcattctcgagttacttgtacagctcgtccatg

SEQ ID NO: 79 - DNA, 58 bp, Synthetic construct
  source 1..58: mol_type = other DNA
gcctgcttcagcaggctgaagttagtagctccgcttccatcctcgtcgctaccgatgg

SEQ ID NO: 80 - DNA, 49 bp, Synthetic construct
  source 1..49: mol_type = other DNA
aggtccagggttctcctccacgtcgccagcctgcttcagcaggctgaag

SEQ ID NO: 81 - DNA, 45 bp, Synthetic construct
  source 1..45: mol_type = other DNA
cgacgtggaggagaaccctggacctatgaacaggaaggtcacagc

SEQ ID NO: 82 - DNA, 45 bp, Synthetic construct
  source 1..45: mol_type = other DNA
cgacgtggaggagaaccctggacctatgagtgtgaagggcatggc

SEQ ID NO: 83 - DNA, 43 bp, Synthetic construct
  source 1..43: mol_type = other DNA
cttaagcttggtacgccgccaccatgctggtcatggcgccccg

SEQ ID NO: 84 - DNA, 41 bp, Synthetic construct
  source 1..41: mol_type = other DNA
cctaggcccgggcccggtaccggagccggcccaggtctcgg

SEQ ID NO: 85 - DNA, 40 bp, Synthetic construct
  source 1..40: mol_type = other DNA
ggtaccgggcccgggcctaggggcctggctgtcctggcag

SEQ ID NO: 86 - DNA, 41 bp, Synthetic construct
  source 1..41: mol_type = other DNA
ctgctcgaagcattctcgagtcaagctgtgagagacacatc

SEQ ID NO: 87 - DNA, 40 bp, Synthetic construct
  source 1..40: mol_type = other DNA
ctgggccggctccggtaccatggtgagcaagggcgaggag

SEQ ID NO: 88 - DNA, 44 bp, Synthetic construct
  source 1..44: mol_type = other DNA
ctaggacagccaggcccctaggcttgtacagctcgtccatgccg

SEQ ID NO: 89 - DNA, 60 bp, Synthetic construct
  source 1..60: mol_type = other DNA
ggctagcgtttaaacttaagcttggtacaatttgtaatacgactcactatagggcggccg

SEQ ID NO: 90 - DNA, 43 bp, Synthetic construct
  source 1..43: mol_type = other DNA
ctgctcgaagcattctcgaggtacgactatggaaccgcggccg

SEQ ID NO: 91 - DNA, 40 bp, Synthetic construct; identical to SEQ ID NO: 87
  source 1..40: mol_type = other DNA

SEQ ID NO: 92 - DNA, 44 bp, Synthetic construct; identical to SEQ ID NO: 88
  source 1..44: mol_type = other DNA

SEQ ID NO: 93 - DNA, 42 bp, Synthetic construct
  source 1..42: mol_type = other DNA
gaggtacctgcaggcccggggcggccgctcgagaatgcttcg

SEQ ID NO: 94 - DNA, 38 bp, Synthetic construct
  source 1..38: mol_type = other DNA
cgggcaccggagcggaaagtccccggaaagtccccgcc

SEQ ID NO: 95 - DNA, 37 bp, Synthetic construct
  source 1..37: mol_type = other DNA
ctttccggggactttccgctccggtgcccgtcagtgg

SEQ ID NO: 96 - DNA, 39 bp, Synthetic construct
  source 1..39: mol_type = other DNA
cccgggcctgcaggtacctcacgacacctgaaatggaag

SEQ ID NO: 97 - DNA, 50 bp, Synthetic construct
  source 1..50: mol_type = other DNA
gcgtttaaacttaagcttggtaccgccatggattccccagatagaccatg

SEQ ID NO: 98 - DNA, 76 bp, Synthetic construct
  source 1..76: mol_type = other DNA
cctcgcttcctccgccttcacttccaccgccctcactgccgccgccggagccgcctctttcagtgactctcaattc

SEQ ID NO: 99 - DNA, 74 bp, Synthetic construct
  source 1..74: mol_type = other DNA
gcttccgccgccgctgcctccaccctcagacccgcctccttcggagcctcctccctcgcttcctccgccttcac

SEQ ID NO: 100 - DNA, 36 bp, Synthetic construct
  source 1..36: mol_type = other DNA
cagcggcggcggaagcatgaacaggaaggtcacagc

SEQ ID NO: 101 - DNA, 36 bp, Synthetic construct
  source 1..36: mol_type = other DNA
cagcggcggcggaagcatgagtgtgaagggcatggc

SEQ ID NO: 102 - DNA, 73 bp, Synthetic construct
  source 1..73: mol_type = other DNA
gtttaaacttaagcttggtacgccaccatgtacaggatgcaactcctgtcttgcattgcactaagtcttgcac

SEQ ID NO: 103 - DNA, 73 bp, Synthetic construct
  source 1..73: mol_type = other DNA
gactagtggatccgagctcggtacctgcactgtttgtgacaagtgcaagacttagtgcaatgcaagacaggag

SEQ ID NO: 104 - DNA, 48 bp, Synthetic construct
  source 1..48: mol_type = other DNA
ctcgaagcattctcgagcggccgcttatctttcagtgactctcaattc

SEQ ID NO: 105 - DNA, 36 bp, Synthetic construct
  source 1..36: mol_type = other DNA
ggtggcgaccggtggtctttcagtgactctcaattc

SEQ ID NO: 106 - DNA, 36 bp, Synthetic construct
  source 1..36: mol_type = other DNA
ccaccggtcgccaccatggtgagcaagggcgaggag

SEQ ID NO: 107 - DNA, 48 bp, Synthetic construct
  source 1..48: mol_type = other DNA
ctcgaagcattctcgagcggccgcttacttgtacagctcgtccatgcc

SEQ ID NO: 108 - DNA, 40 bp, Synthetic construct
  source 1..40: mol_type = other DNA
ggtggcgaccggtggaaaattctttctttcaacttttttg

SEQ ID NO: 109 - DNA, 40 bp, Synthetic construct
  source 1..40: mol_type = other DNA
ggtggcgaccggtggcatgttttgacgccttaaaaaattc

SEQ ID NO: 110 - DNA, 43 bp, Synthetic construct
  source 1..43: mol_type = other DNA
cgcaggccatagctccaatgttcgcacgcgccagggacgttag

SEQ ID NO: 111 - DNA, 44 bp, Synthetic construct
  source 1..44: mol_type = other DNA
gagctatggcctgcgtccgggctgcggtggcccgtctactaaag

SEQ ID NO: 112 - DNA, 40 bp, Synthetic construct
  source 1..40: mol_type = other DNA
ctcatggtgacgagccttcgaatcattaagcctgcggctg

SEQ ID NO: 113 - DNA, 35 bp, Synthetic construct
  source 1..35: mol_type = other DNA
ggctcgtcaccatgagtaaaggagaagaacttttc

SEQ ID NO: 114 - DNA, 42 bp, Synthetic construct
  source 1..42: mol_type = other DNA
cgttctgatttaatctgtttcgaattatttgtatagttcatc

SEQ ID NO: 115 - DNA, 47 bp, Synthetic construct
  source 1..47: mol_type = other DNA
cgtttaaacttaagcttggtaccgccatgggctggtcctgtatcatc

SEQ ID NO: 116 - DNA, 48 bp, Synthetic construct
  source 1..48: mol_type = other DNA
gtctgctcgaagcattctcgagcggccgcttatttacctggagtccgg

SEQ ID NO: 117 - DNA, 45 bp, Synthetic construct
  source 1..45: mol_type = other DNA
cgacgtggaggagaaccctggacctatgctgtgctgtatgagaag

SEQ ID NO: 118 - DNA, 48 bp, Synthetic construct
  source 1..48: mol_type = other DNA
ctaacttcagcctgctgaagcaggctggcgacgtggaggagaaccctg

SEQ ID NO: 119 - DNA, 40 bp, Synthetic construct
  source 1..40: mol_type = other DNA
ggagaaccctggacctatgggctggtcctgtatcatcgtg

SEQ ID NO: 120 - DNA, 40 bp, Synthetic construct
  source 1..40: mol_type = other DNA
cctctgccccagactggccagggacgttagacttggtgtc

SEQ ID NO: 121 - DNA, 36 bp, Synthetic construct
  source 1..36: mol_type = other DNA
catcaccatcaccatggcccgtctactaaagaccac

SEQ ID NO: 122 - DNA, 24 bp, Synthetic construct
  source 1..24: mol_type = other DNA
cagtctggggcagaggtgaaaaag

SEQ ID NO: 123 - DNA, 24 bp, Synthetic construct
  source 1..24: mol_type = other DNA
atggtgatggtgatgatgagatcc

SEQ ID NO: 124 - DNA, 50 bp, Synthetic construct
  source 1..50: mol_type = other DNA
gcaggctgaagttagtagctccgcttcctttacctggagtccgggagaag

SEQ ID NO: 125 - DNA, 76 bp, Synthetic construct
  source 1..76: mol_type = other DNA
ctgatttaatctgtttcgaacctgcaggggcccgggccgcggagtactcctagggtctgtttcctgtgtgaaattg

SEQ ID NO: 126 - DNA, 75 bp, Synthetic construct
  source 1..75: mol_type = other DNA
ctgatttaatctgtttcgaacctgcaggggcccgggccgcggagtactcctaggttcagcatcttttactttcac

SEQ ID NO: 127 - DNA, 105 bp, Synthetic construct
  source 1..105: mol_type = other DNA
atgaaaaaacaacatttccgtgtcgcccttattcccttttttgcggcattttgccttcctgtttttgctcacccagaaacgctggtgaaagtaaaagatgctgaa

SEQ ID NO: 128 - protein, 35 aa, Synthetic construct
MKKQHFRVALIPFFAAFCLPVFAHPETLVKVKDAE

SEQ ID NO: 129 - protein, 21 aa, unidentified
  source 1..21: note = Thosea asigna virus 2A
GSGEGRGSLLTCGDVEENPGP

SEQ ID NO: 130 - protein, 23 aa, Equine rhinitis A virus
GSGQCTNYALLKLAGDVESNPGP

SEQ ID NO: 131 - protein, 25 aa, Foot-and-mouth disease virus
GSGVKQTLNFDLLKLAGDVESNPGP

SEQ ID NO: 132 - DNA, 108 bp, Synthetic construct
  source 1..108: mol_type = other DNA
ggcggctccggcggcggcagtgagggcggtggaagtgaaggcggaggaagcgagggaggaggctccgaaggaggcgggtctgagggtggaggcagcggcggcggaagc

SEQ ID NO: 133 - protein, 36 aa, Synthetic construct
GGSGGGSEGGGSEGGGSEGGGSEGGGSEGGGSGGGS

SEQ ID NO: 134 - DNA, 4056 bp, Synthetic construct
  source 1..4056: mol_type = other DNA
ggatcttccggaagaccttccattctgaaatgagctgttgacaattaatcatccggctcgtataatgtgtggaattgtgagcggataacaatttcacacaggaaacagaccatgaaaaagacagctatcgcgattgcagtggcactggctggtttcgctaccgtagcgcaggccgctccgaaagataacacctggtacgctggtgctaaactgggctggtctcagtaccatgacaccggcttcattcacaatgatggcccgactcatgaaaaccaactgggcgcaggtgcttttggtggttaccaggttaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcgcgtgccaggacggtcgtatgggtttctgcggtggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggcctgctgagcgtaggtgtttcctaccgtttcggccagcaagaagctgctccggtagtagctccggcaccagctccggctccggaagtacagaccaagcacttcactctgaagtctgacgtactgttcaacttcaacaaatctaccctgaagccggaaggccagcaggctctggatcagctgtacagccagctgagcaacctggatccgaaagacggttccgttgtcgttctgggcttcactgaccgtatcggttctgacgcttacaaccagggtctgtccgagaaacgtgctcagtctgttgttgattacctgatctccaaaggtattccgtctgacaaaatctccgcacgtggtatgggcgaatctaacccggttaccggcaacacctgtgacaacgtgaaacctcgcgctgccctgatcgattgcctggctccggatcgtcgcgtagagatcgaagttaaaggcgttaaagacgtggtaactcagccgcaggcttaatgagctcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatggaagatcttccaacatcacaggtaaacagaaacgtcgggtcgatcgggaaattctttcccggacggcgcggggttgggcaagccgcaggcgcgtcagtgcttttagcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtctagactaggccaactggcgcagcattcgacgcagcggctcggcggcgccccataacaactggtcgcctacggtaaacgccgacaagaactctggccccatgttcagcttacgcagacgaccaaccggcgtagtcaacgtgccggtcaccgccgccggggttaattcgcgcatagtgatatcacgatcgttcggcaccactttcgcccacggattatgtgccgccagcagttcttccaccgtcggaatggatacctcttttttcagcttgatggtgaacgcctggctgtgacagcgcagcgcgccgacgcgcacacacaaaccatcaaccggaatcacagaggcagtattgagaatcttgttggtttccgcctggcctttccactcttcgcggctctggccgttatcgagctgtttgtcgatccaggggatcaggcttcccgccagcggtacgccaaagttatcaaccggcagctcgccgctgcgggtcaatgccgtaactttgcgttcaatatcaagaattgcggaagacggcgtcgccagttcatcggcgacatggccatacaactgacccatctgggttaacagctcgcgcatatggcgcgcgccgccgccggaggcggcctgataggtcgcgacggatacccagtcaacgagattatgggcaaagagaccgcccagcgacatcaacatcaggctaacggtacagttaccgcccacaaaggtcttcacgccattgttcaggccgtcggtaatcacgtcctggttgaccgggtcgagaataataatggcatcatctttcatgcgcagcgtagaagccgcatcaatccagtaaccctgccatccgctttcgcgcagctttggataaatttcgttggtataatcgccgccctggcaggtcacgatgatatcgagcgcttttagcgcatccagatcaaaagcgtcctgtagcgtgccggtggaggtgtcgccgaaggtgggcgccgcctgtccaaactgggaggtagaaaagaaaacagggcgaatagcgtcgaaatcgcgctcctctaccatgcgttgcatgagaacagagccgaccattccgcgccagccgataaaaccaacatttttcatagcgtttttttcctgcaaagagatgtgc

SEQ ID NO: 135 - DNA, 8160 bp, Synthetic construct
  source 1..8160: mol_type = other DNA
gactcttcgcgatgtacgggccagatatacgcgttaactgcagtctagattatgcgaaaggccatcctgacggatggcctttttgtttaaacggatccgcgacattgattattgactagttattaatagtaatcaattacggggtcattaggggactttccggggactttcctccccacgcgggggactttccgccacgggcggggactttccggggactttccgttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccggggcatgcaaccagctgtggaatgtgtgtcagttagggtgtggaaagtccccaggctccccagcaggcagaagtatgcaaagcatgtggggatgcggtgggctctatggcttctactgggcggttttatggacagcaagcgaaccggaattgccagctggggcgccctctggtaaggttgggaagccctgcaaagtaaactggatggctttctcgccgccaaggatctgtcgacccctagatttcagtgcaatttatctcttcaaatgtagcacctgaagtcagccccatacgatataagttgttggaagatctagcccgcctaatgagcgggcttttttttaattcgcaattccccgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacgaaactcgctcgggctggccccggtgcattttttaaatactcgcgagaaatagagttgatcgtcaaaaccaacattgcgaccgacggtggcgataggcatccgggtagtgctcaaaagcagcttcgcctgactaatgcgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaacccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccaccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattctgagaacaaactaagtggataaatttcgtgttcaggggccaacgaagctccagggcgaagtcacaatttccggcgctaaaaatgctgctctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaataagaattcaggaaaaaaacgctgtgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtagtctagctgcacgataccgtcgacttgtacatagactcgctccgaaattaaagaacacttaaattatctactaaaggaatctttagtcaagtttatttaagatgacttaactatgaatacacaattgatgggtgagcgtaggaaaaaaaaaccccgcccctgacagggcggggttttttttgatcattctgaaatgagctgttgacaattaatcatccggctcgtataatgtgtggaattgtgagcggataacaatttcacacaggaaacagaccatgaaaaagacagctatcgcgattgcagtggcactggctggtttcgctaccgtagcgcaggccgctccgaaagataacacctggtacgctggtgctaaactgggctggtctcagtaccatgacaccggcttcattcacaatgatggcccgactcatgaaaaccaactgggcgcaggtgcttttggtggttaccaggttaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcgcgtgccaggacggtcgtatgggtttctgcggtggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggcctgctgagcgtaggtgtttcctaccgtttcggccagcaagaagctgctccggtagtagctccggcaccagctccggctccggaagtacagaccaagcacttcactctgaagtctgacgtactgttcaacttcaacaaatctaccctgaagccggaaggccagcaggctctggatcagctgtacagccagctgagcaacctggatccgaaagacggttccgttgtcgttctgggcttcactgaccgtatcggttctgacgcttacaaccagggtctgtccgagaaacgtgctcagtctgttgttgattacctgatctccaaaggtattccgtctgacaaaatctccgcacgtggtatgggcgaatctaacccggttaccggcaacacctgtgacaacgtgaaacctcgcgctgccctgatcgattgcctggctccggatcgtcgcgtagagatcgaagttaaaggcgttaaagacgtggtaactcagccgcaggcttaatgagcttcgaaacagattaaatcagaacgcagaagcggtctgataaaacagtttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatggaagatcttccaacatcacaggtaaacagaaacgtcgggtcgatcgggaaattctttcccggacggcgcggggttgggcaagccgcaggcgcgtcagtgcttttagcgggtgtcggggcagccctgaaccagtcacgggatcgatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttct

SEQ ID NO: 136 - protein, 364 aa, Synthetic construct
MKKTAIAIAVALAGFATVAQAAPKDNTWYAGAKLGWSQYHDTGFIHNDGPTHENQLGAGAFGGYQVNPYVGFEMGYDWLGRMPYKGDNINGAYKAQGVQLTAKLGYPITDDLDVYTRLGGMVWRADTKSNVPGACEHWSYGLRPGCGGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 137 - protein, 614 aa, Synthetic construct
MKKTAIAIAVALAGFATVAQAAPKDNTWYAGAKLGWSQYHDTGFIHNDGPTHENQLGAGAFGGYQVNPYVGFEMGYDWLGRMPYKGDNINGAYKAQGVQLTAKLGYPITDDLDVYTRLGGMVWRADTKSNVPGQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGAAAGGGGSHHHHHHGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 138 - DNA, 7104 bp, Synthetic construct
  source 1..7104: mol_type = other DNA
gactcttcgcgatgtacgggccagatatacgcgttaactgcagtctagattatgcgaaaggccatcctgacggatggcctttttgtttaaacggatccgcgacattgattattgactagttattaatagtaatcaattacggggtcattaggggactttccggggactttcctccccacgcgggggactttccgccacgggcggggactttccggggactttccgttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccggggcatgcaaccagctgtggaatgtgtgtcagttagggtgtggaaagtccccaggctccccagcaggcagaagtatgcaaagcatgtggggatgcggtgggctctatggcttctactgggcggttttatggacagcaagcgaaccggaattgccagctggggcgccctctggtaaggttgggaagccctgcaaagtaaactggatggctttctcgccgccaaggatctgtcgacccctagatttcagtgcaatttatctcttcaaatgtagcacctgaagtcagccccatacgatataagttgttggaagatctagcccgcctaatgagcgggcttttttttaattcgcaattccccgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacgaaactcgctcgggctggccccggtgcattttttaaatactcgcgagaaatagagttgatcgtcaaaaccaacattgcgaccgacggtggcgataggcatccgggtagtgctcaaaagcagcttcgcctgactaatgcgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaacccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccaccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattctgagaacaaactaagtggataaatttcgtgttcaggggccaacgaagctccagggcgaagtcacaatttccggcgctaaaaatgctgctctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaataagaattcaggaaaaaaacgctgtgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtagtctagctgcacgataccgtcgacttgtacatagactcgctccgaaattaaagaacacttaaattatctactaaaggaatctttagtcaagtttatttaagatgacttaactatgaatacacaattgatgggtgagcgtaggaaaaaaaaaccccgcccctgacagggcggggttttttttgatcattctgaaatgagctgttgacaattaatcatccggctcgtataatgtgtggaattgtgagcggataacaatttcacacaggaaacagaccctaggagtactccgcggcccgggcccctgcaggttcgaaacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatggaagatcttccaacatcacaggtaaacagaaacgtcgggtcgatcgggaaattctttcccggacggcgcggggttgggcaagccgcaggcgcgtcagtgcttttagcgggtgtcggggcagccctgaaccagtcacgggatcgatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttct

SEQ ID NO: 139 - DNA, 7210 bp, Synthetic construct
  source 1..7210: mol_type = other DNA
gactcttcgcgatgtacgggccagatatacgcgttaactgcagtctagattatgcgaaaggccatcctgacggatggcctttttgtttaaacggatccgcgacattgattattgactagttattaatagtaatcaattacggggtcattaggggactttccggggactttcctccccacgcgggggactttccgccacgggcggggactttccggggactttccgttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccggggcatgcaaccagctgtggaatgtgtgtcagttagggtgtggaaagtccccaggctccccagcaggcagaagtatgcaaagcatgtggggatgcggtgggctctatggcttctactgggcggttttatggacagcaagcgaaccggaattgccagctggggcgccctctggtaaggttgggaagccctgcaaagtaaactggatggctttctcgccgccaaggatctgtcgacccctagatttcagtgcaatttatctcttcaaatgtagcacctgaagtcagccccatacgatataagttgttggaagatctagcccgcctaatgagcgggcttttttttaattcgcaattccccgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacgaaactcgctcgggctggccccggtgcattttttaaatactcgcgagaaatagagttgatcgtcaaaaccaacattgcgaccgacggtggcgataggcatccgggtagtgctcaaaagcagcttcgcctgactaatgcgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaacccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccaccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattctgagaacaaactaagtggataaatttcgtgttcaggggccaacgaagctccagggcgaagtcacaatttccggcgctaaaaatgctgctctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaataagaattcaggaaaaaaacgctgtgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtagtctagctgcacgataccgtcgacttgtacatagactcgctccgaaattaaagaacacttaaattatctactaaaggaatctttagtcaagtttatttaagatgacttaactatgaatacacaattgatgggtgagcgtaggaaaaaaaaaccccgcccctgacagggcggggttttttttgatcattctgaaatgagctgttgacaattaatcatccggctcgtataatgtgtggaattgtgagcggataacaatttcacacaggaaacagaccatgaaaaaacaacatttccgtgtcgcccttattcccttttttgcggcattttgccttcctgtttttgctcacccagaaacgctggtgaaagtaaaagatgctgaacctaggagtactccgcggcccgggcccctgcaggttcgaaacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatggaagatcttccaacatcacaggtaaacagaaacgtcgggtcgatcgggaaattctttcccggacggcgcggggttgggcaagccgcaggcgcgtcagtgcttttagcgggtgtcggggcagccctgaaccagtcacgggatcgatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttct

SEQ ID NO: 140 - DNA, 1053 bp, Synthetic construct
  source 1..1053: mol_type = other DNA
ttaagcctgcggctgagttaccacgtctttaacgcctttaacttcgatctctacgcgacgatccggagccaggcaatcgatcagggcagcgcgaggtttcacgttgtcacaggtgttgccggtaaccgggttagattcgcccataccacgtgcggagattttgtcagacggaatacctttggagatcaggtaatcaacaacagactgagcacgtttctcggacagaccctggttgtaagcgtcagaaccgatacggtcagtgaagcccagaacgacaacggaaccgtctttcggatccaggttgctcagctggctgtacagctgatccagagcctgctggccttccggcttcagggtagatttgttgaagttgaacagtacgtcagacttcagagtgaagtgcttggtctgtacttccggagccggagctggtgccggagctactaccggagcagcttcttgctggccgaaacggtaggaaacacctacgctcagcaggccgttgtccggacgggtgccgatggtgttggcatcaccgatgttgttagtccactggtattccagacgggttgcgatttcaggggtgatagcatactcgataccgcccgcgaataccggggaaacgccggtgtcgtggtctttagtagacgggccgccagggacgttagacttggtgtctgcacgccataccataccacccagacgggtataaacgtccagatcgtcagtgattggataacccagtttagcggtcaactgaacgccctgagctttataagcgccattgatgttgtcgcctttgtacggcatacggcctaaccagtcgtagcccatttcaaagccaacatacgggttaacctggtaaccaccaaaagcacctgcgcccagttggttttcatgagtcgggccatcattgtgaatgaagccggtgtcatggtactgagaccagcccagtttagcaccagcgtaccaggtgttatctttcggagcggcctgcgctacggtagcgaaaccagccagtgccactgcaatcgcgatagctgtctttttcat

SEQ ID NO: 141 - DNA, 27 bp, Synthetic construct
  source 1..27: mol_type = other DNA
gcagaaacccatacgaccgtcctggca

SEQ ID NO: 142 - DNA, 1104 bp, Synthetic construct
  source 1..1104: mol_type = other DNA
gtgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcg

SEQ ID NO: 143 - DNA, 1107 bp, Synthetic construct
  source 1..1107: mol_type = other DNA
atgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcctag

SEQ ID NO: 144 - protein, 368 aa, Synthetic construct
MKNVGFIGWRGMVGSVLMQRMVEERDFDAIRPVFFSTSQFGQAAPTFGDTSTGTLQDAFDLDALKALDIIVTCQGGDYTNEIYPKLRESGWQGYWIDAASTLRMKDDAIIILDPVNQDVITDGLNNGVKTFVGGNCTVSLMLMSLGGLFAHNLVDWVSVATYQAASGGGARHMRELLTQMGQLYGHVADELATPSSAILDIERKVTALTRSGELPVDNFGVPLAGSLIPWIDKQLDNGQSREEWKGQAETNKILNTASVIPVDGLCVRVGALRCHSQAFTIKLKKEVSIPTVEELLAAHNPWAKVVPNDRDITMRELTPAAVTGTLTTPVGRLRKLNMGPEFLSAFTVGDQLLWGAAEPLRRMLRQLA

SEQ ID NO: 145 - DNA, 1104 bp, Synthetic construct
  source 1..1104: mol_type = other DNA
cgccaactggcgcagcattcgacgcagcggctcggcggcgccccataacaactggtcgcctacggtaaacgccgacaagaactctggccccatgttcagcttacgcagacgaccaaccggcgtagtcaacgtgccggtcaccgccgccggggttaattcgcgcatagtgatatcacgatcgttcggcaccactttcgcccacggattatgtgccgccagcagttcttccaccgtcggaatggatacctcttttttcagcttgatggtgaacgcctggctgtgacagcgcagcgcgccgacgcgcacacacaaaccatcaaccggaatcacagaggcagtattgagaatcttgttggtttccgcctggcctttccactcttcgcggctctggccgttatcgagctgtttgtcgatccaggggatcaggcttcccgccagcggtacgccaaagttatcaaccggcagctcgccgctgcgggtcaatgccgtaactttgcgttcaatatcaagaattgcggaagacggcgtcgccagttcatcggcgacatggccatacaactgacccatctgggttaacagctcgcgcatatggcgcgcgccgccgccggaggcggcctgataggtcgcgacggatacccagtcaacgagattatgggcaaagagaccgcccagcgacatcaacatcaggctaacggtacagttaccgcccacaaaggtcttcacgccattgttcaggccgtcggtaatcacgtcctggttgaccgggtcgagaataataatggcatcatctttcatgcgcagcgtagaagccgcatcaatccagtaaccctgccatccgctttcgcgcagctttggataaatttcgttggtataatcgccgccctggcaggtcacgatgatatcgagcgcttttagcgcatccagatcaaaagcgtcctgtagcgtgccggtggaggtgtcgccgaaggtgggcgccgcctgtccaaactgggaggtagaaaagaaaacagggcgaatagcgtcgaaatcgcgctcctctaccatgcgttgcatgagaacagagccgaccattccgcgccagccgataaaaccaacatttttcac

SEQ ID NO: 146 - DNA, 1257 bp, Synthetic construct
  source 1..1257: mol_type = other DNA
ttcgcctttcacacgctcaatatttgcacctaaagcgcgcagtttgtcttcaatgcgttcgtagccacgatcgatgtgataaatacgatcaaccaccgtcgtcccttccgcaatacagccagccagcaccaggcttgctgatgcacgcagatcggttgccataacctgtgcgccagaaagtttttcaacaccgtgacaaataacggtattgctttcgatttcggcgtgcgcgcccatacggctcagctctggcacatgcataaagcggttttcaaagaccgtttcggtgataaacccggtcccttctgccaccaggttcaacagcgtgaactgggcctgcatatcggtcgggaatgccggatgcggcgcggtacgtacgttaacagccttcggacgtttgccatgcatatccaggctaatccagtcttcgccgacttcgatgtccgctccagcgtcacgcagtttcgccagcacggcgtcgagagtatctggctgcgcgttacggcagataattttgccgcgagaaatcgccgccgccaccaggaaagtaccggtttcgatacgatccggcagaacgcgatagacaccgccgcctaaacgttccacaccttcgatgacgatacgatcggtgccctgaccgctaattttcgcacccagcgtaatcaggaagttcgcggtatcgacgatttccggttcacgcgctgcgttttcaataatcgtggtgccttccgccagggttgcagcacacatgatggtcaccgttgcgccaacgctgactttatccatcacgatatgtgcacctttcaaacgaccatcgacggaagctttaacgtaaccttcttccagtttgatggtcgcgcctaattgttcgaggccagaaatgtgtagatcaaccggacgcgcaccgatcgtacaaccgccaggtagtgaaacttgcccctgaccaaagcgcgctaccagcggccccagcgcccagatagaagcacgcatggttttaaccagatcgtaaggtgcgcagaatacattaacgtcgcgggcatcaatatgcacagaaccattacgttctactttcgcacccagctggcttagcagcttcattgatgtatcgacgtctttcagtttcgggacgttctggatctctaccggttcttccgccagtagtgcggcaaaaaggataggcagagcagcatttttagcgccggaaattgtgacttcgccctggagcttcgttggcccctgaacacgaaatttatccac

SEQ ID NO: 147 - DNA, 876 bp, Synthetic construct
  source 1..876: mol_type = other DNA
atggctgaagcgcaaaatgatcccctgctgccgggatactcgtttaacgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgcattagtcaggcgaagctgcttttgagcactacccggatgcctatcgccaccgtcggtcgcaatgttggttttgacgatcaactctatttctcgcgagtatttaaaaaatgcaccggggccagcccgagcgagtttcgtgccggttgtgaagaaaaagtgaatgatgtagccgtcaagttgtca

SEQ ID NO: 148 - DNA, 105 bp, Synthetic construct
  source 1..105: mol_type = other DNA
ttcagcatcttttactttcaccagcgtttctgggtgagcaaaaacaggaaggcaaaatgccgcaaaaaagggaataagggcgacacggaaatgttgttttttcat

SEQ ID NO: 149 - DNA, 4056 bp, Synthetic construct
  source 1..4056: mol_type = other DNA
gcacatctctttgcaggaaaaaaacgctatgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcctagtctagactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgctaaaagcactgacgcgcctgcggcttgcccaaccccgcgccgtccgggaaagaatttcccgatcgacccgacgtttctgtttacctgtgatgttggaagatcttccattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgagctcattaagcctgcggctgagttaccacgtctttaacgcctttaacttcgatctctacgcgacgatccggagccaggcaatcgatcagggcagcgcgaggtttcacgttgtcacaggtgttgccggtaaccgggttagattcgcccataccacgtgcggagattttgtcagacggaatacctttggagatcaggtaatcaacaacagactgagcacgtttctcggacagaccctggttgtaagcgtcagaaccgatacggtcagtgaagcccagaacgacaacggaaccgtctttcggatccaggttgctcagctggctgtacagctgatccagagcctgctggccttccggcttcagggtagatttgttgaagttgaacagtacgtcagacttcagagtgaagtgcttggtctgtacttccggagccggagctggtgccggagctactaccggagcagcttcttgctggccgaaacggtaggaaacacctacgctcagcaggccgttgtccggacgggtgccgatggtgttggcatcaccgatgttgttagtccactggtattccagacgggttgcgatttcaggggtgatagcatactcgataccgcccgcgaataccggggaaacgccggtgtcgtggtctttagtagacgggccaccgcagaaacccatacgaccgtcctggcacgcgccagggacgttagacttggtgtctgcacgccataccataccacccagacgggtataaacgtccagatcgtcagtgattggataacccagtttagcggtcaactgaacgccctgagctttataagcgccattgatgttgtcgcctttgtacggcatacggcctaaccagtcgtagcccatttcaaagccaacatacgggttaacctggtaaccaccaaaagcacctgcgcccagttggttttcatgagtcgggccatcattgtgaatgaagccggtgtcatggtactgagaccagcccagtttagcaccagcgtaccaggtgttatctttcggagcggcctgcgctacggtagcgaaaccagccagtgccactgcaatcgcgatagctgtctttttcatggtctgtttcctgtgtgaaattgttatccgctcacaattccacacattatacgagccggatgattaattgtcaacagctcatttcagaatggaaggtcttccggaagatcc

SEQ ID NO: 150 - DNA, 8160 bp, Synthetic construct
  source 1..8160: mol_type = other DNA
agaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttagcacgtgctattattgaagcatttatcagggttattgtctcatgagcggatacatatttgaatgtatttagaaaaataaacaaataggggttccgcgcacatttccccgaaaagtgccacctgtatgcggtgtgaaataccgcacagatcgatcccgtgactggttcagggctgccccgacacccgctaaaagcactgacgcgcctgcggcttgcccaaccccgcgccgtccgggaaagaatttcccgatcgacccgacgtttctgtttacctgtgatgttggaagatcttccattattgaagcatttatcagggttattgtctcatgagcggatacatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaactgttttatcagaccgcttctgcgttctgatttaatctgtttcgaagctcattaagcctgcggctgagttaccacgtctttaacgcctttaacttcgatctctacgcgacgatccggagccaggcaatcgatcagggcagcgcgaggtttcacgttgtcacaggtgttgccggtaaccgggttagattcgcccataccacgtgcggagattttgtcagacggaatacctttggagatcaggtaatcaacaacagactgagcacgtttctcggacagaccctggttgtaagcgtcagaaccgatacggtcagtgaagcccagaacgacaacggaaccgtctttcggatccaggttgctcagctggctgtacagctgatccagagcctgctggccttccggcttcagggtagatttgttgaagttgaacagtacgtcagacttcagagtgaagtgcttggtctgtacttccggagccggagctggtgccggagctactaccggagcagcttcttgctggccgaaacggtaggaaacacctacgctcagcaggccgttgtccggacgggtgccgatggtgttggcatcaccgatgttgttagtccactggtattccagacgggttgcgatttcaggggtgatagcatactcgataccgcccgcgaataccggggaaacgccggtgtcgtggtctttagtagacgggccaccgcagaaacccatacgaccgtcctggcacgcgccagggacgttagacttggtgtctgcacgccataccataccacccagacgggtataaacgtccagatcgtcagtgattggataacccagtttagcggtcaactgaacgccctgagctttataagcgccattgatgttgtcgcctttgtacggcatacggcctaaccagtcgtagcccatttcaaagccaacatacgggttaacctggtaaccaccaaaagcacctgcgcccagttggttttcatgagtcgggccatcattgtgaatgaagccggtgtcatggtactgagaccagcccagtttagcaccagcgtaccaggtgttatctttcggagcggcctgcgctacggtagcgaaaccagccagtgccactgcaatcgcgatagctgtctttttcatggtctgtttcctgtgtgaaattgttatccgctcacaattccacacattatacgagccggatgattaattgtcaacagctcatttcagaatgatcaaaaaaaaccccgccctgtcaggggcggggtttttttttcctacgctcacccatcaattgtgtattcatagttaagtcatcttaaataaacttgactaaagattcctttagtagataatttaagtgttctttaatttcggagcgagtctatgtacaagtcgacggtatcgtgcagctagactacgccaactggcgcagcattcgacgcagcggctcggcggcgccccataacaactggtcgcctacggtaaacgccgacaagaactctggccccatgttcagcttacgcagacgaccaaccggcgtagtcaacgtgccggtcaccgccgccggggttaattcgcgcatagtgatatcacgatcgttcggcaccactttcgcccacggattatgtgccgccagcagttcttccaccgtcggaatggatacctcttttttcagcttgatggtgaacgcctggctgtgacagcgcagcgcgccgacgcgcacacacaaaccatcaaccggaatcacagaggcagtattgagaatcttgttggtttccgcctggcctttccactcttcgcggctctggccgttatcgagctgtttgtcgatccaggggatcaggcttcccgccagcggtacgccaaagttatcaaccggcagctcgccgctgcgggtcaatgccgtaactttgcgttcaatatcaagaattgcggaagacggcgtcgccagttcatcggcgacatggccatacaactgacccatctgggttaacagctcgcgcatatggcgcgcgccgccgccggaggcggcctgataggtcgcgacggatacccagtcaacgagattatgggcaaagagaccgcccagcgacatcaacatcaggctaacggtacagttaccgcccacaaaggtcttcacgccattgttcaggccgtcggtaatcacgtcctggttgaccgggtcgagaataataatggcatcatctttcatgcgcagcgtagaagccgcatcaatccagtaaccctgccatccgctttcgcgcagctttggataaatttcgttggtataatcgccgccctggcaggtcacgatgatatcgagcgcttttagcgcatccagatcaaaagcgtcctgtagcgtgccggtggaggtgtcgccgaaggtgggcgccgcctgtccaaactgggaggtagaaaagaaaacagggcgaatagcgtcgaaatcgcgctcctctaccatgcgttgcatgagaacagagccgaccattccgcgccagccgataaaaccaacatttttcacagcgtttttttcctgaattcttattcgcctttcacacgctcaatatttgcacctaaagcgcgcagtttgtcttcaatgcgttcgtagccacgatcgatgtgataaatacgatcaaccaccgtcgtcccttccgcaatacagccagccagcaccaggcttgctgatgcacgcagatcggttgccataacctgtgcgccagaaagtttttcaacaccgtgacaaataacggtattgctttcgatttcggcgtgcgcgcccatacggctcagctctggcacatgcataaagcggttttcaaagaccgtttcggtgataaacccggtcccttctgccaccaggttcaacagcgtgaactgggcctgcatatcggtcgggaatgccggatgcggcgcggtacgtacgttaacagccttcggacgtttgccatgcatatccaggctaatccagtcttcgccgacttcgatgtccgctccagcgtcacgcagtttcgccagcacggcgtcgagagtatctggctgcgcgttacggcagataattttgccgcgagaaatcgccgccgccaccaggaaagtaccggtttcgatacgatccggcagaacgcgatagacaccgccgcctaaacgttccacaccttcgatgacgatacgatcggtgccctgaccgctaattttcgcacccagcgtaatcaggaagttcgcggtatcgacgatttccggttcacgcgctgcgttttcaataatcgtggtgccttccgccagggttgcagcacacatgatggtcaccgttgcgccaacgctgactttatccatcacgatatgtgcacctttcaaacgaccatcgacggaagctttaacgtaaccttcttccagtttgatggtcgcgcctaattgttcgaggccagaaatgtgtagatcaaccggacgcgcaccgatcgtacaaccgccaggtagtgaaacttgcccctgaccaaagcgcgctaccagcggccccagcgcccagatagaagcacgcatggttttaaccagatcgtaaggtgcgcagaatacattaacgtcgcgggcatcaatatgcacagaaccattacgttctactttcgcacccagctggcttagcagcttcattgatgtatcgacgtctttcagtttcgggacgttctggatctctaccggttcttccgccagtagtgcggcaaaaaggataggcagagcagcatttttagcgccggaaattgtgacttcgccctggagcttcgttggcccctgaacacgaaatttatccacttagtttgttctcagaattcgctagcccaaaaaaacgggtatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggttgggttagcgagaagagccagtaaaagacgcagtgacggcaatgtctgatgcaatatggacaattggtttcttctctgaatggtgggagtatgaaaagtatggctgaagcgcaaaatgatcccctgctgccgggatactcgtttaacgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgcattagtcaggcgaagctgcttttgagcactacccggatgcctatcgccaccgtcggtcgcaatgttggttttgacgatcaactctatttctcgcgagtatttaaaaaatgcaccggggccagcccgagcgagtttcgtgccggttgtgaagaaaaagtgaatgatgtagccgtcaagttgtcataattggtaacgaatcagacaattgacggcttgacggagtagcatagggtttgcagaatccctgcttcgtccatttgacaggcacattatgcatcggggaattgcgaattaaaaaaaagcccgctcattaggcgggctagatcttccaacaacttatatcgtatggggctgacttcaggtgctacatttgaagagataaattgcactgaaatctaggggtcgacagatccttggcggcgagaaagccatccagtttactttgcagggcttcccaaccttaccagagggcgccccagctggcaattccggttcgcttgctgtccataaaaccgcccagtagaagccatagagcccaccgcatccccacatgctttgcatacttctgcctgctggggagcctggggactttccacaccctaactgacacacattccacagctggttgcatgccccggatcgatccttatcggattttaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcattctcgagcggccgccactgtgctggatatctgcagaattccaccacactggactagtggatccgagctcggtaccaagcttaagtttaaacgctagccagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaacggaaagtccccggaaagtccccgcccgtggcggaaagtcccccgcgtggggaggaaagtccccggaaagtcccctaatgaccccgtaattgattactattaataactagtcaataatcaatgtcgcggatccgtttaaacaaaaaggccatccgtcaggatggcctttcgcataatctagactgcagttaacgcgtatatctggcccgtacatcgcgaagagtc

SEQ ID NO: 151 - DNA, 1095 bp, Synthetic construct
  source 1..1095: mol_type = other DNA
atgaaaaagacagctatcgcgattgcagtggcactggctggtttcgctaccgtagcgcaggccgctccgaaagataacacctggtacgctggtgctaaactgggctggtctcagtaccatgacaccggcttcattcacaatgatggcccgactcatgaaaaccaactgggcgcaggtgcttttggtggttaccaggttaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcgcgtgcgaacattggagctatggcctgcgtccgggctgcggtggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggcctgctgagcgtaggtgtttcctaccgtttcggccagcaagaagctgctccggtagtagctccggcaccagctccggctccggaagtacagaccaagcacttcactctgaagtctgacgtactgttcaacttcaacaaatctaccctgaagccggaaggccagcaggctctggatcagctgtacagccagctgagcaacctggatccgaaagacggttccgttgtcgttctgggcttcactgaccgtatcggttctgacgcttacaaccagggtctgtccgagaaacgtgctcagtctgttgttgattacctgatctccaaaggtattccgtctgacaaaatctccgcacgtggtatgggcgaatctaacccggttaccggcaacacctgtgacaacgtgaaacctcgcgctgccctgatcgattgcctggctccggatcgtcgcgtagagatcgaagttaaaggcgttaaagacgtggtaactcagccgcaggcttaa

SEQ ID NO: 152 - DNA, 7104 bp, Synthetic construct
  source 1..7104: mol_type = other DNA
agaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttagcacgtgctattattgaagcatttatcagggttattgtctcatgagcggatacatatttgaatgtatttagaaaaataaacaaataggggttccgcgcacatttccccgaaaagtgccacctgtatgcggtgtgaaataccgcacagatcgatcccgtgactggttcagggctgccccgacacccgctaaaagcactgacgcgcctgcggcttgcccaaccccgcgccgtccgggaaagaatttcccgatcgacccgacgtttctgtttacctgtgatgttggaagatcttccattattgaagcatttatcagggttattgtctcatgagcggatacatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtttcgaacctgcaggggcccgggccgcggagtactcctagggtctgtttcctgtgtgaaattgttatccgctcacaattccacacattatacgagccggatgattaattgtcaacagctcatttcagaatgatcaaaaaaaaccccgccctgtcaggggcggggtttttttttcctacgctcacccatcaattgtgtattcatagttaagtcatcttaaataaacttgactaaagattcctttagtagataatttaagtgttctttaatttcggagcgagtctatgtacaagtcgacggtatcgtgcagctagactacgccaactggcgcagcattcgacgcagcggctcggcggcgccccataacaactggtcgcctacggtaaacgccgacaagaactctggccccatgttcagcttacgcagacgaccaaccggcgtagtcaacgtgccggtcaccgccgccggggttaattcgcgcatagtgatatcacgatcgttcggcaccactttcgcccacggattatgtgccgccagcagttcttccaccgtcggaatggatacctcttttttcagcttgatggtgaacgcctggctgtgacagcgcagcgcgccgacgcgcacacacaaaccatcaaccggaatcacagaggcagtattgagaatcttgttggtttccgcctggcctttccactcttcgcggctctggccgttatcgagctgtttgtcgatccaggggatcaggcttcccgccagcggtacgccaaagttatcaaccggcagctcgccgctgcgggtcaatgccgtaactttgcgttcaatatcaagaattgcggaagacggcgtcgccagttcatcggcgacatggccatacaactgacccatctgggttaacagctcgcgcatatggcgcgcgccgccgccggaggcggcctgataggtcgcgacggatacccagtcaacgagattatgggcaaagagaccgcccagcgacatcaacatcaggctaacggtacagttaccgcccacaaaggtcttcacgccattgttcaggccgtcggtaatcacgtcctggttgaccgggtcgagaataataatggcatcatctttcatgcgcagcgtagaagccgcatcaatccagtaaccctgccatccgctttcgcgcagctttggataaatttcgttggtataatcgccgccctggcaggtcacgatgatatcgagcgcttttagcgcatccagatcaaaagcgtcctgtagcgtgccggtggaggtgtcgccgaaggtgggcgccgcctgtccaaactgggaggtagaaaagaaaacagggcgaatagcgtcgaaatcgcgctcctctaccatgcgttgcatgagaacagagccgaccattccgcgccagccgataaaaccaacatttttcacagcgtttttttcctgaattcttattcgcctttcacacgctcaatatttgcacctaaagcgcgcagtttgtcttcaatgcgttcgtagccacgatcgatgtgataaatacgatcaaccaccgtcgtcccttccgcaatacagccagccagcaccaggcttgctgatgcacgcagatcggttgccataacctgtgcgccagaaagtttttcaacaccgtgacaaataacggtattgctttcgatttcggcgtgcgcgcccatacggctcagctctggcacatgcataaagcggttttcaaagaccgtttcggtgataaacccggtcccttctgccaccaggttcaacagcgtgaactgggcctgcatatcggtcgggaatgccggatgcggcgcggtacgtacgttaacagccttcggacgtttgccatgcatatccaggctaatccagtcttcgccgacttcgatgtccgctccagcgtcacgcagtttcgccagcacggcgtcgagagtatctggctgcgcgttacggcagataattttgccgcgagaaatcgccgccgccaccaggaaagtaccggtttcgatacgatccggcagaacgcgatagacaccgccgcctaaacgttccacaccttcgatgacgatacgatcggtgccctgaccgctaattttcgcacccagcgtaatcaggaagttcgcggtatcgacgatttccggttcacgcgctgcgttttcaataatcgtggtgccttccgccagggttgcagcacacatgatggtcaccgttgcgccaacgctgactttatccatcacgatatgtgcacctttcaaacgaccatcgacggaagctttaacgtaaccttcttccagtttgatggtcgcgcctaattgttcgaggccagaaatgtgtagatcaaccggacgcgcaccgatcgtacaaccgccaggtagtgaaacttgcccctgaccaaagcgcgctaccagcggccccagcgcccagatagaagcacgcatggttttaaccagatcgtaaggtgcgcagaatacattaacgtcgcgggcatcaatatgcacagaaccattacgttctactttcgcacccagctggcttagcagcttcattgatgtatcgacgtctttcagtttcgggacgttctggatctctaccggttcttccgccagtagtgcggcaaaaaggataggcagagcagcatttttagcgccggaaattgtgacttcgccctggagcttcgttggcccctgaacacgaaatttatccacttagtttgttctcagaattcgctagcccaaaaaaacgggtatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggttgggttagcgagaagagccagtaaaagacgcagtgacggcaatgtctgatgcaatatggacaattggtttcttctctgaatggtgggagtatgaaaagtatggctgaagcgcaaaatgatcccctgctgccgggatactcgtttaacgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgcattagtcaggcgaagctgcttttgagcactacccggatgcctatcgccaccgtcggtcgcaatgttggttttgacgatcaactctatttctcgcgagtatttaaaaaatgcaccggggccagcccgagcgagtttcgtgccggttgtgaagaaaaagtgaatgatgtagccgtcaagttgtcataattggtaacgaatcagacaattgacggcttgacggagtagcatagggtttgcagaatccctgcttcgtccatttgacaggcacattatgcatcggggaattgcgaattaaaaaaaagcccgctcattaggcgggctagatcttccaacaacttatatcgtatggggctgacttcaggtgctacatttgaagagataaattgcactgaaatctaggggtcgacagatccttggcggcgagaaagccatccagtttactttgcagggcttcccaaccttaccagagggcgccccagctggcaattccggttcgcttgctgtccataaaaccgcccagtagaagccatagagcccaccgcatccccacatgctttgcatacttctgcctgctggggagcctggggactttccacaccctaactgacacacattccacagctggttgcatgccccggatcgatccttatcggattttaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcattctcgagcggccgccactgtgctggatatctgcagaattccaccacactggactagtggatccgagctcggtaccaagcttaagtttaaacgctagccagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaacggaaagtccccggaaagtccccgcccgtggcggaaagtcccccgcgtggggaggaaagtccccggaaagtcccctaatgaccccgtaattgattactattaataactagtcaataatcaatgtcgcggatccgtttaaacaaaaaggccatccgtcaggatggcctttcgcataatctagactgcagttaacgcgtatatctggcccgtacatcgcgaagagtc

SEQ ID NO: 153 - DNA, 7210 bp, Synthetic construct
  source 1..7210: mol_type = other DNA
agaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttagcacgtgctattattgaagcatttatcagggttattgtctcatgagcggatacatatttgaatgtatttagaaaaataaacaaataggggttccgcgcacatttccccgaaaagtgccacctgtatgcggtgtgaaataccgcacagatcgatcccgtgactggttcagggctgccccgacacccgctaaaagcactgacgcgcctgcggcttgcccaaccccgcgccgtccgggaaagaatttcccgatcgacccgacgtttctgtttacctgtgatgttggaagatcttccattattgaagcatttatcagggttattgtctcatgagcggatacatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtttcgaacctgcaggggcccgggccgcggagtactcctaggttcagcatcttttactttcaccagcgtttctgggtgagcaaaaacaggaaggcaaaatgccgcaaaaaagggaataagggcgacacggaaatgttgttttttcatggtctgtttcctgtgtgaaattgttatccgctcacaattccacacattatacgagccggatgattaattgtcaacagctcatttcagaatgatcaaaaaaaaccccgccctgtcaggggcggggtttttttttcctacgctcacccatcaattgtgtattcatagttaagtcatcttaaataaacttgactaaagattcctttagtagataatttaagtgttctttaatttcggagcgagtctatgtacaagtcgacggtatcgtgcagctagactacgccaactggcgcagcattcgacgcagcggctcggcggcgccccataacaactggtcgcctacggtaaacgccgacaagaactctggccccatgttcagcttacgcagacgaccaaccggcgtagtcaacgtgccggtcaccgccgccggggttaattcgcgcatagtgatatcacgatcgttcggcaccactttcgcccacggattatgtgccgccagcagttcttccaccgtcggaatggatacctcttttttcagcttgatggtgaacgcctggctgtgacagcgcagcgcgccgacgcgcacacacaaaccatcaaccggaatcacagaggcagtattgagaatcttgttggtttccgcctggcctttccactcttcgcggctctggccgttatcgagctgtttgtcgatccaggggatcaggcttcccgccagcggtacgccaaagttatcaaccggcagctcgccgctgcgggtcaatgccgtaactttgcgttcaatatcaagaattgcggaagacggcgtcgccagttcatcggcgacatggccatacaactgacccatctgggttaacagctcgcgcatatggcgcgcgccgccgccggaggcggcctgataggtcgcgacggatacccagtcaacgagattatgggcaaagagaccgcccagcgacatcaacatcaggctaacggtacagttaccgcccacaaaggtcttcacgccattgttcaggccgtcggtaatcacgtcctggttgaccgggtcgagaataataatggcatcatctttcatgcgcagcgtagaagccgcatcaatccagtaaccctgccatccgctttcgcgcagctttggataaatttcgttggtataatcgccgccctggcaggtcacgatgatatcgagcgcttttagcgcatccagatcaaaagcgtcctgtagcgtgccggtggaggtgtcgccgaaggtgggcgccgcctgtccaaactgggaggtagaaaagaaaacagggcgaatagcgtcgaaatcgcgctcctctaccatgcgttgcatgagaacagagccgaccattccgcgccagccgataaaaccaacatttttcacagcgtttttttcctgaattcttattcgcctttcacacgctcaatatttgcacctaaagcgcgcagtttgtcttcaatgcgttcgtagccacgatcgatgtgataaatacgatcaaccaccgtcgtcccttccgcaatacagccagccagcaccaggcttgctgatgcacgcagatcggttgccataacctgtgcgccagaaagtttttcaacaccgtgacaaataacggtattgctttcgatttcggcgtgcgcgcccatacggctcagctctggcacatgcataaagcggttttcaaagaccgtttcggtgataaacccggtcccttctgccaccaggttcaacagcgtgaactgggcctgcatatcggtcgggaatgccggatgcggcgcggtacgtacgttaacagccttcggacgtttgccatgcatatccaggctaatccagtcttcgccgacttcgatgtccgctccagcgtcacgcagtttcgccagcacggcgtcgagagtatctggctgcgcgttacggcagataattttgccgcgagaaatcgccgccgccaccaggaaagtaccggtttcgatacgatccggcagaacgcgatagacaccgccgcctaaacgttccacaccttcgatgacgatacgatcggtgccctgaccgctaattttcgcacccagcgtaatcaggaagttcgcggtatcgacgatttccggttcacgcgctgcgttttcaataatcgtggtgccttccgccagggttgcagcacacatgatggtcaccgttgcgccaacgctgactttatccatcacgatatgtgcacctttcaaacgaccatcgacggaagctttaacgtaaccttcttccagtttgatggtcgcgcctaattgttcgaggccagaaatgtgtagatcaaccggacgcgcaccgatcgtacaaccgccaggtagtgaaacttgcccctgaccaaagcgcgctaccagcggccccagcgcccagatagaagcacgcatggttttaaccagatcgtaaggtgcgcagaatacattaacgtcgcgggcatcaatatgcacagaaccattacgttctactttcgcacccagctggcttagcagcttcattgatgtatcgacgtctttcagtttcgggacgttctggatctctaccggttcttccgccagtagtgcggcaaaaaggataggcagagcagcatttttagcgccggaaattgtgacttcgccctggagcttcgttggcccctgaacacgaaatttatccacttagtttgttctcagaattcgctagcccaaaaaaacgggtatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggttgggttagcgagaagagccagtaaaagacgcagtgacggcaatgtctgatgcaatatggacaattggtttcttctctgaatggtgggagtatgaaaagtatggctgaagcgcaaaatgatcccctgctgccgggatactcgtttaacgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgcattagtcaggcgaagctgcttttgagcactacccggatgcctatcgccaccgtcggtcgcaatgttggttttgacgatcaactctatttctcgcgagtatttaaaaaatgcaccggggccagcccgagcgagtttcgtgccggttgtgaagaaaaagtgaatgatgtagccgtcaagttgtcataattggtaacgaatcagacaattgacggcttgacggagtagcatagggtttgcagaatccctgcttcgtccatttgacaggcacattatgcatcggggaattgcgaattaaaaaaaagcccgctcattaggcgggctagatcttccaacaacttatatcgtatggggctgacttcaggtgctacatttgaagagataaattgcactgaaatctaggggtcgacagatccttggcggcgagaaagccatccagtttactttgcagggcttcccaaccttaccagagggcgccccagctggcaattccggttcgcttgctgtccataaaaccgcccagtagaagccatagagcccaccgcatccccacatgctttgcatacttctgcctgctggggagcctggggactttccacaccctaactgacacacattccacagctggttgcatgccccggatcgatccttatcggattttaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcattctcgagcggccgccactgtgctggatatctgcagaattccaccacactggactagtggatccgagctcggtaccaagcttaagtttaaacgctagccagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaacggaaagtccccggaaagtccccgcccgtggcggaaagtcccccgcgtggggaggaaagtccccggaaagtcccctaatgaccccgtaattgattactattaataactagtcaataatcaatgtcgcggatccgtttaaacaaaaaggccatccgtcaggatggcctttcgcataatctagactgcagttaacgcgtatatctggcccgtacatcgcgaagagtc

SEQ ID NO: 154 - protein, 9 aa, Synthetic construct
CQDGRMGFC

SEQ ID NO: 155 - protein, 5 aa, Synthetic construct
PPVAT

SEQ ID NO: 156 - DNA, 1122 bp, Synthetic construct
  source 1..1122: mol_type = other DNA
atgctgtgctgtatgagaagaaccaaacaggttgaaaagaatgatgaggaccaaaagatctccgagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccgacggcatcagccatttcgcccaggagtgcttccccgagggcctgagcatcgaccgcaccgtgcgcttcgagaacgacggcaccatgaccagccaccacacctacgagctggacgacacctgcgtggtgagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgggccacttcgacagcaagatgaccttcaacggcagccgcgccatcgagatccccggcccacacttcgtgaccatcatcaccaagcagatgagggacaccagcgacaagcgcgaccacgtgtgccagcgcgaggtggcctacgcccacagcgtgccccgcatcaccagcgccatcggtagcgacgaggatggaagcggagctactaacttcagcctgctgaagcaggctggcgacgtggaggagaaccctggacctatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaa

SEQ ID NO: 157 - DNA, 1140 bp, Synthetic construct
  source 1..1140: mol_type = other DNA
atgctgtgctgtatgagaagaaccaaacaggttgaaaagaatgatgaggaccaaaagatctccgagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccgacggcatcagccatttcgcccaggagtgcttccccgagggcctgagcatcgaccgcaccgtgcgcttcgagaacgacggcaccatgaccagccaccacacctacgagctggacgacacctgcgtggtgagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgggccacttcgacagcaagatgaccttcaacggcagccgcgccatcgagatccccggcccacacttcgtgaccatcatcaccaagcagatgagggacaccagcgacaagcgcgaccacgtgtgccagcgcgaggtggcctacgcccacagcgtgccccgcatcaccagcgccatcggtagcgacgaggatggaagcggagctactaacttcagcctgctgaagcaggctggcgacgtggaggagaaccctggacctatgaacaggaaggtcacagccatagccctggctgcgatcatctgggccacagctgctcaaggcttccttatgttcaaacaggggcgctgtctttgcatcggccccgggatgaaagccgtcaaaatggcagagatcgagaaagcttctgtaatttacccgagtaacggctgcgacaaagttgaagtgattgttactatgaaggctcataaacgacaaaggtgcctggaccccagatccaagcaagctcgcctcataatgcaggcaatagaaaaaaagaattttttaaggcgtcaaaacatgtaa

SEQ ID NO: 158 - DNA, 1845 bp, Synthetic construct
  source 1..1845: mol_type = other DNA
atgaaaaagacagctatcgcgattgcagtggcactggctggtttcgctaccgtagcgcaggccgctccgaaagataacacctggtacgctggtgctaaactgggctggtctcagtaccatgacaccggcttcattcacaatgatggcccgactcatgaaaaccaactgggcgcaggtgcttttggtggttaccaggttaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggccagtctggggcagaggtgaaaaagcccggggagtctctgaagatctcctgtaagggttctggatacagctttaccagctactggatcgcctgggtgcgccagatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcagtagttccaactgcgcaaagtggcctgaatacttccagcattggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggaggaaccaagctgaccgtcctaggtgcggccgccggcggaggaggatctcatcatcaccatcaccatggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggcctgctgagcgtaggtgtttcctaccgtttcggccagcaagaagctgctccggtagtagctccggcaccagctccggctccggaagtacagaccaagcacttcactctgaagtctgacgtactgttcaacttcaacaaatctaccctgaagccggaaggccagcaggctctggatcagctgtacagccagctgagcaacctggatccgaaagacggttccgttgtcgttctgggcttcactgaccgtatcggttctgacgcttacaaccagggtctgtccgagaaacgtgctcagtctgttgttgattacctgatctccaaaggtattccgtctgacaaaatctccgcacgtggtatgggcgaatctaacccggttaccggcaacacctgtgacaacgtgaaacctcgcgctgccctgatcgattgcctggctccggatcgtcgcgtagagatcgaagttaaaggcgttaaagacgtggtaactcagccgcaggcttaa